(19) United States
(12) Patent Application Publication
(10) Pub. No.: US 2026/0226066 A1
(43) Pub. Date: Aug. 6, 2026

(54) PGDH INHIBITORS AND METHODS OF MAKING AND USING

(71) Applicant: Epirium Bio Inc., San Diego, CA (US)

(72) Inventor: Bruce FAHR, San Diego, CA (US)

(21) Appl. No.: 19/150,765

(22) PCT Filed: Jan. 24, 2024
(86) PCT No.: PCT/US2024/012807

§ 371 Date: Jul. 24, 2025

Related U.S. Application Data
(60) Provisional application No. 63/481,495, filed on Jan. 25, 2023.

(51) Int. Cl. C07D 487/04; A61K 31/437; A61K 31/444; A61K 31/4985; A61K 31/5377; C07D 471/04; C07D 519/00

(52) U.S. Cl. CPC C07D 487/04; A61K 31/437; A61K 31/444; A61K 31/4985; A61K 31/5377; C07D 471/04; C07D 519/00

(57) ABSTRACT

Disclosed herein are 15-hydroxyprostaglandin dehydrogenase (PGDH) inhibitor compounds. Such compounds may be administered to subjects that may benefit from modulation of prostaglandin levels for the treatment of muscle disorders.

### CROSS REFERENCE

This application claims the benefit of U.S. Application No. 63/481,495, filed Jan. 25, 2023, which is hereby incorporated by reference in its entirety.

### BACKGROUND OF THE INVENTION

Prostaglandins are a group of physiologically active lipid compounds with diverse biological effects including vasodilation, inhibition of platelet aggregation, bronchodilation, bronchoconstriction, immune responses, contraction and relaxation of gastrointestinal smooth muscles, gastric acid secretion, gastric mucus secretion, uterus contraction, lipolysis inhibition, neurotransmission, clotting, hyperalgesia, and pyrexia.

Treatment of diseases or disorders may require activation of prostaglandins, or inhibition of inactivation of prostaglandins. Hydroxyprostaglandin dehydrogenases, such as 15-hydroxyprostaglandin dehydrogenase (15-PGDH) are involved in the inactivation of prostaglandins. As such, diseases/disorders associated with prostaglandins can be prevented, treated and/or managed using inhibitors of hydroxyprostaglandin dehydrogenase such as inhibitors of 15-PGDH.

### SUMMARY OF THE INVENTION

In one aspect, provided herein is a compound having the structure of Formula (I), or a pharmaceutically acceptable salt thereof:

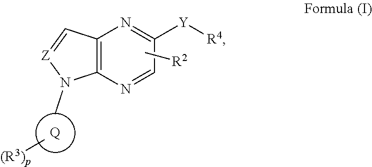

wherein,
ring Q is phenyl or 5- to 10-membered heteroaryl;
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
each R³ is independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
R⁴ is substituted or unsubstituted C₁-C₈ alkyl, substituted or unsubstituted C₂-C₈ alkenyl, substituted or unsubstituted C₁-C₈ aminoalkyl, substituted or unsubstituted C₁-C₈ heteroalkyl, substituted or unsubstituted C₁-C₈ haloalkyl, substituted or unsubstituted C₁-C₈ hydroxyalkyl, substituted or unsubstituted C₃-C₈ cycloalkyl, or substituted or unsubstituted C₃-C₈ heterocycloalkyl, each of which is substituted with one or more R⁶;
or R⁴ is

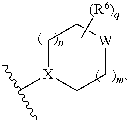

wherein
   X is CH or N;
   W is CR⁶R⁶, —NR^{6a}—, or —O—;
   each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
   or two R⁶ combine together with the atom(s) to which they are attached to form a C₃-C₆ cycloalkyl or C₃-C₆ heterocycloalkyl;
   R^{6a} is H or C₁-C₃ alkyl;
   m and n are each independently 0, 1, or 2;
   q is 0, 1, 2, 3, 4, 5, 6, 7, 8, 9, or 10;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is optionally substituted with one or more R;
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃; and
p is 1, 2, 3, or 4.

In some embodiments, ring Q is a 6-membered monocyclic heteroaryl comprising 1, 2, or 3 N atoms.

In some embodiments, ring Q is a phenyl, pyrimidine, or pyridine.

In some embodiments,

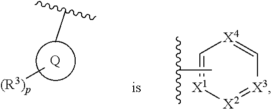

wherein,
X¹, X², X³ and X⁴ are each independently N or CR³;
each R³ is independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a}; and
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

In some embodiments, X¹, X², X³ and X⁴ are each CR³. In some embodiments, X¹ is N; and X², X³, and X⁴ are each CR³. In some embodiments, each R³ is independently selected from H, halogen, —NR⁸R⁹, —OR¹⁰, —C(O)R¹⁰, —C(O)OR¹⁰, —C(O)NR⁸R⁹, —NR¹²C(O)R¹⁰, —NR¹²C(O)OR¹⁰—, —NR¹²C(O)NR⁸R⁹, substituted or unsubstituted C₁-C₆ alkyl, substituted or unsubstituted C₁-C₆ haloalkyl, substituted or unsubstituted phenyl, and substituted or unsubstituted 5- to 10-membered heteroaryl.

In some embodiments, the compound of Formula (I) has the structure of Formula (II), or a pharmaceutically acceptable salt thereof:

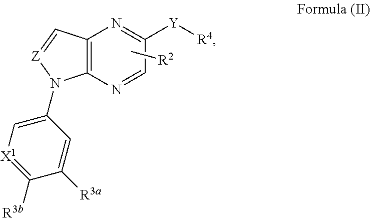

wherein,
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR^{B}, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
R⁴ is substituted or unsubstituted C₁-C₈ alkyl, substituted or unsubstituted C₂-C₈ alkenyl, substituted or unsubstituted C₁-C₈ aminoalkyl, substituted or unsubstituted C₁-C₈ heteroalkyl, substituted or unsubstituted C₁-C₈ haloalkyl, substituted or unsubstituted C₁-C₈ hydroxyalkyl, substituted or unsubstituted C₃-C₈ cycloalkyl, or substituted or unsubstituted C₃-C₈ heterocycloalkyl, each of which is substituted with one or more R⁶;
or R⁴ is

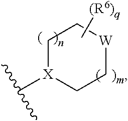

wherein
   X is CH or N;
   W is CR⁶R⁶, —NR^{6a}—, or —O—;
   each R⁶ is independently H, halogen, CN, —NO₂, —NR⁸R⁹, —OR¹⁰, —SR⁸, —C(O)R¹⁰, —C(O)OR¹, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
   or two R⁶ combine together with the atom(s) to which they are attached to form a C₃-C₆ cycloalkyl or C₃-C₆ heterocycloalkyl;
   R^{6a} is H or C₁-C₃ alkyl;
   m and n are each independently 0, 1, or 2;
   q is 0, 1, 2, 3, 4, 5, 6, 7, 8, 9, or 10;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R;
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R;
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a}; and
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

In some embodiments, the compound of Formula (II) has the structure of Formula (III), or a pharmaceutically acceptable salt thereof:

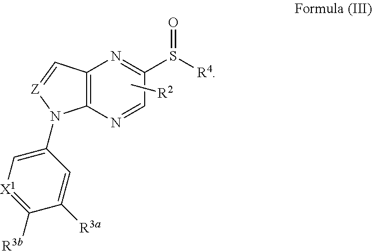

In some embodiments, R⁴ is substituted or unsubstituted C₁-C₈ alkyl, substituted or unsubstituted C₂-C₈ alkenyl, substituted or unsubstituted C₁-C₈ heteroalkyl. In some embodiments, R⁴ is C₁-C₈ alkyl which is substituted or unsubstituted with one or two halogens. In some embodiments, R⁴ is

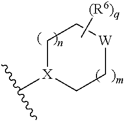

wherein,
X is CH or N;
W is CR⁶R⁶, —NR^{6a}—, or —O—;
each R⁶ is independently H, halogen, CN, —NO₂, —NR⁸R⁹, —OH, —OR¹⁰, —SR⁸, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
R^{6a} is H or C₁-C₃ alkyl;
m and n are each independently 0, 1, or 2; and
q is 0, 1, 2, 3, 4, 5, 6, 7, 8, 9, or 10.

In some embodiments, the compound of Formula (II) has the structure of Formula (IV), or a pharmaceutically acceptable salt thereof:

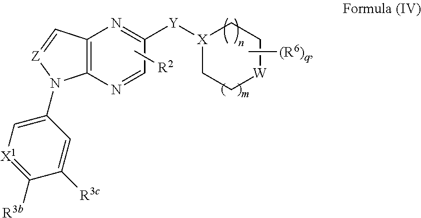

wherein,
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl; X¹ is N or CR^{3a};
R^{3a}, R^{3b}, and R^{3c} are each independently selected from H, halogen, —CN, —NO₂, —NR⁸R⁹, —OR¹⁰, —SR^{B}, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
X is CH or N;
W is CR⁶R⁶, —NR^{6a}—, or —O—;
each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
R^{6a} is H or C₁-C₃ alkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2; and
q is 0, 1, 2, 3, 4, 5, 6, 7, 8, 9, or 10.

In some embodiments, Y is —C(O)—. In some embodiments, Y is —S(O)—. In some embodiments, Y is —S(O)₂—. In some embodiments, X is CH.

In some embodiments, the compound of Formula (IV) has the structure of Formula (Va), or a pharmaceutically acceptable salt thereof:

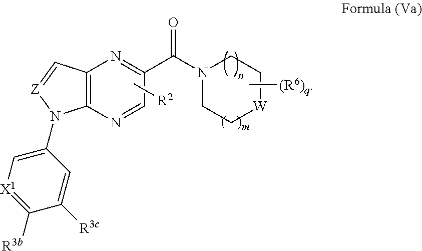

In some embodiments, the compound of Formula (IV) has the structure of Formula (Vb), or a pharmaceutically acceptable salt thereof:

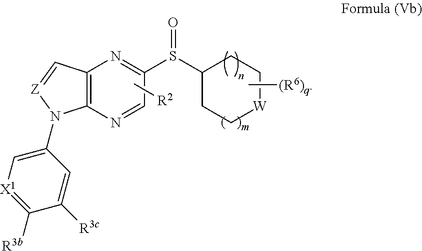

In some embodiments, the compound of Formula (IV) has the structure of Formula (Vc), or a pharmaceutically acceptable salt thereof:

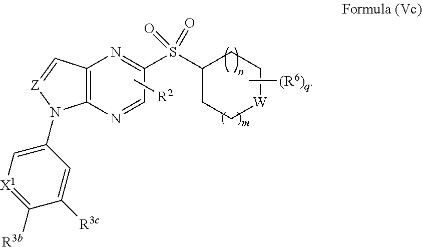

In another aspect, provided herein is a compound having the structure of Formula (VI), or a pharmaceutically acceptable salt thereof:

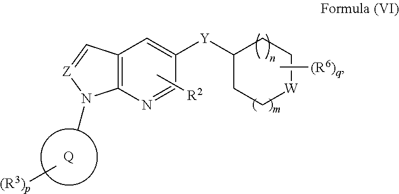

wherein,
Y is —S(O)— or —S(O)₂—;
Z is CR¹ or N;
ring Q is phenyl or 5- to 10-membered heteroaryl;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
each R³ is independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
W is CR⁶R⁶ or —O—;
each R⁶ is independently H, halogen, CN, —NO₂, —NR⁸R⁹, —OR¹⁰, —SR, —C(O)R¹⁰, —C(O)OR¹, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2;
p is 1, 2, 3, or 4; and
q is 0, 1, 2, 3, 4, 5, 6, 7, 8, 9, or 10.

In some embodiments, the compound of Formula (VI) has the structure of Formula (VIIa) or Formula (VIIb), or a pharmaceutically acceptable salt thereof:

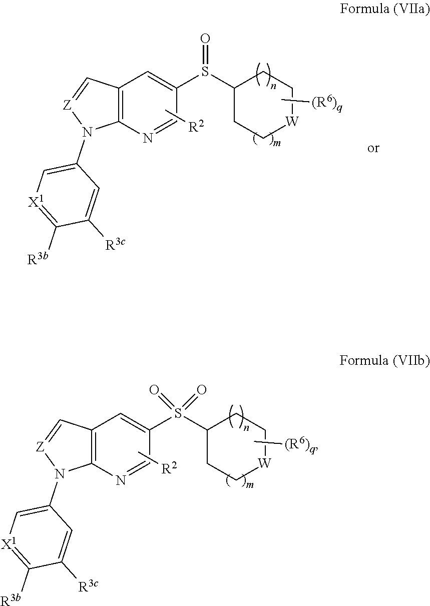

wherein,
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
W is CR⁶R⁶ or —O—;
each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2; and
q is 0, 1, 2, 3, 4, 5, 6, 7, 8, 9, or 10.

In some embodiments, the compound of Formula (VI) has the structure of Formula (VIIIa) or Formula (VIIIb), or a pharmaceutically acceptable salt thereof:

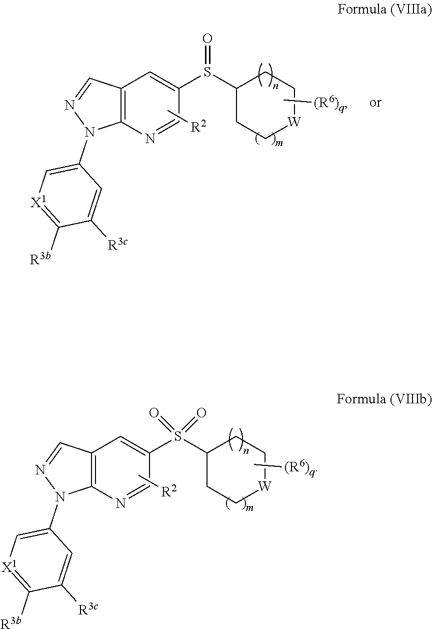

In some embodiments, the compound of Formula (VI) has the structure of Formula (VIIIc) or Formula (VIIId), or a pharmaceutically acceptable salt thereof:

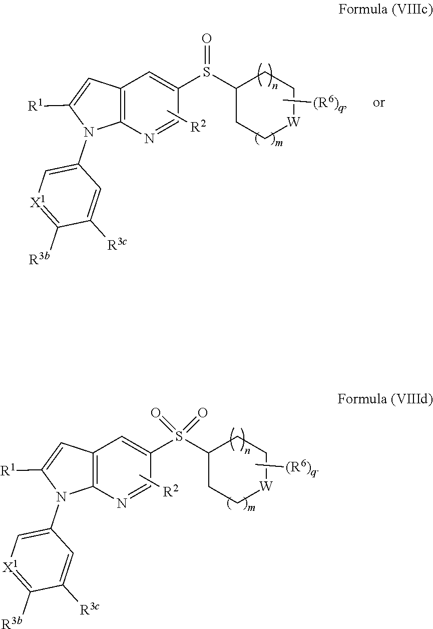

In some embodiments, W is —O—. In some embodiments, W is —CR⁶R⁶—. In some embodiments, m is 0 and n is 1. In some embodiments, m is 1 and n is 1. In some embodiments, each R⁶ is independently H, halogen, or C₁-C₃ alkyl; and q is 1 or 2.

In another aspect, provided herein is a compound having the structure of Formula (IX), or a pharmaceutically acceptable salt thereof:

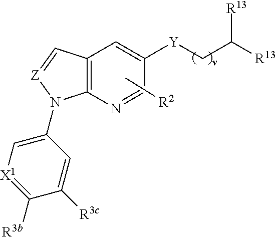

wherein,
Y is —S(O)— or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
each R¹³ is independently H, halogen, —CN, —NO₂, —NR⁸R⁹, —OR¹⁰, substituted or unsubstituted C₁-C₆ alkyl, substituted or unsubstituted C₁-C₆ haloalkyl, substituted or unsubstituted C₃-C₆ cycloalkyl, or substituted or unsubstituted C₃-C₆ heterocycloalkyl, each of which is substituted with one or more R¹³ or two R¹³ combine together with the atoms to which they are attached to form a substituted or unsubstituted C₃-C₆ cycloalkyl or substituted or unsubstituted heterocycloalkyl, each of which is substituted with one or more R^{13a}, wherein each R^{13a} is independently halogen, —CN, —NO₂, —NR₈R₉, —OR¹⁰, alkyl, or haloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃; and
v is 0-20.

In some embodiments, the compound of Formula (IX) has the structure of Formula (Xa) or Formula (Xb), or a pharmaceutically acceptable salt thereof:

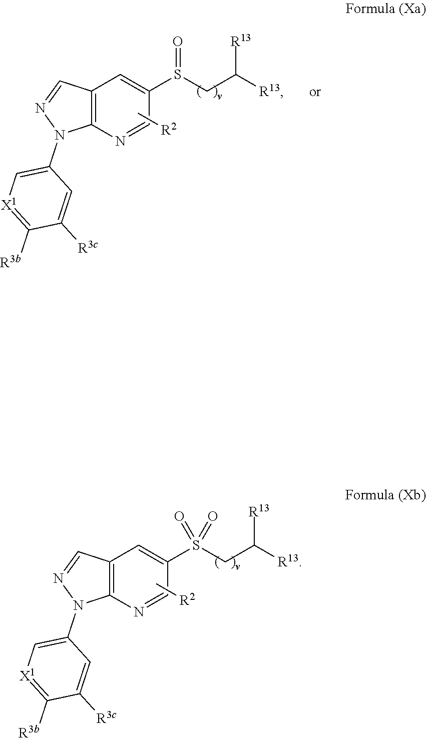

In some embodiments, the compound of Formula (IX) has the structure of Formula (Xc) or Formula (Xd), or a pharmaceutically acceptable salt thereof:

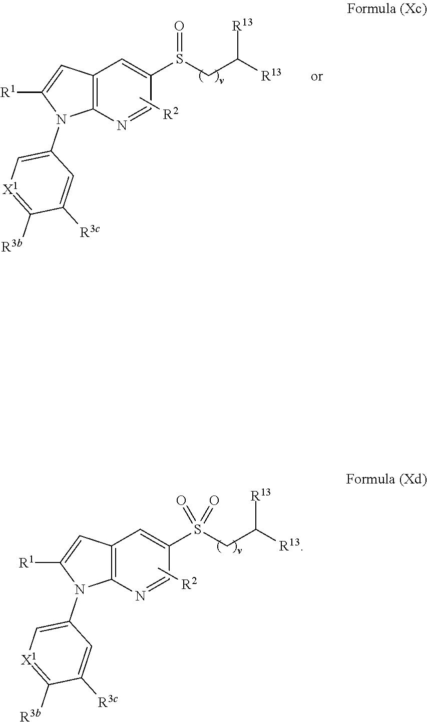

In some embodiments, X¹ is CR^{3a}. In some embodiments, X¹ is N. In some embodiments, R^{3a} and R^{3b} are independently H or halogen; and R^{3c} is selected from halogen, —CN, —NO₂, —NR⁸R⁹, —OR¹⁰, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl. In some embodiments, R^{3a} and R^{3b} are both H. In some embodiments, R^{3c} is a —NR¹²C(O)OR¹⁰ or a 5-membered heteroaryl. In some embodiments, R² is H.

In another aspect, provided herein is a pharmaceutical composition comprising a compound described herein, or a pharmaceutically acceptable salt thereof; and a pharmaceutically acceptable excipient

In another aspect, provided herein is a method of promoting and/or stimulation skin pigmentation, comprising administering one or more of the compositions described herein to a subject in need thereof.

In another aspect, provided herein is a method of inhibiting hair loss, comprising administering one or more of the compositions described herein to a subject in need thereof.

method of preventing and/or treating skin inflammation and/or damage, comprising administering one or more of the compositions described herein to a subject in need thereof.

In another aspect, provided herein is a method of preventing and/or treating vascular insufficiency, comprising administering one or more of the compositions described herein to a subject in need thereof.

In another aspect, provided herein is a method of preventing, treating, minimizing and/or reversing congestive heart failure, cardiomyopathy, comprising administering one or more of the compositions described herein to a subject in need thereof.

In another aspect, provided herein is a method of reducing cardiac ejection fraction, comprising administering one or more of the compositions described herein to a subject in need thereof.

In another aspect, provided herein is a method of preventing and/or treating a gastrointestinal disease, comprising administering one or more of the compositions described herein to a subject in need thereof.

In another aspect, provided herein is a method of preventing and/or treating renal dysfunction, comprising administering one or more of the compositions described herein to a subject in need thereof.

In another aspect, provided herein is a method of stimulation bone resorption and bone formation, comprising administering one or more of the compositions described herein to a subject in need thereof.

In another aspect, provided herein is a method of stimulating tissue regeneration by stimulating, comprising administering one or more of the compositions described herein to a subject in need thereof.

In another aspect, provided herein is a method of modulating cervical ripening, comprising administering one or more of the compositions described herein to a subject in need thereof.

In another aspect, provided herein is a method of promoting neuroprotection and/or stimulating neuronal regeneration, comprising administering one or more of the compositions described herein to a subject in need thereof.

In another aspect, provided herein is a method of treating and/or preventing a neurological disorder, a neuropsychiatric disorder, a neural injury, a neural toxicity disorder, a neuropathic pain, or a neural degenerative disorder, comprising administering one or more of the compositions described herein to a subject in need thereof.

In another aspect, provided herein is a method of treating and/or preventing fibrotic or adhesion disease, disorder or condition, comprising administering one or more of the compositions described herein to a subject in need thereof.

In another aspect, provided herein is a method of reducing and/or preventing scar formation, comprising administering one or more of the compositions described herein to a subject in need thereof.

In another aspect, provided herein is a method of treating and/or preventing muscle disorder, muscle injury and/or muscle atrophy, comprising administering one or more of the compositions described herein to a subject in need thereof.

In another aspect, provided herein is a method of treating and/or preventing fibrosis, comprising administering one or more of the compositions described herein to a subject in need thereof.

In another aspect, provided herein is a method of treating and/or preventing idiopathic pulmonary fibrosis, comprising administering one or more of the compositions described herein to a subject in need thereof.

In another aspect, provided herein is a method of treating and/or preventing kidney fibrosis, comprising administering one or more of the compositions described herein to a subject in need thereof.

In another aspect, provided herein is a method of stimulating muscle regeneration, comprising administering one or more of said compositions described herein to a subject in need thereof.

In another aspect, provided herein is a method of promoting organ fitness, comprising administering one or more of said compositions described herein to a subject in need thereof.

In another aspect, provided herein is a method of promoting wound healing, comprising administering one or more of said compositions described herein to a subject in need thereof.

In another aspect, provided herein is a method of treating acute kidney injury, comprising administering one or more of said compositions described herein to a subject in need thereof.

In another aspect, provided herein is a method of treating sarcopenia, comprising administering one or more of said compositions described herein to a subject in need thereof.

In another aspect, provided herein is a method of treating a neuromuscular disease, comprising administering one or more of said compositions of any of the preceding claims to a subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Unless defined otherwise, all technical and scientific terms used herein have the same meaning as is commonly understood by one of skill in the art to which this invention belongs. All patents and publications referred to herein are incorporated by reference.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —C(O)OH, —C(O)OMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —C(O)OH, —C(O)OMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —C(O)OH, C(O)OMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —C(O)OH, C(O)OMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —C(O)OH, C(O)OMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —C(O)OH, C(O)OMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Carbocycle” refers to a saturated, unsaturated or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle may be optionally substituted.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ fully saturated cycloalkyl or C₃-C₁₅ cycloalkenyl), from three to ten carbon atoms (C₃-C₁₀ fully saturated cycloalkyl or C₃-C₁₀ cycloalkenyl), from three to eight carbon atoms (C₃-C₈ fully saturated cycloalkyl or C₃-C₈ cycloalkenyl), from three to six carbon atoms (C₃-C₆ fully saturated cycloalkyl or C₃-C₆ cycloalkenyl), from three to five carbon atoms (C₃-C₅ fully saturated cycloalkyl or C₃-C₅ cycloalkenyl), or three to four carbon atoms (C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —C(O)OH, C(O)OMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ fully saturated heterocycloalkyl or C₂-C₁₅ heterocycloalkenyl), from two to ten carbon atoms (C₂-C₁₀ fully saturated heterocycloalkyl or C₂-C₁₀ heterocycloalkenyl), from two to eight carbon atoms (C₂-C₈ fully saturated heterocycloalkyl or C₂-C₈ heterocycloalkenyl), from two to seven carbon atoms (C₂-C₇ fully saturated heterocycloalkyl or C₂-C₇ heterocycloalkenyl), from two to six carbon atoms (C₂-C₆ fully saturated heterocycloalkyl or C₂-C₇ heterocycloalkenyl), from two to five carbon atoms (C₂-C₈ fully saturated heterocycloalkyl or C₂-C₈ heterocycloalkenyl), or two to four carbon atoms (C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —C(O)OH, C(O)OMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —C(O)OH, C(O)OMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

Isolation and purification of the chemical entities and intermediates described herein can be separated, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E-forms (or cis- and trans-forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that may induce a particular response in target cells, e.g., reduction of platelet adhesion and/or cell migration. The specific dose may vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

The terms “antagonist” and “inhibitor” are used interchangeably, and they refer to a compound having the ability to inhibit a biological function (e.g., activity, expression, binding, protein-protein interaction) of a target protein or enzyme. Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

Whenever a protein is referred to herein, it will be understood that a single protein can be referred to by different names. For example, “15-PGDH”, “PGDH”, and “hPGDH” all refer to the same protein, 15-hydroxyprostaglandin dehydrogenase.

### Compounds

Provided herein are compounds and methods of inhibiting 15-hydroxyprostaglandin dehydrogenase (15-PGDH).

In one aspect, provided herein is a compound having the structure of Formula (I), or a pharmaceutically acceptable salt thereof:

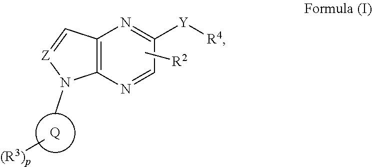

wherein,
ring Q is phenyl or 5- to 10-membered heteroaryl;
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
each R³ is independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
R⁴ is substituted or unsubstituted C₁-C₈ alkyl, substituted or unsubstituted C₂-C₈ alkenyl, substituted or unsubstituted C₁-C₈ aminoalkyl, substituted or unsubstituted C₁-C₈ heteroalkyl, substituted or unsubstituted C₁-C₈ haloalkyl, substituted or unsubstituted C₁-C₈ hydroxyalkyl, substituted or unsubstituted C₃-C₈ cycloalkyl, or substituted or unsubstituted C₃-C₈ heterocycloalkyl, each of which is substituted with one or more R⁶;
or R⁴ is

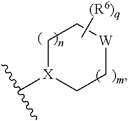

wherein
   X is CH or N;
   W is CR⁶R⁶, —NR^{6a}—, or —O—;
   each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
   or two R⁶ combine together with the atom(s) to which they are attached to form a C₃-C₆ cycloalkyl or C₃-C₆ heterocycloalkyl;
   R^{6a} is H or C₁-C₃ alkyl;
   m and n are each independently 0, 1, or 2;
   q is 0, 1, 2, 3, 4, 5, 6, 7, 8, 9, or 10;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃; and
p is 1, 2, 3, or 4.

In one aspect, provided herein is a compound having the structure of Formula (I), or a pharmaceutically acceptable salt thereof:

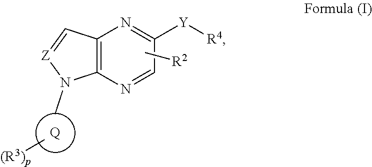

wherein,
ring Q is phenyl or 5- to 10-membered heteroaryl;
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
each R³ is independently selected from H, halogen, —CN, —NO₂, —NR⁸R⁹, —OR¹⁰, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl;
R⁴ is substituted or unsubstituted C₁-C₈ alkyl, substituted or unsubstituted C₂-C₈ alkenyl, substituted or unsubstituted C₁-C₈ heteroalkyl, substituted or unsubstituted C₁-C₈ haloalkyl, substituted or unsubstituted C₁-C₈ hydroxyalkyl, substituted or unsubstituted C₃-C₈ cycloalkyl, or C₄-C₈ heterocycloalkyl, each of which is substituted with one or more R⁶;
or R⁴ is

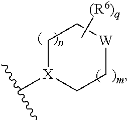

wherein
   X is CH or N;
   W is CR⁶R⁶, —NR^{6a}—, or —O—;
   each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, phenyl, or 5- to 8-membered heteroaryl;
   or two R⁶ combine together with the atom(s) to which they are attached to form a C₃-C₆ cycloalkyl;
   R^{6a} is H or C₁-C₃ alkyl;
   m and n are each independently 0, 1, or 2;
   q is 0, 1, 2, 3, 4, 5, 6, 7, 8, 9, or 10;
each R⁸ and R⁹ is independently selected from H, C₁-C₆ alkyl, C₂-C₆ alkenyl, C₂-C₆ alkynyl, C₁-C₆ heteroalkyl, C₁-C₆ haloalkyl, C₃-C₁₀cycloalkyl, and C₄-C₁₀ heterocycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl; and
p is 1, 2, 3, or 4.

In some embodiments, the compound of Formula (I) has the structure of Formula (II), or a pharmaceutically acceptable salt thereof:

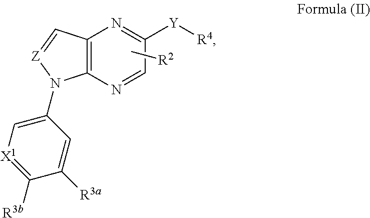

wherein,
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
R⁴ is substituted or unsubstituted C₁-C₈ alkyl, substituted or unsubstituted C₂-C₈ alkenyl, substituted or unsubstituted C₁-C₈ aminoalkyl, substituted or unsubstituted C₁-C₈ heteroalkyl, substituted or unsubstituted C₁-C₈ haloalkyl, substituted or unsubstituted C₁-C₈ hydroxyalkyl, substituted or unsubstituted C₃-C₈ cycloalkyl, or substituted or unsubstituted C₃-C₈ heterocycloalkyl, each of which is substituted with one or more R⁶;
or R⁴ is

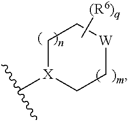

wherein
   X is CH or N;
   W is CR⁶R⁶, —NR^{6a}—, or —O—;
   each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
   or two R⁶ combine together with the atom(s) to which they are attached to form a C₃-C₆ cycloalkyl or C₃-C₆ heterocycloalkyl;
   R^{6a} is H or C₁-C₃ alkyl;
   m and n are each independently 0, 1, or 2;
   q is 0, 1, 2, 3, 4, 5, 6, 7, 8, 9, or 10;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a}; and
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

In some embodiments, the compound of Formula (II) has the structure of Formula (III), or a pharmaceutically acceptable salt thereof:

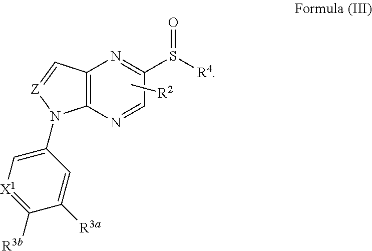

In some embodiments, R⁴ is substituted or unsubstituted C₁-C₆ alkyl, substituted or unsubstituted C₂-C₈ alkenyl, substituted or unsubstituted C₁-C₈ heteroalkyl, substituted or unsubstituted C₁-C₈ hydroxyalkyl, or substituted or unsubstituted C₁-C₈ hydroxyalkyl, each of which is substituted with one or more R⁶. In some embodiments, R⁴ is substituted or unsubstituted C₁-C₈ alkyl, substituted or unsubstituted C₂-C₈ alkenyl, substituted or unsubstituted C₁-C₈ heteroalkyl. In some embodiments, R⁴ is substituted or unsubstituted C₁-C₈ alkyl. In some embodiments, R⁴ is substituted or unsubstituted C₂-C₈ alkenyl. In some embodiments, R⁴ is substituted or unsubstituted C₁-C₈ heteroalkyl. In some embodiments, the heteroalkyl is an alkyl chain wherein one or more of the carbon atoms is replaced with an O or N atom.

In some embodiment, R⁴ is substituted or unsubstituted C₁-C₈ alkyl or substituted or unsubstituted C₁-C₈ heteroalkyl. In some embodiment, R⁴ is substituted or unsubstituted C₁-C₈ alkyl. In some embodiments, the alkyl is a straight chain or branched alkyl. In some embodiment, R⁴ is substituted or unsubstituted C₁-C₈ heteroalkyl. In some embodiments, the heteroalkyl is an alkyl chain wherein one or more of the carbon atoms is replaced with an O or N atom. In some embodiment, R⁴ is substituted or unsubstituted —CH₂CH₂—O—(C₁-C₄ alkyl), —CH₂—O—(C₁-C₄ alkyl), substituted or unsubstituted —CH₂CH₂—O—(C₁-C₄ haloalkyl), —CH₂—O—(C₁-C₄ haloalkyl), —CH₂CH₂—O—(C₃-C₆ cycloalkyl), —CH₂—O—(C₃-C₆ cycloalkyl), —CH₂CH₂—O—(C₃-C₆ heterocycloalkyl), or —CH₂—O—(C₃-C₆ heterocycloalkyl). In some embodiment, R⁴ is substituted or unsubstituted —CH₂CH₂—O—(C₁-C₄ alkyl), —CH₂—O—(C₁-C₄ alkyl), substituted or unsubstituted —CH₂CH₂—O—(C₁-C₄ haloalkyl), or —CH₂—O—(C₁-C₄ haloalkyl). In some embodiments, R⁴ is —CH₂CH₂—O—(C₃-C₆ cycloalkyl), —CH₂—O—(C₃-C₆ cycloalkyl), —CH₂CH₂—O—(C₃-C₆ heterocycloalkyl), or —CH₂—O—(C₃-C₆ heterocycloalkyl). In some embodiments, R⁴ is —CH₂CH₂—O—(C₃-C₆ cycloalkyl). In some embodiments, R⁴ is —CH₂—O—(C₃-C₆ cycloalkyl). In some embodiments, R⁴ is —CH₂CH₂—O—(C₃-C₆ heterocycloalkyl). In some embodiments, R⁴ is —CH₂—O—(C₃-C₆ heterocycloalkyl).

In some embodiments, R⁴ is substituted or unsubstituted C₁-C₈ alkyl, which is substituted with one or more halogen, —OR¹⁰, C₁-C₈ alkyl, or C₃-C₆ cycloalkyl. In some embodiments, R⁴ is C₁-C₈ alkyl which is substituted or unsubstituted with one or two halogen.

In some embodiments, R⁴ is —CH₃, —CH₂CH₃, —CH₂CH₂CH₃, —CH₂(CH₂)₂CH₃, —CH₂CH(CH₃)₂, —CH₂CH₂CH(CH₃)₂, —CH₂(CH₂)₂OCH₃,

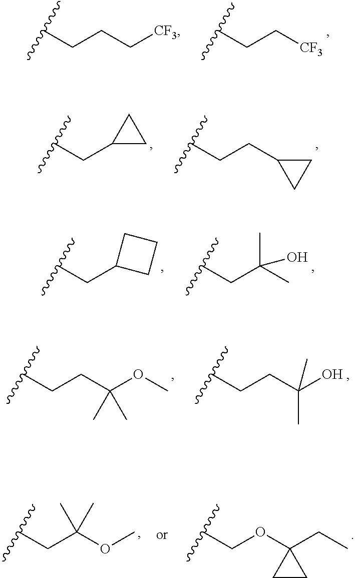

In some embodiments, R⁴ is substituted or unsubstituted C₃-C₈ cycloalkyl or substituted or unsubstituted C₃-C₈ heterocycloalkyl, each of which is substituted with one or more R⁶. In some embodiments, R⁴ is substituted or unsubstituted C₃-C₈ cycloalkyl. In some embodiments, R⁴ is monocyclic, polycyclic, spirocyclic, or bridged cycloalkyl. In some embodiments, R⁴ is cyclopropyl, cyclobutyl, cyclopentyl, cyclohexyl, or cycloheptyl. In some embodiments, R⁴ is cyclobutyl, cyclopentyl, or cyclohexyl. In some embodiments, R⁴ is cyclopropyl. In some embodiments, R⁴ is cyclobutyl. In some embodiments, R⁴ is cyclopentyl. In some embodiments, R⁴ is cyclohexyl. In some embodiments, R⁴ is

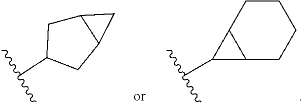 [SMILES: *C1CC2CC2C1 ; *C1C2CCCCC12]

In some embodiments, R⁴ is substituted or unsubstituted C₃-C₈ heterocycloalkyl. In some embodiments, R⁴ is monocyclic, polycyclic, spirocyclic, or bridged heterocycloalkyl. In some embodiments, R⁴ is a 4-membered heterocycloalkyl. In some embodiments, R⁴ is a 5-membered heterocycloalkyl. In some embodiments, R⁴ is a 6-membered heterocycloalkyl. In some embodiments, R⁴ is a 7-membered heterocycloalkyl. In some embodiments, R⁴ is tetrahydrofuran, pyrrolidine, tetrahydropyran, or piperidine. In some embodiments, R⁴ is tetrahydrofuran or tetrahydropyran. In some embodiments, R⁴ is tetrahydrofuran. In some embodiments, R⁴ is tetrahydropyran.

In some embodiments, R⁴ is substituted or unsubstituted C₁-C₈ alkyl, substituted or unsubstituted C₁-C₈ hydroxyalkyl, substituted or unsubstituted C₃-C₈ cycloalkyl, or substituted or unsubstituted C₃-C₈ heterocycloalkyl, each of which is substituted with one or two halogen, —OH, —OCH₃, —OCH₂CH₃, or —CH₃,

In some embodiments, R⁴ is

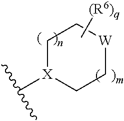

wherein,
X is CH or N;
W is CR⁶R⁶, —NR^{6a}—, or —O—;
each R⁶ is independently H, halogen, CN, —NO₂, —NR⁸R⁹, —OH, —OR¹⁰, —SR⁸, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
R^{6a} is H or C₁-C₃ alkyl;
m and n are each independently 0, 1, or 2; and
q is 0, 1, 2, 3, 4, 5, 6, 7, 8, 9, or 10.

In some embodiments, the compound of Formula (II) has the structure of Formula (IV), or a pharmaceutically acceptable salt thereof:

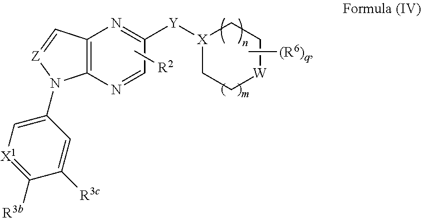

wherein,
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR⁸, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
X is CH or N;
W is CR⁶R⁶, —NR^{6a}—, or —O—;
each R⁶ is independently H, halogen, CN, —NO₂, —NR⁸R⁹, —OR¹⁰, —SR⁸, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
R^{6a} is H or C₁-C₃ alkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a}; and
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2; and
q is 0, 1, 2, 3, 4, 5, 6, 7, 8, 9, or 10.

In some embodiments, Y is —S(O)—, or —S(O)₂—. In some embodiments, —S(O)—. In some embodiments, Y is —S(O)₂—. In some embodiments, Y is —C(O)—.

In some embodiments, X is CH. In some embodiments, X is N.

In some embodiments, Y is —C(O)— and X is CH.

In some embodiments, the compound of Formula (IV) has the structure of Formula (Va), or a pharmaceutically acceptable salt thereof:

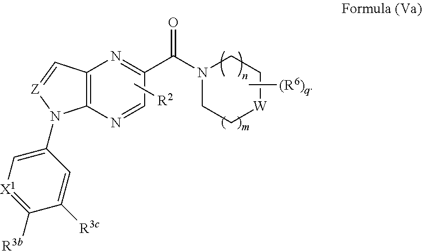

In some embodiments, the compound of Formula (IV) has the structure of Formula (Vb), or a pharmaceutically acceptable salt thereof:

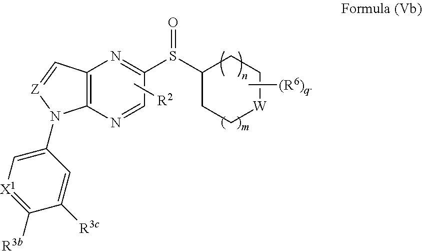

In some embodiments, the compound of Formula (IV) has the structure of Formula (Vc), or a pharmaceutically acceptable salt thereof:

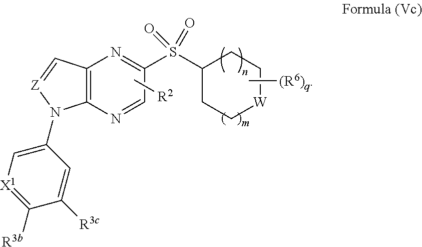

In another aspect, provided herein is a compound having the structure of Formula (VI), or a pharmaceutically acceptable salt thereof:

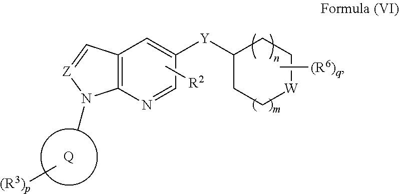

wherein,
Y is —S(O)— or —S(O)₂—;
Z is CR¹ or N;
ring Q is phenyl or 5- to 10-membered heteroaryl;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
each R³ is independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
W is CR⁶R⁶ or —O—;
each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R;
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R;
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R;
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2;
p is 1, 2, 3, or 4; and
q is 0, 1, 2, 3, 4, 5, 6, 7, 8, 9, or 10.

In some embodiments, the compound of Formula (VI) has the structure of Formula (VIIa), or a pharmaceutically acceptable salt thereof:

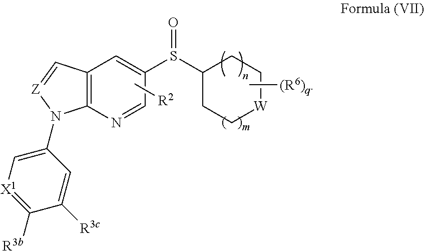

wherein,
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
W is CR⁶R⁶ or —O—;
each R⁶ is independently H, halogen, CN, —NO₂, —NR⁸R⁹, —OR¹⁰, —SR^{B}, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2; and
q is 0, 1, 2, 3, 4, 5, 6, 7, 8, 9, or 10.

In some embodiments, the compound of Formula (VI) has the structure of Formula (VIIb), or a pharmaceutically acceptable salt thereof:

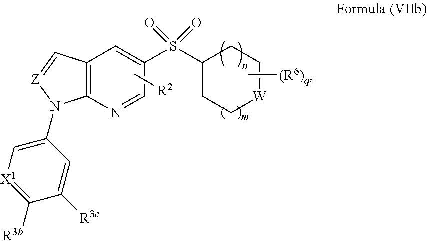

wherein,
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
W is CR⁶R⁶ or —O—;
each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2; and
q is 0, 1, 2, 3, 4, 5, 6, 7, 8, 9, or 10.

In some embodiments, the compound of Formula (VI) has the structure of Formula (VIIIa), or a pharmaceutically acceptable salt thereof:

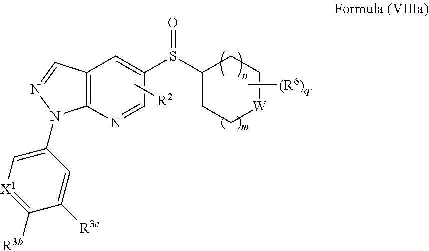

In some embodiments, the compound of Formula (VI) has the structure of Formula (VIIIb), or a pharmaceutically acceptable salt thereof:

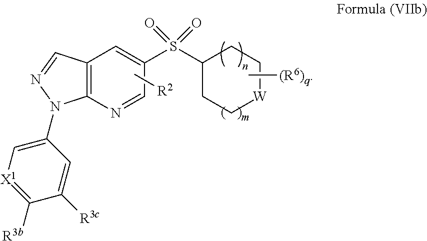

In some embodiments, the compound of Formula (VI) has the structure of Formula (VIIIc), or a pharmaceutically acceptable salt thereof:

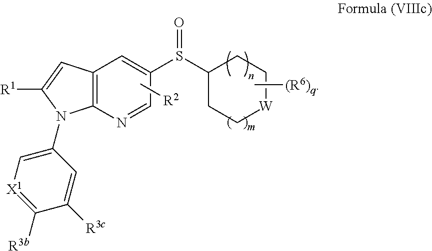

In some embodiments, the compound of Formula (VI) has the structure of Formula (VIIId), or a pharmaceutically acceptable salt thereof:

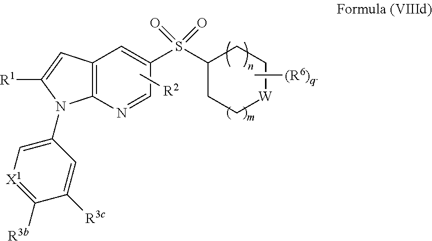

In some embodiments, ring Q is 5- to 10-membered heteroaryl, comprising 1, 2, 3, or 4 heteroatoms selected from N, O, and S. In some embodiments, ring Q is 5- to 8-membered heteroaryl, comprising 1, 2, 3, or 4 heteroatoms selected from N, O, and S. In some embodiments, ring Q is 5- to 8-membered heteroaryl, comprising 1, 2, 3, or 4 heteroatoms selected from N and O. In some embodiments, ring Q is a monocyclic, bicyclic, or polycyclic heteroaryl. In some embodiments, ring Q is a bicyclic heteroaryl comprising 1, 2, 3, or 4 heteroatoms selected from N and O. In some embodiments, ring Q is indole, benzimidazole, benzotriazole, pyrazolo pyridine, imidazopyridine, triazolopyridine, imidazopyridine, or tetrazolo pyridine. In some embodiments, ring Q is [1,2,4]triazolo[1,5-a]pyridine.

In some embodiments, ring Q is a 6-membered monocyclic heteroaryl comprising 1, 2, or 3 N atoms. In some embodiments, ring Q is phenyl. In some embodiments, ring Q is a phenyl, pyrimidine, or pyridine In some embodiments, ring Q is phenyl. In some embodiments, ring Q is pyrimidine In some embodiments, ring Q is pyridine.

In some embodiments, ring Q is phenyl, pyridine, or triazolopyridine.

In some embodiments,

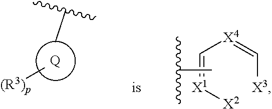

wherein,
X¹, X², X³ and X⁴ are each independently N or CR³;
each R³ is independently selected from H, halogen, —CN, —NO₂, —NR⁸R⁹, —OR¹⁰, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a}; and
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

In some embodiments, X¹, X², X³ and X⁴ are each CR³.

In some embodiments, X¹ is N; and X², X³, and X⁴ are each CR³. In some embodiments, X¹ and X² are N; and X³ and X⁴ are each CR³. In some embodiments, X¹ and X³ are N; and X¹ and X⁴ are each CR³. In some embodiments, X² and X⁴ are N; and X¹ and X³ are each CR³. In some embodiments, X¹, X², and X³ are N; and X⁴ is CR³.

In some embodiments, each R³ is independently selected from H, halogen, —CN, —NO₂, —NR⁸R⁹, —OR¹⁰, —SR⁸, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl. In some embodiments, each R³ is independently selected from H, halogen, —C(O)R¹⁰, —C(O)OR¹⁰, —C(O)NR⁸R⁹, —NR¹²C(O)R¹⁰, —NR¹²C(O)OR¹⁰, substituted or unsubstituted C₁-C₆ alkyl, substituted or unsubstituted C₁-C₆ haloalkyl, substituted or unsubstituted phenyl, and substituted or unsubstituted 5- to 10-membered heteroaryl. In some embodiments, each R³ is independently selected from H, halogen, —C(O)R¹⁰, —C(O)OR¹⁰, —C(O)NR⁸R⁹, —NR¹²C(O)R¹⁰, —NR¹²C(O)OR¹⁰, substituted or unsubstituted phenyl, and substituted or unsubstituted 5- to 6-membered heteroaryl. In some embodiments, each R³ is independently selected from H, halogen, substituted or unsubstituted C₃-C₆ heteroalkyl, substituted or unsubstituted phenyl, and substituted or unsubstituted 5- to 6-membered heteroaryl.

In some embodiments, each R³ is independently selected from H or halogen. In some embodiments, each R³ is independently substituted or unsubstituted phenyl, and substituted or unsubstituted 5- to 6-membered heteroaryl.

In some embodiments, W is —NR^{6a}— or —O—. In some embodiments, W is —NR^{6a}—. In some embodiments, W is —O—. In some embodiments, W is —CR⁶R⁶—. In some embodiments, W is —CH₂—.

In some embodiments, each R⁶ is independently H, halogen, CN, —NR⁸R⁹, —OH, —OR¹⁰, —C(O)R¹⁰, —C(O)OR¹⁰, —C(O)NR⁸R⁹, —NR⁸C(O)R¹¹—SOR¹¹, —SO₂R¹¹, —SR¹¹, substituted or unsubstituted C₁-C₆ alkyl, or C₃-C₈ cycloalkyl. In some embodiments, each R⁶ is independently halogen, CN, —NR⁸R⁹, —OH, —OR¹⁰, —C(O)R¹⁰, —C(O)OR¹⁰, —C(O)NR⁸R⁹, or substituted or unsubstituted C₁-C₆ alkyl. In some embodiments, each R⁶ is independently halogen.

In some embodiments, each R⁶ is independently —F, —NH₂, —OH, —OCH₃, or —CH₃. In some embodiments, each R⁶ is independently —F, —OH, —OCH₃, or —CH₃. In some embodiments, each R⁶ is independently —F or —CH₃. In some embodiments, each R⁶ is independently —CH₃. In some embodiments, each R⁶ is independently F, Cl, or Br. In some embodiments, each R⁶ is independently F. In some embodiments, each R⁶ is independently Br. In some embodiments, each R⁶ is independently Cl.

In some embodiments, each R⁶ is independently H, halogen, or C₁-C₃ alkyl; and q is 1 or 2.

In some embodiments, two R⁶ combine together with the atom(s) to which they are attached to form a C₃-C₆ cycloalkyl or C₃-C₆ heterocycloalkyl. In some embodiments, two R⁶ combine together with the atom(s) to which they are attached to form a C₃-C₆ cycloalkyl. In some embodiments, two R⁶ combine together with the atom(s) to which they are attached to form a cyclopropyl, cyclobutyl, or cyclopropyl. In some embodiments, two R⁶ combine together with the atom(s) to which they are attached to form a C₃-C₆ heterocycloalkyl. In some embodiments, two R⁶ combine together with the atom(s) to which they are attached to form oxetane, pyrrolidine, tetrahydropyran, piperidine, or piperazine.

In some embodiments, R^{6a} is H or C₁-C₃ alkyl. In some embodiments, R^{6a} is C₁-C₃ alkyl. In some embodiments, R^{6a} is methyl, ethyl, or propyl. In some embodiments, R^{6a} is methyl. In some embodiments, R^{6a} is ethyl. In some embodiments, R^{6a} is propyl. In some embodiments, R^{6a} is H.

In some embodiments, n and m are independently 0, 1, or 2. In some embodiments, n and m are independently 0. In some embodiments, n and m are independently 1. In some embodiments, n and m are independently 2. In some embodiments, n is 0, 1, or 2; and m is 1 or 2. In some embodiments, n is 0; and m is 1 or 2. In some embodiments, n is 1; and m is 1 or 2. In some embodiments, n is 2; and m is 1 or 2.

In some embodiments, q is 0, 1, 2, 3, 4, 5, or 6. In some embodiments, q is 1 or 2. In some embodiments, q is 6. In some embodiments, q is 5. In some embodiments, q is 4. In some embodiments, q is 3. In some embodiments, q is 2. In some embodiments, q is 1. In some embodiments, q is 0.

In another aspect, provided herein is a compound having the structure of Formula (IX), or a pharmaceutically acceptable salt thereof:

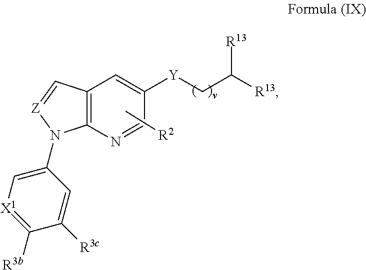

wherein,
Y is —S(O)— or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
each R¹³ is independently H, halogen, —CN, —NO₂, —NR⁸R⁹, —OR¹⁰, substituted or unsubstituted C₁-C₆ alkyl, substituted or unsubstituted C₁-C₆ haloalkyl, substituted or unsubstituted C₃-C₆ cycloalkyl, or substituted or unsubstituted C₃-C₆ heterocycloalkyl, each of which is substituted with one or more R¹³;
or two R¹³ combine together with the atoms to which they are attached to form a substituted or unsubstituted C₃-C₆ cycloalkyl or substituted or unsubstituted heterocycloalkyl, each of which is substituted with one or more R^{13a}, wherein each R^{13a} is independently halogen, —CN, —NO₂, —NR₈R₉, —OR¹⁰, alkyl, or haloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃; and
v is 0-20.

In another aspect, provided herein is a compound having the structure of Formula (IX), or a pharmaceutically acceptable salt thereof:

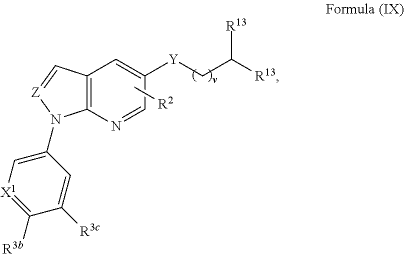

wherein,
Y is —S(O)— or —S(O)₂—;
Z is CR¹ or N;
X¹ is N or CR^{3a};
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
R^{3a}, R^{3b}, and R^{3c} are each independently selected from H, halogen, —CN, —NO₂, —NR⁸R⁹, —OR¹⁰, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl;
each R¹³ is independently H, halogen, —CN, —NO₂, —OR¹⁰, —NR⁸R⁹, substituted or unsubstituted C₁-C₃ alkyl, substituted or unsubstituted C₁-C₃ haloalkyl, or substituted or unsubstituted C₃-C₆ cycloalkyl, each of which is substituted with one or more R^{13a};
two R¹³ combine together to form a C₃-C₆ cycloalkyl which is substituted or unsubstituted with one or more R^{13a}; wherein each R^{13a} is independently halogen, —CN, —NO₂, —NR₈R₉, —OR¹⁰, alkyl, or haloalkyl;
each R⁸ and R⁹ is independently selected from H, C₁-C₆ alkyl, C₂-C₆ alkenyl, C₂-C₆ alkynyl, C₁-C₆ heteroalkyl, C₁-C₆ haloalkyl, C₃-C₁₀ cycloalkyl, and C₄-C₁₀ heterocycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl; and
v is 1-20.

In some embodiments, the compound of Formula (IX) has the structure of Formula (Xa), or a pharmaceutically acceptable salt thereof:

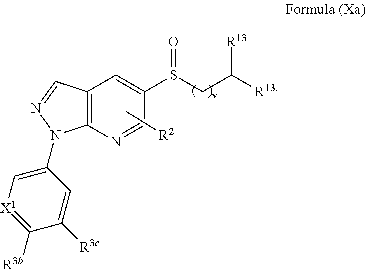

In some embodiments, the compound of Formula (IX) has the structure of Formula (Xb), or a pharmaceutically acceptable salt thereof:

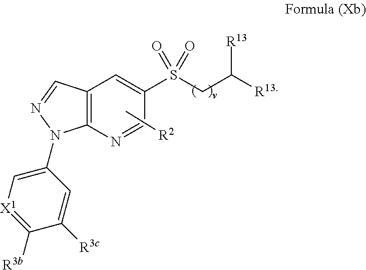

In some embodiments, the compound of Formula (IX) has the structure of Formula (Xc), or a pharmaceutically acceptable salt thereof:

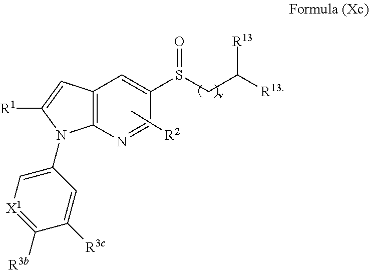

In some embodiments, the compound of Formula (IX) has the structure of Formula (Xd), or a pharmaceutically acceptable salt thereof:

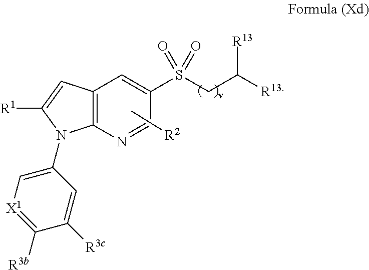

In some embodiments, each R¹³ is independently H, halogen, CN, —NO₂, —NR⁸R⁹, —OR¹⁰, C₁-C₆ alkyl, C₁-C₆ haloalkyl, or C₃-C₆ cycloalkyl. In some embodiments, each R¹³ is independently H, halogen, CN, —NO₂, —NR⁸R⁹, or —OR¹⁰. In some embodiments, each R¹³ is independently halogen. In some embodiments, each R¹³ is independently F or Cl. In some embodiments, each R¹³ is independently H.

In some embodiments, two R¹³ combine together with the atoms to which they are attached to form a C₃-C₆ cycloalkyl ring with is substituted or unsubstituted with one or more R^{13a}. In some embodiments, two R¹³ combine together to form a cyclopropyl, cyclobutyl, cyclopentyl, or cyclohexyl ring. In some embodiments, two R¹³ combine together to form a cyclopropyl, cyclobutyl or cyclopentyl ring. In some embodiments, two R¹³ combine together to form a cyclopropyl ring. In some embodiments, two R¹³ combine together to form a cyclobutyl ring. In some embodiments, two R¹³ combine together to form a cyclopentyl ring. In some embodiments, the cycloalkyl is substituted or unsubstituted with one or more halogen, haloalkyl, or alkyl.

In some embodiments, v is 1-20, 1-15, 1-10, 1-8, 1-7, 1-6, 1-5, 1-4, 1-3, or 1-2. In some embodiments, v is 1-20. In some embodiments, v is 1-15. In some embodiments, v is 1-10. In some embodiments, v is 1-8, in some embodiments, v is 1-6. In some embodiments, v is 1-5. In some embodiments, v is 1-4. In some embodiments, v is 1-3. In some embodiments, v is 1-2. In some embodiments, v is 1, 2, 3, 4, 5, 6, 7, or 8. In some embodiments, v is 8. In some embodiments, v is 7. In some embodiments, v is 6. In some embodiments, v is 5. In some embodiments, v is 4. In some embodiments, v is 3. In some embodiments, v is 2. In some embodiments v is 1. In some embodiments, v is 0.

In some embodiments, Z is N. In some embodiments, Z is CR¹. In some embodiments, Z is CH.

In some embodiments, R¹ is H, halogen, —OR¹⁰, —C(O)R¹⁰, —C(O)OR¹⁰, or substituted or unsubstituted C₁-C₆ alkyl. In some embodiments, R¹ is substituted or unsubstituted C₁-C₆ alkyl. In some embodiments, R¹ is H.

In some embodiments, R² is H, halogen, —OR¹⁰, —C(O)R¹⁰, —C(O)OR¹⁰, or substituted or unsubstituted C₁-C₆ alkyl. In some embodiments, R² is substituted or unsubstituted C₁-C₆ alkyl. In some embodiments, R² is H.

In some embodiments, X¹ is CR^{3a}. In some embodiments, X¹ is N.

In some embodiments, R^{3a}, R^{3b}, and R^{3c} are each independently selected from H, halogen, —CN, —NO₂, —NR⁸R⁹, —OR¹⁰, —C(O)R¹⁰, —C(O)OR¹⁰, —C(O)NR⁸R⁹, —SOR¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl. In some embodiments, R^{3a}, R^{3b}, and R^{3c} are each independently selected from H, halogen, —C(O)R¹⁰, —C(O)OR¹⁰, —C(O)NR⁸R⁹, —NR¹²C(O)R¹⁰, —NR¹²C(O)OR¹⁰, substituted or unsubstituted C₁-C₆ alkyl, substituted or unsubstituted C₁-C₆ haloalkyl, substituted or unsubstituted phenyl, and substituted or unsubstituted 5- to 10-membered heteroaryl. In some embodiments, R^{3a}, R^{3b}, and R^{3c} are each independently selected from H, halogen, —C(O)R¹⁰, —C(O)OR¹⁰, —C(O)NR⁸R⁹, —NR¹²C(O)R¹⁰, —NR¹²C(O)OR¹⁰, substituted or unsubstituted phenyl, and substituted or unsubstituted 5- to 6-membered heteroaryl. In some embodiments, R^{3a}, R^{3b}, and R^{3c} are each independently selected from H, halogen, substituted or unsubstituted C₁-C₈ alkyl, —C(O)OR¹⁰, —C(O)NR⁸R⁹, —NR¹²C(O)R¹⁰, —NR¹²C(O)OR¹⁰, substituted or unsubstituted phenyl, and substituted or unsubstituted 5- to 6-membered heteroaryl.

In some embodiments, R^{3a} and R^{3b} are each H or halogen; and R^{3c} is selected from halogen, —CN, —NO₂, —NR⁸R⁹, —OR¹⁰, —C(O)R¹⁰, —C(O)OR¹⁰, —C(O)NR⁸R⁹, —SOR¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl. In some embodiments, R^{3a} and R^{3b} are each H or halogen; and R^{3c} is selected from halogen, —C(O)R¹⁰, —C(O)OR¹⁰, —C(O)NR⁸R⁹, —NR¹²C(O)R¹⁰, —NR¹²C(O)OR¹⁰, —NR¹²C(O)NR⁸R⁹, substituted or unsubstituted C₁-C₆ alkyl, substituted or unsubstituted C₁-C₆ haloalkyl, substituted or unsubstituted phenyl, and substituted or unsubstituted 5- to 10-membered heteroaryl. In some embodiments, R^{3a} and R^{3b} are each H or halogen; and R^{3c} is selected from halogen, —C(O)R¹⁰, —C(O)OR¹⁰, —C(O)NR⁸R⁹, —NR¹²C(O)R¹⁰, —NR¹²C(O)OR¹⁰, substituted or unsubstituted phenyl, and substituted or unsubstituted 5- to 6-membered heteroaryl. In some embodiments, R^{3a} and R^{3b} are each H or halogen; and R^{3c} is selected from —NR¹²C(O)OR¹⁰, substituted or unsubstituted phenyl, and substituted or unsubstituted 5- to 6-membered heteroaryl. In some embodiments, R^{3a} and R^{3b} are each H or halogen; and R^{3c} is a substituted or unsubstituted 5-membered heteroaryl.

In some embodiments, R^{3a} is halogen and R^{3b} is H. In some embodiments, R^{3a} is Cl or F; and R^{3b} is H. In some embodiments, R^{3b} is halogen and R^{3a} is H. In some embodiments, R^{3b} is Cl or F; and R^{3a} is H.

In some embodiments, R^{3a} and R^{3b} are each H.

In some embodiments, R^{3a} and R^{3c} are independently halogen or H; and R^{3b} is selected from halogen, —CN, —NO₂, —NR⁸R⁹, —OR¹⁰, —C(O)R¹⁰, —C(O)OR¹⁰, —C(O)NR⁸R⁹, —SOR¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl. In some embodiments, R^{3a} and R^{3c} are independently halogen or H; and R^{3b} is selected from halogen, —C(O)R¹⁰, —C(O)OR¹⁰, —C(O)NR⁸R⁹, —NR¹²C(O)R¹⁰, —NR¹²C(O)OR¹⁰, —NR¹²C(O)NR⁸R⁹, substituted or unsubstituted C₁-C₆ alkyl, substituted or unsubstituted C₁-C₆ haloalkyl, substituted or unsubstituted phenyl, and substituted or unsubstituted 5- to 10-membered heteroaryl. In some embodiments, R^{3a} and R^{3c} are independently halogen or H; and R^{3b} is selected from halogen, —C(O)R¹⁰, —C(O)OR¹⁰, —C(O)NR⁸R⁹, —NR¹²C(O)R¹⁰, —NR¹²C(O)OR¹⁰, substituted or unsubstituted phenyl, and substituted or unsubstituted 5- to 6-membered heteroaryl. In some embodiments, R^{3a} and R^{3c} are independently halogen or H; and R^{3b} is selected from —C(O)R¹⁰, —C(O)OR¹⁰, —NR¹²C(O)OR¹⁰, substituted or unsubstituted phenyl, and substituted or unsubstituted 5- to 6-membered heteroaryl. In some embodiments, R^{3a} and R^{3c} are independently halogen or H and R^{3b} is selected from —C(O)R¹⁰, —C(O)OR¹⁰ and substituted or unsubstituted 5-membered heteroaryl.

In some embodiments, R^{3a} is halogen and R^{3c} is H. In some embodiments, R^{3a} is Cl or F; and R^{3c} is H. In some embodiments, R^{3c} is halogen and R^{3a} is H. In some embodiments, R^{3c} is Cl or F; and R^{3a} is H.

In some embodiments, R^{3a} and R^{3c} are each H.

In some embodiments, each R³, R^{3a}, R^{3b}, and R^{3c} is independently a substituted or unsubstituted 5-membered heteroaryl selected from pyrrole, triazole, tetrazole, oxazole, diazole, oxadiazole, thiadiazole, or furanyl. In some embodiments, each R³, R^{3a}, R^{3b}, and R^{3c} is independently a substituted or unsubstituted 5-membered heteroaryl selected from triazole and tetrazole.

In some embodiments, each R³, R^{3a}, R^{3b}, and R^{3c} is independently a substituted or unsubstituted 6-membered heteroaryl selected from pyridine and pyrimidine. In some embodiments, each R³, R^{3a}, R^{3b} and R^{3c} is independently a substituted or unsubstituted 6-membered heteroaryl selected from pyridine.

In some embodiments, each R³, R^{3a}, R^{3b}, or R^{3c} is independently selected from the group consisting of

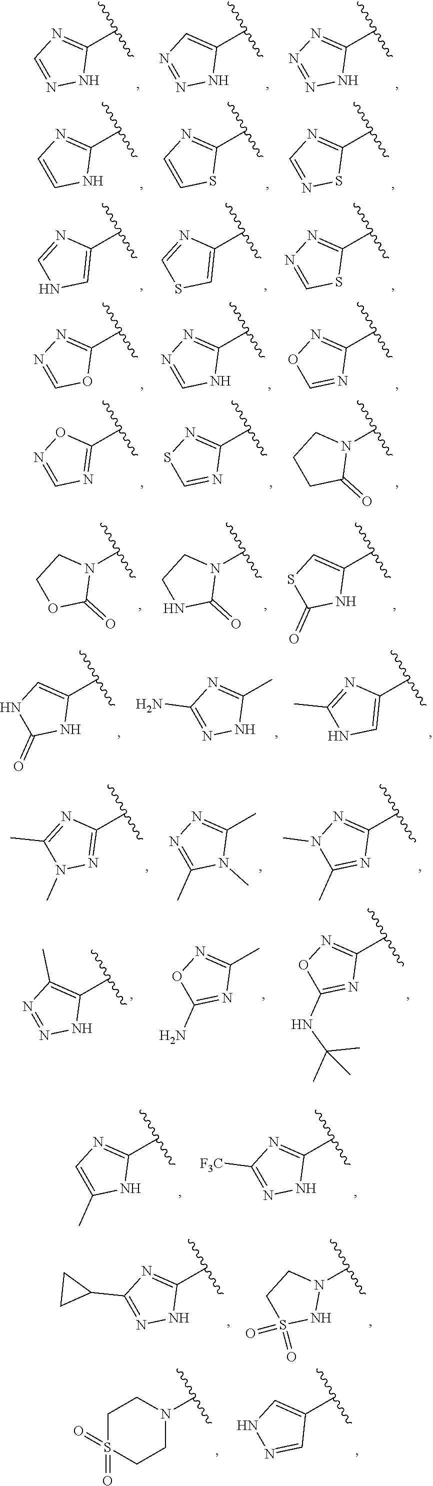

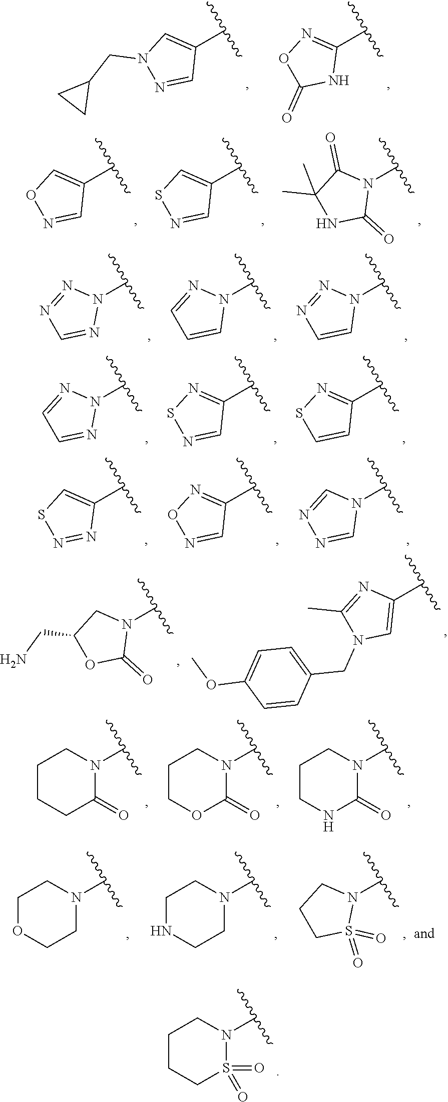

In some embodiments, each R³, R^{3a}, R^{3b}, or R^{3c} is independently selected from the group consisting of

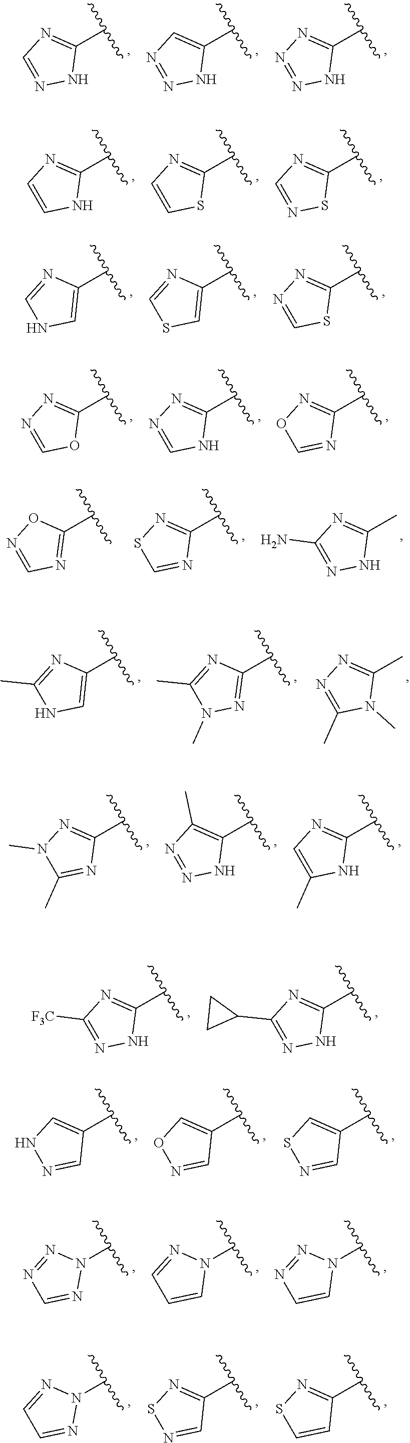

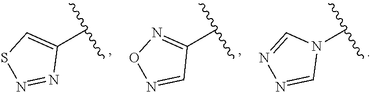 [SMILES: *c1csnn1 ; *c1cnon1 ; *n1cnnc1]

In some embodiments, each R³, R^{3a}, R^{3b}, or R^{3c} is independently selected from the group consisting of

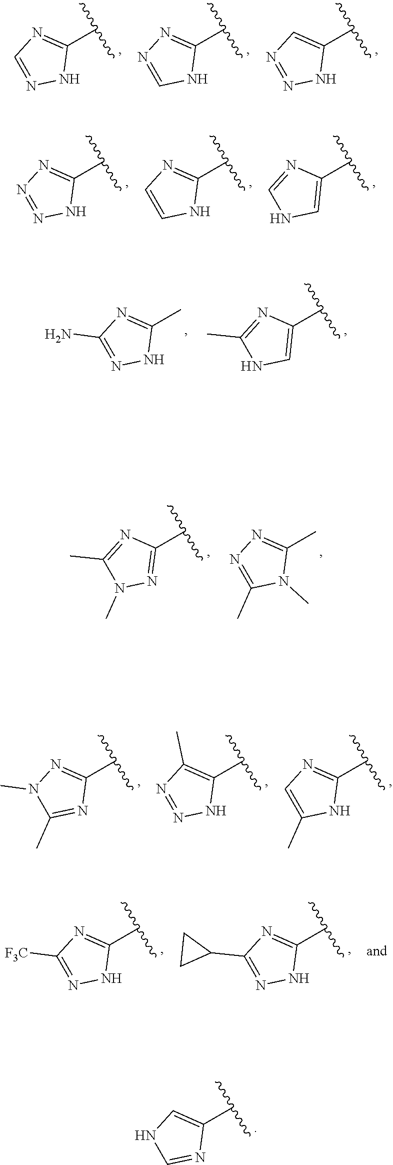

In some embodiments, R^{3a}, R^{3b}, R^{3c} are each independently H, halogen, —OR¹⁰, —C(O)OR¹⁰, —C(O)NR⁸R⁹, —NR¹²C(O)R¹⁰, substituted or unsubstituted C₁-C₆ alkyl, substituted or unsubstituted C₅-C₆ heterocycloalkyl, substituted or unsubstituted phenyl, and substituted or unsubstituted pyridine, and substituted or unsubstituted triazole, each of which is substituted with one or two R^{a}. In some embodiments, R^{3a}, R^{3b}, R^{3c} are each independently H, halogen, —OR¹⁰, —C(O)OR¹⁰, —C(O)NR⁸R⁹, —NR¹²C(O)R¹⁰, substituted or unsubstituted C₁-C₆ alkyl, substituted or unsubstituted C₅-C₆ heterocycloalkyl, substituted or unsubstituted phenyl, and substituted or unsubstituted pyridine, and substituted or unsubstituted triazole, each of which is substituted with one or two R^{a}, wherein one of R^{3a}, R^{3b}, or R^{3c} is not H.

In some embodiments, each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a}. In some embodiments, each R⁸ and R⁹ is independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl. In some embodiments, each R⁸ and R⁹ is independently selected at each occurrence from H, C₁-C₆ alkyl, C₁-C₆ heteroalkyl, C₁-C₆ haloalkyl, C₃-C₁₀ cycloalkyl, and C₃-C₁₀ heterocycloalkyl. In some embodiments, each R⁸ and R⁹ is independently selected at each occurrence from C₃-C₁₀ cycloalkyl and C₃-C₁₀ heterocycloalkyl.

In some embodiments, each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a}. In some embodiments, each R¹⁰ is independently selected from H, C₁-C₆ alkyl, C₂-C₆ alkenyl, C₁-C₆ heteroalkyl, C₁-C₆ haloalkyl, C₃-C₁₀ cycloalkyl, C₃-C₁₀ heterocycloalkyl, C₆-C₁₀ aryl, and 5- to 10-membered heteroaryl. In some embodiments, each R¹⁰ is independently selected from H, C₁-C₆ alkyl, C₃-C₁₀ cycloalkyl, and C₃-C₁₀ heterocycloalkyl. In some embodiments, each R¹⁰ is independently selected from H and C₁-C₆ alkyl. In some embodiments, each R¹⁰ is independently selected from C₃-C₁₀ cycloalkyl and C₃-C₁₀ heterocycloalkyl.

In some embodiments, each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R. In some embodiments, each R¹¹ is independently selected from C₁-C₆ alkyl, C₂-C₆ alkenyl, C₁-C₆ heteroalkyl, C₁-C₆ haloalkyl, C₃-C₁₀ cycloalkyl, C₃-C₁₀ heterocycloalkyl, C₆-C₁₀ aryl, and 5- to 10-membered heteroaryl. In some embodiments, each R¹¹ is independently selected from C₁-C₆ alkyl, C₁-C₆ heteroalkyl, and C₁-C₆ haloalkyl. In some embodiments, each R¹¹ is independently selected from C₃-C₁₀ cycloalkyl and C₃-C₁₀ heterocycloalkyl.

In some embodiments, each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a}. In some embodiments, each R¹² is independently selected from H, straight or branched chain C₁-C₆ alkyl, C₂-C₆ alkenyl, C₁-C₆ haloalkyl, C₃-C₁₀ cycloalkyl, and C₃-C₁₀ heterocycloalkyl. In some embodiments, each R¹² is independently selected from H, straight or branched chain C₁-C₆ alkyl. In some embodiments, each R¹² is independently selected from C₃-C₁₀ cycloalkyl and C₃-C₁₀ heterocycloalkyl.

In some embodiments, each R^{a} is independently selected from halogen, —OH, —CH₃, —CF₃, —OCH₃, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃. In some embodiments, each R^{a} is independently selected from halogen, —OH, —CH₃, —CF₃, —OCH₃, —C(O)OH, —C(O)NH₂, and —NHC(O)CH₃.

In some embodiments, p is 1, 2, 3, or 4. In some embodiments, p is 2 or 3. In some embodiments, p is 1 or 2. In some embodiments, p is 3. In some embodiments, p is 5. In some embodiments, p is 4. In some embodiments, p is 3. In some embodiments, p is 2. In some embodiments, p is 1.

In some embodiments, the PDGH inhibitor is a compound described in Table 1, or a pharmaceutically acceptable salt thereof.

TABLE 1
Compounds of the disclosure.

| Comp. No. | Structure |
|---|---|
| 1A and 1B | 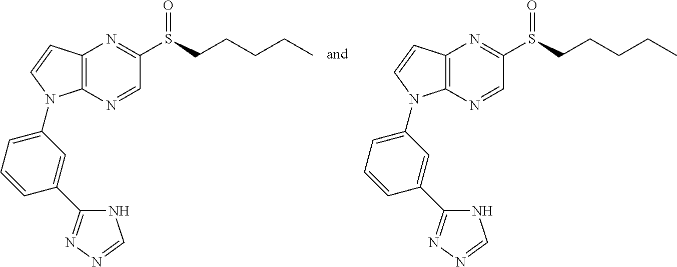 [SMILES: CCCCC[S@](=O)c1cnc2c(ccn2-c2cccc(-c3nnc[nH]3)c2)n1 ; CCCCC[S@](=O)c1cnc2c(ccn2-c2cccc(-c3nnc[nH]3)c2)n1] |
| 2A and 2B | 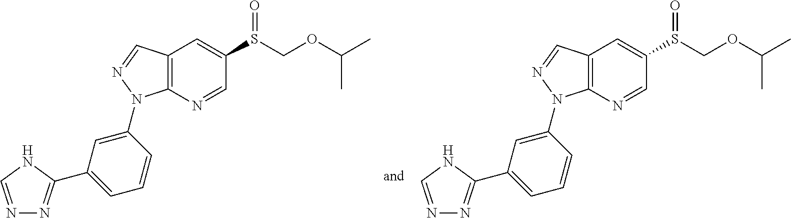 |
| 3A and 3B | 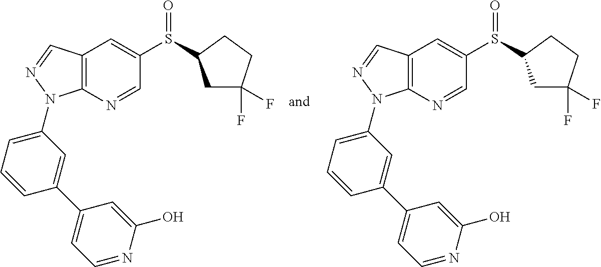 [SMILES: O=[S@](c1cnc2c(cnn2-c2cccc(-c3ccnc(O)c3)c2)c1)[C@H]1CCC(F)(F)C1 ; O=[S@](c1cnc2c(cnn2-c2cccc(-c3ccnc(O)c3)c2)c1)[C@@H]1CCC(F)(F)C1] |
| 4A and 4B | 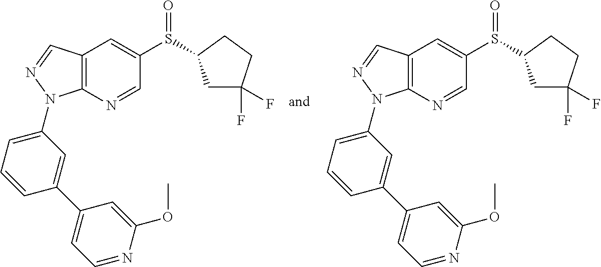 [SMILES: COc1cc(-c2cccc(-n3ncc4cc(S(=O)[C@@H]5CCC(F)(F)C5)cnc43)c2)ccn1 ; COc1cc(-c2cccc(-n3ncc4cc([S@](=O)[C@@H]5CCC(F)(F)C5)cnc43)c2)ccn1] |
| 5A, 5B, 5C, and 5D | 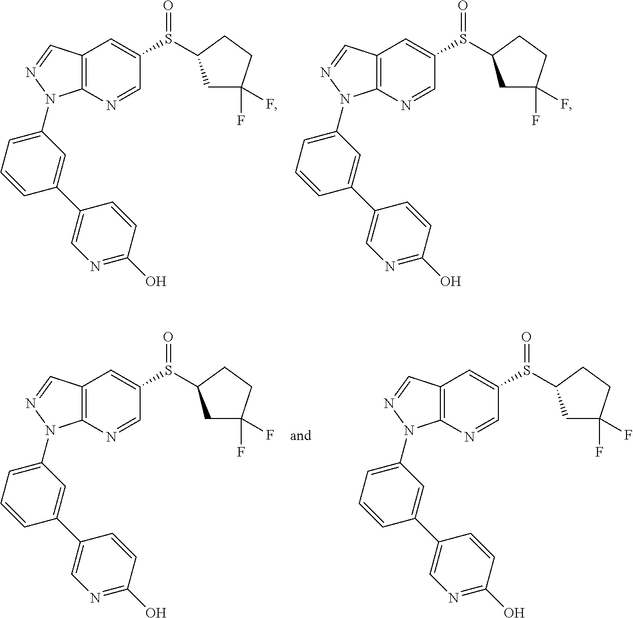 |
| 6 | 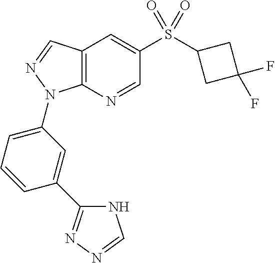 [SMILES: O=S(=O)(c1cnc2c(cnn2-c2cccc(-c3nnc[nH]3)c2)c1)C1CC(F)(F)C1] |
| 7A and 7B | 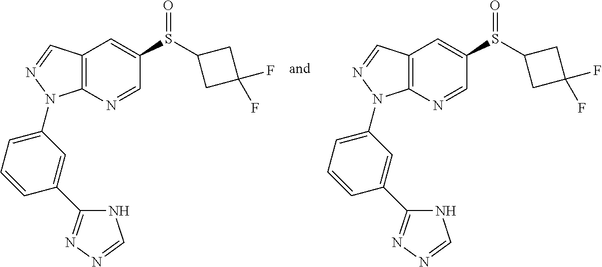 |
| 8A, 8B, 8C, and 8D | 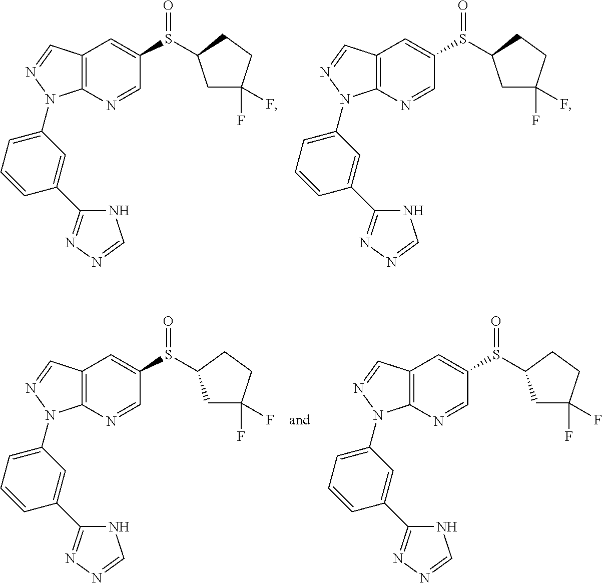 |
| 9A and 9B | 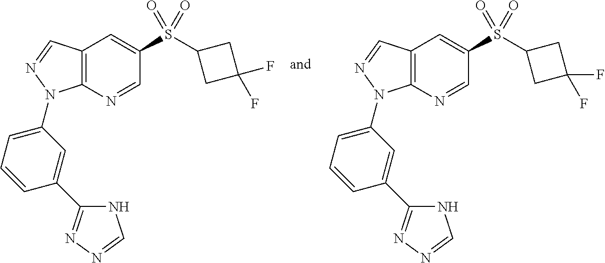 |
| 10A and 10B | 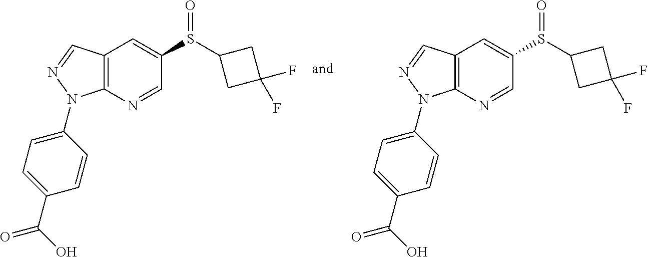 |
| 11A, 11B, 11C, and 11D | 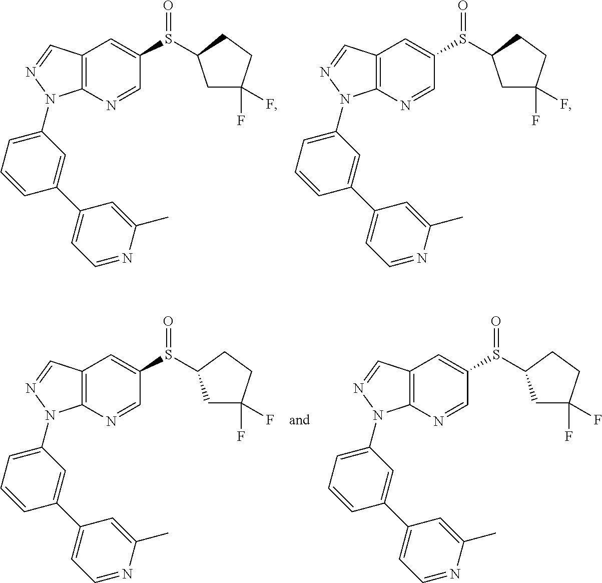 |
| 12A and 12B | 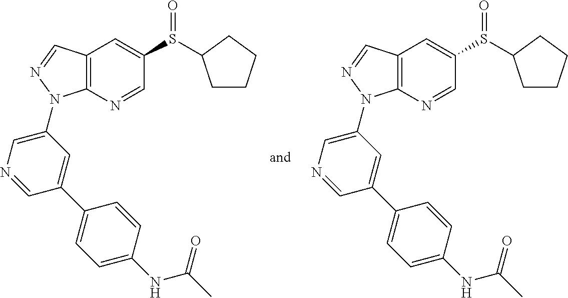 |
| 13A and 13B | 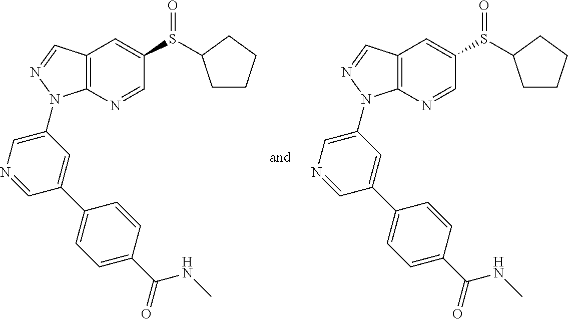 |
| 14A and 14B | 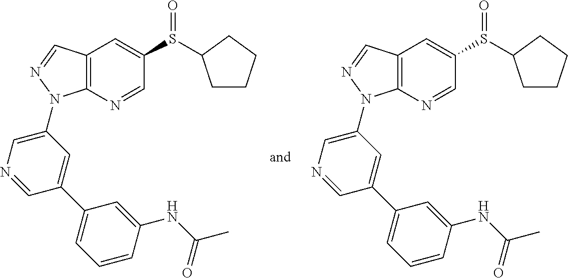 |
| 15 | 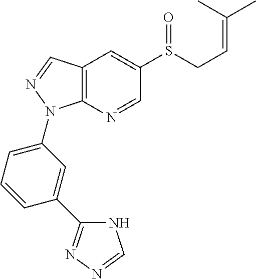 [SMILES: CC(C)=CCS(=O)c1cnc2c(cnn2-c2cccc(-c3nnc[nH]3)c2)c1] |
| 16A and 16B | 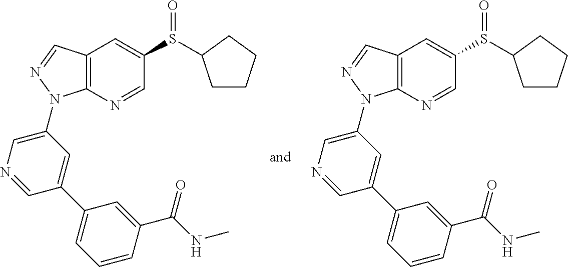 |
| 17A and 17B | 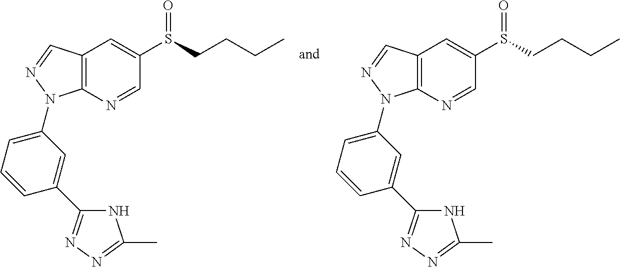 [SMILES: CCCC[S@](=O)c1cnc2c(cnn2-c2cccc(-c3nnc(C)[nH]3)c2)c1 ; CCCC[S@@](=O)c1cnc2c(cnn2-c2cccc(-c3nnc(C)[nH]3)c2)c1] |
| 18A and 18B | 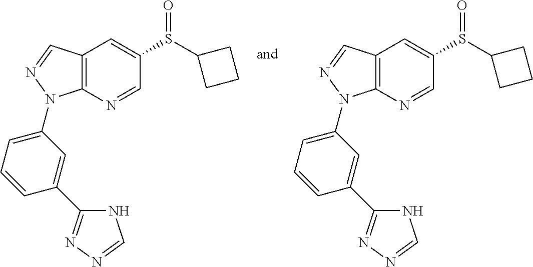 |
| 19A and 19B | 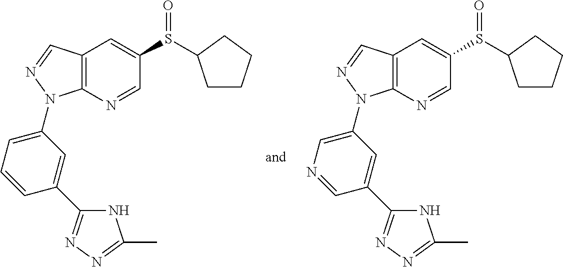 |
| 20A and 20B | 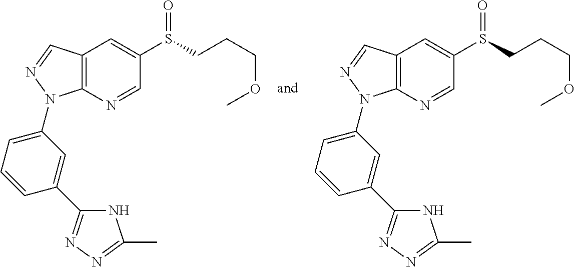 [SMILES: COCCC[S@@](=O)c1cnc2c(cnn2-c2cccc(-c3nnc(C)[nH]3)c2)c1 ; COCCC[S@](=O)c1cnc2c(cnn2-c2cccc(-c3nnc(C)[nH]3)c2)c1] |
| 21A and 21B | 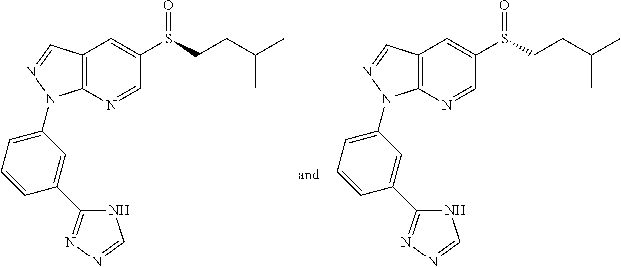 [SMILES: CC(C)CC[S@](=O)c1cnc2c(cnn2-c2cccc(-c3nnc[nH]3)c2)c1 ; CC(C)CC[S@@](=O)c1cnc2c(cnn2-c2cccc(-c3nnc[nH]3)c2)c1] |
| 22A and 22B | 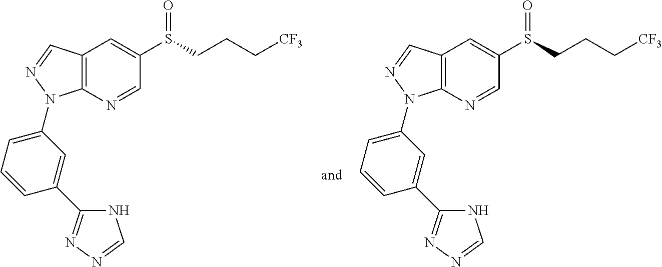 [SMILES: O=[S@](CCCC(F)(F)F)c1cnc2c(cnn2-c2cccc(-c3nnc[nH]3)c2)c1 ; O=[S@@](CCCC(F)(F)F)c1cnc2c(cnn2-c2cccc(-c3nnc[nH]3)c2)c1] |
| 23A and 23B | 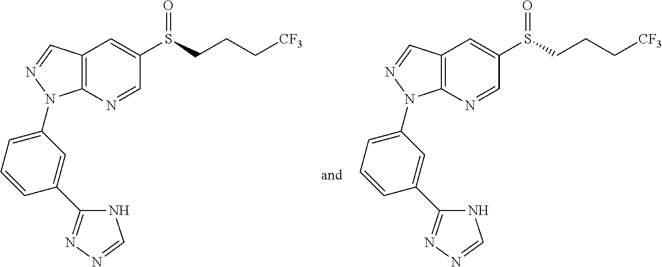 [SMILES: O=[S@@](CCCC(F)(F)F)c1cnc2c(cnn2-c2cccc(-c3nnc[nH]3)c2)c1 ; O=[S@](CCCC(F)(F)F)c1cnc2c(cnn2-c2cccc(-c3nnc[nH]3)c2)c1] |
| 24A and 24B | 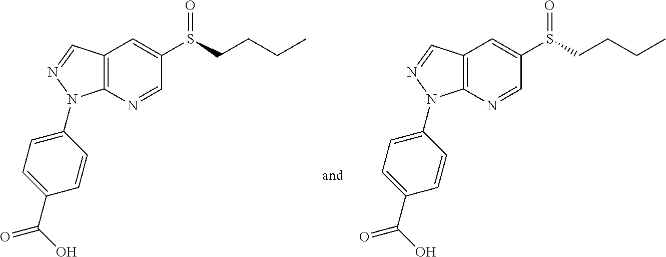 [SMILES: CCCC[S@](=O)c1cnc2c(cnn2-c2ccc(C(=O)O)cc2)c1 ; CCCC[S@@](=O)c1cnc2c(cnn2-c2ccc(C(=O)O)cc2)c1] |
| 25 | 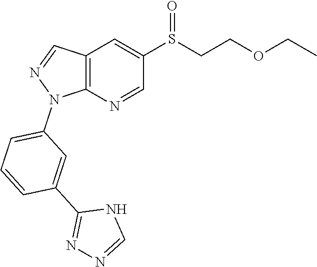 [SMILES: CCOCCS(=O)c1cnc2c(cnn2-c2cccc(-c3nnc[nH]3)c2)c1] |
| 26A and 26B | 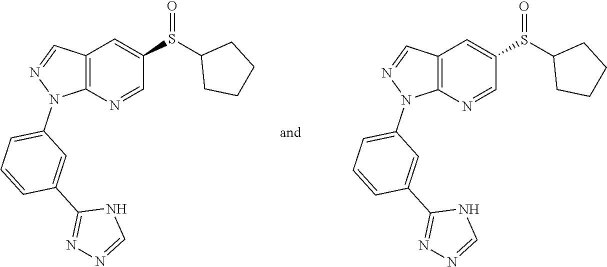 |
| 27A and 27B | 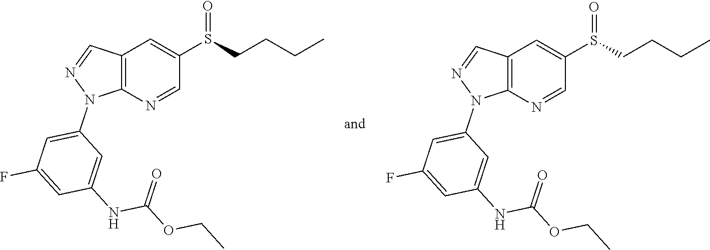 [SMILES: CCCC[S@](=O)c1cnc2c(cnn2-c2cc(F)cc(NC(=O)OCC)c2)c1 ; CCCC[S@@](=O)c1cnc2c(cnn2-c2cc(F)cc(NC(=O)OCC)c2)c1] |
| 28 | 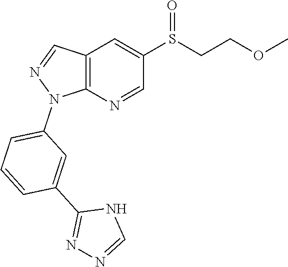 [SMILES: COCCS(=O)c1cnc2c(cnn2-c2cccc(-c3nnc[nH]3)c2)c1] |
| 29 | 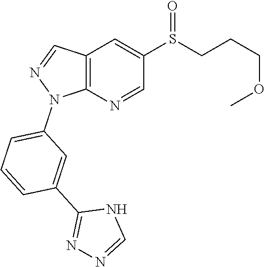 [SMILES: COCCCS(=O)c1cnc2c(cnn2-c2cccc(-c3nnc[nH]3)c2)c1] |
| 30 | 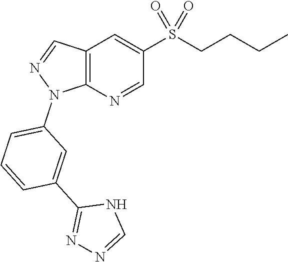 [SMILES: CCCCS(=O)(=O)c1cnc2c(cnn2-c2cccc(-c3nnc[nH]3)c2)c1] |
| 31 | 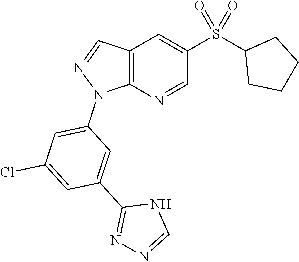 [SMILES: O=S(=O)(c1cnc2c(cnn2-c2cc(Cl)cc(-c3nnc[nH]3)c2)c1)C1CCCC1] |
| 32 | 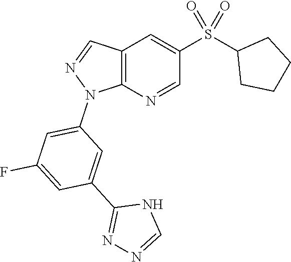 [SMILES: O=S(=O)(c1cnc2c(cnn2-c2cc(F)cc(-c3nnc[nH]3)c2)c1)C1CCCC1] |
| 33 | 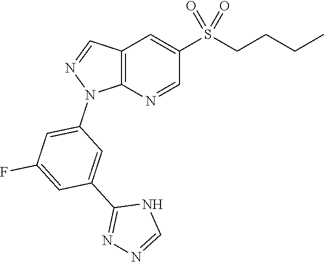 [SMILES: CCCCS(=O)(=O)c1cnc2c(cnn2-c2cc(F)cc(-c3nnc[nH]3)c2)c1] |
| 34 | 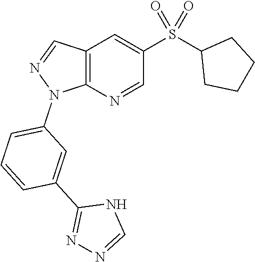 [SMILES: O=S(=O)(c1cnc2c(cnn2-c2cccc(-c3nnc[nH]3)c2)c1)C1CCCC1] |
| 35 | 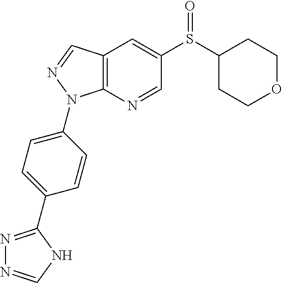 [SMILES: O=S(c1cnc2c(cnn2-c2ccc(-c3nnc[nH]3)cc2)c1)C1CCOCC1] |
| 36A and 36B | 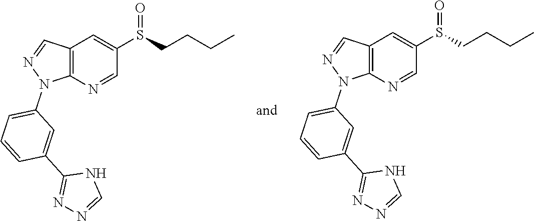 [SMILES: CCCC[S@](=O)c1cnc2c(cnn2-c2cccc(-c3nnc[nH]3)c2)c1 ; CCCC[S@@](=O)c1cnc2c(cnn2-c2cccc(-c3nnc[nH]3)c2)c1] |
| 37 | 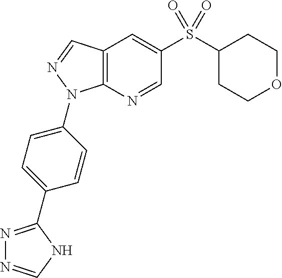 [SMILES: O=S(=O)(c1cnc2c(cnn2-c2ccc(-c3nnc[nH]3)cc2)c1)C1CCOCC1] |
| 38 | 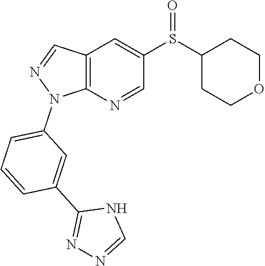 [SMILES: O=S(c1cnc2c(cnn2-c2cccc(-c3nnc[nH]3)c2)c1)C1CCOCC1] |
| 39 | 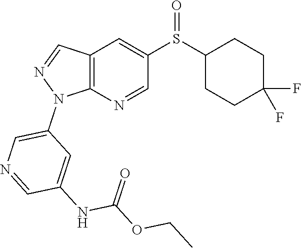 [SMILES: CCOC(=O)Nc1cncc(-n2ncc3cc(S(=O)C4CCC(F)(F)CC4)cnc32)c1] |
| 40 | 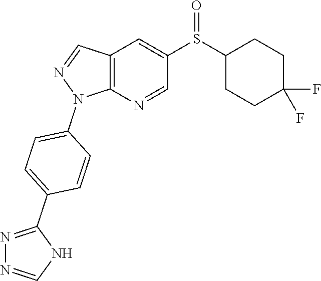 [SMILES: O=S(c1cnc2c(cnn2-c2ccc(-c3nnc[nH]3)cc2)c1)C1CCC(F)(F)CC1] |
| 41 | 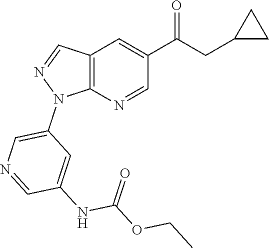 [SMILES: CCOC(=O)Nc1cncc(-n2ncc3cc(C(=O)CC4CC4)cnc32)c1] |
| 42 | 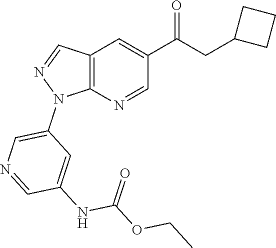 [SMILES: CCOC(=O)Nc1cncc(-n2ncc3cc(C(=O)CC4CCC4)cnc32)c1] |
| 43 | 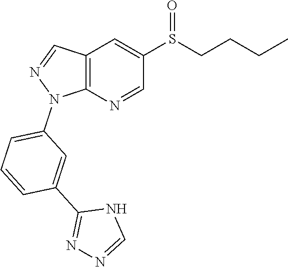 [SMILES: CCCCS(=O)c1cnc2c(cnn2-c2cccc(-c3nnc[nH]3)c2)c1] |
| 44 | 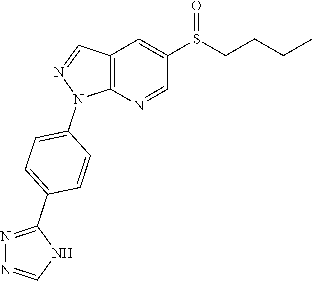 [SMILES: CCCCS(=O)c1cnc2c(cnn2-c2ccc(-c3nnc[nH]3)cc2)c1] |
| 45 | 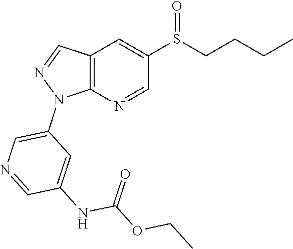 [SMILES: CCCCS(=O)c1cnc2c(cnn2-c2cncc(NC(=O)OCC)c2)c1] |
| 46 | 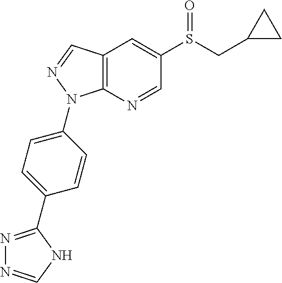 [SMILES: O=S(CC1CC1)c1cnc2c(cnn2-c2ccc(-c3nnc[nH]3)cc2)c1] |
| 47 | 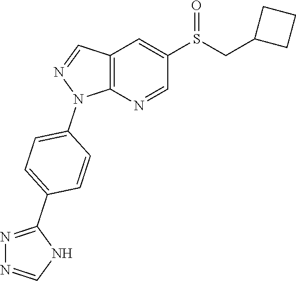 [SMILES: O=S(CC1CCC1)c1cnc2c(cnn2-c2ccc(-c3nnc[nH]3)cc2)c1] |
| 48A and 48B | 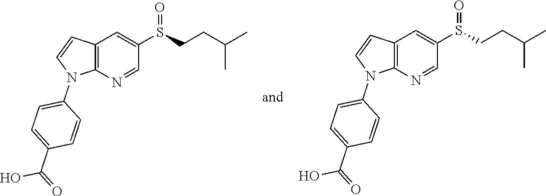 [SMILES: CC(C)CC[S@](=O)c1cnc2c(ccn2-c2ccc(C(=O)O)cc2)c1 ; CC(C)CC[S@@](=O)c1cnc2c(ccn2-c2ccc(C(=O)O)cc2)c1] |
| 49A and 49B | 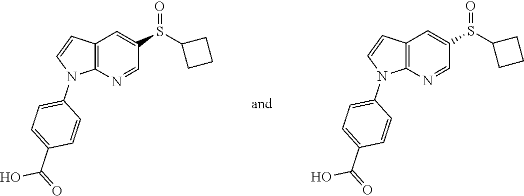 [SMILES: O=C(O)c1ccc(-n2ccc3cc([S@@](=O)C4CCC4)cnc32)cc1 ; O=C(O)c1ccc(-n2ccc3cc([S@](=O)C4CCC4)cnc32)cc1] |
| 50A and 50B | 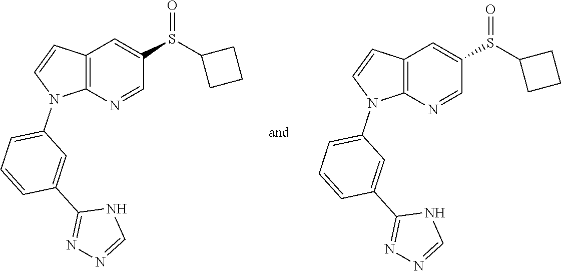 |
| 51A and 51B | 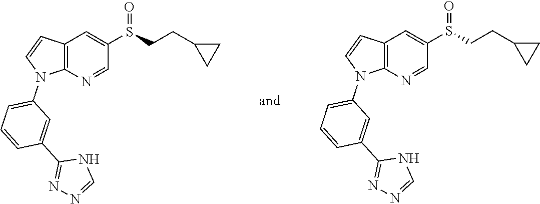 [SMILES: O=[S@@](CCC1CC1)c1cnc2c(ccn2-c2cccc(-c3nnc[nH]3)c2)c1 ; O=[S@](CCC1CC1)c1cnc2c(ccn2-c2cccc(-c3nnc[nH]3)c2)c1] |
| 52A and 52B | 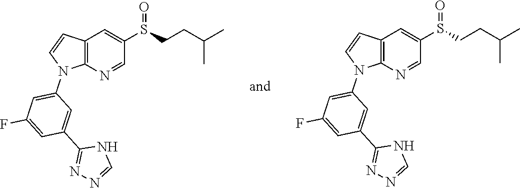 [SMILES: CC(C)CC[S@](=O)c1cnc2c(ccn2-c2cc(F)cc(-c3nnc[nH]3)c2)c1 ; CC(C)CC[S@@](=O)c1cnc2c(ccn2-c2cc(F)cc(-c3nnc[nH]3)c2)c1] |
| 53A and 53B | 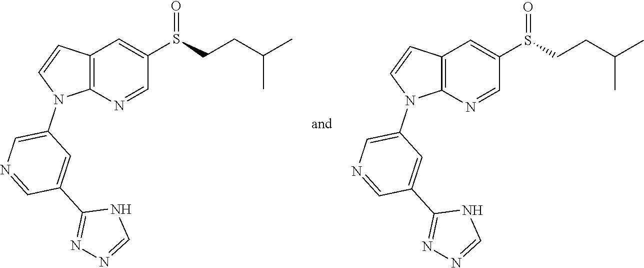 [SMILES: CC(C)CC[S@](=O)c1cnc2c(ccn2-c2cncc(-c3nnc[nH]3)c2)c1 ; CC(C)CC[S@@](=O)c1cnc2c(ccn2-c2cncc(-c3nnc[nH]3)c2)c1] |
| 54A and 54B | 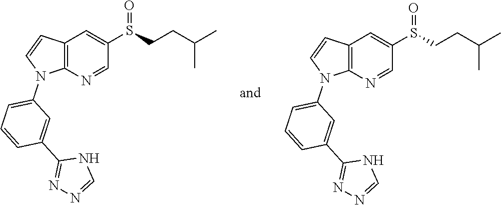 [SMILES: CC(C)CC[S@](=O)c1cnc2c(ccn2-c2cccc(-c3nnc[nH]3)c2)c1 ; CC(C)CC[S@@](=O)c1cnc2c(ccn2-c2cccc(-c3nnc[nH]3)c2)c1] |
| 55 | 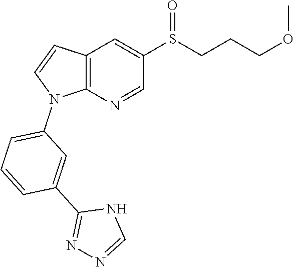 [SMILES: COCCCS(=O)c1cnc2c(ccn2-c2cccc(-c3nnc[nH]3)c2)c1] |
| 56A and 56B | 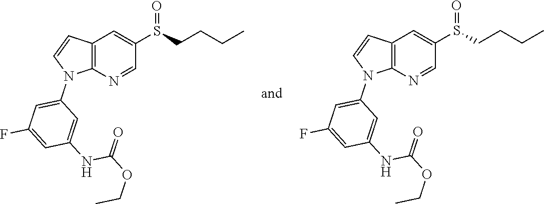 [SMILES: CCCC[S@](=O)c1cnc2c(ccn2-c2cc(F)cc(NC(=O)OCC)c2)c1 ; CCCC[S@@](=O)c1cnc2c(ccn2-c2cc(F)cc(NC(=O)OCC)c2)c1] |
| 57 | 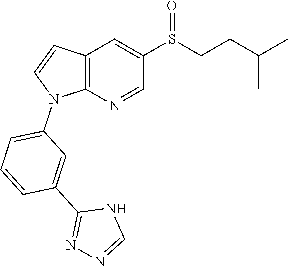 [SMILES: CC(C)CCS(=O)c1cnc2c(ccn2-c2cccc(-c3nnc[nH]3)c2)c1] |
| 58 | 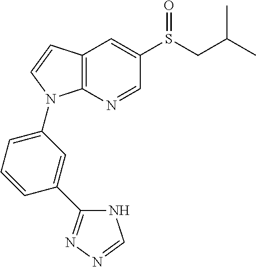 [SMILES: CC(C)CS(=O)c1cnc2c(ccn2-c2cccc(-c3nnc[nH]3)c2)c1] |
| 59A and 59B | 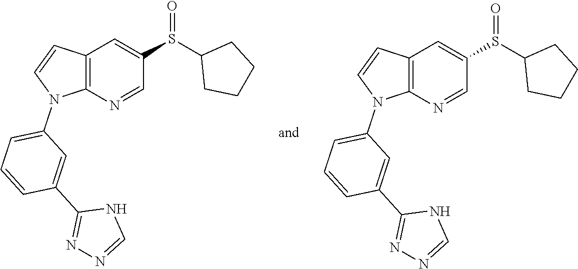 |
| 60 | 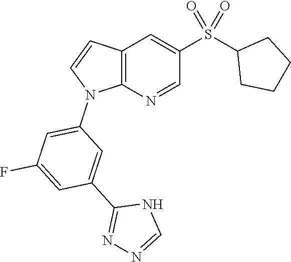 [SMILES: O=S(=O)(c1cnc2c(ccn2-c2cc(F)cc(-c3nnc[nH]3)c2)c1)C1CCCC1] |
| 61 | 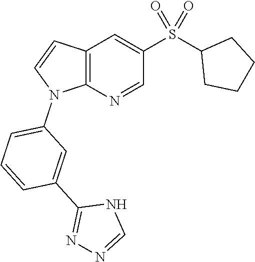 [SMILES: O=S(=O)(c1cnc2c(ccn2-c2cccc(-c3nnc[nH]3)c2)c1)C1CCCC1] |
| 62 | 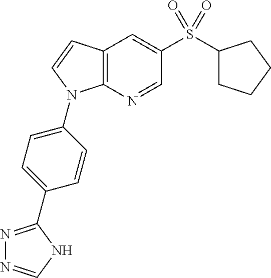 [SMILES: O=S(=O)(c1cnc2c(ccn2-c2ccc(-c3nnc[nH]3)cc2)c1)C1CCCC1] |
| 63 | 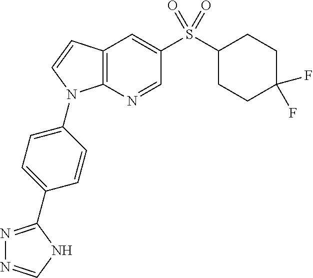 [SMILES: O=S(=O)(c1cnc2c(ccn2-c2ccc(-c3nnc[nH]3)cc2)c1)C1CCC(F)(F)CC1] |
| 64 | 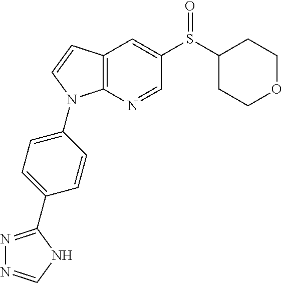 [SMILES: O=S(c1cnc2c(ccn2-c2ccc(-c3nnc[nH]3)cc2)c1)C1CCOCC1] |
| 65 | 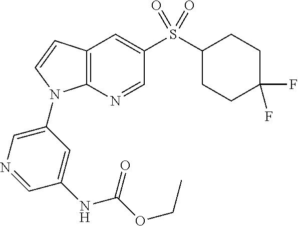 [SMILES: CCOC(=O)Nc1cncc(-n2ccc3cc(S(=O)(=O)C4CCC(F)(F)CC4)cnc32)c1] |
| 66 | 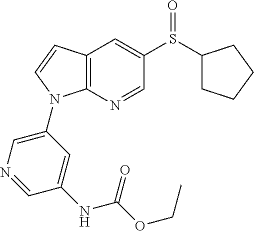 [SMILES: CCOC(=O)Nc1cncc(-n2ccc3cc(S(=O)C4CCCC4)cnc32)c1] |
| 67 | 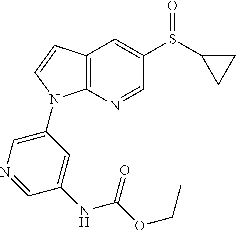 [SMILES: CCOC(=O)Nc1cncc(-n2ccc3cc(S(=O)C4CC4)cnc32)c1] |
| 68 | 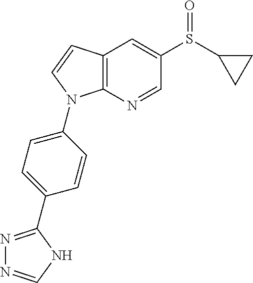 [SMILES: O=S(c1cnc2c(ccn2-c2ccc(-c3nnc[nH]3)cc2)c1)C1CC1] |
| 69 | 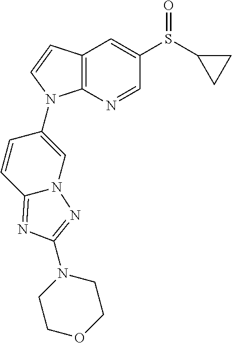 [SMILES: O=S(c1cnc2c(ccn2-c2ccc3nc(N4CCOCC4)nn3c2)c1)C1CC1] |
| 70 | 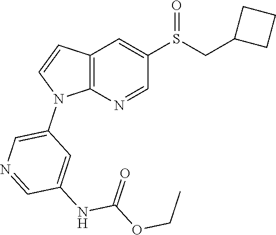 [SMILES: CCOC(=O)Nc1cncc(-n2ccc3cc(S(=O)CC4CCC4)cnc32)c1] |
| 71 | 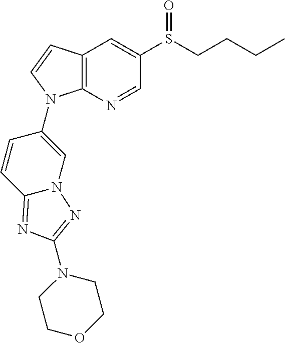 [SMILES: CCCCS(=O)c1cnc2c(ccn2-c2ccc3nc(N4CCOCC4)nn3c2)c1] |
| 72 | 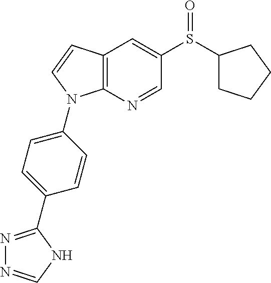 [SMILES: O=S(c1cnc2c(ccn2-c2ccc(-c3nnc[nH]3)cc2)c1)C1CCCC1] |
| 73 | 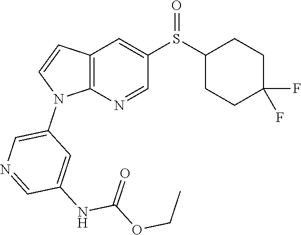 [SMILES: CCOC(=O)Nc1cncc(-n2ccc3cc(S(=O)C4CCC(F)(F)CC4)cnc32)c1] |
| 74 | 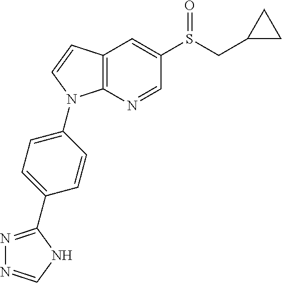 [SMILES: O=S(CC1CC1)c1cnc2c(ccn2-c2ccc(-c3nnc[nH]3)cc2)c1] |
| 75 | 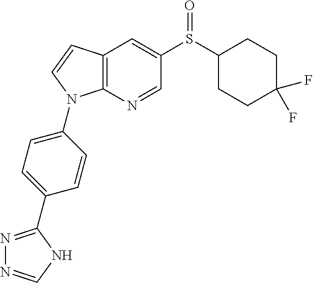 [SMILES: O=S(c1cnc2c(ccn2-c2ccc(-c3nnc[nH]3)cc2)c1)C1CCC(F)(F)CC1] |
| 76 | 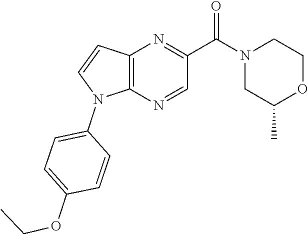 [SMILES: CCOc1ccc(-n2ccc3nc(C(=O)N4CCO[C@H](C)C4)cnc32)cc1] |
| 77 | 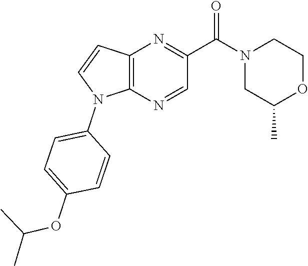 [SMILES: CC(C)Oc1ccc(-n2ccc3nc(C(=O)N4CCO[C@H](C)C4)cnc32)cc1] |
| 78 | 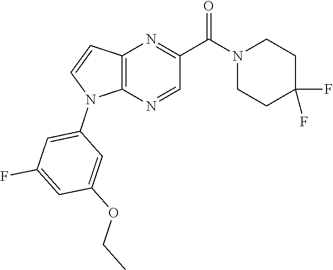 [SMILES: CCOc1cc(F)cc(-n2ccc3nc(C(=O)N4CCC(F)(F)CC4)cnc32)c1] |
| 79 | 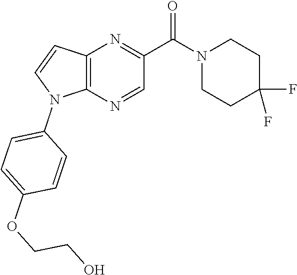 [SMILES: O=C(c1cnc2c(ccn2-c2ccc(OCCO)cc2)n1)N1CCC(F)(F)CC1] |
| 80 | 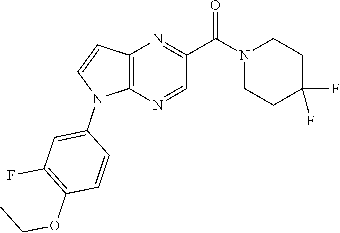 [SMILES: CCOc1ccc(-n2ccc3nc(C(=O)N4CCC(F)(F)CC4)cnc32)cc1F] |
| 81 | 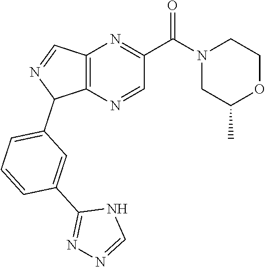 [SMILES: C[C@@H]1CN(C(=O)c2cnc3c(n2)C=NC3c2cccc(-c3nnc[nH]3)c2)CCO1] |
| 82 | 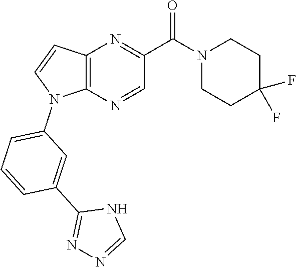 [SMILES: O=C(c1cnc2c(ccn2-c2cccc(-c3nnc[nH]3)c2)n1)N1CCC(F)(F)CC1] |
| 83 | 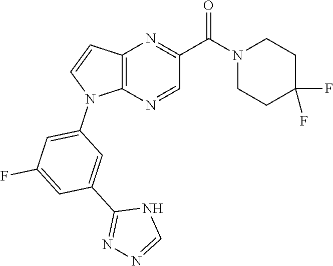 [SMILES: O=C(c1cnc2c(ccn2-c2cc(F)cc(-c3nnc[nH]3)c2)n1)N1CCC(F)(F)CC1] |
| 84 | 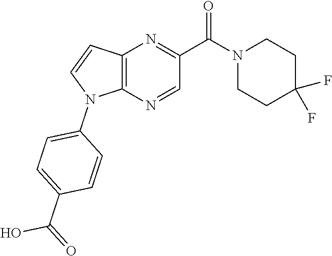 [SMILES: O=C(O)c1ccc(-n2ccc3nc(C(=O)N4CCC(F)(F)CC4)cnc32)cc1] |
| 85 | 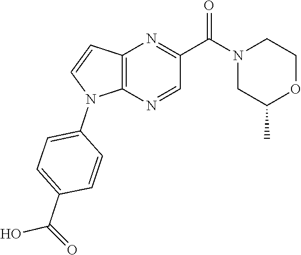 [SMILES: C[C@@H]1CN(C(=O)c2cnc3c(ccn3-c3ccc(C(=O)O)cc3)n2)CCO1] |
| 86 | 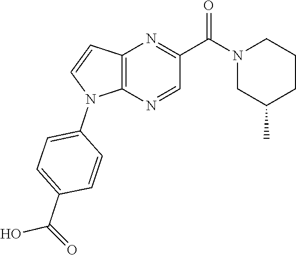 [SMILES: C[C@H]1CCCN(C(=O)c2cnc3c(ccn3-c3ccc(C(=O)O)cc3)n2)C1] |
| 87 | 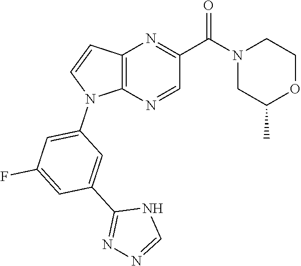 [SMILES: C[C@@H]1CN(C(=O)c2cnc3c(ccn3-c3cc(F)cc(-c4nnc[nH]4)c3)n2)CCO1] |
| 88A and 88B | 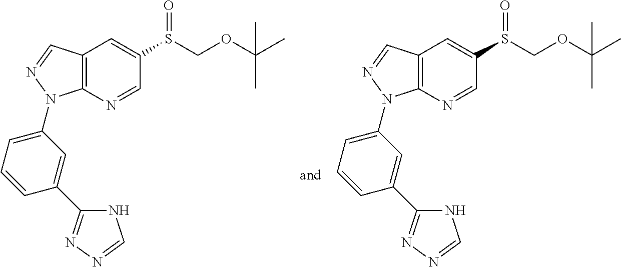 [SMILES: CC(C)(C)OC[S@@](=O)c1cnc2c(cnn2-c2cccc(-c3nnc[nH]3)c2)c1 ; CC(C)(C)OC[S@](=O)c1cnc2c(cnn2-c2cccc(-c3nnc[nH]3)c2)c1] |
| 90A and 90B | 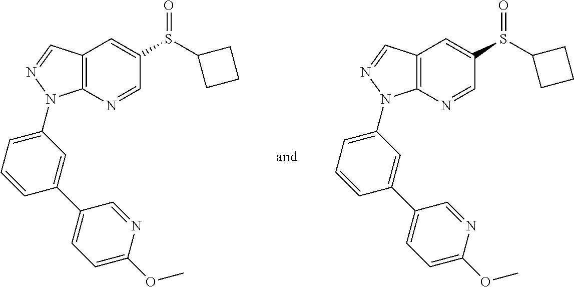 [SMILES: COc1ccc(-c2cccc(-n3ncc4cc([S@](=O)C5CCC5)cnc43)c2)cn1 ; COc1ccc(-c2cccc(-n3ncc4cc([S@@](=O)C5CCC5)cnc43)c2)cn1] |
| 91A and 91B | 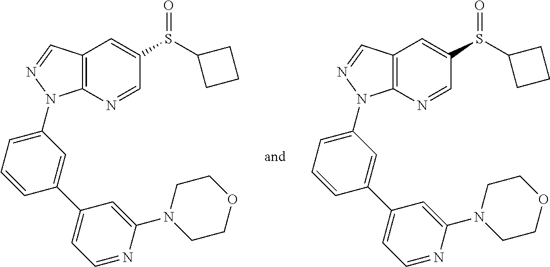 [SMILES: O=[S@@](c1cnc2c(cnn2-c2cccc(-c3ccnc(N4CCOCC4)c3)c2)c1)C1CCC1 ; O=[S@](c1cnc2c(cnn2-c2cccc(-c3ccnc(N4CCOCC4)c3)c2)c1)C1CCC1] |
| 92A and 92B | 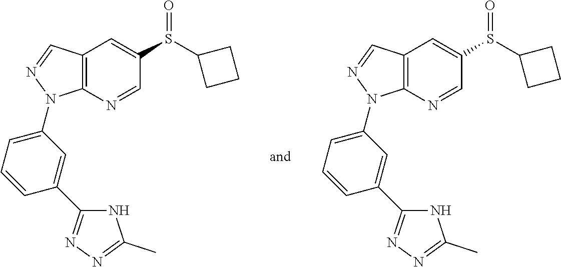 [SMILES: Cc1nnc(-c2cccc(-n3ncc4cc([S@@](=O)C5CCC5)cnc43)c2)[nH]1 ; Cc1nnc(-c2cccc(-n3ncc4cc([S@](=O)C5CCC5)cnc43)c2)[nH]1] |
| 93A and 93B | 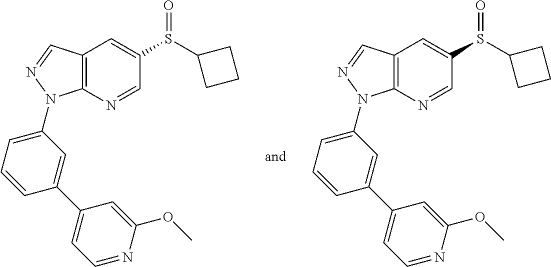 [SMILES: COc1cc(-c2cccc(-n3ncc4cc([S@](=O)C5CCC5)cnc43)c2)ccn1 ; COc1cc(-c2cccc(-n3ncc4cc([S@@](=O)C5CCC5)cnc43)c2)ccn1] |
| 94A and 94B | 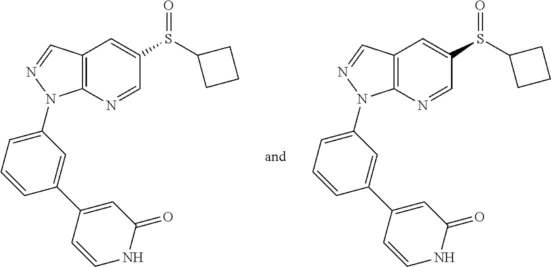 [SMILES: O=c1cc(-c2cccc(-n3ncc4cc([S@](=O)C5CCC5)cnc43)c2)cc[nH]1 ; O=c1cc(-c2cccc(-n3ncc4cc([S@@](=O)C5CCC5)cnc43)c2)cc[nH]1] |
| 95A and 95B | 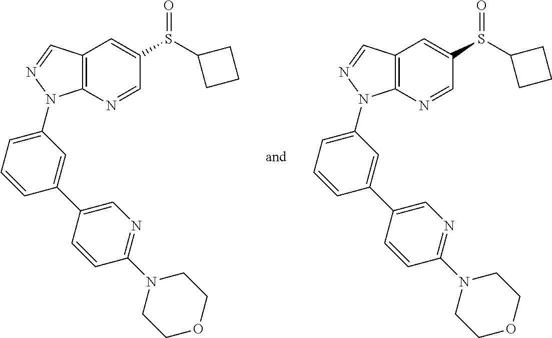 [SMILES: O=[S@@](c1cnc2c(cnn2-c2cccc(-c3ccc(N4CCOCC4)nc3)c2)c1)C1CCC1 ; O=[S@](c1cnc2c(cnn2-c2cccc(-c3ccc(N4CCOCC4)nc3)c2)c1)C1CCC1] |
| 96A and 96B | 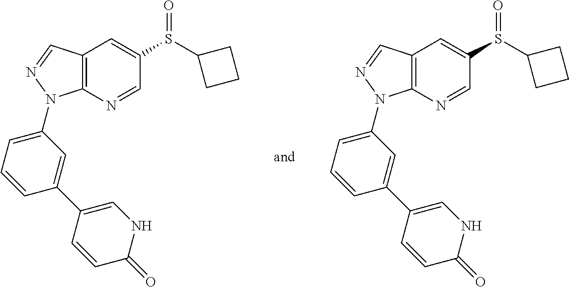 [SMILES: O=c1ccc(-c2cccc(-n3ncc4cc([S@](=O)C5CCC5)cnc43)c2)c[nH]1 ; O=c1ccc(-c2cccc(-n3ncc4cc([S@@](=O)C5CCC5)cnc43)c2)c[nH]1] |
| 97A and 97B | 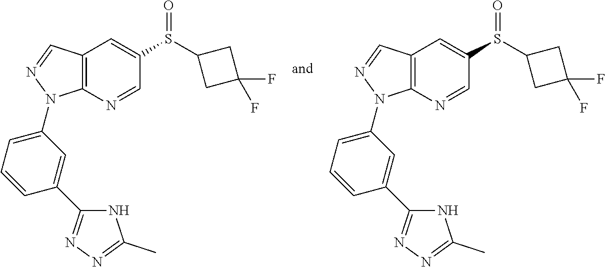 [SMILES: Cc1nnc(-c2cccc(-n3ncc4cc([S@](=O)C5CC(F)(F)C5)cnc43)c2)[nH]1 ; Cc1nnc(-c2cccc(-n3ncc4cc([S@@](=O)C5CC(F)(F)C5)cnc43)c2)[nH]1] |
| 98A and 98B | 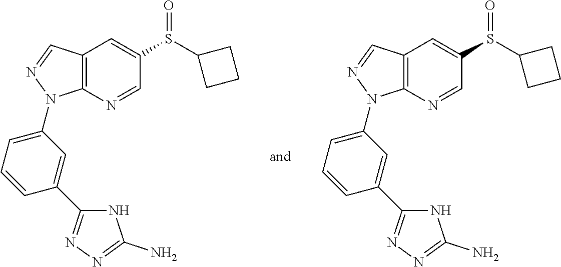 [SMILES: Nc1nnc(-c2cccc(-n3ncc4cc([S@](=O)C5CCC5)cnc43)c2)[nH]1 ; Nc1nnc(-c2cccc(-n3ncc4cc([S@@](=O)C5CCC5)cnc43)c2)[nH]1] |
| 99A and 99B | 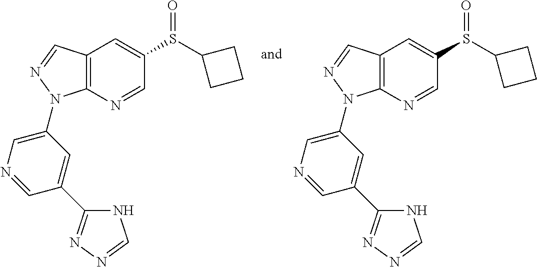 |
| 100A and 100B | 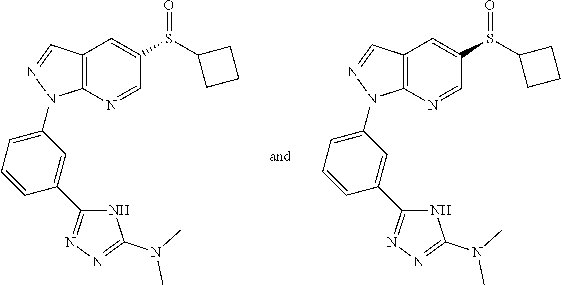 [SMILES: CN(C)c1nnc(-c2cccc(-n3ncc4cc([S@](=O)C5CCC5)cnc43)c2)[nH]1 ; CN(C)c1nnc(-c2cccc(-n3ncc4cc([S@@](=O)C5CCC5)cnc43)c2)[nH]1] |
| 101A and 101B | 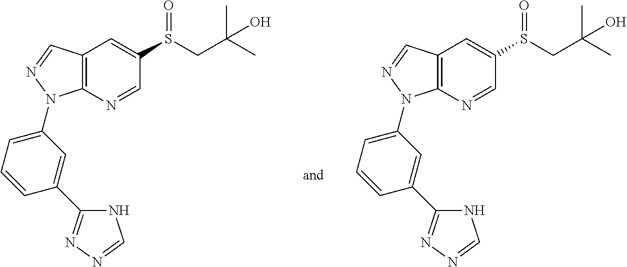 [SMILES: CC(C)(O)C[S@](=O)c1cnc2c(cnn2-c2cccc(-c3nnc[nH]3)c2)c1 ; CC(C)(O)C[S@@](=O)c1cnc2c(cnn2-c2cccc(-c3nnc[nH]3)c2)c1] |
| 102A and 102B | 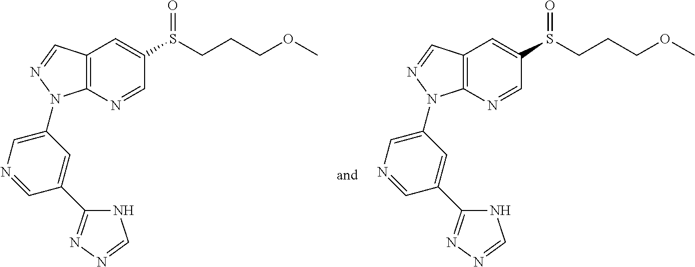 [SMILES: COCCC[S@@](=O)c1cnc2c(cnn2-c2cncc(-c3nnc[nH]3)c2)c1 ; COCCC[S@](=O)c1cnc2c(cnn2-c2cncc(-c3nnc[nH]3)c2)c1] |
| 103A and 103B | 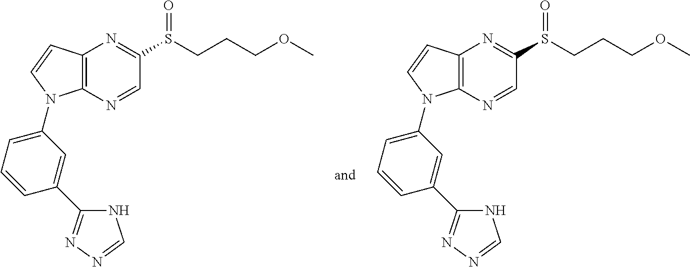 [SMILES: COCCC[S@@](=O)c1cnc2c(ccn2-c2cccc(-c3nnc[nH]3)c2)n1 ; COCCC[S@](=O)c1cnc2c(ccn2-c2cccc(-c3nnc[nH]3)c2)n1] |
| 104A and 104B | 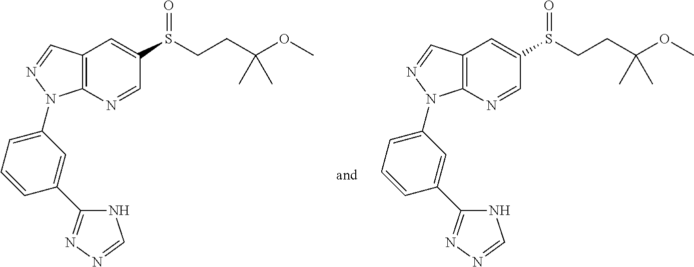 [SMILES: COC(C)(C)CC[S@](=O)c1cnc2c(cnn2-c2cccc(-c3nnc[nH]3)c2)c1 ; COC(C)(C)CC[S@@](=O)c1cnc2c(cnn2-c2cccc(-c3nnc[nH]3)c2)c1] |
| 105A, 105B, 105C, and 105D | 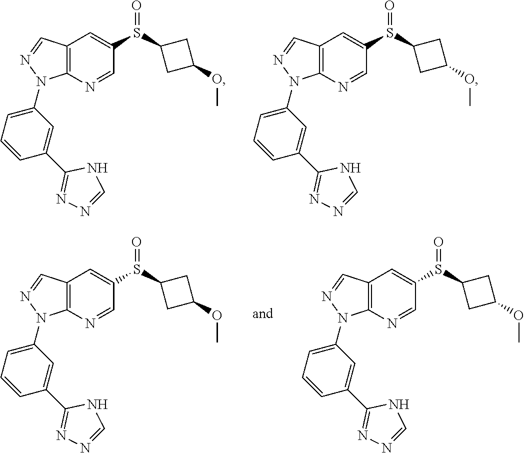 |
| 107A and 107B | 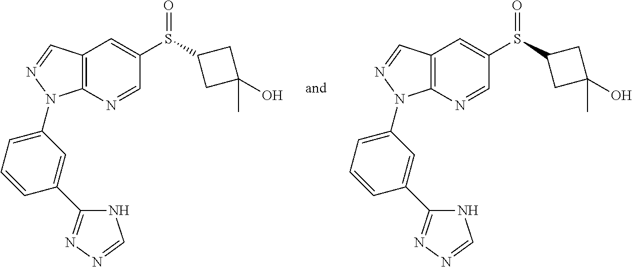 [SMILES: CC1(O)CC([S@@](=O)c2cnc3c(cnn3-c3cccc(-c4nnc[nH]4)c3)c2)C1 ; CC1(O)CC([S@](=O)c2cnc3c(cnn3-c3cccc(-c4nnc[nH]4)c3)c2)C1] |
| 108A and 109B | 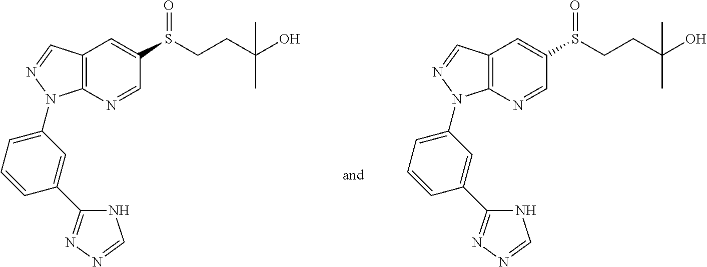 [SMILES: CC(C)(O)CC[S@](=O)c1cnc2c(cnn2-c2cccc(-c3nnc[nH]3)c2)c1 ; CC(C)(O)CC[S@@](=O)c1cnc2c(cnn2-c2cccc(-c3nnc[nH]3)c2)c1] |
| 109A, 109B, 109C, and 109D | 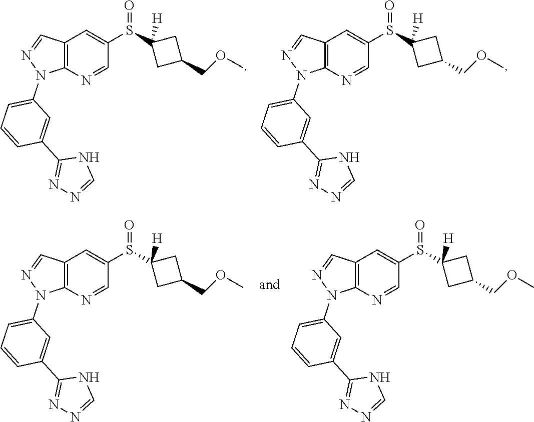 [SMILES: COC[C@H]1C[C@@H]([S@](=O)c2cnc3c(cnn3-c3cccc(-c4nnc[nH]4)c3)c2)C1 ; COC[C@H]1C[C@H]([S@](=O)c2cnc3c(cnn3-c3cccc(-c4nnc[nH]4)c3)c2)C1 ; COC[C@H]1C[C@H]([S@@](=O)c2cnc3c(cnn3-c3cccc(-c4nnc[nH]4)c3)c2)C1 ; COC[C@H]1C[C@@H]([S@@](=O)c2cnc3c(cnn3-c3cccc(-c4nnc[nH]4)c3)c2)C1] |
| 110A and 110B | 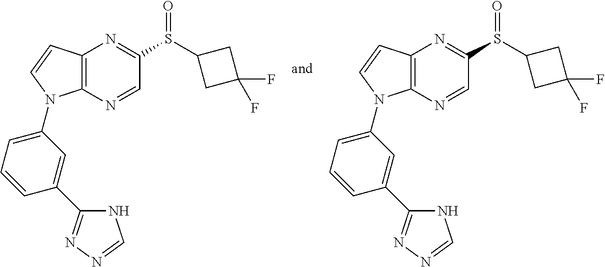 [SMILES: O=[S@@](c1cnc2c(ccn2-c2cccc(-c3nnc[nH]3)c2)n1)C1CC(F)(F)C1 ; O=[S@](c1cnc2c(ccn2-c2cccc(-c3nnc[nH]3)c2)n1)C1CC(F)(F)C1] |
| 111A and 111B | 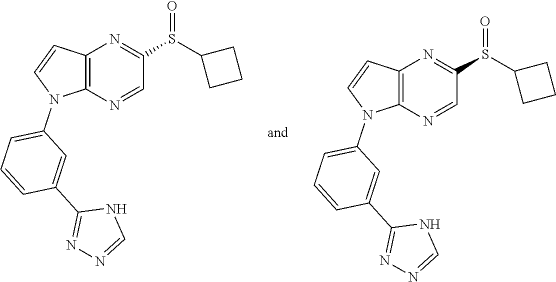 [SMILES: O=[S@@](c1cnc2c(ccn2-c2cccc(-c3nnc[nH]3)c2)n1)C1CCC1 ; O=[S@](c1cnc2c(ccn2-c2cccc(-c3nnc[nH]3)c2)n1)C1CCC1] |
| 112A and 112B | 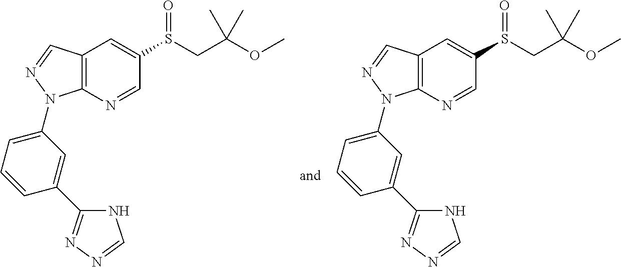 [SMILES: COC(C)(C)C[S@@](=O)c1cnc2c(cnn2-c2cccc(-c3nnc[nH]3)c2)c1 ; COC(C)(C)C[S@](=O)c1cnc2c(cnn2-c2cccc(-c3nnc[nH]3)c2)c1] |
| 113A and 113B | 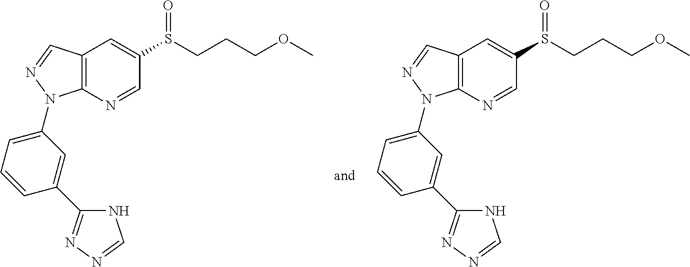 [SMILES: COCCC[S@@](=O)c1cnc2c(cnn2-c2cccc(-c3nnc[nH]3)c2)c1 ; COCCC[S@](=O)c1cnc2c(cnn2-c2cccc(-c3nnc[nH]3)c2)c1] |
| 114A and 114B | 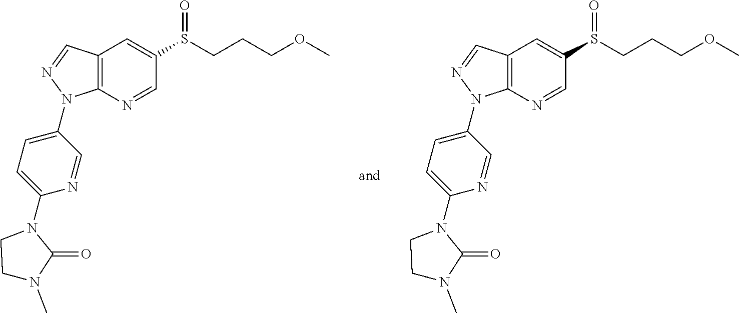 [SMILES: COCCC[S@@](=O)c1cnc2c(cnn2-c2ccc(N3CCN(C)C3=O)nc2)c1 ; COCCC[S@](=O)c1cnc2c(cnn2-c2ccc(N3CCN(C)C3=O)nc2)c1] |
| 115A and 115B | 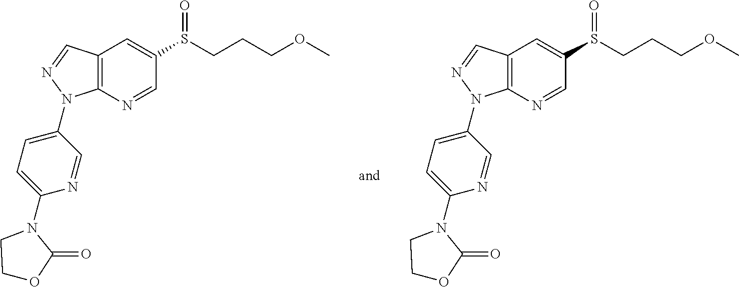 [SMILES: COCCC[S@@](=O)c1cnc2c(cnn2-c2ccc(N3CCOC3=O)nc2)c1 ; COCCC[S@](=O)c1cnc2c(cnn2-c2ccc(N3CCOC3=O)nc2)c1] |
| 116A and 116B | 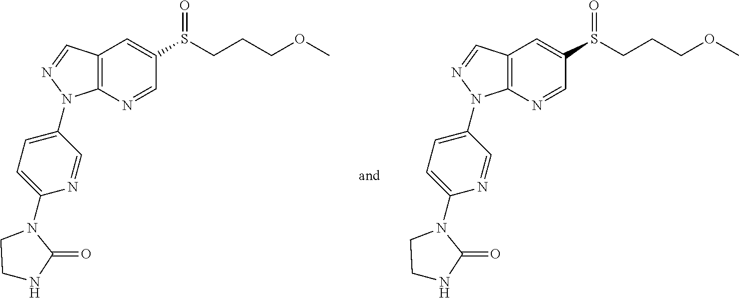 [SMILES: COCCC[S@@](=O)c1cnc2c(cnn2-c2ccc(N3CCNC3=O)nc2)c1 ; COCCC[S@](=O)c1cnc2c(cnn2-c2ccc(N3CCNC3=O)nc2)c1] |
| 117A and 117B | 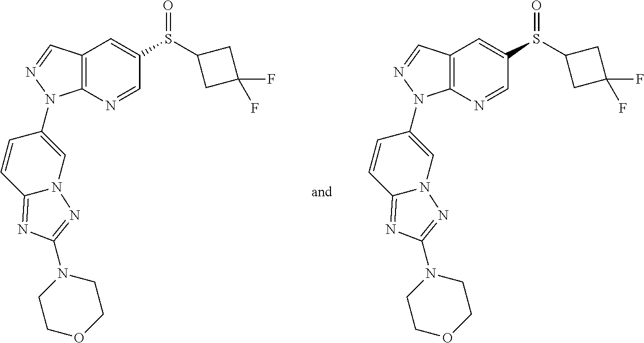 [SMILES: O=[S@@](c1cnc2c(cnn2-c2ccc3nc(N4CCOCC4)nn3c2)c1)C1CC(F)(F)C1 ; O=[S@](c1cnc2c(cnn2-c2ccc3nc(N4CCOCC4)nn3c2)c1)C1CC(F)(F)C1] |
| 118A and 118B | 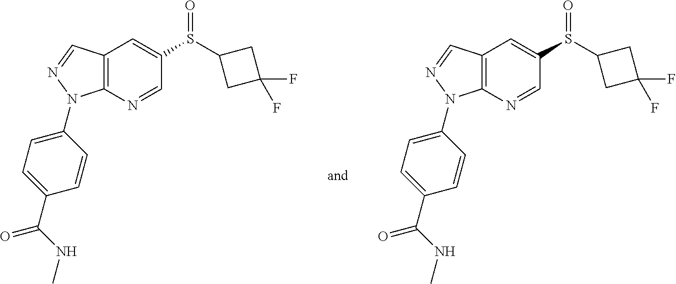 [SMILES: CNC(=O)c1ccc(-n2ncc3cc([S@](=O)C4CC(F)(F)C4)cnc32)cc1 ; CNC(=O)c1ccc(-n2ncc3cc([S@@](=O)C4CC(F)(F)C4)cnc32)cc1] |
| 119A and 119B | 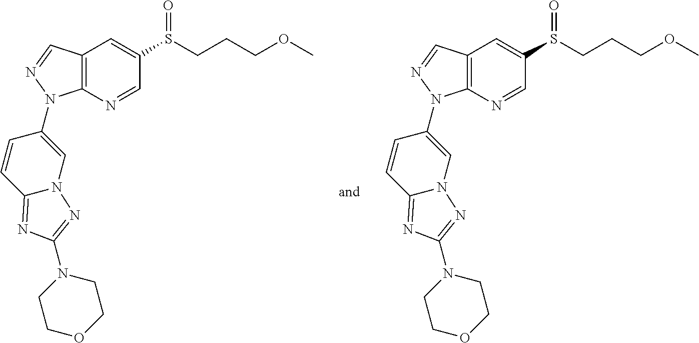 [SMILES: COCCC[S@@](=O)c1cnc2c(cnn2-c2ccc3nc(N4CCOCC4)nn3c2)c1 ; COCCC[S@](=O)c1cnc2c(cnn2-c2ccc3nc(N4CCOCC4)nn3c2)c1] |
| 120A, 120B, 120C, and 120D | 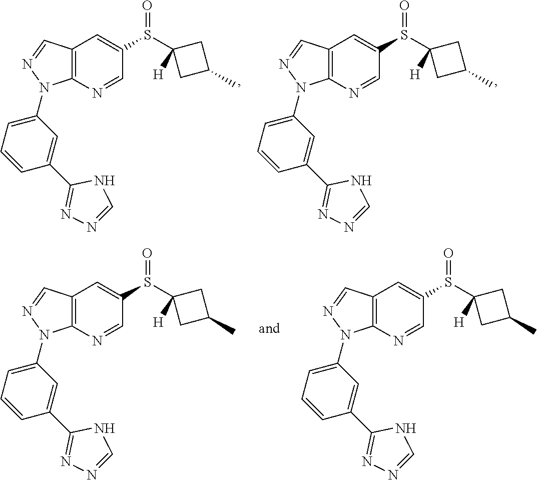 [SMILES: C[C@H]1C[C@@H]([S@@](=O)c2cnc3c(cnn3-c3cccc(-c4nnc[nH]4)c3)c2)C1 ; C[C@H]1C[C@@H]([S@](=O)c2cnc3c(cnn3-c3cccc(-c4nnc[nH]4)c3)c2)C1 ; C[C@H]1C[C@H]([S@](=O)c2cnc3c(cnn3-c3cccc(-c4nnc[nH]4)c3)c2)C1 ; C[C@H]1C[C@H]([S@@](=O)c2cnc3c(cnn3-c3cccc(-c4nnc[nH]4)c3)c2)C1] |
| 121A and 121B | 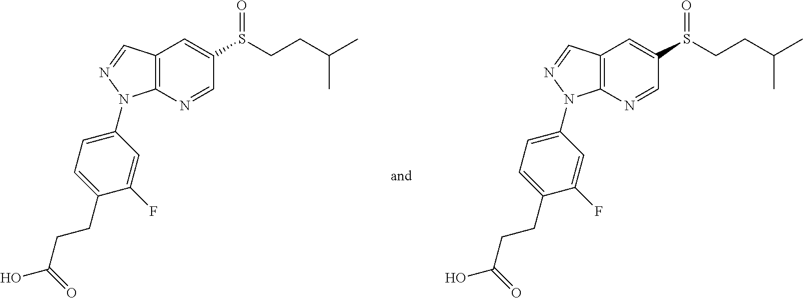 [SMILES: CC(C)CC[S@@](=O)c1cnc2c(cnn2-c2ccc(CCC(=O)O)c(F)c2)c1 ; CC(C)CC[S@](=O)c1cnc2c(cnn2-c2ccc(CCC(=O)O)c(F)c2)c1] |
| 122A and 122B | 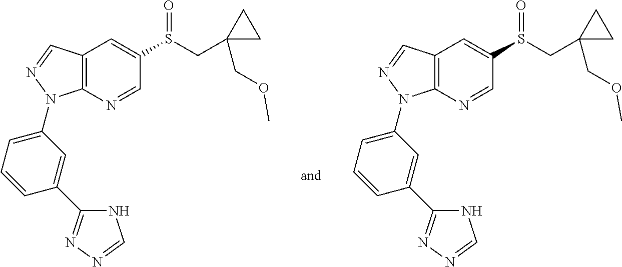 [SMILES: COCC1(C[S@@](=O)c2cnc3c(cnn3-c3cccc(-c4nnc[nH]4)c3)c2)CC1 ; COCC1(C[S@](=O)c2cnc3c(cnn3-c3cccc(-c4nnc[nH]4)c3)c2)CC1] |
| 123A, 123B. 123C, and 123D | 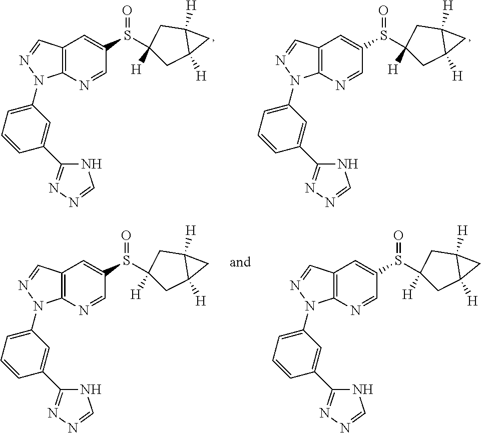 [SMILES: O=[S@](c1cnc2c(cnn2-c2cccc(-c3nnc[nH]3)c2)c1)[C@@H]1C[C@H]2C[C@H]2C1 ; O=[S@@](c1cnc2c(cnn2-c2cccc(-c3nnc[nH]3)c2)c1)[C@@H]1C[C@H]2C[C@H]2C1 ; O=[S@](c1cnc2c(cnn2-c2cccc(-c3nnc[nH]3)c2)c1)[C@H]1C[C@H]2C[C@H]2C1 ; O=[S@@](c1cnc2c(cnn2-c2cccc(-c3nnc[nH]3)c2)c1)[C@H]1C[C@H]2C[C@H]2C1] |
| 124A and 124B | 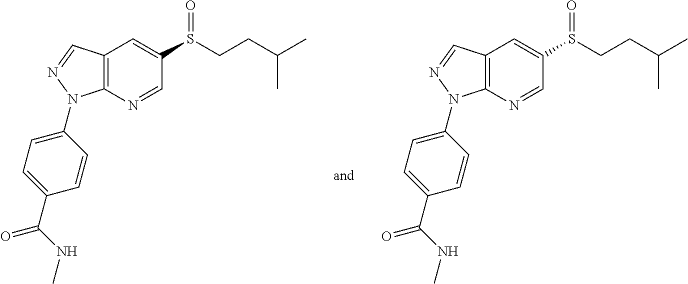 [SMILES: CNC(=O)c1ccc(-n2ncc3cc([S@@](=O)CCC(C)C)cnc32)cc1 ; CNC(=O)c1ccc(-n2ncc3cc([S@](=O)CCC(C)C)cnc32)cc1] |
| 125A and 125B | 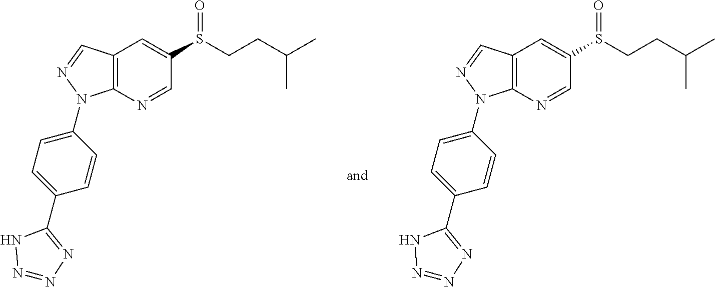 [SMILES: CC(C)CC[S@](=O)c1cnc2c(cnn2-c2ccc(-c3nnn[nH]3)cc2)c1 ; CC(C)CC[S@@](=O)c1cnc2c(cnn2-c2ccc(-c3nnn[nH]3)cc2)c1] |
| 126A and 126B | 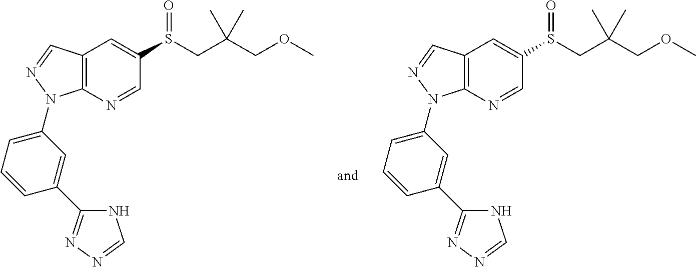 [SMILES: COCC(C)(C)C[S@](=O)c1cnc2c(cnn2-c2cccc(-c3nnc[nH]3)c2)c1 ; COCC(C)(C)C[S@@](=O)c1cnc2c(cnn2-c2cccc(-c3nnc[nH]3)c2)c1] |
| 127A and 127B | 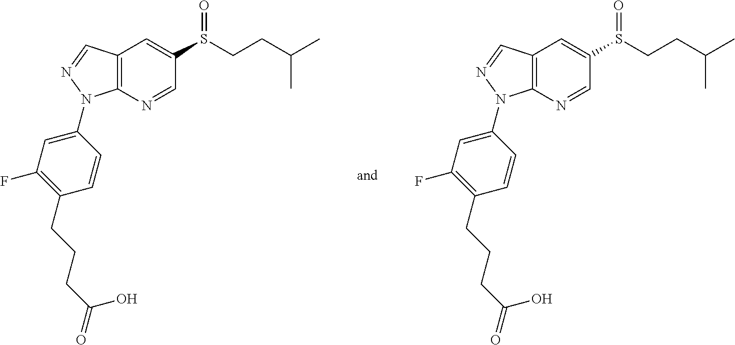 [SMILES: CC(C)CC[S@](=O)c1cnc2c(cnn2-c2ccc(CCCC(=O)O)c(F)c2)c1 ; CC(C)CC[S@@](=O)c1cnc2c(cnn2-c2ccc(CCCC(=O)O)c(F)c2)c1] |

### Methods of Use

In one aspect, provided herein are methods for treating various disorders in a subject in need thereof, comprising administering to said subject a compound described herein. In some embodiments, the inhibitors of hydroxyprostaglandin dehydrogenase provided herein may be used for the prevention or treatment of a disease or a disorder that is associated with hydroxyprostaglandin dehydrogenase (such as 15-PGDH) and/or decreased levels of prostaglandins. In some embodiments, the inhibitors of hydroxyprostaglandin dehydrogenase provided herein may be used for the prevention or treatment of a disease or a disorder in which it is desirable to increase prostaglandin levels in the subject having said disease or disorder.

In some embodiments, the methods for treating the disorders comprises administering to said subject a 15-PGDH inhibitor. In some embodiments, a compound described herein is the 15-PGDH inhibitor. In some embodiments, a compound of Formula (I), (II), (III), (IV), (Va), (Vb), (Vc), (Vd), (VI), (VII), (VIIIa), (VIIIb), (VIIIc), (VIIId), (IX), (Xa), (Xb), (Xc), or (Xd) is the 15-PGDH inhibitor. In some embodiments, the methods comprise administering a therapeutically effective amount of a compound described herein. In some embodiments, the methods comprise administering a therapeutically effective amount of a compound of Formula (I), (II), (III), (IV), (Va), (Vb), (Vc), (Vd), (VI), (VII), (VIIIa), (VIIIb), (VIIIc), (VIIId), (IX), (Xa), (Xb), (Xc), or (Xd). In some embodiments, the compound described herein is a 15-PGDH inhibitor. In some embodiments, the compound of Formula (I), (II), (III), (IV), (Va), (Vb), (Vc), (Vd), (VI), (VII), (VIIIa), (VIIIb), (VIIIc), (VIIId), (IX), (Xa), (Xb), (Xc), or (Xd) is a 15-PGDH inhibitor. In some embodiments, the administration takes place in vitro. In other embodiments, the administration takes place in vivo.

As used herein, a therapeutically effective amount of a 15-PGDH inhibitor refers to an amount sufficient to effect the intended application, including but not limited to, disease treatment, as defined herein. Also contemplated in the subject methods is the use of a sub-therapeutic amount of a 15-PGDH inhibitor for treating an intended disease condition.

The amount of the 15-PGDH inhibitor administered may vary depending upon the intended application (in vitro or in vivo), or the subject and disease condition being treated, e.g., the weight and age of the subject, the severity of the disease condition, the manner of administration and the like, which can readily be determined by one of ordinary skill in the art.

Measuring inhibition of biological effects of 15-PGDH can comprise performing an assay on a biological sample, such as a sample from a subject. Any of a variety of samples may be selected, depending on the assay. Examples of samples include, but are not limited to, blood samples (e.g., blood plasma or serum), exhaled breath condensate samples, bronchoalveolar lavage fluid, sputum samples, urine samples, and tissue samples.

A subject being treated with a 15-PGDH inhibitor may be monitored to determine the effectiveness of treatment, and the treatment regimen may be adjusted based on the subject's physiological response to treatment. For example, if inhibition of a biological effect of 15-PGDH is above or below a threshold, the dosing amount or frequency may be decreased or increased, respectively. The methods can further comprise continuing the therapy if the therapy is determined to be efficacious. The methods can comprise maintaining, tapering, reducing, or stopping the administered amount of a compound in the therapy if the therapy is determined to be efficacious. The methods can comprise increasing the administered amount of a compound in the therapy if it is determined not to be efficacious. Alternatively, the methods can comprise stopping therapy if it is determined not to be efficacious. In some embodiments, treatment with a 15-PGDH inhibitor is discontinued if inhibition of the biological effect is above or below a threshold, such as in a lack of response or an adverse reaction. The biological effect may be a change in any of a variety of physiological indicators.

In general, a 15-PGDH inhibitor is a compound that inhibits one or more biological effects of 15-PGDH. Such biological effects may be inhibited by about or more than about 10%, 20%, 30%, 40%, 50%, 60%, 70%, 80%, 90%, 95%, or more.

In some other embodiments, the subject methods are useful for treating a disease condition associated with 15-PGDH. Any disease condition that results directly or indirectly from an abnormal activity or expression level of 15-PGDH can be an intended disease condition.

In one aspect, provided herein is a method of promoting and/or stimulation skin pigmentation, comprising administering one or more of the compositions described herein to a subject in need thereof. Inhibitors of 15-PGDH are known to promote skin pigmentation (Markowitz et. al., WO 2015/065716). The hydroxyprostaglandin dehydrogenase inhibitors described herein can be used for promoting and/or inducing and/or stimulating pigmentation of the skin and/or skin appendages, and/or as an agent for preventing and/or limiting depigmentation and/or whitening of the skin and/or skin appendages, in particular as an agent for preventing and/or limiting canities. In some embodiments, the 15-PGDH inhibitors provided herein can be applied to skin of a subject, e.g., in a topical application, to promote and/or stimulate pigmentation of the skin and/or hair growth, inhibit hair loss, and/or treat skin damage or inflammation, such as skin damage caused by physical or chemical irritants and/or UV-exposure.

In another aspect, provided herein is a method of inhibiting hair loss, comprising administering one or more of the compositions described herein to a subject in need thereof. It is known that prostaglandins play an important role in hair growth. Prostaglandins such as prostaglandin A1, F2a and E2 are stored in hair follicles or adjacent skin environments and have been shown to be essential in maintaining and increasing hair density (Colombe L et. al, 2007, Exp. Dermatol, 16(9), 762-9). It has been reported that 15-PGDH, which is involved in the degradation of prostaglandins is present in the hair follicle dermal papillae, inactivates prostaglandins, especially, PGF2a and PGE2, to cause scalp damage and alopecia (Michelet J F et. al., 2008, Exp. Dermatol, 17(10), 821-8). Thus, the hydroxyprostaglandin dehydrogenase inhibitors described herein that have a suppressive or inhibitory activity against 15-PGDH can improve scalp damage, prevent alopecia and promote hair growth and be used in a pharmaceutical composition for the prevention of alopecia and the promotion of hair growth.

In another aspect, provided herein is a method of preventing and/or treating skin inflammation and/or damage, comprising administering one or more of the compositions described herein to a subject in need thereof.

In another aspect, provided herein is a method of preventing and/or treating vascular insufficiency, comprising administering one or more of the compositions described herein to a subject in need thereof. Prostaglandins including prostaglandin homologues produced in the body have been known to maintain the proper action of the blood vessel wall, especially to contribute to vasodilation for blood flow, preventing platelet aggregation and modulating the proliferation of smooth muscle that surrounds blood vessel walls (Yan. Cheng et. al., 2006, J. Clin., Invest). In addition, the inhibition of prostaglandins production or the loss of their activity causes the degeneration of the endothelium in the blood vessel walls, platelet aggregation and the dysfunction of cellular mechanism in the smooth muscle. Among others, the production of prostaglandins in blood vessels was shown to be decreased in hypertension patients, including pulmonary artery hypertension. the 15-PGDH inhibitors described herein can be used in a pharmaceutical composition for the prevention or the treatment of cardiovascular disease and/or diseases of vascular insufficiency, such as Raynaud's disease, Buerger's disease, diabetic neuropathy, and pulmonary artery hypertension.

In another aspect, provided herein is a method of preventing, treating, minimizing and/or reversing congestive heart failure, cardiomyopathy, comprising administering one or more of the compositions described herein to a subject in need thereof. In another aspect, provided herein is a method of reducing cardiac ejection fraction, comprising administering one or more of the compositions described herein to a subject in need thereof. It has been shown that administration of a 15-PGDH inhibitor can be used to treat, prevent, minimize, and/or reverse congestive heart failure, cardiomyopathy, and/or reduction of cardiac ejection fraction (Markowitz et. al., WO2018/187810). As such, the hydroxyprostaglandin dehydrogenase inhibitors described herein can be administered to a subject in need to treat, prevent, minimize and/or reverse congestive heart failure, cardiomyopathy, and/or reduction of cardiac ejection fraction.

In another aspect, provided herein is a method of preventing and/or treating a gastrointestinal disease, comprising administering one or more of the compositions described herein to a subject in need thereof. Prostaglandins are essential for maintaining the mechanism for protecting and defending gastric mucus membrane (Wallace J L., 2008, Physiol Rev., 88(4), 1547-65, S. J. Konturek et al., 2005, Journal of Physiology and Pharmacology, 56(5)). The inhibitors of hydroxyprostaglandin dehydrogenase described herein show a suppressive or inhibitory activity against 15-PGDH, which degrades prostaglandins that protect gastric mucus membranes. As such, the hydroxyprostaglandin dehydrogenase inhibitors can be effective for the prevention or the treatment of gastrointestinal diseases, inter alia, gastritis and gastric ulcer. In addition, the hydroxyprostaglandin dehydrogenase inhibitors provided herein may be used to prevent and/or treat other forms of intestinal injury including toxicity from radiation and/or chemotherapy, and chemotherapy-induced mucositis.

Additionally, it has been shown that administration of 15-PGDH inhibitors, alone or in combination with corticosteroids and/or TNF inhibitors can treat intestinal, gastrointestinal, or bowel disorders such as oral ulcers, gum disease, gastritis, colitis, ulcerative colitis, gastric ulcers, inflammatory bowel disease, and Crohn's disease (Markowitz et. al., WO 2018/102552). As such, the hydroxyprostaglandin dehydrogenase inhibitors provided herein can be used to treat and/or prevent treat intestinal, gastrointestinal, or bowel disorders such as oral ulcers, gum disease, gastritis, colitis, ulcerative colitis, gastric ulcers, inflammatory bowel disease, and Crohn's disease.

In another aspect, provided herein is a method of preventing and/or treating renal dysfunction, comprising administering one or more of the compositions described herein to a subject in need thereof. In the kidney, prostaglandins modulate renal blood flow and may serve to regulate urine formation by both renovascular and tubular effects. In clinical studies, inhibitors of prostaglandin have been used to improve creatinine clearance in patients with chronic renal disease, to prevent graft rejection and cyclosporine toxicity in renal transplant patients, to reduce the urinary albumin excretion rate and N-acetyl-beta-D-glucosaminidase levels in patients with diabetic nephropathy (Porter, Am., 1989, J. Cardiol., 64: 22E-26E). Furthermore, it has been reported that prostaglandins serve as vasodilators in the kidney, and, thus, the inhibition of prostaglandin production in the kidney results in renal dysfunction (Hao. C M, 2008, Annu Rev Physiol, 70, 357.about. 77). The hydroxyprostaglandin dehydrogenase inhibitors described herein have a suppressive or inhibitory activity against 15-PGDH that degrades prostaglandins and can be used for the prevention and/or treatment of renal diseases that are associated with renal dysfunction.

In another aspect, provided herein is a method of stimulation bone resorption and bone formation, comprising administering one or more of the compositions described herein to a subject in need thereof. Prostaglandins have been shown to stimulate bone resorption and bone formation to increase the volume and the strength of the bone (H. Kawaguchi et. al., Clinical Orthop. Rel. Res., 313, 1995; J. Keller et al., Eur. Jr. Exp. Musculoskeletal Res., 1, 1992, 8692). Furthermore, inhibition of 15-PGDH increases callus size and mineralization after bone fracture (Collier et. al., ORS 2017 Annual Meeting Paper No. 0190). Considering that 15-PGDH inhibits the activities of prostaglandins as mentioned in the above, the inhibition of 15-PGDH activity may lead to the promotion of bone resorption and bone formation that are inhibited by 15-PGDH. Thus, the inhibitors of hydroxyprostaglandin dehydrogenase described herein can be effective for the promotion of bone resorption and bone formation by inhibiting 15-PGDH activity. The hydroxyprostaglandin dehydrogenase inhibitors provided herein can also be used to increase bone density, treat osteoporosis, promote healing of fractures, promote healing after bone surgery or joint replacement, and/or to promote healing of bone to bone implants, bone to artificial implants, dental implants, and bone grafts.

In another aspect, provided herein is a method of stimulating tissue regeneration by stimulating, comprising administering one or more of the compositions described herein to a subject in need thereof. Prostaglandin PGE2 supports expansion of several types of tissue stem cells. Inhibition of 15-hydroxyprostaglandin dehydrogenase (15-PGDH), a prostaglandin-degrading enzyme, potentiates tissue regeneration in multiple organs. Studies show that inhibition of 15-PGDH increases prostaglandin PGE2 levels in bone marrow and other tissues; accelerates hematopoietic recovery following a bone marrow transplant; promotes tissue regeneration of colon and liver injury (Zhang, Y. et. al. Science 2015, 348 (6240)). The hydroxyprostaglandin dehydrogenase inhibitors provided herein can be used for tissue regeneration by supporting the expansion of tissue stem cells.

In another aspect, provided herein is a method of modulating cervical ripening, comprising administering one or more of the compositions described herein to a subject in need thereof. Prostaglandin E2 (PGE2) is a known cervical ripening agent that mediates EP2-receptor-signaling pathways in human cervical stromal cells; targets its own synthesis by increasing COX-2 and PTGES expression; and decreases its metabolism by loss of its degradative enzyme 15-PGDH (Word et. Al., WO2019010482) Downregulation of 15-PGDH was also found to be crucial for PGE2-induced cervical ripening and preterm birth. Modulation of 15-PDGH activity can be used to modulate cervical ripening; and induce or prevent preterm labor. The hydroxyprostaglandin dehydrogenase inhibitors provided herein can be used to induce cervical ripening and labor, alone or in combination with another labor inducing agent.

In another aspect, provided herein is a method of promoting neuroprotection and/or stimulating neuronal regeneration, comprising administering one or more of the compositions described herein to a subject in need thereof. Prostaglandins, via their specific G protein coupled receptors, have a variety of physiological functions in the central nervous system. The major prostaglandin, prostaglandin E2 (PGE2) can activate receptor types EP1, 2, 3, and 4. Activation of EP2 and EP4 receptors can regulate adenylate cyclase and the generation of 3, 5′-cyclic adenosine monophosphate (cAMP), whereas the activation of EP1 and EP3 receptors can regulate Ca2+ signaling. Studies show that the EP1 and EP2 receptors are expressed in neurons and microglia as well as neurons of the cerebral cortex, striatum, and hippocampus. In addition, activation of the EP2 receptor by PGE2 is involved in long-term synaptic plasticity and cognitive function (Chemtob et al. Semin Perinatol. 1994 February; 18(1):23-9; Yang et al., J Neurochem. 2009 January; 108(1):295-304). Studies also show that following activation, different PGE2 receptors can contribute or protect against N-methyl-D-aspartate (NMDA) neurotoxicity and ischemic stroke (Ahmad et al., Exp Transl Stroke Med. 2010 Jul. 8; 2(1):12). Other studies show that activation of the EP2 receptors protected neurons from amyloid β-peptide neurotoxicity in vitro (Echeverria et al., Eur J Neurosci. 2005 November; 22(9):2199-206). Several studies suggest that the mechanism by which PGE2 affords neuroprotection is through EP2 or EP4 receptors, as they both increases cAMP, followed by a protein kinase A (PKA)-dependent pathway (Echeverria et al. Eur J Neurosci. 2005 November; 22(9):2199-206; McCullough et al., J Neurosci. 2004 Jan. 7; 24(1):257-68). Stimulation of these receptors with PGE2 by administration of a compound that inhibits, reduces, and/or antagonizes 15-PGDH activity, such as the hydroxyprostaglandin dehydrogenase inhibitors that can inhibit 15-PGDH described herein, can promote neuroprotection in a subject from axonal degeneration, neuronal cell death, and/or glia cell damage after injury, augment neuronal signaling underlying learning and memory, stimulate neuronal regeneration after injury, and/or treat diseases, disorders, and/or conditions of the nervous system.

In another aspect, provided herein is a method of treating and/or preventing a neurological disorder, a neuropsychiatric disorder, a neural injury, a neural toxicity disorder, a neuropathic pain, or a neural degenerative disorder, comprising administering one or more of the compositions described herein to a subject in need thereof. In some embodiments, the disease, disorder, and/or condition of the nervous system, which can be treated with hydroxyprostaglandin dehydrogenase inhibitors provided herein, can include at least one of a neurological disorder, a neuropsychiatric disorder, a neural injury, a neural toxicity disorder, a neuropathic pain, or a neural degenerative disorder. For example, the neurological disorder can include at least one of traumatic or toxic injuries to peripheral or cranial nerves, spinal cord or brain, such as traumatic brain injury, stroke, cerebral aneurism, and spinal cord injury. The neurological disorder can also include at least one of Alzheimer's disease, dementias related to Alzheimer's disease, Parkinson's, Lewy diffuse body diseases, senile dementia, Huntington's disease, Gilles de Ia Tourette's syndrome, multiple sclerosis, amyotrophic lateral sclerosis, hereditary motor and sensory neuropathy, diabetic neuropathy, progressive supranuclear palsy, epilepsy, or Jakob-Creutzfieldt disease.

In some embodiments, the neural injury can be caused by or associated with at least one of epilepsy, cerebrovascular diseases, autoimmune diseases, sleep disorders, autonomic disorders, urinary bladder disorders, abnormal metabolic states, disorders of the muscular system, infectious and parasitic diseases, neoplasms, endocrine diseases, nutritional and metabolic diseases, immunological diseases, diseases of the blood and blood-forming organs, mental disorders, diseases of the nervous system, diseases of the sense organs, diseases of the circulatory system, diseases of the respiratory system, diseases of the digestive system, diseases of the genitourinary system, diseases of the skin and subcutaneous tissue, diseases of the musculoskeletal system and connective tissue, congenital anomalies, or conditions originating in the perinatal period.

In certain embodiments, the hydroxyprostaglandin dehydrogenase inhibitors can be administered to a subject or neurons of the subject to promote the survival, growth, development and/or function of the neurons, particularly, the central nervous system (CNS), brain, cerebral, and hippocampal neurons. In certain embodiments, the hydroxyprostaglandin dehydrogenase inhibitors can be used stimulate hippocampal neurogenesis, for the treatment of neuropsychiatric and neurodegenerative diseases, including (but not limited to) schizophrenia, major depression, bipolar disorder, normal aging, epilepsy, traumatic brain injury, post-traumatic stress disorder, Parkinson's disease, Alzheimer's disease, Down syndrome, spinocerebellar ataxia, amyotrophic lateral sclerosis, Huntington's disease, stroke, radiation therapy, chronic stress, and abuse of neuro-active drugs, such as alcohol, opiates, methamphetamine, phencyclidine, and cocaine.

In another aspect, provided herein is a method of treating and/or preventing fibrotic or adhesion disease, disorder or condition, comprising administering one or more of the compositions described herein to a subject in need thereof. It has been shown that inhibitors of short-chain dehydrogenase activity, such as 15-PGDH inhibitors, can be administered to a subject in need thereof to decrease fibrotic symptoms, such as collagen deposition, collagen accumulation, collagen fiber formation, inflammatory cytokine expression, and inflammatory cell infiltration, and treat and/or prevent various fibrotic diseases, disorders, and conditions characterized, in whole or in part, by the excess production of fibrous material, including excess production of fibrotic material within the extracellular matrix, or the replacement of normal tissue elements by abnormal, non-functional, and/or excessive accumulation of matrix-associated components (Markowitz et. al., WO2016/144958).

Fibrotic diseases, disorders and conditions characterized, in whole or in part, by excess production of fibrotic material can include systemic sclerosis, multifocal fibrosclerosis, nephrogenic systemic fibrosis, scleroderma (including morphea, generalized morphea, or linear scleroderma), sclerodermatous graft-vs-host-disease, kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g. pulmonary fibrosis, glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fasciitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, myelodysplastic syndrome, myeloproliferative syndrome, collagenous colitis, acute fibrosis, organ specific fibrosis, and the like. The hydroxyprostaglandin dehydrogenase inhibitors provided herein can be used to treat or prevent a fibrotic disease, disorder or condition.

The hydroxyprostaglandin dehydrogenase inhibitors provided herein can be used to treat or prevent kidney fibrosis, including kidney fibrosis resulting from dialysis following kidney failure, catheter placement, a nephropathy, glomerulosclerosis, glomerulonephritis, chronic renal insufficiency, acute kidney injury, end stage renal disease or renal failure, or combinations thereof.

The hydroxyprostaglandin dehydrogenase inhibitors provided herein can be used to treat or prevent liver fibrosis, including liver fibrosis resulting from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, alpha-1-antitrypsin deficiency, primary biliary cirrhosis, drug reaction and exposure to toxins, or combinations thereof.

The hydroxyprostaglandin dehydrogenase inhibitors provided herein can be used to treat or prevent heart fibrosis such as cardiac fibrosis, endomyocardial fibrosis, idiopathic pulmonary fibrosis, and kidney fibrosis.

The hydroxyprostaglandin dehydrogenase inhibitors provided herein can be used to treat or prevent systemic sclerosis.

The hydroxyprostaglandin dehydrogenase inhibitors provided herein can be used to treat or prevent fibrotic diseases, disorders or conditions caused by post-surgical adhesion formation.

The hydroxyprostaglandin dehydrogenase inhibitors provided herein can be used to reduce in intensity, severity, or frequency, and/or delay onset of one or more symptoms or features of a fibrotic disease, disorder or condition, or other related diseases, disorders or conditions.

The hydroxyprostaglandin dehydrogenase inhibitors provided herein can be used to decrease or reduce collagen secretion, or collagen deposition, or collagen fiber accumulation, in a tissue or organ, such as the lung, the liver, the intestines, the colon, the skin or the heart, or a combination thereof.

Studies have shown that 15-PGDH inhibition ameliorates inflammatory pathology and fibrosis in pulmonary fibrosis (Smith et. al., bioRxiv 2019.12.16.878215; Barnthaler et. al., J. Allergy Clin. Immunol. 2019, 145 (3), 818-833). In some embodiments, the hydroxyprostaglandin dehydrogenase inhibitors described herein can be used to treat or prevent lung fibrosis, including pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations thereof.

In another aspect, provided herein is a method of reducing and/or preventing scar formation, comprising administering one or more of the compositions described herein to a subject in need thereof. The hydroxyprostaglandin dehydrogenase inhibitors provided herein can used for reducing or preventing scar formation in a subject. The hydroxyprostaglandin dehydrogenase inhibitors provided herein can be used to reduce or prevent scar formation on skin or scleroderma.

In another aspect, provided herein is a method of treating and/or preventing muscle disorder, muscle injury and/or muscle atrophy, comprising administering one or more of the compositions described herein to a subject in need thereof. Studies have shown that inhibition of PGE2 degrading enzymes such as 15-PGDH, enable muscle regeneration and muscle repair after injury (Ho et al., PNAS 2017; Dong et al., Stem cell research and therapy 2020). The inhibitors of hydroxyprostaglandin dehydrogenase provided herein can be used to treat muscle disorder, muscle injury and/or muscle atrophy in a subject. In some cases, said subject suffering from a muscle disorder, muscle injury and/or muscle atrophy may have Duchenne muscular dystrophy (DMD), Becker muscular dystrophy, Fukuyama congenital muscular dystrophy (FCMD), limb girdle muscular dystrophy, congenital muscular dystrophy, facioscapulohumeral muscular dystrophy (FHMD), amyotrophic lateral sclerosis (ALS), distal muscular dystrophy (DD), an inherited myopathy, myotonic muscular dystrophy (MDD), oculopharyngeal muscular dystrophy, distal muscular dystrophy, Emery-Dreifuss muscular dystrophy, myotonia congenita, mitochondrial myopathy (DD), myotubular myopathy (MM), myasthenia gravis (MG), periodic paralysis, polymyositis, rhabdomyolysis, dermatomyositis, cancer cachexia, AIDS cachexia, stress induced urinary incontinence, urethral sphincter deficiency, sarcopenia, or a combination thereof.

In some embodiments, the inhibitors of hydroxyprostaglandin dehydrogenase provided herein can be used to treat sarcopenia. In another embodiment, the inhibitors of hydroxyprostaglandin dehydrogenase provided herein can be used to treat diaphragmatic atrophy or limb muscle atrophy due to the use of a mechanical ventilator. In some embodiments, the inhibitors of hydroxyprostaglandin dehydrogenase provided herein can be used to treat genetic disorders or neuromuscular disorders such as Spinal Muscular Atrophy (SMA). In some embodiments, the inhibitors of hydroxyprostaglandin dehydrogenase provided herein can be used to treat ptosis, rotator cuff muscle atrophy, immobilization related muscle atrophy, surgical procedure related muscle atrophy, sarcopenia, or a combination thereof.

### Pharmaceutical Compositions

The inhibitors of hydroxyprostaglandin dehydrogenase can be formulated into pharmaceutical compositions to treat diseases and disorders described herein. In some embodiments, a pharmaceutical composition may comprise a therapeutically effective amount of one or more inhibitors of hydroxyprostaglandin dehydrogenase provided herein.

The pharmaceutical composition described herein may be administered in such oral dosage forms as tablets, capsules (each of which includes sustained release or timed release formulations), pills, powders, micronized compositions, granules, elixirs, tinctures, suspensions, ointments, vapors, liposomal particles, nanoparticles, syrups and emulsions. In some embodiments, the pharmaceutical composition may also be administered in intravenous (bolus or infusion), subcutaneous injection, suppository, intraperitoneal, topical (e.g., dermal epidermal, transdermal), ophthalmically such as ocular eyedrop, intranasally, subcutaneous, inhalation, intramuscular or transdermal (e.g., patch) form, all using forms well known to those of ordinary skill in the pharmaceutical arts.

In some embodiments, a compound provided herein can be administered as part of a therapeutic regimen that comprises administering one or more second agents (e.g., 1, 2, 3, 4, 5, or more second agents), either simultaneously or sequentially with the compound provided herein. When administered sequentially, the compound provided herein may be administered before or after the one or more second agents. When administered simultaneously, the compound provided herein and the one or more second agents may be administered by the same route (e.g., injections to the same location; tablets taken orally at the same time), by a different route (e.g., a tablet taken orally while receiving an intravenous infusion), or as part of the same combination (e.g., a solution comprising a compound provided herein and one or more second agents).

A combination treatment according to the disclosure may be effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. The exact dosage will depend upon the agent selected, the route of administration, the form in which the compound is administered, the subject to be treated, the body weight of the subject to be treated, and the preference and experience of the attending physician.

### EXAMPLES

### Synthesis and Characterization of Compounds

The following examples are offered to illustrate, but not to limit the claimed invention. The following examples further illustrate the invention but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

The compounds and salts of Formulas (I), (II), (III), (IV), (Va), (Vb), (Vc), (Vd), (VI), (VII), (VIIIa), (VIIIb), (VIIIc), (VIIId), (IX), (Xa), (Xb), (Xc), or (Xd), can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in the synthesis schemes below, the steps in some cases may be performed in a different order than the order shown below. Numberings or R groups in each scheme do not necessarily correspond to that of the claims or other schemes or tables herein.

Described herein are exemplary synthesis schemes that can be used to synthesize the inhibitors described herein. The following abbreviations are used:

| Abbreviation | Description |
|---|---|
| AIBN | azobisisobutyronitrile |
| DCM | dichloromethane |
| DIAD | diisopropyl azodicarboxylate |
| DIPEA | N,N′-diisopropylethylamine |
| DMAP | 4-dimethylaminopyridine |
| DMF | dimethylformamide |
| EA | ethyl acetate |
| EDCI | 1-ethyl-3-(3-dimethylaminopropyl)carbodiimide |
| HATU | 1-[Bis(dimethylamino)methylene]-1H-1,2,3-triazolo[4,5-b]pyridinium 3-oxide |
| | hexafluorophosphate |
| HOBt | hydroxybenzotriazole |
| m-CPBA | Meta-chloroperoxybenzoic acid |
| NBS | N-bromosuccinimide |
| NCS | N-chlorosuccinimide |
| NIS | N-iodosuccinimide |
| p-TSA | para-toluenesulfonic acid |
| TEA | triethylamine |
| TFA | trifluoroacetic acid |
| THF | tetrahydrofuran |
| TPP | triphenylphosphine |
| mmol | Milli molar |
| vol | Volume |
| g | Gram |
| kg | Kilogram |
| L | Liter |
| mL | Milli liter |
| ° C. | Degree Celsius |
| TLC | Thin Layer Chromatography |
| HPLC | High-performance liquid chromatography |
| LCMS | Liquid chromatography - mass spectrometry |
| min | Minutes |
| h | Hour |
| eq | Equivalents |
| RT | Room temperature |
| Rf | Retention factor |
| RP | Reversed phase |
| NMR | Nuclear magnetic resonance |
| Ppm | Parts per million |

### Example 1. Representative Procedure A: Synthesis of (R)-1-(3-(4H-1,2,4-triazol-3-yl)phenyl)-5-(butylsulfinyl)-1H-pyrazolo[3,4-b]pyridine and (S)-1-(3-(4H-1,2,4-triazol-3-yl)phenyl)-5-(butylsulfinyl)-1H-pyrazolo[3,4-b]pyridine (Compounds 36A and 36B)

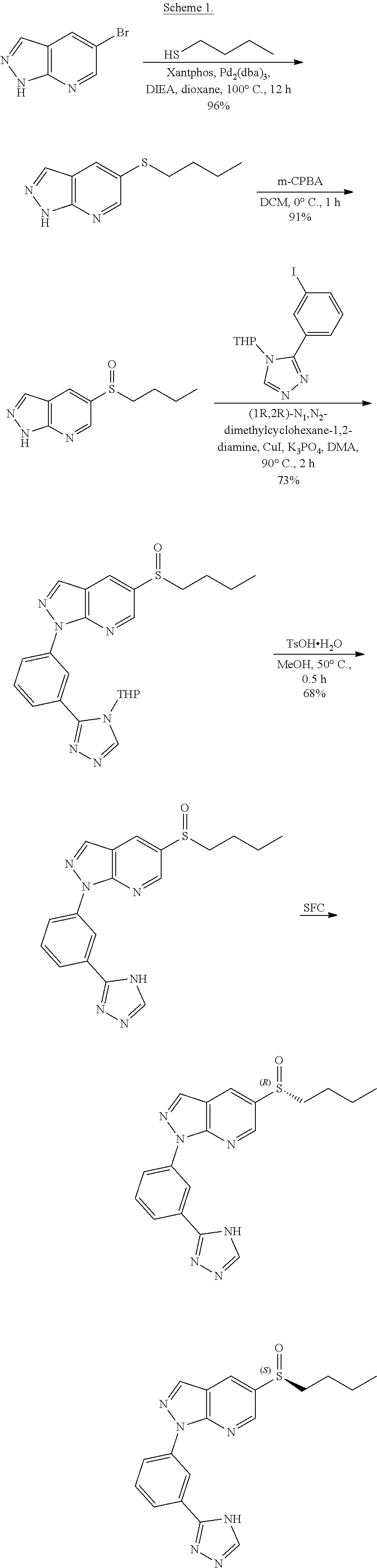

A mixture of 5-bromo-1H-pyrazolo[3,4-b]pyridine (10.0 g, 50.5 mmol, 1.00 eq.), butane-1-thiol (5.92 g, 65.7 mmol, 7.03 mL, 1.30 eq.), Pd₂(dba)₃ (4.62 g, 5.05 mmol, 0.10 eq.), Xantphos (5.84 g, 10.1 mmol, 0.20 eq.) and DIEA (13.1 g, 101 mmol, 17.6 mL, 2.00 eq.) in dioxane (100 mL) was degassed and purged with nitrogen 3 times, then stirred at 100° C. for 12 hours under nitrogen atmosphere (15 psi). The reaction mixture was diluted with ethyl acetate (200 mL) and washed with brine (200 mL×3), the organic layer was dried over anhydrous sodium sulfate, filtered and concentrated under reduced pressure to give a residue. The residue was purified by silica column chromatography (petroleum ether/ethyl acetate=10/1 to 1/1) to give 5-butylsulfanyl-1H-pyrazolo[3,4-b]pyridine (10.0 g, 48.2 mmol, 96% yield) as a yellow oil. ¹H NMR (400 MHz, DMSO-d6) δ=13.74 (br s, 1H), 8.52 (d, J=2.0 Hz, 1H), 8.31 (d, J=2.0 Hz, 1H), 8.11 (s, 1H), 2.94 (t, J=7.2 Hz, 2H), 1.55-1.44 (m, 2H), 1.33-1.42 (m, 2H), 0.85 (t, J=7.2 Hz, 3H).

To a solution of 5-butylsulfanyl-1H-pyrazolo[3,4-b]pyridine (9.00 g, 43.4 mmol, 1.00 eq.) in DCM (110 mL) was slowly added a solution of m-CPBA (8.81 g, 43.4 mmol, 85% purity, 1.00 eq.) in DCM (40.0 mL) at 0° C. The mixture was stirred at 0° C. for 1 hour. The reaction mixture was diluted with ethyl acetate (400 mL) and washed with brine (400 mL×2), the organic layer was dried over anhydrous sodium sulfate, filtered and concentrated under reduced pressure to give compound 5-butylsulfinyl-1H-pyrazolo[3,4-b]pyridine (8.80 g, 39.4 mmol, 91% yield) as a light yellow solid. ¹H NMR (400 MHz, DMSO-d6) δ=14.01 (br s, 1H), 8.75 (d, J=2.0 Hz, 1H), 8.56 (d, J=2.0 Hz, 1H), 8.29 (s, 1H), 3.12-2.85 (m, 2H), 1.69-1.28 (m, 4H), 0.96-0.75 (m, 3H).

A mixture of 5-butylsulfinyl-1H-pyrazolo[3,4-b]pyridine (6.80 g, 30.5 mmol, 1.00 eq.), 3-(3-iodophenyl)-4-tetrahydropyran-2-yl-1,2,4-triazole (11.4 g, 32.0 mmol, 1.05 eq.), CuI (2.90 g, 15.2 mmol, 0.50 eq.), (1R,2R)—N1,N2-dimethylcyclohexane-1,2-diamine (2.17 g, 15.2 mmol, 0.50 eq.) and K₃PO₄ (12.9 g, 60.9 mmol, 2.00 eq.) in DMAC (70.0 mL) was degassed and purged with nitrogen 3 times, then the mixture was stirred at 90° C. for 2 hours under nitrogen atmosphere (15 psi). The reaction mixture was diluted with ethyl acetate (200 mL) and washed with brine (200 mL×3), the organic layer was dried over anhydrous sodium sulfate, filtered and concentrated under reduced pressure to give a residue. The reaction mixture was purified by silica column chromatography (PE/EA=5/1 to 1/1). 5-butylsulfinyl-1-[3-(4-tetrahydropyran-2-yl-1,2,4-triazol-3-yl)phenyl]pyrazolo[3,4-b]pyridine (10.4 g, 22.3 mmol, 74% yield, 97% purity) was obtained as a light yellow solid. LCMS [ESI, M−83]: 367.0.

To a solution of 5-butylsulfinyl-1-[3-(4-tetrahydropyran-2-yl-1,2,4-triazol-3-yl)phenyl]pyrazolo[3,4-b]pyridine (10.0 g, 22.2 mmol, 1.00 eq.) in MeOH (100 mL) was added TsOH·H₂O (5.07 g, 26.6 mmol, 1.20 eq.), and the mixture was stirred at 50° C. for 0.5 h. The reaction mixture was diluted with ethyl acetate (400 mL) and washed with aqueous NaHCO₃ solution (400 mL×2) and brine (400 mL×2). The organic layer was dried over anhydrous sodium sulfate, filtered and concentrated under reduced pressure to give a residue. The residue was triturated with PE/EA (2/1, 100 mL) and filtered to give 1-(3-(4H-1,2,4-triazol-3-yl)phenyl)-5-(butylsulfinyl)-1H-pyrazolo[3,4-b]pyridine (5.60 g, 15.2 mmol, 68% yield, 99% purity) as a white solid. LCMS [ESI, M+1]: 367.1. ¹H NMR (400 MHz, DMSO-d6) δ=14.26 (br s, 1H), 8.97 (s, 1H), 8.95 (d, J=2.0 Hz, 1H), 8.73 (d, J=2.0 Hz, 1H), 8.65 (s, 1H), 8.57 (br s, 1H), 8.35 (br d, J=7.6 Hz, 1H), 8.03 (d, J=7.6 Hz, 1H), 7.71 (t, J=7.6 Hz, 1H), 3.20-3.08 (m, 1H), 3.04-2.93 (m, 1H), 1.72-1.57 (m, 1H), 1.52-1.31 (m, 3H), 0.91-0.82 (m, 3H).

The racemic compound (400 mg, 99% purity) was purified by SFC (column: DAICEL CHIRALCEL OD (250 mm*30 mm, 10 um); mobile phase: [60% EtOH (base) in Supercritical CO₂]; B %: 60%-60%, 7 min) to give Peak 1 (Compound 36A) (160 mg, 435 μmol, 40% yield, 99% purity) as a white solid and Peak 2 (Compound 36B) (167 mg, 454 μmol, 42% yield, 99% purity) as a light yellow solid.

Compound 36A: LCMS [ESI, M+1]: 367.1. ¹H NMR (400 MHz, DMSO-d6) δ=14.29 (br s, 1H), 8.97 (s, 1H), 8.95 (d, J=2.0 Hz, 1H), 8.72 (d, J=2.0 Hz, 1H), 8.65 (s, 1H), 8.56 (br s, 1H), 8.35 (br d, J=7.6 Hz, 1H), 8.03 (d, J=7.6 Hz, 1H), 7.71 (t, J=7.6 Hz, 1H), 3.20-3.08 (m, 1H), 3.03-2.93 (m, 1H), 1.73-1.58 (m, 1H), 1.52-1.31 (m, 3H), 0.91-0.82 (m, 3H).

Compound 36B: LCMS [ESI, M+1]: 367.1. ¹H NMR (400 MHz, DMSO-d6) δ=14.27 (br s, 1H), 8.97 (s, 1H), 8.94 (s, 1H), 8.71 (s, 1H), 8.63 (s, 1H), 8.56 (br s, 1H), 8.34 (br d, J=7.6 Hz, 1H), 8.03 (br d, J=7.6 Hz, 1H), 7.70 (t, J=7.6 Hz, 1H), 3.20-3.06 (m, 1H), 3.04-2.87 (m, 1H), 1.74-1.56 (m, 1H), 1.52-1.27 (m, 3H), 0.87-0.83 (m, 3H).

### Example 2. Representative Procedure B: Synthesis of (R)-1-(3-(4H-1,2,4-triazol-3-yl)phenyl)-5-(cyclobutylsulfinyl)-1H-pyrazolo[3,4-b]pyridine and (S)-1-(3-(4H-1,2,4-triazol-3-yl)phenyl)-5-(cyclobutylsulfinyl)-1H-pyrazolo[3,4-b]pyridine (Compounds 18A and 18B)

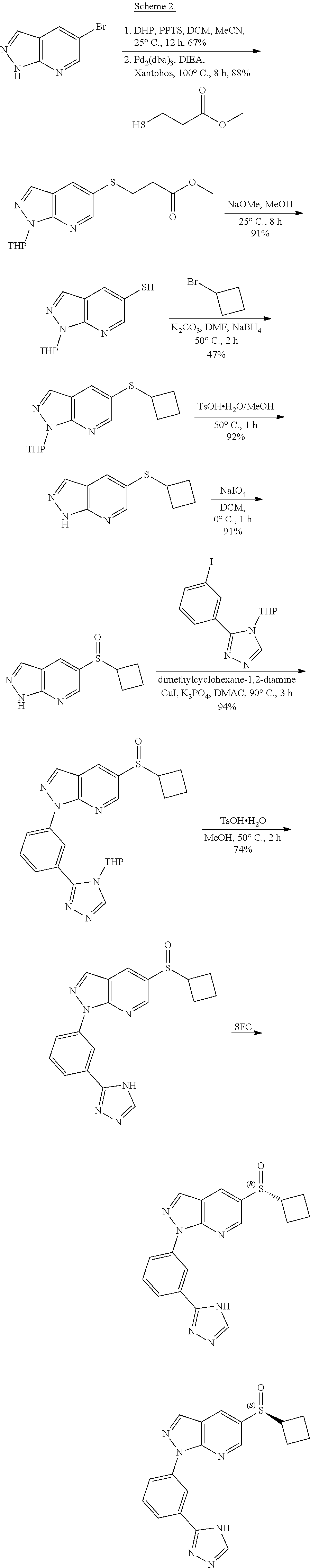

A mixture of 5-bromo-1H-pyrazolo[3,4-b]pyridine (100 mg, 505 μmol, 1.00 eq.), DHP (127 mg, 1.51 mmol, 139 μL, 3.00 eq.), PPTS (12.7 mg, 50.5 μmol, 0.10 eq.) in DCM (2.50 mL) and MeCN (4.50 mL) was stirred at 25° C. for 8 hours. The reaction mixture was concentrated under vacuum. Then the mixture was poured into H₂O (10 mL) and extracted with DCM (10 mL×3). The combined organic layer was washed with brine (20 mL), dried over Na₂SO₄, the mixture was filtered, and the filtrate was concentrated. The residue was purified by column chromatography (SiO₂, PE/EA=1/0 to 3/1) to give compound 5-bromo-1-tetrahydropyran-2-yl-pyrazolo[3,4-b]pyridine (100 mg, 337 μmol, 67% yield) as a yellow oil. LCMS (ESI, M+1): m/z=282.1

A mixture of 5-bromo-1-tetrahydropyran-2-yl-pyrazolo[3,4-b]pyridine (6.00 g, 21.3 mmol, 1.00 eq.), methyl 3-sulfanylpropanoate (3.07 g, 25.5 mmol, 2.76 mL, 1.20 eq.), Pd₂(dba)₃ (1.95 g, 2.13 mmol, 0.10 eq.), Xantphos (2.46 g, 4.25 mmol, 0.20 eq.) and DIEA (5.50 g, 42.5 mmol, 7.41 mL, 2.00 eq.) in 1,4-dioxane (100 mL) was degassed and purged with N₂ for 3 times, and then the mixture was stirred at 100° C. for 8 hours under N₂ atmosphere (15 psi). The reaction mixture was filtered, and the filtrate were diluted with EA (500 mL) and washed with water (500 mL×3). The organic layers were dried over anhydrous sodium sulfate, filtered and concentrated under reduced pressure to give a residue. The residue was purified by column chromatography (SiO₂, Petroleum ether/Ethyl acetate=5/1 to 1/1). The desired fractions were concentrated to give compound methyl 3-(1-tetrahydropyran-2-ylpyrazolo[3,4-b]pyridin-5-yl)sulfanylpropanoate (6.0 g, 18.7 mmol, 88% yield) as a yellow oil. LCMS [ESI, M+1]: 322.1.

To a solution of methyl 3-(1-tetrahydropyran-2-yl pyrazolo [3,4-b]pyridin-5-yl)sulfanylpropanoate (6.00 g, 18.7 mmol, 1.00 eq.) in MeOH (120 mL) was added NaOMe (2.02 g, 37.3 mmol, 2.00 eq.). The mixture was stirred at 25° C. for 8 hours. The reaction mixture was quenched with 20% citric acid solution at 0° C. to obtain pH=3, then extracted with EA (150 mL×3). The combined organic layers were washed with brine (300 mL), dried over Na₂SO₄, filtered and concentrated under reduced pressure to give a residue. The residue was purified by column chromatography (SiO₂, Petroleum ether/Ethyl acetate=5/1 to 2/1) to give compound 1-tetrahydropyran-2-ylpyrazolo [3,4-b]pyridine-5-thiol (4.00 g, 17.0 mmol, 91% yield) as a colorless oil. LCMS [ESI, M+1]: 236.1.

To a solution of 1-tetrahydropyran-2-ylpyrazolo[3,4-b]pyridine-5-thiol (400 mg, 1.70 mmol, 1.00 eq.) and bromocyclobutane (1.38 g, 10.2 mmol, 963 μL, 6.00 eq.) in DMF (4 mL) was added K₂CO₃ (940 mg, 6.80 mmol, 4.00 eq.). The mixture was stirred at 50° C. for 2 hours. The mixture was poured into water (30 mL) and extracted with EA (10 mL×3). The combined organic layers were washed with brine (20 mL), dried over Na₂SO₄, then the mixture was filtered, and the filtrate was concentrated. The residue was purified by column chromatography (SiO₂, Petroleum ether/Ethyl acetate=10/1-1/1). Compound 5-cyclobutylsulfanyl-1-tetrahydropyran-2-yl-pyrazolo[3,4-b]pyridine (240 mg, 804 μmol, 47% yield, 97% purity) was obtained as a yellow oil. LCMS [ESI, M+1]: 290.1. ¹H NMR (400 MHz, DMSO-d₆) δ=8.56-8.52 (m, 1H), 8.26 (s, 1H), 8.22-8.19 (m, 1H), 5.97 (s, 1H), 3.97-3.88 (m, 2H), 3.73-3.65 (m, 1H), 2.36-2.30 (m, 2H), 2.07-2.01 (m, 1H), 1.99-1.83 (m, 6H), 1.82-1.74 (m, 1H), 1.60-1.53 (m, 2H).

To a solution of 5-cyclobutylsulfanyl-1-tetrahydropyran-2-yl-pyrazolo [3,4-b]pyridine (220 mg, 760 mol, 1.00 eq.) in MeOH (2 mL) was added TsOH·H₂O (217 mg, 1.14 mmol, 1.50 eq.). The mixture was stirred at 50° C. for 2 hours. The mixture was poured into saturated NaHCO₃ aqueous solution (30 mL) and extracted with EA (10 mL×3). The combined organic layer was washed with brine (20 mL), dried over Na₂SO₄, then the mixture was filtered, and the filtrate was concentrated. Compound 5-cyclobutylsulfanyl-1H-pyrazolo[3,4-b]pyridine (160 mg, 701 μmol, 92% yield, 90% purity) was obtained as a yellow oil. LCMS [ESI, M+1]: 206.1. ¹H NMR (400 MHz, DMSO-d₆) δ=13.75 (br d, J=1.6 Hz, 1H), 8.47 (d, J=2.0 Hz, 1H), 8.25 (d, J=2.0 Hz, 1H), 8.12 (s, 1H), 3.91-3.83 (m, 1H), 2.35-2.29 (m, 2H), 1.95-1.85 (m, 4H).

To a solution of 5-cyclobutylsulfanyl-1H-pyrazolo[3,4-b]pyridine (160 mg, 779 μmol, 1.00 eq.) in DCM (6 mL) was added NaIO₄ (183 mg, 857 μmol, 47.5 μL, 1.10 eq.). The mixture was stirred at 0° C. for 1 hour. The reaction mixture was diluted with saturated Na₂SO₃ (20 mL) and extracted with EA (15 mL×3). The combined organic layers were washed with brine (50 mL×1), dried over Na₂SO₄, filtered and concentrated under reduced pressure to give a residue. The residue was purified by column chromatography (SiO₂, Petroleum ether/Ethyl acetate=10/1 to 0/1). Compound 5-cyclobutylsulfinyl-1H-pyrazolo [3,4-b]pyridine (160 mg, 709 μmol, 91% yield, 98% purity) was obtained as yellow oil. LCMS [ESI, M+1]: 222.1.

A mixture of 5-cyclobutylsulfinyl-1H-pyrazolo[3,4-b]pyridine (140 mg, 632 μmol, 1.00 eq.), 3-(3-iodophenyl)-4-tetrahydropyran-2-yl-1,2,4-triazole (270 mg, 759 mol, 1.20 eq.), CuI (60.3 mg, 316 mol, 0.50 eq.), K₃PO₄ (269 mg, 1.27 mmol, 2.00 eq.) and (1R,2R)—N₁,N₂-dimethylcyclohexane-1,2-diamine (180 mg, 1.27 mmol, 2.00 eq.) in DMA (2 mL) was degassed and purged with N₂ (15 psi) 3 times, and then the mixture was stirred at 90° C. for 3 hours under N₂ (15 psi) atmosphere. The mixture was poured into saturated H₂O aqueous solution (30 mL) and extracted with EA (10 mL×3). The combined organic layer was washed with brine (20 mL), dried over Na₂SO₄, then the mixture was filtered, and the filtrate was concentrated. The residue was purified by column chromatography (SiO₂, Petroleum ether/Ethyl acetate=10/1 to 1/1). Racemic compound 5-cyclobutylsulfinyl-1-[3-(4-tetrahydropyran-2-yl-1,2,4-triazol-3-yl)phenyl]pyrazolo[3,4-b]pyridine (280 mg, 593 mol, 94% yield, 95% purity) was obtained as a white oil. LCMS [ESI, M+1]: 365.1.

To a solution of 5-cyclobutylsulfinyl-1-[3-(4-tetrahydropyran-2-yl-1,2,4-triazol-3-yl)phenyl]pyrazolo[3,4-b]pyridine (260 mg, 580 μmol, 1.00 eq.) in MeOH (8 mL) was added TsOH·H₂O (165 mg, 869 mol, 1.50 eq.). The mixture was stirred at 50° C. for 2 hours. The mixture was poured into saturated NaHCO₃ aqueous solution (30 mL) and extracted with EA (10 mL×3). The combined organic layer was washed with brine (20 mL), dried over Na₂SO₄, then the mixture was filtered, and the filtrate was concentrated. The residue was purified by prep-HPLC (0.1% NH₃·H₂O) to give desired compound as a white solid which was further separated by SFC (column: DAICEL CHIRALCEL OD (250 mm*30 mm, 10 um); mobile phase: [CO₂-EtOH (0.1% NH₃H₂O)]; B %: 55%-55%, 5.3 min). Peak 1 (Compound 18A): (40 mg, 105 mol, 38% yield, 96% purity) was obtained as a white solid. Peak 2 (Compound 18B): (48.8 mg, 130 mol, 47% yield, 97% purity) was obtained as a white solid.

Compound 18A: LCMS [ESI, M+1]: 365.1. ¹H NMR (400 MHz, DMSO-d₆) δ=14.48-14.10 (m, 1H), 8.96 (t, J=1.6 Hz, 1H), 8.89 (d, J=2.1 Hz, 1H), 8.66 (d, J=2.0 Hz, 1H), 8.64 (s, 1H), 8.62-8.46 (m, 1H), 8.37-8.31 (m, 1H), 8.02 (d, J=7.6 Hz, 1H), 7.70 (t, J=8.0 Hz, 1H), 3.92 (quin, J=8.4 Hz, 1H), 2.40 (s, 1H), 2.36-2.33 (m, 1H), 2.25-2.12 (m, 1H), 1.99-1.88 (m, 1H), 1.87-1.74 (m, 1H), 1.68-1.55 (m, 1H).

Compound 18B: LCMS [ESI, M+1]: 365.1 ¹H NMR (400 MHz, DMSO-d₆) δ=14.44-14.11 (m, 1H), 8.96 (t, J=1.6 Hz, 1H), 8.89 (d, J=2.0 Hz, 1H), 8.66 (d, J=2.0 Hz, 1H), 8.64 (s, 1H), 8.61-8.45 (m, 1H), 8.34 (br d, J=8.0 Hz, 1H), 8.05-8.00 (m, 1H), 7.70 (t, J=8.0 Hz, 1H), 3.92 (quin, J=8.4 Hz, 1H), 2.40-2.36 (m, 1H), 2.36-2.32 (m, 1H), 2.18 (ddt, J=3.6, 8.0, 11.6 Hz, 1H), 2.00-1.88 (m, 1H), 1.87-1.77 (m, 1H), 1.66-1.55 (m, 1H).

### Example 3. Representative Procedure C: Synthesis of THP-Protected Aryl Iodides

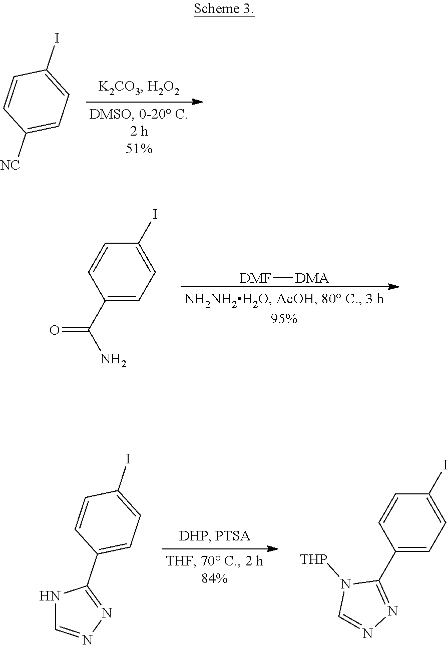

To a solution of 4-iodobenzonitrile (15 g, 65.5 mmol, 1 eq) in DMSO (100 mL) was added K₂CO₃ (13.6 g, 98.2 mmol, 1.5 eq) and H₂O₂ (14.2 g, 125 mmol, 12 mL, 30% purity, 1.91 eq) at 0° C. The mixture was stirred at 20° C. for 2 hr. Water (200 ml) was added to the mixture, and the mixture weas cooled to rt. The precipitate was filtered, and the filter cake was dried under vacuum to obtain a yellow solid. The residue was purified by column chromatography (SiO₂, PE/EA=10/1 to 0/1) to provide 4-iodobenzamide (8.3 g, 33.6 mmol, 51% yield) as a white solid. LCMS (ESI, M+1)=247.9.

A solution of 4-iodobenzamide (8 g, 32.4 mmol, 1 eq) in DMF-DMA (50 mL) was stirred at 80° C. for 1 h, and the reaction mixture was concentrated under vacuum. The residue was dissolved in AcOH (50 mL) followed by addition of NH₂NH₂·H₂O (4.14 g, 81.0 mmol, 4.02 mL, 98% purity, 2.5 eq) at 0° C., and the reaction was then heated and stirred at 80° C. for 2 h. The mixture was poured into saturated NaHCO₃ aqueous solution (100 mL) and extracted with EA (30 ml×3). The combined organic layer was washed with brine (50 mL), dried over Na₂SO₄, then the mixture was filtered, and the filtrate was concentrated. The crude product was triturated with H₂O at 0° C. for 10 min, then filtered and dried to provide 3-(4-iodophenyl)-4H-1,2,4-triazole (8.3 g, 30.6 mmol, 95% yield) as a white solid. ¹H NMR (400 MHz, DMSO-d₆) δ=14.66-13.84 (m, 1H), 8.60-8.35 (m, 1H), 7.88-7.83 (m, 2H), 7.82-7.76 (m, 2H).

To a solution of 3-(4-iodophenyl)-4H-1,2,4-triazole (1 g, 3.69 mmol, 1 eq) in THF (5 mL) was added DHP (1.55 g, 18.5 mmol, 1.69 mL, 5 eq) and PTSA (63.5 mg, 369 μmol, 0.1 eq). The mixture was stirred at 70° C. for 2 hr. The mixture was poured into H₂O (20 mL) and extracted with EA (20 ml×3). The combined organic layers were washed with brine (50 mL), dried over Na₂SO₄, then the mixture was filtered, and the filtrate was concentrated. The residue was purified by column chromatography (SiO₂, PE/EA=10/1 to 0/1) to obtain 3-(4-iodophenyl)-4-tetrahydropyran-2-yl-1,2,4-triazole (1.1 g, 3.10 mmol, 84% yield) as a white solid. ¹H NMR (400 MHz, chloroform-d) 6=8.29 (s, 1H), 7.91-7.83 (m, 2H), 7.81-7.72 (m, 2H), 5.60-5.39 (m, 1H), 3.83-3.66 (m, 1H), 2.21-2.06 (m, 3H), 1.79-1.63 (m, 3H).

### Example 4. Representative Procedure D for Synthesis of Non-Triazole Sulfoxides: Ethyl N-[5-[5-(4,4-difluorocyclohexyl)sulfinylpyrazolo[3,4-b]pyridin-1-yl]-3-pyridyl]carbamate (Compound 39)

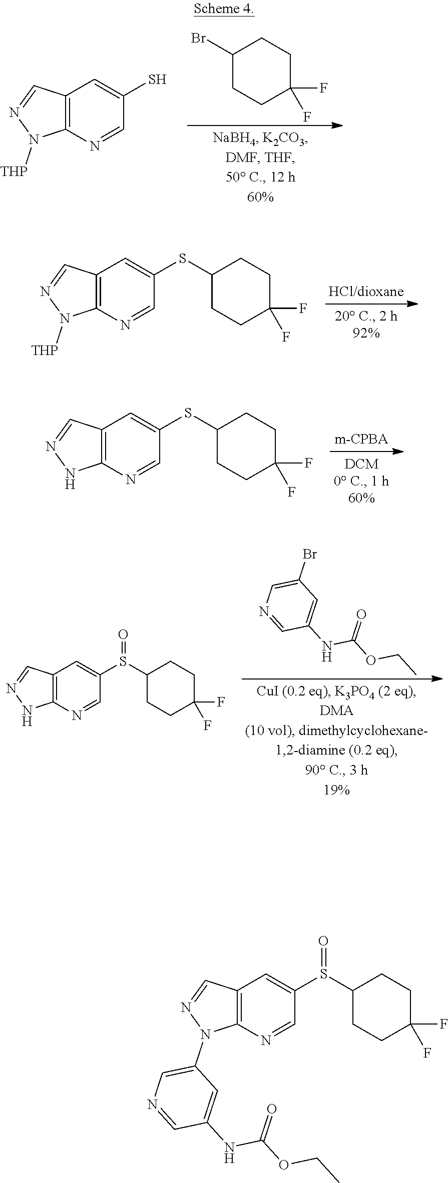

To a solution of 1-tetrahydropyran-2-ylpyrazolo[3,4-b]pyridine-5-thiol (800 mg, 3.40 mmol, 1.00 eq.) and 4-bromo-1,1-difluorocyclohexane (677 mg, 3.40 mmol, 1.00 eq.) in THF (16.0 mL) and DMF (8.0 mL) was added K₂CO₃ (940 mg, 6.80 mmol, 2.00 eq.) at 0° C. The mixture was heated and stirred at 50° C. for 12 hours. The reaction mixture was quenched with water (20.0 mL) at 0° C., and then extracted with EA (100 mL×3). The combined organic layers were washed with water (100 mL×2) and brine (100 mL×2), dried over Na₂SO₄, filtered and concentrated under reduced pressure to give a residue. The residue was purified by column chromatography (SiO₂, Petroleum ether/Ethyl acetate=10/1 to 1/1). The desired fractions were concentrated to give compound 5-(4,4-difluorocyclohexyl)sulfanyl-1-tetrahydropyran-2-yl-pyrazolo[3,4-b]pyridine (0.80 g, 2.04 mmol, 60% yield, 90% purity) as a colorless gum. LCMS [ESI, M+1]: 354.1.

To a solution of 5-(4,4-difluorocyclohexyl)sulfanyl-1-tetrahydropyran-2-yl-pyrazolo[3,4-b]pyridine (0.40 g, 1.13 mmol, 1.00 eq.) in 1,4-dioxane (4.00 mL) was added HCl/dioxane (4 M, 283 μL, 1.00 eq.). The mixture was stirred at 0° C. for 1 hour. The reaction mixture was then diluted with ethyl acetate (120 mL) and washed with water (100 mL×2). The organic layers were dried over anhydrous sodium sulfate, filtered and concentrated under reduced pressure to give a residue. The residue was purified by column chromatography (SiO₂, Petroleum ether/Ethyl acetate=10/1 to 5/1). The desired fractions were concentrated to give compound 5-(4,4-difluorocyclohexyl)sulfanyl-1H-pyrazolo[3,4-b]pyridine (280 mg, 1.04 mmol, 92% yield) as a colorless oil. LCMS [ESI, M+1]: 270.0.

To a solution of 5-(4,4-difluorocyclohexyl)sulfanyl-1H-pyrazolo[3,4-b]pyridine (250 mg, 928 μmol, 1.00 eq.) in DCM (10 mL) was added m-CPBA (188 mg, 929 μmol, 85% purity, 1.00 eq.) at 0° C. The mixture was stirred at 0° C. for 1 hour. The reaction mixture was quenched with saturated sodium sulfite solution (70.0 mL) at 0° C., and extracted with DCM (70 mL×3). The combined organic layers were washed with brine (200 mL), dried over anhydrous sodium sulfate, filtered and concentrated under reduced pressure to give a residue. The residue was purified by column chromatography (SiO₂, Petroleum ether/Ethyl acetate=1/1 to 1/4) and concentrated to give racemic compound 5-(4,4-difluorocyclohexyl)sulfinyl-1H-pyrazolo[3,4-b]pyridine (160 mg, 561 μmol, 60% yield) as a yellow solid. LCMS [ESI, M+1]: 286.0. ¹H NMR (400 MHz, chloroform-d) 6=8.73 (d, J=1.6 Hz, 1H), 8.52 (d, J=2.0 Hz, 1H), 8.25 (s, 1H), 2.82-2.71 (m, 1H), 2.35-2.16 (m, 2H), 2.02-1.68 (m, 6H).

A mixture of 5-(4,4-difluorocyclohexyl)sulfinyl-1H-pyrazolo[3,4-b]pyridine (70.0 mg, 245 μmol, 1.00 eq.), ethyl N-(5-bromo-3-pyridyl)carbamate (72.2 mg, 294 μmol, 1.20 eq.), CuI (9.35 mg, 49.1 μmol, 0.20 eq.), K₃PO₄ (104 mg, 491 μmol, 2.00 eq.) and N₁,N₂-dimethylcyclohexane-1,2-diamine (34.9 mg, 245 μmol, 1.00 eq.) in DMAC (2.50 mL) was degassed and purged with N₂ three times, then the mixture was stirred at 90° C. for 3 hours under N₂ atmosphere (15 psi). The reaction mixture was filtered and diluted with ethyl acetate (80.0 mL), then washed with water (80.0 mL×3). The organic layers were dried over anhydrous sodium sulfate, filtered and concentrated under reduced pressure to give a residue. The residue was purified by prep-HPLC (FA condition: column: Phenomenex luna C18 150×25 mm×10 um; mobile phase: [water (FA)-ACN]; B %: 30%-60%, 10 min). The desired fractions were lyophilized to provide the title compound (Compound 39) (21.02 mg, 46.34 μmol, 19% yield, 99.1% purity) as a white solid.

LCMS [ESI, M+1]: 450.0.

¹H NMR (400 MHz, DMSO-d₆) δ=10.17 (s, 1H), 9.18 (br s, 1H), 8.98-8.85 (m, 2H), 8.70 (d, J=2.0 Hz, 1H), 8.69-8.64 (m, 1H), 8.61 (br s, 1H), 4.19 (q, J=7.2 Hz, 2H), 3.16-3.06 (m, 1H), 2.21-2.01 (m, 3H), 2.00-1.75 (m, 2H), 1.72-1.55 (m, 3H), 1.28 (t, J=7.2 Hz, 3H).

¹⁹F NMR (377 MHz, DMSO-d₆) δ=−90.83, −99.82.

### Example 5. Representative Procedure E for Synthesis of alkoxy-substituted sulfoxides: 1-(3-(4H-1,2,4-triazol-3-yl)phenyl)-5-((isopropoxymethyl)sulfinyl)-1H-pyrazolo[3,4-b]pyridine (Compounds 2A and 2B)

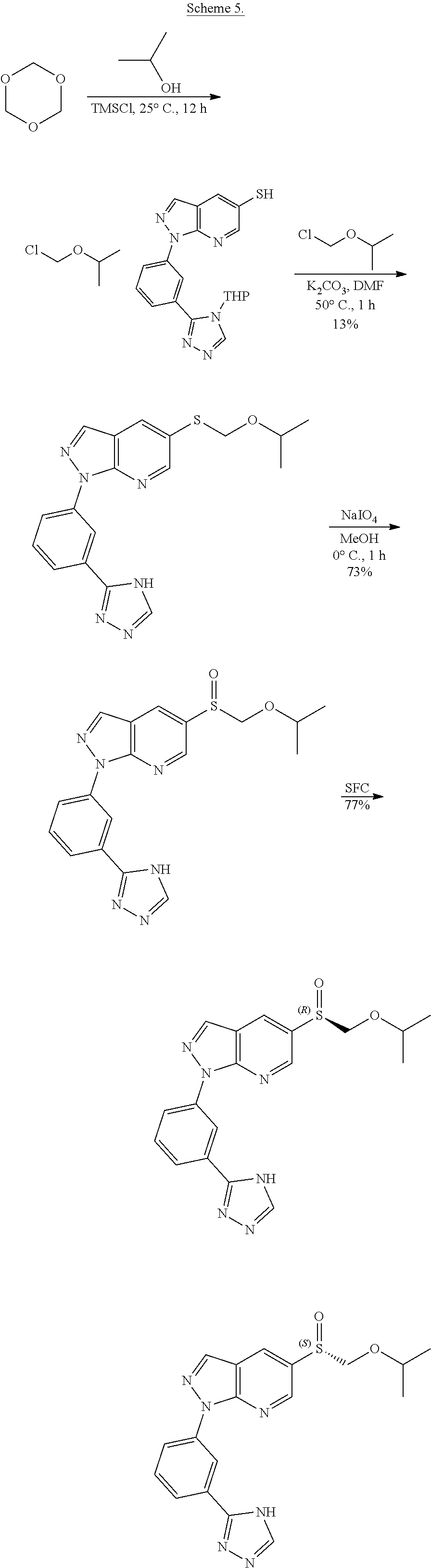

To a solution of propan-2-ol (1.50 g, 25.0 mmol, 1.91 mL, 1.00 eq.) was added TMSCl (5.42 g, 49.9 mmol, 6.34 mL, 2.00 eq.) and 1,3,5-trioxane (1.12 g, 12.5 mmol, 0.50 eq.), the mixture was stirred at 20° C. for 2 hours. The mixture was added to PE (50 mL), then was added saturated NaHCO₃ solution, and the organic phase was concentrated to obtain the crude 2-(chloromethoxy) propane (1.2 g, crude) as a colorless oil. The crude intermediate was used for next step.

To a solution of 1-[3-(4-tetrahydropyran-2-yl-1,2,4-triazol-3-yl) phenyl]pyrazolo[3,4-b]pyridine-5-thiol (1.00 g, 2.64 mmol, 1.00 eq.) in DMF (25.0 mL) was added 2-(chloromethoxy)propane (430 mg, 3.96 mmol, 1.50 eq.) and K₂CO₃ (545 mg, 3.96 mmol, 1.50 eq.), the mixture was stirred at 50° C. for 0.5 hour. The mixture was added to H₂O (30 mL), then extracted with EA (30 mL×3), and the organic phase was concentrated to obtain the crude product which was purified by silica gel chromatography (ISCO®; X g SepaFlash® Silica Flash Column, Ethyl acetate/Petroleum ether gradient=1/2-1/1). 1-(3-(4H-1,2,4-triazol-3-yl)phenyl)-5-((isopropoxymethyl)thio)-1H-pyrazolo[3,4-b]pyridine (160 mg, 349 mol, 13% yield) was obtained as a yellow solid. LCMS [ESI, M+1]: 367.1.

To a solution of 5-(isopropoxymethylsulfanyl)-1-[3-(4H-1,2,4-triazol-3-yl) phenyl]pyrazolo[3,4-b]pyridine (100 mg, 273 μmol, 1.00 eq.) in MeOH (5 mL) was added NaIO₄ (117 mg, 546 μmol, 2.00 eq.) in H₂O (5.00 mL). The mixture was stirred at 25° C. for 2 hours. The reaction mixture was diluted with saturated Na₂SO₃ (30 mL) and extracted with EA (20 mL×3). The combined organic layers were washed with brine (100 mL×1), dried over Na₂SO₄, filtered and concentrated under reduced pressure to give a residue. The crude product was purified by reversed-phase HPLC (0.1% FA condition) to give compound 5-(isopropoxymethylsulfinyl)-1-[3-(4H-1,2,4-triazol-3-yl)phenyl]pyrazolo[3,4-b]pyridine (80.0 mg, 199 mol, 73% yield) as a white solid. LCMS [ESI, M+1]: 383.1.

5-(isopropoxymethylsulfinyl)-1-[3-(4H-1,2,4-triazol-3-yl) phenyl]pyrazolo[3,4-b]pyridine (70.0 mg, 183 mol, 1.00 eq.) was separated by SFC (column: DAICEL CHIRALPAK AS (250 mm×30 mm, 10 μm); mobile phase: [CO₂-ACN/MeOH (0.1% NH₃·H₂O)]; B %:40%, isocratic elution mode) to give Peak 1 (Compound 2A) (25.0 mg, 64.1 mol, 35% yield) as a white solid and Peak 2 (Compound 2B) (30.0 mg, 76.9 mol, 42% yield) as a white solid.

Peak 1-2A: LCMS [ESI, M+1]: 383.1. ¹H NMR (400 MHz, DMSO-d₆) δ=14.55-13.84 (m, 1H), 9.00-8.91 (m, 2H), 8.75 (d, J=2.0 Hz, 1H), 8.67 (s, 1H), 8.64-8.45 (m, 1H), 8.35 (br d, J=7.6 Hz, 1H), 8.03 (d, J=7.6 Hz, 1H), 7.71 (t, J=8.0 Hz, 1H), 4.89-4.60 (m, 2H), 4.00 (td, J=6.0, 12.4 Hz, 1H), 1.16 (d, J=6.0 Hz, 6H).

Peak 2-2B: LCMS [ESI, M+1]: 383.1. ¹H NMR (400 MHz, DMSO-d₆) δ=14.66-13.90 (m, 1H), 9.00-8.92 (m, 2H), 8.75 (d, J=2.0 Hz, 1H), 8.67 (s, 1H), 8.63-8.48 (m, 1H), 8.41-8.27 (m, 1H), 8.03 (d, J=7.6 Hz, 1H), 7.71 (t, J=8.0 Hz, 1H), 4.89-4.60 (m, 2H), 4.00 (td, J=6.0, 12.2 Hz, 1H), 1.16 (d, J=6.0 Hz, 6H).

### Example 6. Representative Procedure F for Synthesis of Sulfones: 1-[3-chloro-5-(4-tetrahydropyran-2-yl-1,2,4-triazol-3-yl)phenyl]-5-cyclopentylsulfonyl-pyrazolo[3,4-b]pyridine (Compound 31)

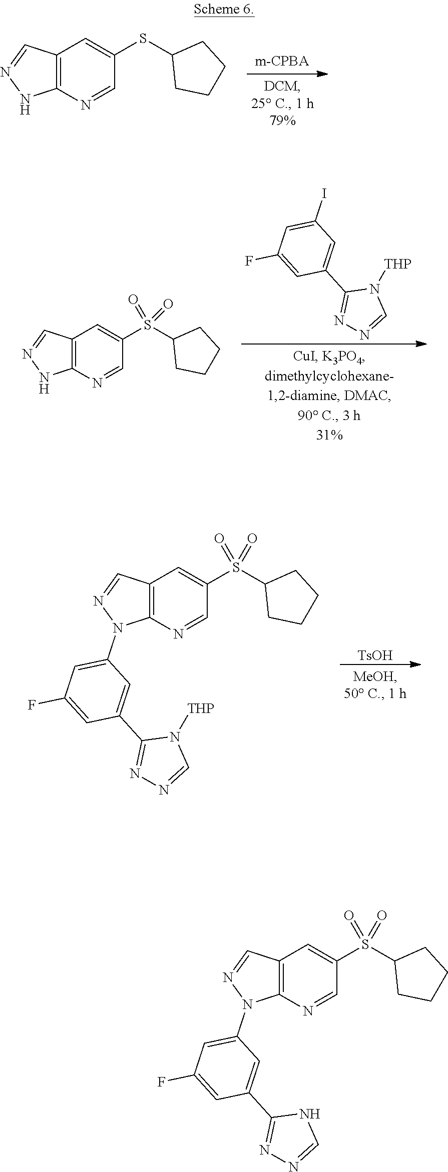

A mixture of 5-cyclopentylsulfonyl-1H-pyrazolo[3,4-b]pyridine (230 mg, 915 μmol, 1.00 eq.), 3-(3-chloro-5-iodo-phenyl)-4-tetrahydropyran-2-yl-1,2,4-triazole (428 mg, 1.10 mmol, 1.20 eq.), CuI (174 mg, 915 μmol, 1.00 eq.), K₃PO₄ (389 mg, 1.83 mmol, 2.00 eq.) and dimethylcyclohexane-1,2-diamine (130 mg, 915 μmol, 1.00 eq.) in DMAC (3 mL) was degassed and purged with N₂ three times, then the mixture was stirred at 90° C. for 3 hours under N₂ atmosphere. The reaction mixture was diluted with water (50 mL) and extracted with EA (100 mL×3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=10/1 to 0/1) to give 1-[3-chloro-5-(4-tetrahydropyran-2-yl-1,2,4-triazol-3-yl)phenyl]-5-cyclopentylsulfonyl-pyrazolo [3,4-b]pyridine (250 mg, 36% yield, 68% purity) as a yellow oil. LCMS (ESI, M+1): m/z=429.0.

To a solution of 1-[3-chloro-5-(4-tetrahydropyran-2-yl-1,2,4-triazol-3-yl)phenyl]-5-cyclopentylsulfonyl-pyrazolo[3,4-b]pyridine (167 mg, 195 (mol, 605 purity, 1.00 eq.) in MOH (1 mL) was added TsOH (33.6 mg, 195.0 μmol, 1.00 eq.). The mixture was stirred at 50° C. for 1 hour. The reaction mixture was quenched by addition NaHCO₃ (10 mL) and extracted with EA (10 mL×3). The combined organic layers were washed with brine (10 mL), dried over Na₂SO₄, filtered and concentrated under reduced pressure to give a residue. The crude product was triturated with MeOH (2 mL) at 25° C. for 30 min to give compound the title compound (Compound 31) (47.2 mg, 105 μmol, 54% yield, 96% purity) as a white solid. LCMS (ESI, M+1): m/z=429.1. ¹H NMR (400 MHz, DMSO-d₆) δ=14.37 (br s, 1H), 9.17 (s, 1H), 8.99 (s, 1H), 8.94 (br s, 1H), 8.75 (br s, 2H), 8.47 (br s, 1H), 8.02 (br s, 1H), 4.03-3.88 (m, 1H), 2.03-1.77 (m, 422), 1.76-1.47 (m, 4H).

The following compounds were made by Examples 1-6.

TABLE 2
Analytical data for representative compounds of the disclosure.

| Comp. | Chiral | | |
|---|---|---|---|
| No. | SFC | ¹H NMR | LCMS |
| 3B | | ¹H NMR (400 MHz, DMSO-d₆) δ = 11.55-10.58 (m, 1H), | LCMS [ESI, |
| | | 8.94 (d, J = 2.0 Hz, 1H), 8.71 (d, J = 2.0 Hz, 1H), 8.64 (s, | M + 1]: 441.1 |
| | | 1H), 8.56 (s, 1H), 8.36-8.26 (m, 1H), 7.78-7.65 (m, 2H), | |
| | | 7.53 (d, J = 6.8 Hz, 1H), 6.63 (s, 1H), 6.56 (br d, J = 6.4 | |
| | | Hz, 1H), 3.82 (br t, J = 7.6 Hz, 1H), 2.46-2.31 (m, 1H), | |
| | | 2.29-2.08 (m, 4H), 2.07-1.93 (m, 1H). | |
| 4B | | ¹H NMR (400 MHz, DMSO-d₆) δ = 10.85 (s, 1H), 8.96 (s, | LCMS [ESI, |
| | | 1H), 8.74 (s, 1H), 8.65 (s, 1H), 8.56 (s, 1H), 8.31 (br s, | M + 1]: 441.1 |
| | | 1H), 7.71 (br d, J = 4.8 Hz, 2H), 7.53 (br d, J = 6.8 Hz, | |
| | | 1H), 6.64 (s, 1H), 6.56 (br d, J = 6.4 Hz, 1H), 3.82-3.75 | |
| | | (m, 1H), 2.64-2.57 (m, 1H), 2.38 (dq, J = 8.3, 15.6 Hz, | |
| | | 1H), 2.22-2.00 (m, 3H), 1.72-1.60 (m, 1H). | |
| 3A | | ¹H NMR (400 MHz, DMSO-d₆) δ = 11.70 (br d, J = 2.4 Hz, | LCMS [ESI, |
| | | 1H), 8.94 (d, J = 2.0 Hz, 1H), 8.71 (d, J = 2.0 Hz, 1H), 8.65 | M + 1]: 441.1 |
| | | (s, 1H), 8.57 (s, 1H), 8.31 (dt, J = 2.0, 4.8 Hz, 1H), 7.72 (d, | |
| | | J = 5.2 Hz, 2H), 7.51 (d, J = 6.8 Hz, 1H), 6.66 (d, J = 1.6 | |
| | | Hz, 1H), 6.57 (dd, J = 1.6, 6.8 Hz, 1H), 3.89-3.75 (m, | |
| | | 1H), 2.45-2.35 (m, 1H), 2.28-2.09 (m, 4H), 2.08-1.93 | |
| | | (m, 1H). | |
| 4A | | ¹H NMR (400 MHz, DMSO-d₆) δ = 11.85-11.48 (m, 1H), | LCMS [ESI, |
| | | 8.96 (d, J = 2.0 Hz, 1H), 8.74 (d, J = 2.0 Hz, 1H), 8.66 (s, | M + 1]: 441.2 |
| | | 1H), 8.57 (s, 1H), 8.32 (dt, J = 2.4, 4.4 Hz, 1H), 7.72 (d, | |
| | | J = 5.2 Hz, 2H), 7.51 (d, J = 6.8 Hz, 1H), 6.66 (s, 1H), 6.57 | |
| | | (dd, J = 1.6, 6.8 Hz, 1H), 3.78 (quin, J = 8.0 Hz, 1H), | |
| | | 2.63-2.54 (m, 1H), 2.37 (td, J = 7.8, 16.0 Hz, 1H), 2.22-2.02 | |
| | | (m, 3H), 1.71-1.58 (m, 1H). | |
| 5C | | ¹H NMR (400 MHz, DMSO-d₆) δ = 11.91 (br s, 1H), 8.91 | LCMS [ESI, |
| | | (d, J = 2.0 Hz, 1H), 8.75-8.66 (m, 1H), 8.62 (s, 1H), 8.36 | M + 1]: 441.1 |
| | | (s, 1H), 8.13 (br d, J = 7.6 Hz, 1H), 7.89 (dd, J = 2.4, 9.6 | |
| | | Hz, 1H), 7.80 (d, J = 2.4 Hz, 1H), 7.66-7.60 (m, 1H), | |
| | | 7.60-7.55 (m, 1H), 6.48 (d, J = 9.6 Hz, 1H), 3.91-3.70 (m, | |
| | | 1H), 2.47-2.29 (m, 1H), 2.29-2.08 (m, 4H), 2.07-2.00 | |
| | | (m, 1H) | |
| 5A | | ¹H NMR (400 MHz, DMSO-d₆) δ = 11.91 (br s, 1H), 8.92 | LCMS [ESI, |
| | | (d, J = 2.0 Hz, 1H), 8.70 (d, J = 2.0 Hz, 1H), 8.62 (s, 1H), | M + 1]: 441.1 |
| | | 8.36 (s, 1H), 8.14 (d, J = 8.0 Hz, 1H), 7.90 (dd, J = 2.8, | |
| | | 9.6 Hz, 1H), 7.80 (d, J = 2.4 Hz, 1H), 7.70-7.61 (m, 1H), | |
| | | 7.61-7.55 (m, 1H), 6.48 (d, J = 9.6 Hz, 1H), 3.91-3.75 (m, | |
| | | 1H), 2.45-2.30 (m, 1H), 2.28-2.08 (m, 4H), 2.07-2.00 | |
| | | (m, 1H). | |
| 5D | | ¹H NMR (400 MHz, DMSO-d₆) δ = 11.86 (br s, 1H), 8.94 | LCMS [ESI, |
| | | (d, J = 2.0 Hz, 1H), 8.74 (d, J = 2.0 Hz, 1H), 8.63 (s, 1H), | M + 1]: 441.1 |
| | | 8.41-8.32 (m, 1H), 8.14 (dd, J = 2.0, 7.6 Hz, 1H), 7.90 | |
| | | (dd, J = 2.8, 9.6 Hz, 1H), 7.81 (d, J = 2.8 Hz, 1H), 7.67- | |
| | | 7.61 (m, 1H), 7.61-7.58 (m, 1H), 6.48 (d, J = 9.6 Hz, 1H), | |
| | | 3.84-3.69 (m, 1H), 2.64-2.54 (m, 1H), 2.46-2.27 (m, | |
| | | 1H), 2.23-2.00 (m, 3H), 1.71-1.61 (m, 1H). | |
| 5B | | ¹H NMR (400 MHz, DMSO-d₆) δ = 11.89 (br s, 1H), 8.94 | LCMS [ESI, |
| | | (d, J = 2.0 Hz, 1H), 8.74 (d, J = 2.0 Hz, 1H), 8.63 (s, 1H), | M + 1]: 441.1 |
| | | 8.37 (s, 1H), 8.14 (br d, J = 7.6 Hz, 1H), 7.90 (dd, J = 2.8, | |
| | | 9.6 Hz, 1H), 7.81 (d, J = 2.4 Hz, 1H), 7.67-7.61 (m, 1H), | |
| | | 7.61-7.57 (m, 1H), 6.48 (d, J = 9.6 Hz, 1H), 3.77 (quin, | |
| | | J = 8.0 Hz, 1H), 2.64-2.53 (m, 1H), 2.44-2.31 (m, 1H), | |
| | | 2.20-2.00 (m, 3H), 1.72-1.61 (m, 1H). | |
| 6 | | ¹H NMR (400 MHz, DMSO-d₆) δ = 14.56-13.89 (m, 1H), 9.16 | LCMS [ESI, |
| | | (d, J = 2.0 Hz, 1H), 9.04 (d, J = 2.0 Hz, 1H), 8.91 (d, J = | M + 1]: 417.1 |
| | | 1.6 Hz, 1H), 8.75 (s, 1H), 8.62-8.50 (m, 1H), 8.31 (br d, | |
| | | J = 8.4 Hz, 1H), 8.07 (d, J = 7.6 Hz, 1H), 7.73 (t, J = 8.0 | |
| | | Hz, 1H), 4.38-4.25 (m, 1H), 3.15-2.94 (m, 4H) | |
| 7B | 2nd peak | ¹H NMR (400 MHz, DMSO-d₆) δ = 14.46-14.05 (m, 1H), | LCMS (ESI, |
| | | 8.98-8.91 (m, 2H), 8.71 (d, J = 1.6 Hz, 1H), 8.65 (s, 2H), | M + 1): m/z = |
| | | 8.33 (br d, J = 7.2 Hz, 1H), 8.03 (d, J = 7.6 Hz, 1H), 7.71 | 401.1 |
| | | (br t, J = 7.6 Hz, 1H), 3.89 (dt, J = 4.4, 8.4 Hz, 1H), 3.03- | |
| | | 2.86 (m, 3H), 2.47-2.34 (m, 1H) | |
| 7A | 1st peak | ¹H NMR (400 MHz, DMSO-d₆) δ = 14.56-14.01 (m, 1H), | LCMS (ESI, |
| | | 8.99-8.90 (m, 2H), 8.71 (d, J = 2.0 Hz, 1H), 8.65 (s, 2H), | M + 1): m/z = |
| | | 8.33 (br d, J = 7.2 Hz, 1H), 8.03 (d, J = 7.6 Hz, 1H), 7.71 | 401.1 |
| | | (br t, J = 8.0 Hz, 1H), 3.89 (dt, J = 4.4, 8.4 Hz, 1H), 3.02- | |
| | | 2.86 (m, 3H), 2.47-2.35 (m, 1H) | |
| 8B | 2nd peak | ¹H NMR (400 MHz, DMSO-d₆) δ = 8.96 (d, J = 2.0 Hz, | LCMS [ESI, |
| | | 2H), 8.74 (d, J = 2.0 Hz, 1H), 8.66 (s, 1H), 8.53 (s, 1H), | M + 1]: 415.2. |
| | | 8.34 (dd, J = 1.2, 8.0 Hz, 1H), 8.03 (d, J = 7.6 Hz, 1H), | |
| | | 7.71 (t, J = 8.0 Hz, 1H), 3.78 (quin, J = 8.4 Hz, 1H), | |
| | | 2.65-2.52 (m, 1H), 2.45-2.28 (m, 1H), 2.22-2.00 (m, 3H), | |
| | | 1.74-1.53 (m, 1H). | |
| 8A | 1 st peak | ¹H NMR (400 MHz, DMSO-d₆) δ = 9.02-8.90 (m, 2H), | LCMS [ESI, |
| | | 8.72 (d, J = 2.0 Hz, 1H), 8.65 (s, 1H), 8.52 (s, 1H), 8.33 | M + 1]: 415.2. |
| | | (td, J = 1.2, 8.0 Hz, 1H), 8.03 (d, J = 8.0 Hz, 1H), 7.70 (t, | |
| | | J = 8.0 Hz, 1H), 3.92-3.73 (m, 1H), 2.46-2.30 (m, 1H), | |
| | | 2.29-2.10 (m, 4H), 2.09-1.91 (m, 1H) | |
| 9B | 2nd peak | ¹H NMR (400 MHz, DMSO-d6) δ = 9.52 (d, J = 2.4 Hz, | LCMS (ESI, |
| | | 1H), 9.26 (t, J = 2.0 Hz, 1H), 9.18 (d, J = 1.6 Hz, 1H), 8.97 | M + 1): m/z = |
| | | (d, J = 2.0 Hz, 1H), 8.78-8.63 (m, 3H), 3.97-3.82 (m, | 402.1 |
| | | 1H), 3.05-2.82 (m, 3H), 2.46-2.31 (m, 1H) | |
| 9A | 1st peak | ¹H NMR (400 MHz, DMSO-d6) δ = 9.52 (d, J = 2.4 Hz, | LCMS (ESI, |
| | | 1H), 9.26 (t, J = 2.0 Hz, 1H), 9.18 (d, J = 1.6 Hz, 1H), 8.98 | M + 1): m/z = |
| | | (d, J = 2.0 Hz, 1H), 8.74 (d, J = 2.0 Hz, 1H), 8.71 (s, 1H), | 402.1 |
| | | 8.69 (s, 1H), 3.90 (dt, J = 4.4, 8.2 Hz, 1H), 3.07-2.83 (m, | |
| | | 3H), 2.46-2.30 (m, 1H) | |
| 10B | 2nd peak | ¹H NMR (400 MHz, DMSO-d6) δ = 8.94 (d, J = 2.0 Hz, | LCMS [ESI, |
| | | 1H), 8.72 (d, J = 2.0 Hz, 1H), 8.67 (s, 1H), 8.46 (d, J = 8.8 | M + 1]: 378.0 |
| | | Hz, 2H), 8.16 (d, J = 8.8 Hz, 2H), 3.88 (dt, J = 4.4, 8.0 Hz, | |
| | | 1H), 3.09-2.78 (m, 3H), 2.35-2.44 (m, 1H). | |
| 10A | 1st peak | ¹H NMR (400 MHz, DMSO-d₆) δ = 13.07 (br s, 1H), 8.94 | LCMS [ESI, |
| | | (d, J = 2.0 Hz, 1H), 8.72 (d, J = 2.0 Hz, 1H), 8.68 (s, 1H), | M + 1]: 378.0 |
| | | 8.47 (d, J = 8.8 Hz, 2H), 8.16 (d, J = 8.8 Hz, 2H), 3.99- | |
| | | 3.75 (m, 1H), 3.06-2.80 (m, 3H), 2.46-2.32 (m, 1H). | |
| 8D | 4th peak | ¹H NMR (400 MHz, DMSO-d₆) δ = 9.00-8.91 (m, 2H), 8.74 | LCMS [ESI, |
| | | (d, J = 2.0 Hz, 1H), 8.65 (s, 1H), 8.43 (s, 1H), 8.29 | M + 1]: 415.1. |
| | | (dd, J = 1.2, 8.0 Hz, 1H), 8.03 (d, J = 7.6 Hz, 1H), 7.68 (t, | |
| | | J = 8.0 Hz, 1H), 3.78 (td, J = 8.0, 16.0 Hz, 1H), 2.61-2.54 | |
| | | (m, 1H), 2.46-2.30 (m, 1H), 2.20-2.00 (m, 3H), 1.71- | |
| | | 1.57 (m, 1H). | |
| 8C | 3rd peak | ¹H NMR (400 MHz, DMSO-d₆) δ = 14.32 (br s, 1H), 9.00- | LCMS [ESI, |
| | | 8.91 (m, 2H), 8.71 (d, J = 2.0 Hz, 1H), 8.65 (s, 1H), 8.55 | M + 1]: 415.1. |
| | | (s, 1H), 8.34 (dd, J = 1.2, 8.0 Hz, 1H), 8.03 (d, J = 8.0 Hz, | |
| | | 1H), 7.71 (t, J = 8.0 Hz, 1H), 3.82 (quin, J = 7.6 Hz, 1H), | |
| | | 2.47-2.31 (m, 1H), 2.31-2.09 (m, 4H), 2.09-1.90 (m, | |
| | | 1H). | |
| 11D | 4th peak | ¹H NMR (400 MHz, DMSO-d₆) δ = 8.96 (d, J = 2.0 Hz, | LCMS [ESI, |
| | | 1H), 8.75 (d, J = 2.0 Hz, 1H), 8.66 (s, 1H), 8.60 (s, 1H), | M + 1]: 439.3. |
| | | 8.56 (d, J = 5.2 Hz, 1H), 8.35 (br d, J = 8.0 Hz, 1H), 7.84- | |
| | | 7.78 (m, 1H), 7.78-7.72 (m, 1H), 7.66 (s, 1H), 7.57 (br d, | |
| | | J = 5.2 Hz, 1H), 3.78 (quin, J = 8.0 Hz, 1H), 2.70-2.58 | |
| | | (m, 1H), 2.57 (s, 3H), 2.38 (dq, J = 8.0, 15.6 Hz, 1H), | |
| | | 2.24-2.00 (m, 3H), 1.71-1.55 (m, 1H). | |
| 11C | 3rd peak | ¹H NMR (400 MHz, DMSO-d₆) δ = 8.96 (d, J = 2.0 Hz, 1H), | LCMS [ESI, |
| | | 8.75 (d, J = 2.0 Hz, 1H), 8.66 (s, 1H), 8.60 (t, J = 1.6 | M + 1]: 439.1. |
| | | Hz, 1H), 8.56 (d, J = 5.2 Hz, 1H), 8.35 (d, J = 8.0 Hz, 1H), | |
| | | 7.85-7.79 (m, 1H), 7.78-7.71 (m, 1H), 7.66 (s, 1H), 7.58 | |
| | | (dd, J = 1.2, 5.2 Hz, 1H), 3.85-3.70 (m, 1H), 2.57 (s, 3H), | |
| | | 2.48-2.27 (m, 2H), 2.24-2.01 (m, 3H), 1.74-1.55 (m, | |
| | | 1H). | |
| 11B | 2nd peak | ¹H NMR (400 MHz, DMSO-d₆) δ = 8.94 (d, J = 2.0 Hz, | LCMS [ESI, |
| | | 1H), 8.72 (d, J = 2.0 Hz, 1H), 8.65 (s, 1H), 8.60 (s, 1H), | M + 1]: 439.1. |
| | | 8.56 (d, J = 5.2 Hz, 1H), 8.35 (br d, J = 8.0 Hz, 1H), 7.86- | |
| | | 7.78 (m, 1H), 7.78-7.71 (m, 1H), 7.66 (s, 1H), 7.57 (d, J = | |
| | | 5.2 Hz, 1H), 3.93-3.69 (m, 1H), 2.57 (s, 3H), 2.47-2.31 | |
| | | (m, 1H), 2.29-2.09 (m, 4H), 2.08-1.91 (m, 1H). | |
| 11A | 1st peak | ¹H NMR (400 MHz, DMSO-d₆) δ = 8.94 (s, 1H), 8.72 (s, | LCMS [ESI, |
| | | 1H), 8.65 (s, 1H), 8.60 (br s, 1H), 8.56 (br d, J = 4.8 Hz, | M + 1]: 439.1. |
| | | 1H), 8.35 (br d, J = 8.0 Hz, 1H), 7.85-7.78 (m, 1H), 7.78- | |
| | | 7.71 (m, 1H), 7.66 (s, 1H), 7.57 (br d, J = 4.8 Hz, 1H), | |
| | | 3.91-3.69 (m, 1H), 2.57 (s, 3H), 2.44-2.29 (m, 1H), | |
| | | 2.27-2.09 (m, 4H), 2.06-1.89 (m, 1H). | |
| 12B | 2nd peak | ¹H NMR (400 MHz, DMSO-d₆) δ = 10.14 (s, 1H), 9.40 (d, | LCMS [ESI, |
| | | J = 2.4 Hz, 1H), 8.97 (d, J = 2.0 Hz, 1H), 8.90 (d, J = 2.0 | M + 1]:446.2 |
| | | Hz, 1H), 8.84 (t, J = 2.1 Hz, 1H), 8.75 (d, J = 2.0 Hz, 1H), | |
| | | 8.70 (s, 1H), 7.81-7.75 (m, 4H), 3.49 (br s, 1H), 2.09 (s, | |
| | | 3H), 1.96-1.76 (m, 4H), 1.65-1.55 (m, 4H). | |
| 12A | 1st peak | ¹H NMR (400 MHz, DMSO-d₆) δ = 10.14 (s, 1H), 9.40 (d, | LCMS [ESI, |
| | | J = 2.3 Hz, 1H), 8.97 (d, J = 2.0 Hz, 1H), 8.90 (d, J = 2.0 | M + 1]:446.2 |
| | | Hz, 1H), 8.83 (t, J = 2.2 Hz, 1H), 8.75 (d, J = 2.0 Hz, 1H), | |
| | | 8.70 (s, 1H), 7.80-7.75 (m, 4H), 3.53-3.45 (m, 1H), 2.09 | |
| | | (s, 3H), 2.01-1.70 (m, 4H), 1.66-1.54 (m, 4H). | |
| 13 | 2nd peak | ¹H NMR (400 MHz, DMSO-d6) δ = 9.49 (d, J = 2.4 Hz, | LCMS [ESI, |
| | | 1H), 8.98 (t, J = 1.6 Hz, 2H), 8.92 (t, J = 2.0 Hz, 1H), 8.75 | M + 1]: 446.2 |
| | | (d, J = 2.0 Hz, 1H), 8.71 (s, 1H), 8.57 (br d, J = 4.4 Hz, | |
| | | 1H), 8.04-7.99 (m, 2H), 7.97-7.90 (m, 2H), 3.56-3.44 | |
| | | (m, 1H), 2.83 (d, J = 4.4 Hz, 3H), 1.94-1.76 (m, 3H), | |
| | | 1.66-1.47 (m, 5H). | |
| 14B | 2nd peak | ¹H NMR (400 MHz, DMSO-d₆) δ = 10.16 (s, 1H), 9.51 (d, | LCMS (ESI, |
| | | J = 2.4 Hz, 1H), 8.97 (d, J = 2.0 Hz, 1H), 8.85 (d, J = 2.0 | M + 1): m/z = |
| | | Hz, 1H), 8.84-8.80 (m, 1H), 8.76 (d, J = 2.0 Hz, 1H), 8.71 | 446.2 |
| | | (s, 1H), 8.03 (s, 1H), 7.73-7.67 (m, 1H), 7.52-7.47 (m, | |
| | | 2H), 3.49 (br t, J = 7.6 Hz, 1H), 2.09 (s, 3H), 1.94-1.79 | |
| | | (m, 3H), 1.65-1.50 (m, 5H). | |
| 14A | 1st peak | ¹H NMR (400 MHz, DMSO-d₆) δ = 10.14 (s, 1H), 9.51 (d, | LCMS (ESI, |
| | | J = 2.4 Hz, 1H), 8.97 (d, J = 2.0 Hz, 1H), 8.85 (d, J = 2.0 | M + 1): m/z = |
| | | Hz, 1H), 8.82 (t, J = 2.0 Hz, 1H), 8.75 (d, J = 2.0 Hz, 1H), | 446.2 |
| | | 8.71 (s, 1H), 8.03 (s, 1H), 7.74-7.65 (m, 1H), 7.55-7.43 | |
| | | (m, 2H), 3.49 (br t, J = 7.6 Hz, 1H), 2.09 (s, 3H), | |
| | | 1.96-1.78 (m, 3H), 1.67-1.47 (m, 5H). | |
| 16B | 2nd peak | ¹H NMR (400 MHz, DMSO-d₆) δ = 9.50 (d, J = 2.4 Hz, 1H), | LCMS (ESI, |
| | | 8.98 (dd, J = 2.0, 4.4 Hz, 2H), 8.91 (t, J = 2.0 Hz, 1H), | M + 1): m/z = |
| | | 8.76 (d, J = 2.0 Hz, 1H), 8.72 (s, 1H), 8.63 (br d, J = 4.4 | 446.2 |
| | | Hz, 1H), 8.26 (s, 1H), 7.99 (br d, J = 7.6 Hz, 1H), 7.94 (d, | |
| | | J = 8.0 Hz, 1H), 7.66 (t, J = 7.6 Hz, 1H), 3.50 (br t, J = 7.6 | |
| | | Hz, 1H), 2.83 (d, J = 4.4 Hz, 3H), 1.97-1.74 (m, 3H), | |
| | | 1.69-1.45 (m, 5H). | |
| 16A | 1st peak | ¹H NMR (400 MHz, DMSO-d₆) δ = 9.50 (d, J = 2.0 Hz, 1H), | LCMS (ESI, |
| | | 9.02-8.95 (m, 2H), 8.91 (s, 1H), 8.76 (d, J = 1.6 Hz, | M + 1): m/z = |
| | | 1H), 8.72 (s, 1H), 8.63 (br d, J = 4.0 Hz, 1H), 8.26 (s, 1H), | 446.2 |
| | | 7.99 (d, J = 8.0 Hz, 1H), 7.94 (d, J = 7.6 Hz, 1H), 7.66 (t, | |
| | | J = 8.0 Hz, 1H), 3.50 (br t, J = 7.6 Hz, 1H), 2.83 (d, J = 4.4 | |
| | | Hz, 3H), 1.99-1.74 (m, 3H), 1.68-1.44 (m, 5H). | |
| 17B | 2nd peak | ¹H NMR (400 MHz, DMSO-d₆) δ = 14.01-13.59 (m, 1H), 8.95 | LCMS [ESI, |
| | | (d, J = 2.0 Hz, 1H), 8.91 (s, 1H), 8.72 (d, J = 2.0 Hz, | M + 1]: 381.1 |
| | | 1H), 8.64 (s, 1H), 8.32 (br d, J = 7.6 Hz, 1H), 7.98 (d, J = | |
| | | 7.6 Hz, 1H), 7.67 (br t, J = 8.0 Hz, 1H), 3.21-3.07 (m, | |
| | | 1H), 3.04-2.92 (m, 1H), 2.43 (s, 3H), 1.72-1.56 (m, 1H), | |
| | | 1.52-1.30 (m, 3H), 0.91-0.82 (m, 3H) | |
| 17A | 1st peak | ¹H NMR (400 MHz, DMSO-d₆) δ = 13.97-13.66 (m, 1H), 8.95 | LCMS [ESI, |
| | | (d, J = 2.0 Hz, 1H), 8.91 (s, 1H), 8.72 (d, J = 1.6 Hz, | M + 1]: 381.1 |
| | | 1H), 8.64 (s, 1H), 8.32 (br d, J = 8.0 Hz, 1H), 7.98 (d, J = | |
| | | 7.6 Hz, 1H), 7.67 (t, J = 8.0 Hz, 1H), 3.14 (br d, J = 4.0 Hz, | |
| | | 1H), 3.03-2.91 (m, 1H), 2.43 (s, 3H), 1.75-1.58 (m, 1H), | |
| | | 1.52-1.30 (m, 3H), 0.97-0.77 (m, 3H). | |
| 18B | 2nd peak | ¹H NMR (400 MHz, DMSO-d₆) δ = 14.44-14.11 (m, 1H), 8.96 | LCMS [ESI, |
| | | (t, J = 1.6 Hz, 1H), 8.89 (d, J = 2.0 Hz, 1H), 8.66 (d, J = | M + 1]: 365.1 |
| | | 2.0 Hz, 1H), 8.64 (s, 1H), 8.61-8.45 (m, 1H), 8.34 (br d, | |
| | | J = 8.0 Hz, 1H), 8.05-8.00 (m, 1H), 7.70 (t, J = 8.0 Hz, | |
| | | 1H), 3.92 (quin, J = 8.4 Hz, 1H), 2.40-2.36 (m, 1H), 2.36- | |
| | | 2.32 (m, 1H), 2.18 (ddt, J = 3.6, 8.0, 11.6 Hz, 1H), | |
| | | 2.00-1.88 (m, 1H), 1.87-1.77 (m, 1H), 1.66-1.55 (m, 1H). | |
| 18A | 1st peak | ¹H NMR (400 MHz, DMSO-d₆) δ = 14.48-14.10 (m, 1H), 8.96 | LCMS [ESI, |
| | | (t, J = 1.6 Hz, 1H), 8.89 (d, J = 2.1 Hz, 1H), 8.66 (d, J = | M + 1]: 365.1 |
| | | 2.0 Hz, 1H), 8.64 (s, 1H), 8.62-8.46 (m, 1H), 8.37-8.31 | |
| | | (m, 1H), 8.02 (d, J = 7.6 Hz, 1H), 7.70 (t, J = 8.0 Hz, 1H), | |
| | | 3.92 (quin, J = 8.4 Hz, 1H), 2.40 (s, 1H), 2.36-2.33 (m, | |
| | | 1H), 2.25-2.12 (m, 1H), 1.99-1.88 (m, 1H), 1.87-1.74 | |
| | | (m, 1H), 1.68-1.55 (m, 1H). | |
| 19B | 2nd peak | ¹H NMR (400 MHz, DMSO-d₆) δ = 14.65-13.93 (m, 1H), 9.01-8.90 | LCMS [ESI, |
| | | (m, 2H), 8.72 (d, J = 2.0 Hz, 1H), 8.64 (d, J = 1.6 | M + 1]: 421.0 |
| | | Hz, 1H), 8.62-8.47 (m, 1H), 8.33 (br d, J = 7.6 Hz, 1H), | |
| | | 8.02 (d, J = 7.6 Hz, 1H), 7.69 (t, J = 8.0 Hz, 1H), | |
| | | 3.27-3.21 (m, 1H), 3.12-2.98 (m, 1H), 2.45-2.33 (m, 2H), | |
| | | 1.97-1.83 (m, 1H), 1.75-1.57 (m, 1H). | |
| 19A | 1st peak | ¹H NMR (400 MHz, DMSO-d₆) δ = 14.52-14.03 (m, 1H), | LCMS [ESI, |
| | | 9.00-8.91 (m, 2H), 8.74 (s, 1H), 8.66 (s, 1H), 8.63-8.47 | M + 1]: 421.0 |
| | | (m, 1H), 8.35 (br d, J = 7.6 Hz, 1H), 8.03 (d, J = 7.6 Hz, | |
| | | 1H), 7.71 (t, J = 8.0 Hz, 1H), 3.29-3.23 (m, 1H), 3.14- | |
| | | 3.02 (m, 1H), 2.46-2.36 (m, 2H), 1.99-1.85 (m, 1H), | |
| | | 1.75-1.57 (m, 1H). | |
| 20B | 2nd peak | ¹H NMR (400 MHz, DMSO-d₆) δ = 14.58-13.60 (m, 1H), | LCMS [ESI, |
| | | 8.59-8.53 (m, 3H), 8.44 (d, J = 2.0 Hz, 1H), 8.18 (d, J = | M + 1]: 382.2. |
| | | 2.4 Hz, 1H), 8.04 (d, J = 7.6 Hz, 1H), 7.91 (br d, J = 7.6 | |
| | | Hz, 1H), 7.76-7.63 (m, 1H), 6.92 (d, J = 3.6 Hz, 1H), | |
| | | 3.40-3.36 (m, 2H), 3.19 (s, 3H), 3.14-3.04 (m, 1H), | |
| | | 3.03-2.89 (m, 1H), 1.92-1.79 (m, 1H), 1.73-1.58 (m, 1H) | |
| 20A | 1st peak | ¹H NMR (400 MHz, DMSO-d₆) δ = 14.60-13.86 (m, 1H), | LCMS [ESI, |
| | | 8.57-8.53 (m, 3H), 8.43 (d, J = 2.0 Hz, 1H), 8.17 (d, J = | M + 1]: 382.2. |
| | | 3.6 Hz, 1H), 8.04 (d, J = 8.0 Hz, 1H), 7.91 (br d, J = 8.0 | |
| | | Hz, 1H), 7.77-7.59 (m, 1H), 6.91 (d, J = 3.6 Hz, 1H), 3.38 | |
| | | (t, J = 6.4 Hz, 3H), 3.16-3.04 (m, 1H), 3.03-2.91 (m, | |
| | | 1H), 1.93-1.79 (m, 1H), 1.74-1.58 (m, 1H) | |
| 21B | 2nd peak | ¹H NMR (400 MHz, DMSO-d₆) δ = 14.37-14.09 (m, 1H), | LCMS (ESI, |
| | | 9.03-8.92 (m, 2H), 8.72 (s, 1H), 8.72-8.59 (m, 2H), 8.35 | M + 1): m/z = |
| | | (br s, 1H), 8.03 (br d, J = 7.2 Hz, 1H), 7.71 (br d, J = 6.4 | 381.1 |
| | | Hz, 1H), 3.20-3.09 (m, 1H), 3.02-2.90 (m, 1H), 1.68-1.52 | |
| | | (m, 2H), 1.33 (dt, J = 5.6, 11.6 Hz, 1H), 0.85 (br t, J = | |
| | | 6.8 Hz, 6H) | |
| 21A | 1st peak | ¹H NMR (400 MHz, DMSO-d₆) δ = 14.44-13.89 (m, 1H), | LCMS (ESI, |
| | | 9.02-8.94 (m, 2H), 8.72 (d, J = 1.2 Hz, 1H), 8.72-8.61 | M + 1): m/z = |
| | | (m, 2H), 8.45-8.26 (m, 1H), 8.03 (br d, J = 7.6 Hz, 1H), | 381.1 |
| | | 7.77-7.62 (m, 1H), 3.21-3.09 (m, 1H), 3.03-2.92 (m, | |
| | | 1H), 1.69-1.50 (m, 2H), 1.38-1.27 (m, 1H), 0.86 (t, J = | |
| | | 6.8 Hz, 6H). | |
| 22B | 2nd peak | ¹H NMR (400 MHz, DMSO-d₆) δ = 14.52-14.03 (m, 1H), | LCMS [ESI, |
| | | 9.00-8.91 (m, 2H), 8.74 (s, 1H), 8.66 (s, 1H), 8.63-8.47 | M + 1]: 421.0 |
| | | (m, 1H), 8.35 (br d, J = 7.6 Hz, 1H), 8.03 (d, J = 7.6 Hz, | |
| | | 1H), 7.71 (t, J = 8.0 Hz, 1H), 3.29-3.23 (m, 1H), 3.14- | |
| | | 3.02 (m, 1H), 2.46-2.36 (m, 2H), 1.99-1.85 (m, 1H), | |
| | | 1.75-1.57 (m, 1H). | |
| 22A | 1st peak | ¹H NMR (400 MHz, DMSO-d₆) δ = 14.52-14.03 (m, 1H), | LCMS [ESI, |
| | | 9.00-8.91 (m, 2H), 8.74 (s, 1H), 8.66 (s, 1H), 8.63-8.47 | M + 1]: 421.0 |
| | | (m, 1H), 8.35 (br d, J = 7.6 Hz, 1H), 8.03 (d, J = 7.6 Hz, | |
| | | 1H), 7.71 (t, J = 8.0 Hz, 1H), 3.29-3.23 (m, 1H), 3.14- | |
| | | 3.02 (m, 1H), 2.46-2.36 (m, 2H), 1.99-1.85 (m, 1H), | |
| | | 1.75-1.57 (m, 1H). | |
| 23B | 2nd peak | ¹H NMR (400 MHz, DMSO-d6) δ = 14.70-13.83 (m, 1H), | LCMS [ESI, |
| | | 8.97 (d, J = 2.0 Hz, 2H), 8.74 (d, J = 2.0 Hz, 1H), 8.65 | M + H]: 407.0 |
| | | (s, 1H), 8.63-8.46 (m, 1H), 8.34 (br d, J = 8.0 Hz, 1H), | |
| | | 8.03 (d, J = 8.0 Hz, 1H), 7.70 (t, J = 8.0 Hz, 1H), 3.49 | |
| | | (ddd, J = 4.8, 10.4, 13.6 Hz, 1H), 3.22 (ddd, J = 5.2, 10.4, | |
| | | 13.6 Hz, 1H), 2.76-2.66 (m, 1H), 2.44-2.30 (m, 1H). | |
| 23A | 1st peak | ¹H NMR (400 MHz, DMSO-d6) δ = 14.79-13.74 (m, 1H), | LCMS [ESI, |
| | | 9.02-8.94 (m, 2H), 8.74 (d, J = 2.0 Hz, 1H), 8.66 (s, 1H), | M + H]: 407.0 |
| | | 8.56 (br s, 1H), 8.35 (dd, J = 1.2, 8.0 Hz, 1H), 8.03 (td, J = | |
| | | 1.2, 7.6 Hz, 1H), 7.71 (t, J = 8.0 Hz, 1H), 3.52-3.47 (m, | |
| | | 1H), 3.26-3.21 (m, 1H), 2.77-2.66 (m, 1H), 2.41 (tdd, J = | |
| | | 5.2, 10.4, 15.6 Hz, 1H). | |
| 24B | 2nd peak | ¹H NMR (400 MHz, DMSO-d6) δ = 13.06 (br s, 1H), 8.94 (d, | LCMS [ESI, |
| | | J = 1.6 Hz, 1H), 8.72 (d, J = 2.0 Hz, 1H), 8.66 (s, 1H), | M + 1]: 344.1 |
| | | 8.49 (s, 1H), 8.47 (s, 1H), 8.16 (s, 1H), 8.14 (s, 1H), | |
| | | 3.17-3.10 (m, 1H), 3.01-2.92 (m, 1H), 1.70-1.60 (m, 1H), | |
| | | 1.46-1.31 (m, 3H), 0.89-0.82 (m, 3H). | |
| 24A | 1st peak | ¹H NMR (400 MHz, DMSO-d6) δ = 13.08 (br s, 1H), 8.95 | LCMS [ESI, |
| | | (d, J = 2.0 Hz, 1H), 8.72 (d, J = 2.0 Hz, 1H), 8.67 (s, 1H), | M + 1]: 344.1 |
| | | 8.49 (s, 1H), 8.47 (s, 1H), 8.17 (s, 1H), 8.16-8.13 (m, 1H), | |
| | | 3.17-3.11 (m, 1H), 3.01-2.93 (m, 1H), 1.70-1.59 (m, | |
| | | 1H), 1.48-1.32 (m, 3H), 0.90-0.83 (m, 3H). | |
| 25 | racemic | ¹H NMR (400 MHz, DMSO-d6) δ = 14.21 (br s, 1H), 8.97 | LCMS [ESI, |
| | | (d, J = 2.0 Hz, 2H), 8.75 (d, J = 2.0 Hz, 1H), 8.72-8.50 | M + 1]: 383.0 |
| | | (m, 1H), 8.44-8.28 (m, 1H), 8.28-8.28 (m, 1H), 8.03 (br | |
| | | d, J = 7.2 Hz, 1H), 7.70 (br d, J = 7.2 Hz, 1H), 3.81-3.73 | |
| | | (m, 1H), 3.72-3.65 (m, 1H), 3.49-3.37 (m, 3H), 3.23- | |
| | | 3.15 (m, 1H), 1.02 (t, J = 7.2 Hz, 3H). | |
| 26B | 2nd peak | ¹H NMR (400 MHz, DMSO-d6) δ = 14.23 (br d, J = 2.0 Hz, | LCMS (ESI, |
| | | 1H), 9.13-8.89 (m, 2H), 8.83-8.53 (m, 3H), 8.34 (br | M + 1): m/z = |
| | | d, J = 2.8 Hz, 1H), 8.04 (br d, J = 7.6 Hz, 1H), 7.71 (br s, | 379.1. |
| | | 1H), 3.50 (br t, J = 7.2 Hz, 1H), 3.46-3.44 (m, 1H), 1.98- | |
| | | 1.73 (m, 3H), 1.71-1.44 (m, 5H). | |
| 26A | 1st peak | ¹H NMR (400 MHz, DMSO-d6) δ = 14.88-13.84 (m, 1H), | LCMS (ESI, |
| | | 9.12-8.87 (m, 2H), 8.85-8.42 (m, 3H), 8.35 (br d, J = 7.6 | M + 1): m/z = |
| | | Hz, 1H), 8.03 (br d, J = 7.6 Hz, 1H), 7.87-7.62 (m, 1H), | 379.1. |
| | | 3.59-3.39 (m, 1H), 2.07-1.76 (m, 3H), 1.75-1.36 (m, | |
| | | 5H). | |
| 27B | 2nd peak | ¹H NMR (400 MHz, DMSO-d₆) δ = 10.14 (s, 1H), 8.93 (d, | LCMS [ESI, |
| | | J = 2.0 Hz, 1H), 8.71 (d, J = 2.0 Hz, 1H), 8.62 (s, 1H), 8.31 | M + 1]: 405.2. |
| | | (s, 1H), 7.91 (dd, J = 2.0, 10.4 Hz, 1H), 7.45-7.27 (m, | |
| | | 1H), 4.17 (d, J = 7.2 Hz, 2H), 3.18-3.10 (m, 1H), 2.97 (dt, | |
| | | J = 5.2, 8.4 Hz, 1H), 1.73-1.58 (m, 1H), 1.48-1.34 (m, | |
| | | 3H), 1.27 (t, J = 7.2 Hz, 3H), 0.92-0.79 (m, 3H) | |
| 27A28 | 1st peak | ¹H NMR (400 MHz, DMSO-d₆) δ = 10.14 (s, 1H), 8.93 (d, | LCMS [ESI, |
| | | J = 2.0 Hz, 1H), 8.71 (d, J = 2.0 Hz, 1H), 8.63 (s, 1H), 8.31 | M + 1]: 405.3. |
| | | (s, 1H), 7.90 (br d, J = 10.4 Hz, 1H), 7.35 (br d, J = 11.2 | |
| | | Hz, 1H), 4.17 (d, J = 6.8 Hz, 2H), 3.19-3.07 (m, 1H), | |
| | | 3.02-2.90 (m, 1H), 1.72-1.57 (m, 1H), 1.47-1.31 (m, 3H), | |
| | | 1.27 (t, J = 7.2 Hz, 3H), 0.91-0.80 (m, 3H) | |
| 28 | racemic | ¹H NMR (400 MHz, DMSO-d₆) δ = 14.99-13.52 (m, 1H), | LCMS [ESI, |
| | | 8.96 (d, J = 2.0 Hz, 2H), 8.75 (d, J = 2.0 Hz, 1H), 8.65 (s, | M + 1]: 369.0 |
| | | 1H), 8.56 (br s, 1H), 8.39-8.31 (m, 1H), 8.03 (dd, J = 1.2, | |
| | | 7.8 Hz, 1H), 7.71 (t, J = 8.0 Hz, 1H), 3.76 (qd, J = 4.4, 10.8 | |
| | | Hz, 1H), 3.66-3.59 (m, 1H), 3.26 (s, 3H), 3.21 (t, J = 4.4 | |
| | | Hz, 1H), 3.19-3.15 (m, 1H). | |
| 29 | racemic | ¹H NMR (400 MHz, DMSO-d₆) δ = 14.67-13.85 (m, 1H), | LCMS [ESI, |
| | | 8.97 (s, 1H), 8.95 (d, J = 2.0 Hz, 1H), 8.73 (d, J = 2.0 Hz, | M + 1]: 383.0 |
| | | 1H), 8.65 (s, 1H), 8.62-8.48 (m, 1H), 8.35 (br d, J = 8.0 | |
| | | Hz, 1H), 8.03 (d, J = 7.6 Hz, 1H), 7.71 (t, J = 8.0 Hz, 1H), | |
| | | 3.40-3.36 (m, 3H), 3.23-3.18 (m, 2H), 3.16 (br d, J = 5.6 | |
| | | Hz, 1H), 3.08-2.94 (m, 1H), 1.96-1.81 (m, 1H), 1.72- | |
| | | 1.58 (m, 1H). | |
| 30 | racemic | ¹H NMR (400 MHz, DMSO-d₆) δ = 14.55-14.06 (m, 1H), 9.13 | LCMS [ESI, |
| | | (d, J = 2.4 Hz, 1H), 8.97 (d, J = 2.0 Hz, 1H), 8.92 (t, | M + 1]: 383.0 |
| | | J = 1.6 Hz, 1H), 8.74 (s, 1H), 8.66-8.48 (m, 1H), 8.33 (br d, | |
| | | J = 7.6 Hz, 1H), 8.06 (td, J = 1.2, 7.6 Hz, 1H), 7.73 (t, J = | |
| | | 8.0 Hz, 1H), 3.52-3.43 (m, 2H), 1.63-1.53 (m, 2H), 1.41- | |
| | | 1.29 (m, 2H), 0.84 (t, J = 7.2 Hz, 3H) | |
| 31 | racemic | ¹H NMR (400 MHz, DMSO-d₆) δ = 14.37 (br s, 1H), 9.17 | LCMS (ESI, |
| | | (s, 1H), 8.99 (s, 1H), 8.94 (br s, 1H), 8.75 (br s, 2H), 8.47 | M + 1): m/z = |
| | | (br s, 1H), 8.02 (br s, 1H), 4.03-3.88 (m, 1H), 2.03-1.77 | 429.1. |
| | | (m, 4H), 1.76-1.47 (m, 4H). | |
| 32 | racemic | ¹H NMR (400 MHz, DMSO-d₆) δ = 14.89-13.73 (m, 1H), | LCMS (ESI, |
| | | 9.17 (d, J = 2.4 Hz, 1H), 9.00 (d, J = 2.0 Hz, 1H), 8.87 (s, | M + 1): m/z |
| | | 1H), 8.82-8.54 (m, 2H), 8.27 (br d, J = 10.8 Hz, 1H), 7.79 | =413.1. |
| | | (br d, J = 9.6 Hz, 1H), 4.00-3.91 (m, 1H), 1.99-1.82 (m, | |
| | | 4H), 1.73-1.48 (m, 4H). | |
| 33 | racemic | ¹H NMR (400 MHz, DMSO-d₆) δ = 14.37 (br s, 1H), 9.17 | LCMS [ESI, |
| | | (d, J = 2.0 Hz, 1H), 8.98 (d, J = 2.0 Hz, 1H), 8.88 (s, 1H), | M + 1]: 401.0. |
| | | 8.76 (s, 1H), 8.64 (s, 1H), 8.26 (td, J = 2.0, 10.4 Hz, 1H), | |
| | | 7.79 (dd, J = 1.2, 9.2 Hz, 1H), 3.53-3.44 (m, 2H), 1.74- | |
| | | 1.49 (m, 2H), 1.35 (qt, J = 7.2 Hz, 2H), 0.84 (t, J = 7.2 Hz, | |
| | | 3H). | |
| 34 | racemic | ¹H NMR (400 MHz, DMSO-d6) δ = 14.69-13.57 (m, 1H), | LCMS [ESI, |
| | | 9.21-9.07 (m, 1H), 9.06-8.86 (m, 2H), 8.81-8.45 (m, | M + 1]: 395.1. |
| | | 2H), 8.41-8.26 (m, 1H), 8.14-7.99 (m, 1H), 7.81-7.64 | |
| | | (m, 1H), 4.12-3.80 (m, 1H), 1.97-1.80 (m, 4H), 1.73- | |
| | | 1.53 (m, 4H) | |
| 35 | racemic | ¹H NMR (400 MHz, DMSO-d₆) δ = 14.50-13.95 (m, 1H), | LCMS (ESI, |
| | | 8.90 (d, J = 2.0 Hz, 1H), 8.68 (d, J = 2.0 Hz, 1H), 8.65 (s, | M + 1): m/z = |
| | | 1H), 8.60-8.46 (m, 1H), 8.42 (br d, J = 8.4 Hz, 2H), 8.23 | 395.0 |
| | | (d, J = 8.8 Hz, 2H), 3.97-3.87 (m, 2H), 3.32 (br s, 2H), | |
| | | 3.28 (br s, 1H), 1.83-1.72 (m, 1H), 1.71-1.53 (m, 2H), | |
| | | 1.39 (br d, J = 13.2 Hz, 1H). | |
| 36B | | ¹H NMR (400 MHz, DMSO-d6) δ = 14.27 (br s, 1H), 8.97 | LCMS [ESI, |
| | | (s, 1H), 8.94 (s, 1H), 8.71 (s, 1H), 8.63 (s, 1H), 8.56 (br s, | M + 1]: 367.1 |
| | | 1H), 8.34 (br d, J = 7.6 Hz, 1H), 8.03 (br d, J = 7.6 Hz, | |
| | | 1H), 7.70 (t, J = 7.6 Hz, 1H), 3.20-3.06 (m, 1H), 3.04- | |
| | | 2.87 (m, 1H), 1.74-1.56 (m, 1H), 1.52-1.27 (m, 3H), | |
| | | 0.87-0.83 (m, 3H). | |
| 36A | | ¹H NMR (400 MHz, DMSO-d6) δ = 14.29 (br s, 1H), 8.97 | LCMS [ESI, |
| | | (s, 1H), 8.95 (d, J = 2.0 Hz, 1H), 8.72 (d, J = 2.0 Hz, 1H), | M + 1]: 367.1 |
| | | 8.65 (s, 1H), 8.56 (br s, 1H), 8.35 (br d, J = 7.6 Hz, 1H), | |
| | | 8.03 (d, J = 7.6 Hz, 1H), 7.71 (t, J = 7.6 Hz, 1H), 3.20- | |
| | | 3.08 (m, 1H), 3.03-2.93 (m, 1H), 1.73-1.58 (m, 1H), | |
| | | 1.52-1.31 (m, 3H), 0.91-0.82 (m, 3H). | |
| 37 | racemic | ¹H NMR (400 MHz, DMSO-d₆) δ = 14.04 (s, 1H), 9.05 (d, | LCMS (ESI, |
| | | J = 2.4 Hz, 1H), 8.93 (d, J = 2.4 Hz, 1H), 8.73 (s, 1H), 8.51 | M + 1): m/z = |
| | | (s, 1H), 8.39 (d, J = 8.8 Hz, 2H), 8.25 (d, J = 8.8 Hz, 2H), | 411.1 |
| | | 3.92 (br dd, J = 3.6, 11.2 Hz, 2H), 3.75-3.67 (m, 1H), 3.28 | |
| | | (br s, 2H), 1.88-1.74 (m, 2H), 1.61 (dq, J = 4.4, 12.4 Hz, | |
| | | 2H). | |
| 38 | racemic | ¹H NMR (400 MHz, DMSO-d₆) δ = 14.27 (br s, 1H), 8.97 | LCMS (ESI, |
| | | (t, J = 1.6 Hz, 1H), 8.91 (d, J = 2.0 Hz, 1H), 8.73-8.64 (m, | M + 1): m/z = |
| | | 2H), 8.63-8.45 (m, 1H), 8.35 (br d, J = 8.4 Hz, 1H), 8.03 | 395.1 |
| | | (d, J = 8.0 Hz, 1H), 7.71 (t, J = 8.0 Hz, 1H), 3.97-3.86 (m, | |
| | | 2H), 3.30 (s, 2H), 3.25-3.18 (m, 1H), 1.81-1.74 (m, 1H), | |
| | | 1.71-1.55 (m, 2H), 1.39 (br d, J = 11.2 Hz, 1H). | |
| 39 | racemic | ¹H NMR (400 MHz, DMSO-d₆) δ = 10.17 (s, 1H), 9.18 (br | LCMS [ESI, |
| | | s, 1H), 8.98-8.85 (m, 2H), 8.70 (d, J = 2.0 Hz, 1H), 8.69- | M + 1]: 450.0 |
| | | 8.64 (m, 1H), 8.61 (br s, 1H), 4.19 (q, J = 7.2 Hz, 2H), | |
| | | 3.16-3.06 (m, 1H), 2.21-2.01 (m, 3H), 2.00-1.75 (m, 2H), | |
| | | 1.72-1.55 (m, 3H), 1.28 (t, J = 7.2 Hz, 3H). | |
| 40 | racemic | ¹H NMR (400 MHz, DMSO-d₆) δ = 14.21 (br s, 1H), 8.91 | LCMS [ESI, |
| | | (d, J = 1.6 Hz, 1H), 8.70 (d, J = 1.6 Hz, 1H), 8.65 (s, 1H), | M + 1]: 429.0. |
| | | 8.63-8.44 (m, 1H), 8.42 (d, J = 8.8 Hz, 2H), 8.23 (d, J = | |
| | | 8.8 Hz, 2H), 3.17-3.03 (m, 1H), 2.21-2.00 (m, 3H), 1.99- | |
| | | 1.75 (m, 2H), 1.72-1.55 (m, 3H). | |
| 41 | racemic | ¹H NMR (400 MHz, DMSO-d₆) δ = 10.16 (s, 1H), 9.18 (d, | LCMS [ESI, |
| | | J = 2.0 Hz, 1H), 8.94 (d, J = 2.0 Hz, 1H), 8.89 (s, 1H), 8.74 | M + 1]: 386.1. |
| | | (d, J = 2.0 Hz, 1H), 8.66 (s, 1H), 8.61 (d, J = 2.0 Hz, 1H), | |
| | | 8.14 (s, 1H), 4.19 (q, J = 7.2 Hz, 2H), 3.07-2.94 (m, 2H), | |
| | | 1.28 (t, J = 7.2 Hz, 3H), 1.02-0.89 (m, 1H), 0.60-0.48 | |
| | | (m, 2H), 0.33-0.32 (m, 1H), 0.31-0.24 (m, 1H). | |
| 42 | racemic | ¹H NMR (400 MHz, DMSO-d₆) δ = 10.16 (s, 1H), 9.18 (d, | LCMS [ESI, |
| | | J = 2.0 Hz, 1H), 8.93 (d, J = 2.0 Hz, 1H), 8.89 (s, 1H), 8.72 | M + 1]: 400.1 |
| | | (d, J = 2.0 Hz, 1H), 8.66 (s, 1H), 8.61 (d, J = 1.6 Hz, 1H), | |
| | | 4.19 (q, J = 7.2 Hz, 2H), 3.28-3.22 (m, 1H), 3.18-3.08 | |
| | | (m, 1H), 2.76-2.65 (m, 1H), 2.10-1.99 (m, 1H), 1.92- | |
| | | 1.74 (m, 4H), 1.72-1.60 (m, 1H), 1.28 (t, J = 7.2 Hz, 3H). | |
| 43 | racemic | ¹H NMR (400 MHz, DMSO-d6) δ = 14.26 (br s, 1H), 8.97 | LCMS [ESI, |
| | | (s, 1H), 8.95 (d, J = 2.0 Hz, 1H), 8.73 (d, J = 2.0 Hz, 1H), | M + 1]: 367.1 |
| | | 8.65 (s, 1H), 8.57 (br s, 1H), 8.35 (br d, J = 7.6 Hz, 1H), | |
| | | 8.03 (d, J = 7.6 Hz, 1H), 7.71 (t, J = 7.6 Hz, 1H), 3.20- | |
| | | 3.08 (m, 1H), 3.04-2.93 (m, 1H), 1.72-1.57 (m, 1H), 1.52- | |
| | | 1.31 (m, 3H), 0.91-0.82 (m, 3H). | |
| 44 | racemic | ¹H NMR (400 MHz, DMSO-d₆) δ = 8.94-8.93 (m, 1H), | LCMS (ESI, |
| | | 8.72-8.71 (m, 1H), 8.64 (s, 1H), 8.50-8.43 (m, 3H), 8.23 | M + 1): m/z = |
| | | (d, J = 8.8 Hz, 2H), 3.17-3.10 (m, 1H), 3.00-2.94 (m, | 367.1 |
| | | 1H), 1.70-1.59 (m, 1H), 1.39-1.36 (m, 3H), 0.88-0.85 | |
| | | (m, 3H). | |
| 45 | racemic | ¹H NMR (400 MHz, DMSO-d₆) δ = 10.19 (br s, 1H), 8.94- | LCMS (ESI, |
| | | 8.86 (m, 3H), 8.72-8.67 (m, 3H), 4.21-4.15 (m, 2H), 3.16- | M + 1): m/z = |
| | | 3.11 (m, 1H), 2.99-2.97(m, 1H), 1.63 (m, 1H), 1.43- | 388.2 |
| | | 1.37 (m, 3H), 1.29-1.26 (m, 3H), 0.87 (t, J = 7.2 Hz, 3H) | |
| 46 | racemic | ¹H NMR (400 MHz, DMSO-d₆) δ = 14.25 (br d, J = 1.6 Hz, | LCMS [ESI, |
| | | 1H), 8.94 (d, J = 2.0 Hz, 1H), 8.74 (d, J = 2.0 Hz, 1H), 8.64 | M + 1]: 365.1. |
| | | (s, 1H), 8.50 (br s, 1H), 8.42 (d, J = 8.8 Hz, 2H), 8.23 | |
| | | (d, J = 8.8 Hz, 2H), 3.06-2.93 (m, 2H), 1.00-0.95 (m, 1H), | |
| | | 0.56-0.53 (m, 2H), 0.32-0.29 (m, 1H), 0.25-0.23 (m, | |
| | | 1H) | |
| 47 | racemic | ¹H NMR (400 MHz, DMSO-d₆) δ = 14.27 (br s, 1H), 8.93 | LCMS [ESI, |
| | | (s, 1H), 8.71 (s, 1H), 8.63 (s, 1H), 8.50 (br s, 1H), 8.41 (d, | M + 1]: 379.0. |
| | | J = 8.8 Hz, 2H), 8.23 (d, J = 8.8 Hz, 2H), 3.27-3.20 (m, | |
| | | 1H), 3.17-3.10 (m, 1H), 2.68 (quin, J = 7.6 Hz, 1H), | |
| | | 2.10-1.99 (m, 1H), 1.94-1.71 (m, 4H), 1.70-1.66 (m, 1H). | |
| 88A | 1^{st} peak | ¹H NMR (400 MHz, DMSO-d₆) δ = 8.90 (d, J = 2.0 Hz, | LCMS [ESI, |
| | | 1H), 8.84 (s, 1H), 8.72 (d, J = 2.0 Hz, 1H), 8.62 (s, 1H), | M + 1]: 397.1 |
| | | 8.09 (br d, J = 8.0 Hz, 1H), 8.00-7.93 (m, 2H), 7.55 (t, J = | |
| | | 7.6 Hz, 1H), 4.68 (d, J = 10 Hz, 1H), 4.57 (d, J = 10 Hz, | |
| | | 1H), 1.18 (s, 9H). | |
| 88B | 2^{nd} peak | ¹H NMR (400 MHz, DMSO-d₆) δ = 8.91 (d, J = 2.0 Hz, | LCMS [ESI, |
| | | 1H), 8.87 (s, 1H), 8.73 (d, J = 2.0 Hz, 1H), 8.64 (s, 1H), | M + 1]: 397.1 |
| | | 8.18-8.05 (m, 2H), 8.00 (d, J = 8.0 Hz, 1H), 7.65-7.54 | |
| | | (m, 1H), 4.71-4.67 (m, 1H), 4.60-4.56 (m, 1H), 1.19 (s, | |
| | | 9H) | |
| 90A | 1^{st} peak | ¹H NMR (400 MHz, DMSO-d₆) δ = 8.86 (d, J = 2.0 Hz, 1H), | LCMS (ESI, |
| | | 8.65 (d, J = 2.0 Hz, 1H), 8.62 (s, 1H), 8.57 (d, J = 2.0 | M + 1): m/z = |
| | | Hz, 1H), 8.50 (s, 1H), 8.21 (dt, J = 2.1, 4.4 Hz, 1H), 8.10 | 405.2 |
| | | (dd, J = 2.6, 8.4 Hz, 1H), 7.77-7.65 (m, 2H), 6.97 (d, J = | |
| | | 8.4 Hz, 1H), 3.92 (s, 3H), 3.91-3.85 (m, 1H), 2.44-2.26 | |
| | | (m, 2H), 2.25-2.09 (m, 1H), 2.02-1.87 (m, 1H), 1.86- | |
| | | 1.72 (m, 1H), 1.68-1.51 (m, 1H) | |
| 90B | 2^{nd} peak | ¹H NMR (400 MHz, DMSO-d₆) δ = 8.87 (d, J = 2.0 Hz, | LCMS (ESI, |
| | | 1H), 8.65 (d, J = 2.0 Hz, 1H), 8.63 (s, 1H), 8.57 (d, J = 2.8 | M + 1): m/z = |
| | | Hz, 1H), 8.50 (s, 1H), 8.21 (dt, J = 2.1, 4.8 Hz, 1H), 8.10 | 405.2 |
| | | (dd, J = 2.6, 8.4 Hz, 1H), 7.69 (d, J = 4.8 Hz, 2H), 6.97 (d, | |
| | | J = 8.8 Hz, 1H), 3.92 (s, 3H), 3.91 (br s, 1H), 2.43-2.28 | |
| | | (m, 2H), 2.18 (dtd, J = 4.2, 7.9, 11.6 Hz, 1H), 2.00-1.88 | |
| | | (m, 1H), 1.87-1.76 (m, 1H), 1.66-1.53 (m, 1H) | |
| 91A | 1^{st} peak | ¹H NMR (400 MHz, DMSO-d₆) δ = 8.86 (d, J = 2.0 Hz, | LCMS [ESI, |
| | | 1H), 8.69-8.59 (m, 2H), 8.54 (s, 1H), 8.29 (br d, J = 7.6 | M + 1]: m/z = |
| | | Hz, 1H), 8.24 (d, J = 5.2 Hz, 1H), 7.82-7.74 (m, 1H), 7.74- | 460.2 |
| | | 7.65 (m, 1H), 7.12 (s, 1H), 7.04 (d, J = 4.8 Hz, 1H), 3.90 | |
| | | (quin, J = 8.4 Hz, 1H), 3.76-3.68 (m, 4H), 3.57-3.50 (m, | |
| | | 4H), 2.43-2.28 (m, 2H), 2.24-2.10 (m, 1H), 1.99-1.87 | |
| | | (m, 1H), 1.87-1.75 (m, 1H), 1.69-1.52 (m, 1H). | |
| 91B | 2^{nd} peak | ¹H NMR (400 MHz, DMSO-d₆) δ = 8.86 (d, J = 2.0 Hz, | LCMS [ESI, |
| | | 1H), 8.65 (d, J = 2.0 Hz, 1H), 8.63 (s, 1H), 8.54 (t, J = 1.6 | M + 1]: m/z = |
| | | Hz, 1H), 8.34-8.27 (m, 1H), 8.24 (d, J = 5.2 Hz, 1H), 7.81- | 460.2 |
| | | 7.75 (m, 1H), 7.74-7.67 (m, 1H), 7.12 (s, 1H), 7.04 (dd, | |
| | | J = 0.8, 5.2 Hz, 1H), 3.90 (t, J = 8.0 Hz, 1H), 3.79-3.65 | |
| | | (m, 4H), 3.61-3.50 (m, 4H), 2.43-2.29 (m, 2H), 2.23-2.10 | |
| | | (m, 1H), 2.00-1.87 (m, 1H), 1.87-1.75 (m, 1H), 1.67- | |
| | | 1.54 (m, 1H). | |
| 92A | 1^{st} peak | ¹H NMR (400 MHz, DMSO-d₆) δ = 13.98-13.44 (m, 1H), | LCMS [ESI, |
| | | 9.03-8.82 (m, 2H), 8.78-8.51 (m, 2H), 8.31 (br d, J = 6.0 | M + 1]: 379.1 |
| | | Hz, 1H), 7.98 (d, J = 8.0 Hz, 1H), 7.67 (br t, J = 7.6 Hz, | |
| | | 1H), 4.07-3.81 (m, 1H), 2.44 (br s, 3H), 2.40-2.31 (m, | |
| | | 2H), 2.24-2.11 (m, 1H), 2.00-1.88 (m, 1H), 1.88-1.76 | |
| | | (m, 1H), 1.68-1.53 (m, 1H). | |
| 92B | 2^{nd} peak | ¹H NMR (400 MHz, DMSO-d₆) δ = 14.19-13.45 (m, 1H), | LCMS [ESI, |
| | | 9.03-8.82 (m, 2H), 8.73-8.57 (m, 2H), 8.31 (br d, J = 7.6 | M + 1]: 379.1 |
| | | Hz, 1H), 7.98 (br d, J = 7.6 Hz, 1H), 7.78-7.61 (m, 1H), | |
| | | 4.03-3.75 (m, 1H), 2.43 (s, 3H), 2.39-2.29 (m, 2H), 2.23- | |
| | | 2.10 (m, 1H), 1.98-1.88 (m, 1H), 1.86-1.76 (m, 1H), | |
| | | 1.66-1.51 (m, 1H). | |
| 93A | 1^{st} peak | ¹H NMR (400 MHz, DMSO-d₆) δ = 8.87 (d, J = 2.1 Hz, | LCMS (ESI, |
| | | 1H), 8.67-8.59 (m, 3H), 8.34-8.26 (m, 2H), 7.81-7.77 | M + 1): 405.1 |
| | | (m, 1H), 7.76-7.69 (m, 1H), 7.38 (dd, J = 1.5, 5.4 Hz, | |
| | | 1H), 7.17 (s, 1H), 3.95-3.91 (m, 3H), 3.89 (s, 1H), 2.40- | |
| | | 2.30 (m, 2H), 2.22-2.13 (m, 1H), 1.97-1.88 (m, 1H), 1.86- | |
| | | 1.76 (m, 1H), 1.66-1.54 (m, 1H). | |
| 93B | 2^{nd} peak | ¹H NMR (400 MHz, DMSO-d₆) δ = 9.42-9.41 (m, 1H), | LCMS [ESI, |
| | | 8.88 (d, J = 2.0 Hz, 1H), 8.66-8.61 (m, 3H), 8.33-8.28 | M + 1]: 405.1 |
| | | (m, 2H), 7.81-7.78 (m, 1H), 7.75-7.71 (m, 1H), 7.38 (dd, | |
| | | J = 1.5, 5.4 Hz, 1H), 7.17 (d, J = 0.9 Hz, 1H), 3.92 (s, 3H), | |
| | | 3.89 (s, 1H), 2.39-2.32 (m, 2H), 2.21-2.14 (m, 1H), 1.97- | |
| | | 1.88 (m, 1H), 1.85-1.78 (m, 1H), 1.63-1.56 (m, 1H). | |
| 94A | 1^{st} peak | 1H NMR (400 MHz, DMSO-d₆) δ = 11.83-11.53 (m, 1H), | LCMS (ESI, |
| | | 8.88 (d, J = 2.0 Hz, 1H), 8.66 (d, J = 2.0 Hz, 1H), 8.63 (s, | M + 1): m/z = |
| | | 1H), 8.56 (s, 1H), 8.31 (dt, J = 2.0, 4.6 Hz, 1H), 7.74-7.68 | 391.1 |
| | | (m, 2H), 7.51 (d, J = 6.8 Hz, 1H), 6.66 (d, J = 1.2 Hz, 1H), | |
| | | 6.57 (dd, J = 1.6, 6.8 Hz, 1H), 3.91 (quin, J = 8.0 Hz, 1H), | |
| | | 2.42-2.29 (m, 2H), 2.23-2.11 (m, 1H), 1.99-1.87 (m, | |
| | | 1H), 1.87-1.74 (m, 1H), 1.67-1.54 (m, 1H) | |
| 94B | 2^{nd} peak | 1H NMR (400 MHz, DMSO-d₆) δ = 12.08-11.33 (m, 1H), | LCMS (ESI, |
| | | 8.88 (d, J = 2.0 Hz, 1H), 8.66 (d, J = 2.0 Hz, 1H), 8.63 (s, | M + 1): m/z = |
| | | 1H), 8.56 (s, 1H), 8.38-8.25 (m, 1H), 7.71 (d, J = 5.2 Hz, | 391.2 |
| | | 2H), 7.51 (d, J = 6.8 Hz, 1H), 6.66 (d, J = 1.2 Hz, 1H), 6.57 | |
| | | (dd, J = 1.6, 6.8 Hz, 1H), 3.91 (quin, J = 8.0 Hz, 1H), | |
| | | 2.40-2.30 (m, 2H), 2.22-2.12 (m, 1H), 1.98-1.88 (m, 1H), | |
| | | 1.86-1.77 (m, 1H), 1.66-1.53 (m, 1H) | |
| 95A | | — | LCMS [ESI, |
| | | | M + 11: 460.2 |
| 95B | | — | LCMS [ESI, |
| | | | M + 1]: 460.2 |
| 96A | 1^{st} peak | ¹H NMR (400 MHz, DMSO-d₆) δ = 11.91 (br s, 1H), 8.85 | LCMS [ESI, |
| | | (d, J = 2.0 Hz, 1H), 8.64 (d, J = 2.0 Hz, 1H), 8.61 (s, 1H), | M + 1]: 391.1. |
| | | 8.38-8.32 (m, 1H), 8.13 (td, J = 1.6, 7.6 Hz, 1H), 7.90 (dd, | |
| | | J = 2.8, 9.6 Hz, 1H), 7.80 (d, J = 2.8 Hz, 1H), 7.66-7.60 | |
| | | (m, 1H), 7.60-7.56 (m, 1H), 6.48 (d, J = 9.6 Hz, 1H), 3.90 | |
| | | (q, J = 8.4 Hz, 1H), 2.41-2.31 (m, 2H), 2.23-2.11 (m, | |
| | | 1H), 2.00-1.87 (m, 1H), 1.87-1.75 (m, 1H), 1.69-1.54 | |
| | | (m, 1H). | |
| 96B | 2^{nd} peak | ¹H NMR (400 MHz, DMSO-d₆) δ = 11.91 (br s, 1H), 8.85 | LCMS [ESI, |
| | | (d, J = 2.0 Hz, 1H), 8.65 (d, J = 1.6 Hz, 1H), 8.61 (s, 1H), | M + 1]: 391.1. |
| | | 8.36 (s, 1H), 8.13 (br d, J = 7.6 Hz, 1H), 7.90 (dd, J = 2.8, | |
| | | 9.6 Hz, 1H), 7.80 (br d, J = 2.0 Hz, 1H), 7.69-7.60 (m, | |
| | | 1H), 7.60-7.54 (m, 1H), 6.48 (d, J = 9.6 Hz, 1H), 3.97- | |
| | | 3.85 (m, 1H), 2.40-2.32 (m, 2H), 2.23-2.11 (m, 1H), 1.98- | |
| | | 1.88 (m, 1H), 1.86-1.76 (m, 1H), 1.66-1.55 (m, 1H). | |
| 97A | 1^{st} peak | ¹H NMR (400 MHz, DMSO-d₆) δ = 14.12-13.57 (m, 1H), | LCMS [ESI, |
| | | 8.94 (d, J = 2.0 Hz, 1H), 8.90 (s, 1H), 8.71 (d, J = 2.0 Hz, | M + 1]: 415.1 |
| | | 1H), 8.64 (s, 1H), 8.30 (br d, J = 7.6 Hz, 1H), 7.98 (d, J = | |
| | | 7.6 Hz, 1H), 7.67 (t, J = 8.0 Hz, 1H), 3.96-3.83 (m, 1H), | |
| | | 3.08-2.81 (m, 3H), 3.10-2.78 (m, 1H), 2.43 (s, 3H). | |
| 97B | 2^{nd} peak | ¹H NMR (400 MHz, DMSO-d₆) δ = 14.05-13.56 (m, 1H), 8.94 | LCMS [ESI, |
| | | (d, J = 2.0 Hz, 1H), 8.90 (s, 1H), 8.71 (d, J = 2.0 Hz, | M + 1]: 415.1 |
| | | 1H), 8.64 (s, 1H), 8.30 (br d, J = 7.6 Hz, 1H), 7.98 (br d, | |
| | | J = 7.6 Hz, 1H), 7.67 (br t, J = 8.0 Hz, 1H), 3.95-3.86 (m, | |
| | | 1H), 3.09-2.79 (m, 4H), 2.43 (s, 3H) | |
| 98A | 1^{st} peak | ¹H NMR (400 MHz, DMSO-d₆) δ = 12.40-12.00 (m, 1H), | LCMS [ESI, |
| | | 8.86 (d, J = 1.6 Hz, 1H), 8.81 (s, 1H), 8.65 (d, J = 1.6 Hz, | M + 1]: 380.1 |
| | | 1H), 8.61 (d, J = 1.2 Hz, 1H), 8.23 (br d, J = 8.0 Hz, 1H), | |
| | | 7.91-7.86 (m, 1H), 7.61 (t, J = 8.0 Hz, 1H), 6.25-5.95 | |
| | | (m, 2H), 3.97-3.84 (m, 1H), 2.38-2.31 (m, 2H), 2.21- | |
| | | 2.14 (m, 1H), 1.99-1.88 (m, 1H), 1.87-1.74 (m, 1H), | |
| | | 1.66-1.54 (m, 1H). | |
| 98B | 2^{nd} peak | ¹H NMR (400 MHz, DMSO-d₆) δ = 12.49-11.81 (m, 1H), | LCMS (ESI, |
| | | 8.86 (d, J = 2.0 Hz, 1H), 8.81 (s, 1H), 8.65 (d, J = 2.0 Hz, | M + 1): 380.1 |
| | | 1H), 8.61 (s, 1H), 8.23 (br d, J = 6.8 Hz, 1H), 8.13 (s, 1H), | |
| | | 7.88 (d, J = 7.6 Hz, 1H), 7.61 (s, 1H), 6.45-5.62 (m, 2H), | |
| | | 3.91 (t, J = 8.0 Hz, 1H), 2.35-2.29 (m, 2H), 2.22-2.12 | |
| | | (m, 1H), 1.95-1.86 (m, 1H), 1.85-1.76 (m, 1H), 1.63- | |
| | | 1.60 (m, 1H) | |
| 99A | 1^{st} peak | ¹H NMR (400 MHz, DMSO-d₆) δ = 14.45 (br s, 1H), 9.54 | LCMS [ESI, |
| | | (d, J = 2.4 Hz, 1H), 9.27 (t, J = 2.0 Hz, 1H), 9.18 (d, J = | M + 1]: m/z = |
| | | 1.6 Hz, 1H), 8.93 (d, J = 2.0 Hz, 1H), 8.74-8.65 (m, 3H), | 366.2 |
| | | 3.92 (quin, J = 8.4 Hz, 1H), 2.36 (qd, J = 9.2, 18.0 Hz, 2H), | |
| | | 2.24-2.13 (m, 1H), 1.99-1.88 (m, 1H), 1.88-1.76 (m, | |
| | | 1H), 1.61 (ddd, J = 4.0, 7.6, 11.6 Hz, 1H) | |
| 99B | 2^{nd} peak | ¹H NMR (400 MHz, DMSO-d₆) δ = 14.47 (br s, 1H), 9.54 | LCMS [ESI, |
| | | (d, J = 2.4 Hz, 1H), 9.27 (t, J = 2.0 Hz, 1H), 9.18 (d, J = | M + 1]: m/z = |
| | | 1.6 Hz, 1H), 8.93 (d, J = 2.0 Hz, 1H), 8.74-8.66 (m, 3H), | 366.2 |
| | | 3.92 (quin, J = 8.4 Hz, 1H), 2.36 (qd, J = 8.8, 18.0 Hz, 2H), | |
| | | 2.24-2.13 (m, 1H), 1.99-1.88 (m, 1H), 1.87-1.76 (m, | |
| | | 1H), 1.66-1.55 (m, 1H) | |
| 100A | 1^{st} peak | ¹H NMR (400 MHz, DMSO-d₆) δ = 12.68-12.50 (m, 1H), | LCMS [ESI, |
| | | 8.89-8.85 (m, 1H), 8.79-8.73 (m, 1H), 8.63 (br d, J = | M + 1]: 408.2 |
| | | 12.4 Hz, 2H), 8.32-8.24 (m, 1H), 7.92 (br d, J = 7.6 Hz, | |
| | | 1H), 7.67-7.59 (m, 1H), 3.96-3.87 (m, 1H), 3.00 (s, 6H), | |
| | | 2.39-2.31 (m, 2H), 2.22-2.13 (m, 1H), 1.98-1.89 (m, | |
| | | 1H), 1.87-1.78 (m, 1H), 1.65-1.54 (m, 1H). | |
| 100B | 2^{nd} peak | ¹H NMR (400 MHz, DMSO-d₆) δ = 12.80-12.36 (m, 1H), | LCMS [ESI, |
| | | 8.87 (d, J = 1.6 Hz, 1H), 8.76 (s, 1H), 8.67-8.58 (m, 2H), | M + 1]: 408.2 |
| | | 8.30 (br d, J = 7.6 Hz, 1H), 7.93 (br d, J = 7.6 Hz, 1H), | |
| | | 7.63 (br t, J = 8.0 Hz, 1H), 3.95-3.85 (m, 1H), 3.00 (s, | |
| | | 6H), 2.39-2.30 (m, 2H), 2.22-2.11 (m, 1H), 1.99-1.87 | |
| | | (m, 1H), 1.86-1.73 (m, 1H), 1.68-1.55 (m, 1H). | |
| 101A | 1^{st} peak | ¹H NMR (400 MHz, DMSO-d₆) δ = 8.96-8.90 (m, 2H), | LCMS [ESI, |
| | | 8.74 (d, J = 2.0 Hz, 1H), 8.64 (s, 1H), 8.41 (s, 1H), 8.32- | M + 1]: m/z = |
| | | 8.25 (m, 1H), 8.02 (d, J = 7.6 Hz, 1H), 7.68 (t, J = 8.0 Hz, | 383.2. |
| | | 1H), 3.14-3.04 (m, 2H), 1.41 (s, 3H), 1.28 (s, 3H). | |
| 101B | 2^{nd} peak | ¹H NMR (400 MHz, DMSO-d₆) δ = 8.95-8.86 (m, 2H), | LCMS [ESI, |
| | | 8.73 (d, J = 2.0 Hz, 1H), 8.63 (s, 1H), 8.36 (s, 1H), 8.29- | M + 1]: m/z = |
| | | 8.22 (m, 1H), 8.02 (d, J = 7.6 Hz, 1H), 7.66 (t, J = 8.0 Hz, | 383.2. |
| | | 1H), 3.14-3.05 (m, 2H), 1.40 (s, 3H), 1.28 (s, 3H). | |
| 102A | 1^{st} peak | ¹H NMR (400 MHz, DMSO-d₆) δ = 14.44 (br s, 1H), 9.55 | LCMS (ESI, |
| | | (d, J = 2.4 Hz, 1H), 9.27 (t, J = 2.4 Hz, 1H), 9.19 (d, J = | M + 1): m/z = |
| | | 2.0 Hz, 1H), 8.99 (d, J = 2.0 Hz, 1H), 8.75 (d, J = 2.0 Hz, | 384.1 |
| | | 1H), 8.72 (s, 1H), 8.69 (s, 1H), 3.38 (t, J = 6.4 Hz, 2H), | |
| | | 3.25-3.20 (m, 1H), 3.19 (s, 3H), 3.06-2.96 (m, 1H), 1.96- | |
| | | 1.83 (m, 1H), 1.71-1.59 (m, 1H). | |
| 102B | 2^{nd} peak | ¹H NMR (400 MHz, DMSO-d₆) δ = 14.38 (s, 1H), 9.53 (d, | LCMS (ESI, |
| | | J = 2.4 Hz, 1H), 9.27 (t, J = 2.4 Hz, 1H), 9.19 (d, J = 1.6 | M + 1): m/z = |
| | | Hz, 1H), 8.99 (d, J = 2.0 Hz, 1H), 8.76 (d, J = 2.0 Hz, 1H), | 384.1 |
| | | 8.72 (s, 1H), 8.64 (s, 1H), 3.39 (t, J = 6.0 Hz, 2H), 3.25- | |
| | | 3.20 (m, 1H), 3.19 (s, 3H), 3.05-2.97 (m, 1H), 1.95-1.84 | |
| | | (m, 1H), 1.70-1.59 (m, 1H). | |
| 103A | 1^{st} peak | ¹H NMR (400 MHz, DMSO-d₆) δ = 14.71-13.80 (m, 1H), | LCMS [ESI, |
| | | 8.74 (s, 1H), 8.66 (d, J = 3.6 Hz, 1H), 8.59-8.47 (m, 2H), | M + 1]: 383.2 |
| | | 8.08 (br d, J = 8.0 Hz, 1H), 7.93 (br d, J = 8.0 Hz, 1H), | |
| | | 7.79-7.64 (m, 1H), 7.07 (d, J = 3.6 Hz, 1H), 3.46-3.40 | |
| | | (m, 2H), 3.28-3.22 (m, 1H), 3.18 (s, 3H), 3.10-2.99 (m, | |
| | | 1H), 2.07-1.84 (m, 1H), 1.74-1.56 (m, 1H). | |
| 103B | 2^{nd} peak | ¹H NMR (400 MHz, DMSO-d₆) δ = 14.86-13.68 (m, 1H), | LCMS [ESI, |
| | | 8.74 (s, 1H), 8.65 (d, J = 3.6 Hz, 1H), 8.61-8.48 (m, 2H), | M + 1]: 383.2 |
| | | 8.08 (d, J = 8.0 Hz, 1H), 7.93 (br d, J = 8.0 Hz, 1H), 7.80- | |
| | | 7.59 (m, 1H), 7.07 (d, J = 3.6 Hz, 1H), 3.44-3.37 (m, 2H), | |
| | | 3.28-3.23 (m, 1H), 3.18 (s, 3H), 3.11-2.98 (m, 1H), 2.04- | |
| | | 1.88 (m, 1H), 1.76-1.55 (m, 1H). | |
| 104A | 1^{st} peak | ¹H NMR (400 MHz, DMSO-d₆) δ = 8.96 (d, J = 1.6 Hz, | LCMS [ESI, |
| | | 2H), 8.72 (d, J = 2.0 Hz, 1H), 8.64 (s, 1H), 8.42 (br d, J = | M + 1]: 411.2. |
| | | 2.8 Hz, 1H), 8.30 (br d, J = 8.4 Hz, 1H), 8.02 (d, J = 7.6 | |
| | | Hz, 1H), 7.67 (t, J = 8.0 Hz, 1H), 3.16 (dt, J = 4.4, 12.8 Hz, | |
| | | 1H), 3.00 (s, 3H), 2.99-2.86 (m, 1H), 1.88-1.73 (m, 1H), | |
| | | 1.56-1.44 (m, 1H), 1.06 (d, J = 8.0 Hz, 6H). | |
| 104B | 2^{nd} peak | ¹H NMR (400 MHz, DMSO-d₆) δ = 8.94 (d, J = 1.6 Hz, | LCMS [ESI, |
| | | 1H), 8.90 (s, 1H), 8.71 (d, J = 2.0 Hz, 1H), 8.62 (s, 1H), | M + 1]: 411.2. |
| | | 8.15 (br d, J = 8.0 Hz, 1H), 8.07 (s, 1H), 8.01 (d, J = 7.6 | |
| | | Hz, 1H), 7.58 (t, J = 8.0 Hz, 1H), 3.16 (dt, J = 4.4, 12.4 Hz, | |
| | | 1H), 3.00 (s, 3H), 2.99-2.89 (m, 1H), 1.89-1.73 (m, 1H), | |
| | | 1.57-1.40 (m, 1H), 1.06 (d, J = 8.0 Hz, 6H). | |
| 105A | 1^{st} peak | ¹H NMR (400 MHz, DMSO-d₆) δ = 8.89 (t, J = 2.0 Hz, | LCMS [ESI, |
| | | 2H), 8.69 (d, J = 2.0 Hz, 1H), 8.62 (s, 1H), 8.18 (br s, 2H), | M + 1]: 395.1. |
| | | 8.00 (br d, J = 8.0 Hz, 1H), 7.61 (t, J = 8.0 Hz, 1H), 3.94- | |
| | | 3.88 (m, 1H), 3.82 (td, J = 4.8, 9.6 Hz, 1H), 3.11 (s, 3H), | |
| | | 2.46 (br s, 1H), 2.38-2.25 (m, 2H), 1.97-1.80 (m, 1H). | |
| 105B | 2^{nd} peak | ¹H NMR (400 MHz, DMSO-d₆) δ = 8.87 (br d, J = 2.4 Hz, | LCMS [ESI, |
| | | 2H), 8.63 (d, J = 2.0 Hz, 1H), 8.61 (s, 1H), 8.15 (br s, 2H), | M + 1]: 395.1. |
| | | 8.00 (br d, J = 7.6 Hz, 1H), 7.60 (br t, J = 8.0 Hz, 1H), 3.78 | |
| | | (td, J = 7.2, 14.4 Hz, 1H), 3.53-3.45 (m, 1H), 3.11 (s, 3H), | |
| | | 2.48-2.41 (m, 1H), 2.24-2.03 (m, 2H), 1.90-1.74 (m, | |
| | | 1H). | |
| 105C | 3^{rd} peak | ¹H NMR (400 MHz, DMSO-d₆) δ = 8.91 (br s, 1H), 8.87 | LCMS [ESI, |
| | | (d, J = 2.0 Hz, 1H), 8.63 (d, J = 2.0 Hz, 1H), 8.62 (s, 1H), | M + 1]: 395.1. |
| | | 8.25 (br s, 1H), 8.19 (br s, 1H), 8.01 (br d, J = 7.6 Hz, 1H), | |
| | | 7.62 (t, J = 8.0 Hz, 1H), 3.78 (quin, J = 6.8 Hz, 1H), 3.54- | |
| | | 3.43 (m, 1H), 3.10 (s, 3H), 2.48-2.41 (m, 1H), 2.24-2.02 | |
| | | (m, 2H), 1.90-1.73 (m, 1H). | |
| 105D | 4^{th} peak | ¹H NMR (400 MHz, DMSO-d₆) δ = 14.21 (br s, 1H), 8.96 | LCMS [ESI, |
| | | (s, 1H), 8.91 (d, J = 1.6 Hz, 1H), 8.70 (d, J = 1.6 Hz, 1H), | M + 1]: 395.1. |
| | | 8.64 (s, 1H), 8.55 (br s, 1H), 8.34 (br d, J = 7.6 Hz, 1H), | |
| | | 8.03 (d, J = 7.6 Hz, 1H), 7.70 (t, J = 8.0 Hz, 1H), 3.91 | |
| | | (quin, J = 6.0 Hz, 1H), 3.87-3.77 (m, 1H), 3.11 (s, 3H), | |
| | | 2.65-2.54 (m, 2H), 2.41-2.25 (m, 1H), 1.95-1.83 (m, | |
| | | 1H). | |
| 108A | 1^{st} peak | ¹H NMR (400 MHz, DMSO-d₆) δ = 8.95 (d, J = 2.0 Hz, | LCMS [ESI, |
| | | 2H), 8.71 (d, J = 2.0 Hz, 1H), 8.64 (s, 1H), 8.38 (s, 1H), | M + 1]: m/z = |
| | | 8.28 (dd, J = 1.2, 8.4 Hz, 1H), 8.02 (d, J = 7.6 Hz, 1H), | 397.2. |
| | | 7.67 (t, J = 8.0 Hz, 1H), 3.21 (br d, J = 3.2 Hz, 1H), 3.01 | |
| | | (br dd, J = 8.0, 12.4 Hz, 1H), 1.70 (dt, J = 4.4, 12.8 Hz, | |
| | | 1H), 1.45 (dt, J = 4.8, 12.8 Hz, 1H), 1.06 (d, J = 3.6 Hz, | |
| | | 6H) | |
| 108A | 2^{nd} peak | ¹H NMR (400 MHz, DMSO-d₆) δ = 8.95 (d, J = 2.0 Hz, | LCMS [ESI, |
| | | 2H), 8.71 (d, J = 2.0 Hz, 1H), 8.64 (s, 1H), 8.42 (s, 1H), | M + 1]: m/z = |
| | | 8.30 (dd, J = 1.2, 8.0 Hz, 1H), 8.02 (d, J = 7.6 Hz, 1H), | 397.2. |
| | | 7.68 (t, J = 8.0 Hz, 1H), 3.21 (br dd, J = 8.4, 12.4 Hz, 1H), | |
| | | 3.04-2.97 (m, 1H), 1.70 (dt, J = 4.4, 12.4 Hz, 1H), 1.45 | |
| | | (dt, J = 4.8, 12.4 Hz, 1H), 1.06 (d, J = 3.6 Hz, 6H) | |
| 109C | | ¹H NMR (400 MHz, DMSO-d₆) δ = 8.96 (s, 1H), 8.88 (d, J = | LCMS [ESI, |
| | | 2.0 Hz, 1H), 8.67 (d, J = 2.0 Hz, 1H), 8.63 (s, 1H), 8.53 | M + 1]: m/z = |
| | | (s, 1H), 8.36-8.29 (m, 1H), 8.02 (br d, J = 7.6 Hz, 1H), | 409.2 |
| | | 7.70 (t, J = 7.6 Hz, 1H), 3.93-3.82 (m, 1H), 3.34 (br d, J = | |
| | | 4.8 Hz, 2H), 3.21 (s, 3H), 2.46-2.37 (m, 3H), 2.13-1.92 | |
| | | (m, 1H), 1.50 (br t, J = 8.0 Hz, 1H). | |
| 109D | | ¹H NMR (400 MHz, DMSO-d₆) δ = 14.33 (br s, 1H), 8.96 | LCMS [ESI, |
| | | (s, 1H), 8.87 (d, J = 2.0 Hz, 1H), 8.64 (d, J = 2.0 Hz, 1H), | M + 1]: m/z = |
| | | 8.63 (s, 1H), 8.56 (br s, 1H), 8.34 (br d, J = 8.8 Hz, 1H), | 409.2 |
| | | 8.02 (d, J = 7.6 Hz, 1H), 7.70 (t, J = 8.0 Hz, 1H), 3.81 | |
| | | (quin, J = 8.4 Hz, 1H), 3.25 (d, J = 6.4 Hz, 2H), 3.20 (s, | |
| | | 3H), 2.47-2.38 (m, 1H), 2.23 (dtd, J = 3.6, 8.0, 11.6 Hz, | |
| | | 1H), 2.15-1.96 (m, 2H), 1.63 (dtd, J = 3.6, 8.0, 11.6 Hz, | |
| | | 1H) | |
| 109A | | ¹H NMR (400 MHz, DMSO-d₆) δ = 8.96 (s, 1H), 8.89 (d, J = | LCMS [ESI, |
| | | 2.0 Hz, 1H), 8.67 (d, J = 2.0 Hz, 1H), 8.63 (s, 1H), 8.54 | M + 1]: m/z = |
| | | (br s, 1H), 8.33 (br d, J = 9.2 Hz, 1H), 8.02 (br d, J = 7.6 | 409.2 |
| | | Hz, 1H), 7.70 (t, J = 8.0 Hz, 1H), 3.97-3.80 (m, 1H), 3.36- | |
| | | 3.35 (m, 2H), 3.21 (s, 3H), 2.43 (br d, J = 6.4 Hz, 3H), | |
| | | 2.14-1.93 (m, 1H), 1.50 (br t, J = 7.2 Hz, 1H). | |
| 109B | | ¹H NMR (400 MHz, DMSO-d₆) δ = 8.98-8.93 (m, 1H), | LCMS [ESI, |
| | | 8.88 (d, J = 2.0 Hz, 1H), 8.65 (d, J = 2.0 Hz, 1H), 8.63 (s, | M + 1]: m/z = |
| | | 1H), 8.54 (s, 1H), 8.34 (dd, J = 1.2, 8.0 Hz, 1H), 8.03 (d, | 409.2 |
| | | J = 7.6 Hz, 1H), 7.70 (t, J = 8.0 Hz, 1H), 3.82 (quin, J = 8.4 | |
| | | Hz, 1H), 3.26 (br d, J = 6.4 Hz, 2H), 3.21 (s, 3H), 2.47- | |
| | | 2.39 (m, 1H), 2.29-2.17 (m, 1H), 2.14-1.98 (m, 2H), | |
| | | 1.70-1.57 (m, 1H). | |
| 110A | 1^{st} peak | ¹H NMR (400 MHz, DMSO-d₆) δ = 8.75-8.69 (m, 1H), | LCMS [ESI, |
| | | 8.69-8.63 (m, 1H), 8.58-8.47 (m, 2H), 8.15-8.04 (m, | M + 1]: 401.0 |
| | | 1H), 7.95-7.84 (m, 1H), 7.79-7.68 (m, 1H), 7.14-7.04 | |
| | | (m, 1H), 3.88-3.86 (m, 1H), 3.09-3.00 (m, 4H). | |
| 110B | 2^{nd} peak | ¹H NMR (400 MHz, DMSO-d₆) δ = 8.70 (s, 1H), 8.66 (br | LCMS [ESI, |
| | | d, J = 4.0 Hz, 1H), 8.50 (br d, J = 6.4 Hz, 2H), 8.08 (br d, | M + 1]: 401.0 |
| | | J = 7.6 Hz, 1H), 7.89 (br d, J = 7.6 Hz, 1H), 7.71 (br t, J = | |
| | | 8.0 Hz, 1H), 7.07 (d, J = 3.6 Hz, 1H), 3.87-3.83 (m, 1H), | |
| | | 3.11-3.00 (m, 4H). | |
| 111A | 1^{st} peak | ¹H NMR (400 MHz, DMSO-d₆) δ = 8.69-8.57 (m, 2H), | LCMS [ESI, |
| | | 8.51 (d, J = 2.8 Hz, 2H), 8.08 (d, J = 8.0 Hz, 1H), 7.90 (dd, | M + 1]: 365.0 |
| | | J = 1.2, 8.0 Hz, 1H), 7.76-7.63 (m, 1H), 7.09 (d, J = 4.0 | |
| | | Hz, 1H), 3.96 (br t, J = 8.4 Hz, 1H), 2.46-2.31 (m, 2H), | |
| | | 2.30-2.17 (m, 1H), 2.03-1.89 (m, 1H), 1.88-1.75 (m, | |
| | | 1H), 1.60 (br dd, J = 3.4, 8.0 Hz, 1H). | |
| 111B | 2^{nd} peak | ¹H NMR (400 MHz, DMSO-d₆) δ = 8.72-8.59 (m, 2H), | LCMS [ESI, |
| | | 8.51 (s, 2H), 8.08 (d, J = 7.6 Hz, 1H), 7.90 (dd, J = 1.6, 7.6 | M + 1]: 365.0 |
| | | Hz, 1H), 7.78-7.65 (m, 1H), 7.04 (d, J = 3.6 Hz, 1H), 3.95 | |
| | | (td, J = 8.4, 16.8 Hz, 1H), 2.47-2.31 (m, 2H), 2.30-2.15 | |
| | | (m, 1H), 2.03-1.90 (m, 1H), 1.89-1.76 (m, 1H), 1.68- | |
| | | 1.53 (m, 1H). | |
| 112A | 1^{st} peak | ¹H NMR (400 MHz, DMSO-d₆) δ = 14.30 (br s, 1H), 8.97 | LCMS [ESI, |
| | | (s, 1H), 8.95 (d, J = 2.0 Hz, 1H), 8.77 (d, J = 2.0 Hz, 1H), | M + 1]: 397.2. |
| | | 8.65 (s, 1H), 8.56 (br s, 1H), 8.35 (br d, J = 7.6 Hz, 1H), | |
| | | 8.03 (d, J = 8.0 Hz, 1H), 7.71 (t, J = 8.0 Hz, 1H), 3.28- | |
| | | 3.15 (m, 5H), 1.41 (s, 3H), 1.29 (s, 3H). | |
| 112B | 2^{nd} peak | ¹H NMR (400 MHz, DMSO-d₆) δ = 8.94 (d, J = 2.0 Hz, | LCMS [ESI, |
| | | 1H), 8.92 (br d, J = 1.2 Hz, 1H), 8.75 (d, J = 2.0 Hz, 1H), | M + 1]: 397.2. |
| | | 8.63 (s, 1H), 8.32-8.25 (m, 1H), 8.23 (br d, J = 8.0 Hz, | |
| | | 1H), 8.02 (br d, J = 7.6 Hz, 1H), 7.64 (t, J = 7.6 Hz, 1H), | |
| | | 3.29-3.14 (m, 5H), 1.41 (s, 3H), 1.29 (s, 3H). | |
| 113A | 1^{st} peak | ¹H NMR (400 MHz, DMSO-d₆) δ = 15.02-13.09 (m, 1H), | LCMS [ESI, |
| | | 8.96 (br s, 1H), 8.94 (d, J = 1.6 Hz, 1H), 8.72 (d, J = 1.6 | M + 1]: 383.1 |
| | | Hz, 1H), 8.71-8.54 (m, 2H), 8.35 (br d, J = 5.2 Hz, 1H), | |
| | | 8.03 (br d, J = 7.6 Hz, 1H), 7.70 (br t, J = 7.6 Hz, 1H), | |
| | | 3.41-3.38 (m, 2H), 3.37 (br s, 1H), 3.18 (s, 3H), | |
| | | 3.07-2.93 (m, 1H), 1.97-1.79 (m, 1H), 1.76-1.57 (m, 1H). | |
| 113B | 2^{nd} peak | ¹H NMR (400 MHz, DMSO-d₆) δ = 15.24-13.36 (m, 1H), | LCMS [ESI, |
| | | 8.97 (br s, 1H), 8.95 (d, J = 2.0 Hz, 1H), 8.73 (d, J = 2.0 | M + 1]: 383.1 |
| | | Hz, 1H), 8.65 (s, 2H), 8.35 (br d, J = 6.4 Hz, 1H), 8.03 (br | |
| | | d, J = 7.6 Hz, 1H), 7.71 (br t, J = 7.6 Hz, 1H), 3.38 (t, J = | |
| | | 6.0 Hz, 2H), 3.30 (br s, 1H), 3.19 (s, 3H), 3.01 (ddd, J = | |
| | | 5.2, 9.6, 13.6 Hz, 1H), 1.97-1.81 (m, 1H), 1.74-1.56 (m, | |
| | | 1H). | |
| 114A | 1^{st} peak | ¹H NMR (400 MHz, DMSO-d₆) δ = 9.05 (d, J = 2.0 Hz, | LCMS (ESI, |
| | | 1H), 8.89 (d, J = 1.6 Hz, 1H), 8.70 (d, J = 1.2 Hz, 1H), 8.61 | M + 1): m/z = |
| | | (s, 1H), 8.49-8.35 (m, 2H), 4.01 (br t, J = 8.0 Hz, 2H), | 415.1 |
| | | 3.50 (br t, J = 8.0 Hz, 2H), 3.38 (br s, 2H), 3.22-3.12 (m, | |
| | | 4H), 3.03-2.95 (m, 1H), 2.83 (s, 3H), 1.95-1.82 (m, 1H), | |
| | | 1.73-1.59 (m, 1H). | |
| 114B | 2^{nd} peak | ¹H NMR (400 MHz, DMSO-d₆) δ = 9.05 (s, 1H), 8.89 (s, | LCMS (ESI, |
| | | 1H), 8.70 (s, 1H), 8.61 (s, 1H), 8.48-8.36 (m, 2H), 4.00 | M + 1): m/z = |
| | | (br t, J = 8.0 Hz, 2H), 3.49 (br t, J = 8.0 Hz, 2H), 3.40- | 415.1 |
| | | 3.36 (m, 2H), 3.20-3.13 (m, 4H), 3.00 (br dd, J = 5.2, 9.6 | |
| | | Hz, 1H), 2.83 (s, 3H), 1.93-1.83 (m, 1H), 1.70-1.59 (m, | |
| | | 1H). | |
| 115A | 1^{st} peak | ¹H NMR (400 MHz, DMSO-d₆) δ = 9.17 (br s, 1H), 8.90 (s, | LCMS [ESI, |
| | | 1H), 8.71 (s, 1H), 8.63 (s, 1H), 8.59 (br dd, J = 1.6, 9.2 Hz, | M + 1]: 402.1 |
| | | 1H), 8.29 (br d, J = 9.2 Hz, 1H), 4.50 (br t, J = 7.6 Hz, 2H), | |
| | | 4.34-4.13 (m, 2H), 3.38 (br t, J = 6.0 Hz, 2H), 3.18 (s, | |
| | | 4H), 3.05-2.93 (m, 1H), 1.97-1.77 (m, 1H), 1.73-1.53 | |
| | | (m, 1H). | |
| 115B | 2^{nd} peak | ¹H NMR (400 MHz, DMSO-d₆) δ = 9.18 (d, J = 2.4 Hz, | LCMS [ESI, |
| | | 1H), 8.91 (d, J = 2.4 Hz, 1H), 8.71 (d, J = 2.0 Hz, 1H), 8.64 | M + 1]: 402.1 |
| | | (s, 1H), 8.60 (dd, J = 2.4, 9.2 Hz, 1H), 8.30 (d, J = 9.2 Hz, | |
| | | 1H), 4.57-4.45 (m, 2H), 4.31-4.16 (m, 2H), 3.38 (t, J = | |
| | | 6.4 Hz, 2H), 3.23-3.12 (m, 4H), 3.03-2.95 (m, 1H), 1.96- | |
| | | 1.81 (m, 1H), 1.72-1.56 (m, 1H). | |
| 116A | 1^{st} peak | ¹H NMR (400 MHz, DMSO-d₆) δ = 9.05 (d, J = 1.6 Hz, | LCMS (ESI, |
| | | 1H), 8.90 (d, J = 2.0 Hz, 1H), 8.71 (d, J = 2.0 Hz, 1H), 8.62 | M + 1): m/z = |
| | | (s, 1H), 8.47-8.36 (m, 2H), 7.35 (s, 1H), 4.07 (t, J = 8.0 | 401.2 |
| | | Hz, 2H), 3.45 (br t, J = 8.4 Hz, 2H), 3.40-3.36 (m, 2H), | |
| | | 3.22-3.13 (m, 4H), 3.05-2.93 (m, 1H), 1.94-1.81 (m, | |
| | | 1H), 1.64 (s, 1H). | |
| 116B | 2^{nd} peak | ¹H NMR (400 MHz, DMSO-d₆) δ = 9.05 (d, J = 1.6 Hz, | LCMS (ESI, |
| | | 1H), 8.90 (d, J = 2.0 Hz, 1H), 8.71 (d, J = 2.0 Hz, 1H), 8.62 | M + 1): m/z = |
| | | (s, 1H), 8.45-8.37 (m, 2H), 7.35 (s, 1H), 4.07 (t, J = 8.0 | 401.3 |
| | | Hz, 2H), 3.45 (br t, J = 8.4 Hz, 2H), 3.40-3.36 (m, 2H), | |
| | | 3.21-3.13 (m, 4H), 3.04-2.95 (m, 1H), 1.94-1.83 (m, | |
| | | 1H), 1.64 (s, 1H). | |
| 117A | 1^{st} peak | ¹H NMR (400 MHz, DMSO-d₆) δ = 9.52 (d, J = 2.0 Hz, | LCMS (ESI, |
| | | 1H), 8.93 (d, J = 2.0 Hz, 1H), 8.72 (d, J = 2.0 Hz, 1H), 8.66 | M + 1): 460.1 |
| | | (s, 1H), 8.25 (dd, J = 2.0, 9.6 Hz, 1H), 7.71 (d, J = 9.6 Hz, | |
| | | 1H), 3.88 (dt, J = 4.8, 8.0 Hz, 1H), 3.75-3.71 (m, 4H), | |
| | | 3.52-3.49 (m, 4H), 3.01-2.87 (m, 3H), 2.45-2.34 (m, | |
| | | 1H). | |
| 117B | 2^{nd} peak | ¹H NMR (400 MHz, DMSO-d₆) δ = 9.52 (d, J = 2.0 Hz, | LCMS (ESI, |
| | | 1H), 8.93 (d, J = 2.0 Hz, 1H), 8.73 (d, J = 2.0 Hz, 1H), 8.66 | M + 1): 460.1 |
| | | (s, 1H), 8.26 (dd, J = 2.0, 9.6 Hz, 1H), 7.72 (d, J = 9.6 Hz, | |
| | | 1H), 3.89 (dt, J = 4.4, 8.4 Hz, 1H), 3.77-3.71 (m, 4H), | |
| | | 3.53-3.49 (m, 4H), 3.03-2.89 (m, 3H), 2.45-2.36 (m, | |
| | | 1H). | |
| 118A | 1^{st} peak | ¹H NMR (400 MHz, DMSO-d₆) δ = 8.93 (d, J = 2.0 Hz, | LCMS [ESI, |
| | | 1H), 8.71 (d, J = 2.0 Hz, 1H), 8.65 (s, 1H), 8.54 (br d, J = | M + 1]: 391.1 |
| | | 4.4 Hz, 1H), 8.38 (d, J = 8.6 Hz, 2H), 8.06 (d, J = 8.8 Hz, | |
| | | 2H), 3.93-3.82 (m, 1H), 3.00-2.87 (m, 3H), 2.82 (d, J = | |
| | | 4.4 Hz, 3H), 2.40 (dtd, J = 4.4, 9.2, 13.6 Hz, 1H). | |
| 118B | 2^{nd} peak | ¹H NMR (400 MHz, DMSO-d₆) δ = 8.93 (d, J = 2.0 Hz, | LCMS [ESI, |
| | | 1H), 8.71 (d, J = 2.0 Hz, 1H), 8.65 (s, 1H), 8.54 (br d, J = | M + 1]: 391.0 |
| | | 4.4 Hz, 1H), 8.38 (d, J = 8.8 Hz, 2H), 8.08-8.03 (m, 2H), | |
| | | 3.92-3.83 (m, 1H), 2.99-2.88 (m, 3H), 2.82 (d, J = 4.4 | |
| | | Hz, 3H), 2.45-2.34 (m, 1H). | |
| 119B | | ¹H NMR (400 MHz, CHLOROFORM-d) δ = 9.60 (d, J = 1.6 | LCMS [ESI, |
| | | Hz, 1H), 8.75 (d, J = 2.0 Hz, 1H), 8.58 (d, J = 1.6 Hz, | M + 1]: m/z = |
| | | 1H), 8.39 (dd, J = 2.0, 9.6 Hz, 1H), 8.34 (s, 1H), 7.57 (d, | 442.2 |
| | | J = 9.6 Hz, 1H), 3.91-3.84 (m, 4H), 3.71-3.62 (m, 4H), | |
| | | 3.58-3.46 (m, 2H), 3.33 (s, 3H), 3.13-3.05 (m, 1H), | |
| | | 3.04-2.94 (m, 1H), 2.18-2.02 (m, 1H), 1.98-1.85 (m, 1H) | |
| 119A | | ¹H NMR (400 MHz, DMSO-d₆) δ = 9.54 (d, J = 1.6 Hz, | LCMS [ESI, |
| | | 1H), 8.94 (d, J = 2.0 Hz, 1H), 8.73 (d, J = 2.0 Hz, 1H), 8.65 | M + 1]: m/z = |
| | | (s, 1H), 8.26 (dd, J = 2.0, 9.6 Hz, 1H), 7.71 (d, J = 9.6 Hz, | 442.2 |
| | | 1H), 3.76-3.71 (m, 4H), 3.53-3.49 (m, 4H), 3.41-3.37 | |
| | | (m, 3H), 3.19 (s, 3H), 3.14 (br s, 2H), 1.87 (br dd, J = | |
| | | 6.0, 9.6 Hz, 1H) | |
| 120A | | ¹H NMR (400 MHz, DMSO-d₆) δ = 8.96 (s, 1H), 8.87 (d, J = | LCMS [ESI, |
| | | 2.0 Hz, 1H), 8.64-8.61 (m, 2H), 8.54 (s, 1H), 8.35-8.31 | M + 1]: 379.2 |
| | | (m, 1H), 8.02 (d, J = 7.6 Hz, 1H), 7.70 (t, J = 8.0 Hz, 1H), | |
| | | 3.77-3.71 (m, 1H), 2.35-2.23 (m, 2H), 1.97-1.84 (m, | |
| | | 2H), 1.72-1.63 (m, 1H), 1.00 (d, J = 6.0 Hz, 3H). | |
| 120B | | ¹H NMR (400 MHz, DMSO-d₆) δ = 8.96 (s, 1H), 8.87 (d, J = | LCMS [ESI, |
| | | 2.0 Hz, 1H), 8.66-8.61 (m, 2H), 8.53 (s, 1H), 8.36-8.31 | M + 1]: 379.2 |
| | | (m, 1H), 8.03 (br d, J = 7.6 Hz, 1H), 7.76 (t, J = 8.0 Hz, | |
| | | 1H), 3.76-3.72 (m, 1H), 2.36-2.23 (m, 2H), 1.99-1.83 | |
| | | (m, 2H), 1.74-1.63 (m, 1H), 1.01 (br d, J = 6.0 Hz, 3H). | |
| 120C | | ¹H NMR (400 MHz, DMSO-d₆) δ = 14.42-14.13 (m, 1H), | LCMS [ESI, |
| | | 8.96 (s, 1H), 8.91-8.86 (m, 1H), 8.69-8.65 (m, 1H), 8.63 | M + 1]: 379.1 |
| | | (s, 1H), 8.61-8.51 (m, 1H), 8.34 (br d, J = 8.8 Hz, 1H), | |
| | | 8.03 (d, J = 8.0 Hz, 1H), 7.70 (t, J = 8.0 Hz, 1H), 3.98- | |
| | | 3.87 (m, 1H), 2.49-2.43 (m, 2H), 2.36-2.28 (m, 1H), 1.90- | |
| | | 1.79 (m, 1H), 1.43-1.31 (m, 1H), 1.09 (d, J = 6.8 Hz, | |
| | | 3H). | |
| 120D | | ¹H NMR (400 MHz, DMSO-d₆) δ = 8.96 (s, 1H), 8.89 (d, J = | LCMS [ESI, |
| | | 2.0 Hz, 1H), 8.68 (d, J = 2.0 Hz, 1H), 8.64 (s, 1H), 8.53 | M + 1]: 379.1 |
| | | (s, 1H), 8.33 (br d, J = 7.6 Hz, 1H), 8.03 (br d, J = 7.6 Hz, | |
| | | 1H), 7.70 (t, J = 8.0 Hz, 1H), 3.97-3.90 (m, 1H), 2.49- | |
| | | 2.44 (m, 2H), 2.39-2.25 (m, 1H), 1.91-1.82 (m, 1H), 1.38- | |
| | | 1.35 (m, 1H), 1.10 (d, J = 6.8 Hz, 3H). | |
| 122A | 1^{st} peak | ¹H NMR (400 MHz, DMSO-d₆) δ = 9.01-8.88 (m, 2H), | LCMS [ESI, |
| | | 8.75 (d, J = 2.0 Hz, 1H), 8.64 (s, 1H), 8.53 (s, 1H), 8.34 | M + 1]: 409.2 |
| | | (dd, J = 1.2, 8.0 Hz, 1H), 8.03 (d, J = 7.6 Hz, 1H), 7.70 (t, | |
| | | J = 7.6 Hz, 1H), 3.46 (br d, J = 10.0 Hz, 1H), 3.28-3.26 (m, | |
| | | 3H), 3.25 (br s, 1H), 3.16-3.12 (m, 1H), 3.03-2.97 (m, | |
| | | 1H), 0.75-0.66 (m, 1H), 0.65-0.54 (m, 1H), 0.53-0.36 | |
| | | (m, 2H). | |
| 122B | 2^{nd} peak | ¹H NMR (400 MHz, DMSO-d₆) δ = 8.99-8.90 (m, 2H), | LCMS [ESI, |
| | | 8.75 (d, J = 2.0 Hz, 1H), 8.64 (s, 1H), 8.48 (s, 1H), 8.32 | M + 1]: 409.2 |
| | | (dd, J = 1.2, 8.0 Hz, 1H), 8.03 (d, J = 7.6 Hz, 1H), 7.69 (t, | |
| | | J = 8.0 Hz, 1H), 3.48-3.46 (m, 1H), 3.27-3.27 (m, 3H), | |
| | | 3.25 (s, 1H), 3.14 (br d, J = 13.6 Hz, 1H), 3.03-2.99 (m, | |
| | | 1H), 0.75-0.65 (m, 1H), 0.58 (br d, J = 5.6 Hz, 1H), 0.45 | |
| | | (br s, 2H). | |
| 123A | | ¹H NMR (400 MHz, DMSO-d₆) δ = 14.57-13.96 (m, 1H), | LCMS [ESI, |
| | | 8.98 (s, 1H), 8.90 (s, 1H), 8.65 (br d, J = 7.6 Hz, 2H), | M + 1]: 391.1 |
| | | 8.57-8.49 (m, 1H), 8.36-8.29 (m, 1H), 8.03 (br d, J = 8.0 | |
| | | Hz, 1H), 7.73-7.65 (m, 1H), 3.23 (br s, 1H), 2.07-1.94 (m, | |
| | | 3H), 1.39-1.27 (m, 3H), 0.41-0.30 (m, 1H), 0.15 (br d, | |
| | | J = 4.0 Hz, 1H) | |
| 123B | | ¹H NMR (400 MHz, DMSO-d₆) δ = 14.64-13.84 (m, 1H), | LCMS [ESI, |
| | | 8.98 (t, J = 1.6 Hz, 1H), 8.89 (d, J = 2.0 Hz, 1H), 8.65 (d, | M + 1]: 391.2 |
| | | J = 2.0 Hz, 1H), 8.64 (s, 1H), 8.56 (br s, 1H), 8.34 (dd, J = | |
| | | 1.2, 8.0 Hz, 1H), 8.03 (d, J = 8.0 Hz, 1H), 7.70 (t, J = 8.0 | |
| | | Hz, 1H), 3.25-3.18 (m, 1H), 2.14-1.89 (m, 3H), 1.44- | |
| | | 1.22 (m, 3H), 0.41-0.28 (m, 1H), 0.14 (q, J = 4.0 Hz, 1H) | |
| 123C | | ¹H NMR (400 MHz, DMSO-d₆) δ = 14.72-13.77 (m, 1H), | LCMS [ESI, |
| | | 8.98-8.89 (m, 2H), 8.73 (d, J = 2.0 Hz, 1H), 8.63 (s, 1H), | M + 1]: 391.2 |
| | | 8.55 (br s, 1H), 8.36-8.29 (m, 1H), 8.03 (d, J = 8.0 Hz, | |
| | | 1H), 7.71 (t, J = 8.0 Hz, 1H), 3.83-3.74 (m, 1H), 2.33- | |
| | | 2.24 (m, 1H), 2.11-1.99 (m, 2H), 1.81 (dd, J = 3.3, 14.5 | |
| | | Hz, 1H), 1.40-1.28 (m, 2H), 0.62-0.46 (m, 2H) | |
| 123D | | ¹H NMR (400 MHz, DMSO-d₆) δ = 14.71-13.98 (m, 1H), 8.95 | LCMS [ESI, |
| | | (br s, 2H), 8.74 (s, 1H), 8.64 (s, 1H), 8.55 (br s, 1H), | M + 1]: 391.2 |
| | | 8.34 (br d, J = 8.0 Hz, 1H), 8.03 (br d, J = 7.2 Hz, 1H), | |
| | | 7.71 (br t, J = 8.0 Hz, 1H), 3.78 (br t, J = 9.6 Hz, 1H), | |
| | | 2.36-2.26 (m, 1H), 2.14-2.00 (m, 2H), 1.82 (br d, J = 13.2 | |
| | | Hz, 1H), 1.35 (br d, J = 4.0 Hz, 2H), 0.64-0.45 (m, 2H). | |
| 124A | 1^{st} peak | ¹H NMR (400 MHz, DMSO-d₆) δ = 8.95 (d, J = 2.0 Hz, | LCMS [ESI, |
| | | 1H), 8.72 (d, J = 2.4 Hz, 1H), 8.65 (s, 1H), 8.54 (br d, J = | M + 1]: m/z = |
| | | 4.8 Hz, 1H), 8.40 (d, J = 8.8 Hz, 2H), 8.06 (d, J = 8.8 Hz, | 371.2 |
| | | 2H), 3.21-3.08 (m, 1H), 3.02-2.89 (m, 1H), 2.82 (d, J = | |
| | | 4.4 Hz, 3H), 1.70-1.48 (m, 2H), 1.38-1.25 (m, 1H), 0.85 | |
| | | (t, J = 7.2 Hz, 6H). | |
| 124B | 2^{nd} peak | ¹H NMR (400 MHz, DMSO-d₆) δ = 8.95 (d, J = 2.0 Hz, | LCMS [ESI, |
| | | 1H), 8.72 (d, J = 2.0 Hz, 1H), 8.65 (s, 1H), 8.58-8.49 (m, | M + 1]: m/z = |
| | | 1H), 8.40 (d, J = 8.8 Hz, 2H), 8.06 (d, J = 8.8 Hz, 2H), | 371.2 |
| | | 3.22-3.09 (m, 1H), 3.03-2.90 (m, 1H), 2.82 (d, J = 4.4 Hz, | |
| | | 3H), 1.69-1.48 (m, 2H), 1.39-1.24 (m, 1H), 0.85 (t, J = | |
| | | 7.2 Hz, 6H). | |
| 125A | 1^{st} peak | ¹H NMR (400 MHz, DMSO-d₆) δ = 8.97 (d, J = 2.0 Hz, | LCMS [ESI, |
| | | 1H), 8.73 (d, J = 2.0 Hz, 1H), 8.69-8.64 (m, 1H), 8.58- | M + 1]: 382.1 |
| | | 8.52 (m, 2H), 8.29-8.22 (m, 2H), 3.21-3.15 (m, 1H), 3.02- | |
| | | 2.93 (m, 1H), 1.67-1.51 (m, 2H), 1.37-1.28 (m, 1H), | |
| | | 0.86 (br t, J = 6.8 Hz, 6H). | |
| 125B | 2^{nd} peak | ¹H NMR (400 MHz, DMSO-d₆) δ = 8.97 (d, J = 2.0 Hz, | LCMS [ESI, |
| | | 1H), 8.73 (d, J = 2.0 Hz, 1H), 8.68 (s, 1H), 8.57 (d, J = 8.4 | M + 1]: 382.1 |
| | | Hz, 2H), 8.27 (br d, J = 8.4 Hz, 2H), 3.17-3.11 (m, 1H), | |
| | | 3.00-2.93 (m, 1H), 1.70-1.51 (m, 2H), 1.39-1.27 (m, | |
| | | 1H), 0.86 (t, J = 6.8 Hz, 6H). | |
| 126A | 1^{st} peak | ¹H NMR (400 MHz, DMSO-d₆) δ = 8.96-8.90 (m, 2H), | LCMS [ESI, |
| | | 8.75 (d, J = 1.6 Hz, 1H), 8.64 (s, 1H), 8.44 (s, 1H), 8.30 | M + 1]: 411.2 |
| | | (dd, J = 1.2, 8.0 Hz, 1H), 8.03 (d, J = 8.0 Hz, 1H), 7.68 | |
| | | (t, J = 8.0 Hz, 1H), 3.41-3.31 (m, 2H), 3.26-3.21 (m, 3H), | |
| | | 2.94 (s, 2H), 1.15 (d, J = 10.8 Hz, 6H). | |
| 126B | 2^{nd} peak | ¹H NMR (400 MHz, DMSO-d₆) δ = 8.92 (br d, J = 1.6 Hz, | LCMS [ESI, |
| | | 2H), 8.75 (d, J = 1.6 Hz, 1H), 8.64 (s, 1H), 8.41-8.35 (m, | M + 1]: 411.2 |
| | | 1H), 8.28 (br d, J = 8.0 Hz, 1H), 8.02 (d, J = 8.0 Hz, 1H), | |
| | | 7.67 (t, J = 8.0 Hz, 1H), 3.40-3.31 (m, 2H), 3.25 (br s, | |
| | | 3H), 2.94 (s, 2H), 1.15 (d, J = 10.4 Hz, 6H). | |
| 127A | 1^{st} peak | ¹H NMR (400 MHz, DMSO-d₆) δ = 12.28-11.97 (m, 1H), | LCMS [ESI, |
| | | 8.89 (d, J = 2.0 Hz, 1H), 8.66 (d, J = 1.6 Hz, 1H), 8.56 (s, | M + 1]: 418.2 |
| | | 1H), 8.09-7.96 (m, 2H), 7.45 (br t, J = 8.4 Hz, 1H), 3.15- | |
| | | 3.08 (m, 1H), 2.97-2.89 (m, 1H), 2.67 (br t, J = 7.6 Hz, | |
| | | 2H), 2.27 (t, J = 7.2 Hz, 2H), 1.83 (quin, J = 7.2 Hz, 2H), | |
| | | 1.66-1.48 (m, 2H), 1.37-1.25 (m, 1H), 0.83 (t, J = 6.8 | |
| | | Hz, 6H). | |
| 127B | 2^{nd} peak | ¹H NMR (400 MHz, DMSO-d₆) δ = 12.26-11.93 (m, 1H), | LCMS [ESI, |
| | | 8.93 (s, 1H), 8.70 (s, 1H), 8.61 (s, 1H), 8.15-8.04 (m, 2H), | M + 1]: 418.2 |
| | | 7.50 (br t, J = 8.4 Hz, 1H), 3.19-3.09 (m, 1H), 3.01-2.91 | |
| | | (m, 1H), 2.70 (br t, J = 7.6 Hz, 2H), 2.28 (t, J = 7.2 Hz, | |
| | | 2H), 2.07 (s, 1H), 1.84 (quin, J = 7.2 Hz, 2H), 1.68-1.49 | |
| | | (m, 2H), 1.37-1.26 (m, 1H), 0.85 (t, J = 6.8 Hz, 6H). | |

### Example 7. Representative Procedure G for Synthesis of Pyrrolopyrazine Sulfoxides: 2-[butylsulfinyl]-5-[3-(4H-1,2,4-triazol-3-yl)phenyl]pyrrolo[2,3-b]pyrazine (Compounds 1A and 1B)

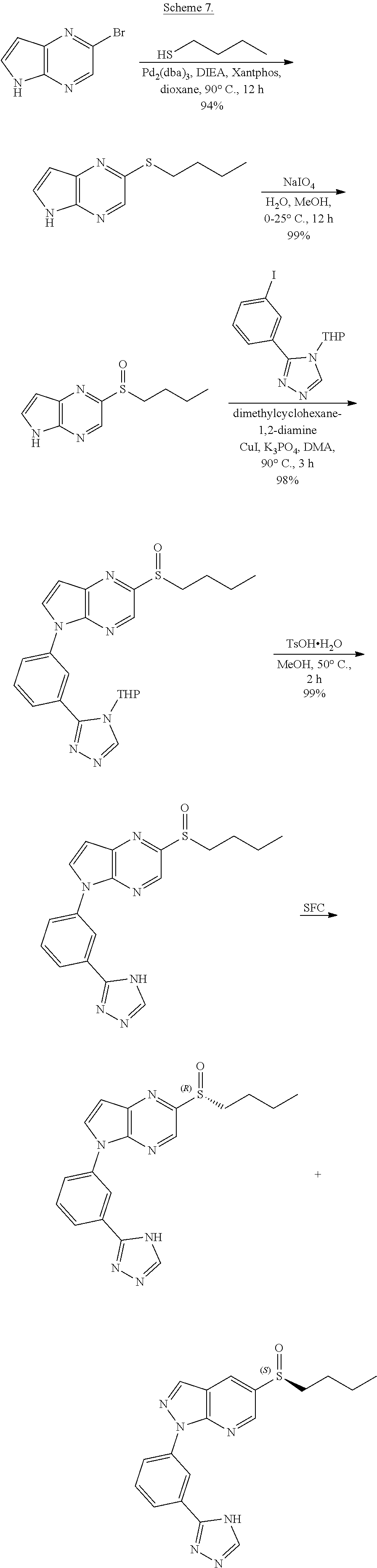

To a solution of 2-bromo-5H-pyrrolo[2,3-b]pyrazine (2.00 g, 10.1 mmol, 1.00 eq.) in dioxane (20.0 mL) under N₂ (15 psi) was added butane-1-thiol (2.26 g, 25.1 mmol, 2.68 mL, 2.48 eq.), Pd₂(dba)₃ (462 mg, 505 μmol, 0.05 eq.), DIEA (3.92 g, 30.3 mmol, 5.28 mL, 3.00 eq.) and Xantphos (584 mg, 1.01 mmol, 0.10 eq.). The mixture was stirred at 90° C. for 12 hours, then extracted with EtOAc (20 mL×3). The combined organic layers were washed with brine (25 mL), dried over Na₂SO₄, filtered and concentrated under reduced pressure to give a residue. The residue was purified by column chromatography (SiO₂, Petroleum ether/Ethyl acetate=1/0 to 3/1) to give 2-butylsulfanyl-5H-pyrrolo[2,3-b]pyrazine (2.00 g, 9.46 mmol, 94% yield, 98% purity) as a white solid.

¹H NMR (400 MHz, DMSO-d₆) δ=12.01 (br s, 1H), 8.13 (s, 1H), 7.80 (d, J=3.6 Hz, 1H), 6.55 (d, J=3.6 Hz, 1H), 3.18 (t, J=7.2 Hz, 2H), 1.62 (q, J=7.2 Hz, 2H), 1.41 (m, J=7.2 Hz, 2H), 0.88 (t, J=7.2 Hz, 3H).

To a solution of 2-butylsulfanyl-5H-pyrrolo[2,3-b]pyrazine (1.00 g, 4.82 mmol, 1.00 eq.) in MeOH (25.0 mL) was slowly added NaIO₄ (2.06 g, 9.65 mmol, 2.00 eq.) in H₂O (25.0 mL) at 0° C. The mixture was stirred at 25° C. for 12 hours, then quenched with saturated Na₂SO₃ (40 mL) at 0° C. The mixture was extracted with DCM (3×20 mL). The combined organic layers were washed with brine (35 mL), dried over Na₂SO₄, filtered and concentrated under reduced pressure to give 2-butylsulfinyl-5H-pyrrolo[2,3-b]pyrazine (1.19 g, 4.80 mmol, 99% yield, 90% purity) as a yellow solid. LCMS (ESI, M+1): m/z=224.1.

To a solution of 2-butylsulfinyl-5H-pyrrolo[2,3-b]pyrazine (200 mg, 896 μmol, 1.00 eq.) in DMA (2.00 mL) under N₂ was added 3-(3-iodophenyl)-4-tetrahydropyran-2-yl-1,2,4-triazole (350 mg, 985 mol, 1.10 eq.), K₃PO₄ (380 mg, 1.79 mmol, 2.00 eq.), (1R,2R)—N1,N2-dimethylcyclohexane-1,2-diamine (63.7 mg, 448 mol, 0.50 eq.) and CuI (85.3 mg, 448 mol, 0.50 eq.). The mixture was stirred at 90° C. for 3 hours under N₂ (15 psi). The reaction mixture was extracted with EtOAc (15 mL×3) and washed with H₂O (20 mL×3). The combined organic layers were washed with brine (15 mL×2), dried over Na₂SO₄, filtered and concentrated under reduced pressure to give a residue. The residue was purified by column chromatography (SiO₂, Petroleum ether/Ethyl acetate=1/0 to 1/2) to give 2-butylsulfinyl-5-[3-(4-tetrahydropyran-2-yl-1,2,4-triazol-3-yl)phenyl]pyrrolo[2,3-b]pyrazine (400 mg, 879 mol, 98% yield, 99% purity) as a colorless oil. LCMS (ESI, M+1): m/z=451.3.

To a solution of 2-butylsulfinyl-5-[3-(4-tetrahydropyran-2-yl-1,2,4-triazol-3-yl)phenyl]pyrrolo[2,3-b]pyrazine (500 mg, 1.11 mmol, 1.00 eq.) in MeOH (5.00 mL) was added TsOH·H₂O (317 mg, 1.66 mmol, 1.50 eq.). Then the mixture was stirred at 50° C. for 2 hours. The reaction mixture was quenched with NaHCO₃ (10 mL) and extracted with EtOAc (5 mL×3). The combined organic layers were washed with brine (15 mL), dried over Na₂SO₄, filtered and concentrated under reduced pressure to give 2-butylsulfinyl-5-[3-(4H-1,2,4-triazol-3-yl)phenyl]pyrrolo[2,3-b]pyrazine (440 mg, 1.10 mmol, 99% yield, 92% purity) as a colorless oil.

The racemic material was separated by SFC (column: DAICEL CHIRALPAK AD (250 mm×30 mm, 10 μm); mobile phase: [CO₂-MeOH (0.1% NH₃·H₂O)]; B %: 45%, isocratic elution mode) to give Peak 1 (Compound 1A) (153 mg, 413 mol, 34% yield, 99% purity) as a white solid and Peak 2 (Compound 1B) (144 mg, 390 mol, 33% yield, 99% purity) as a white solid.

Peak 1-1A: LCMS (ESI, M+1): m/z=367.2.

¹H NMR (400 MHz, DMSO-d6) δ=14.53-14.10 (m, 1H), 8.74 (s, 1H), 8.65 (d, J=4.0 Hz, 1H), 8.60-8.49 (m, 2H), 8.08 (d, J=7.6 Hz, 1H), 7.93 (br d, J=7.2 Hz, 1H), 7.77-7.66 (m, 1H), 7.07 (d, J=4.0 Hz, 1H), 3.23-3.12 (m, 1H), 3.10-2.97 (m, 1H), 1.73 (br dd, J=5.6, 9.2 Hz, 1H), 1.52-1.31 (m, 3H), 0.91-0.82 (m, 3H).

Peak 2-11B: LCMS (ESI, M+1): m/z=367.2

¹H NMR (400 MHz, DMSO-d6) δ=14.23 (br s, 1H), 8.74 (s, 1H), 8.72-8.62 (m, 2H), 8.53 (br s, 1H), 8.09 (br d, J=7.2 Hz, 1H), 7.90 (br d, J=6.4 Hz, 1H), 7.78-7.63 (m, 1H), 7.07 (br s, 1H), 3.23-3.13 (m, 1H), 3.08-2.99 (m, 1H), 1.83-1.61 (m, 1H), 1.52-1.30 (m, 3H), 0.87 (t, J=7.2 Hz, 3H).

The following were made by Example 7.

TABLE 3
Analytical data for representative compounds of the disclosure.

| Comp. | | | |
|---|---|---|---|
| No. | Chiral SFC | ¹H NMR | LCMS |
| 1B | 2nd peak | ¹H NMR (400 MHz, DMSO-d6) δ = 14.23 (br s, 1H), 8.74 | LCMS (ESI, |
| | | (s, 1H), 8.72-8.62 (m, 2H), 8.53 (br s, 1H), 8.09 (br d, J = | M + 1): m/z = |
| | | 7.2 Hz, 1H), 7.90 (br d, J = 6.4 Hz, 1H), 7.78-7.63 (m, | 367.2 |
| | | 1H), 7.07 (br s, 1H), 3.23-3.13 (m, 1H), 3.08-2.99 (m, | |
| | | 1H), 1.83-1.61 (m, 1H), 1.52-1.30 (m, 3H), 0.87 (t, J = | |
| | | 7.2 Hz, 3H). | |
| 1A | 1st peak | ¹H NMR (400 MHz, DMSO-d6) δ = 14.53-14.10 (m, | LCMS (ESI, |
| | | 1H), 8.74 (s, 1H), 8.65 (d, J = 4.0 Hz, 1H), 8.60-8.49 (m, | M + 1): m/z = |
| | | 2H), 8.08 (d, J = 7.6 Hz, 1H), 7.93 (br d, J = 7.2 Hz, 1H), | 367.2 |
| | | 7.77-7.66 (m, 1H), 7.07 (d, J = 4.0 Hz, 1H), 3.23-3.12 | |
| | | (m, 1H), 3.10-2.97 (m, 1H), 1.73 (br dd, J = 5.6, 9.2 Hz, | |
| | | 1H), 1.52-1.31 (m, 3H), 0.91-0.82 (m, 3H). | |
| 76 | | ¹H NMR (400 MHz, DMSO-d6) δ = 8.55 (d, J = 4.2 Hz, | LCMS [ESI, |
| | | 1H), 8.37 (d, J = 3.6 Hz, 1H), 7.73 (d, J = 8.8 Hz, 2H), | M + 1]: 367.1 |
| | | 7.12 (d, J = 8.8 Hz, 2H), 6.94 (br s, 1H), 4.49-4.32 (m, | |
| | | 1H), 4.10 (q, J = 7.2 Hz, 2H), 3.97-3.72 (m, 2H), 3.61- | |
| | | 3.44 (m, 2H), 3.31-2.61 (m, 2H), 1.36 (t, J = 7.2 Hz, 3H), | |
| | | 1.21-0.96 (m, 3H). | |
| 78 | | ¹H NMR (400 MHz, DMSO-d₆) δ = 8.64 (s, 1 H), 8.57 (d, | LCMS [ESI, |
| | | J = 3.6 Hz, 1 H), 7.48 (dt, J = 10.4, 2.0 Hz, 1H), 7.44 (s, | M + 1]: 405.1 |
| | | 1H), 7.00 (d, J = 3.6 Hz, 1H), 6.89 (dt, J = 11.6, 2.2 Hz, | |
| | | 1H), 4.14 (q, J = 7.2 Hz, 2H), 3.81 (br s, 2H), 3.65 (br s, | |
| | | 2H), 2.01-2.17 (m, 4 H), 1.37 (t, J = 7.2 Hz, 3H). | |
| 81 | | ¹H NMR (400 MHz, DMSO-d₆) δ = 14.02-14.55 (m, 1H), | LCMS [ESI, |
| | | 8.42-8.73 (m, 4H), 8.07 (d, J = 7.60 Hz, 1H), 7.80-8.00 | M + 1]: 390.1 |
| | | (m, 1H), 7.62-7.78 (m, 1H), 7.02 (br s, 1H), 4.30-4.49 | |
| | | (m, 1H), 3.69-4.02 (m, 2H), 3.42-3.64 (m, 2H), 3.21- | |
| | | 3.29 (m, 1H), 2.88-3.09 (m, 1H), 0.96-1.21 (m, 3H). | |
| 82 | | ¹H NMR (400 MHz, DMSO-d₆) δ = 14.30 (br s, 1H), 8.64 | LCMS [ESI, |
| | | (s, 1H), 8.57 (d, J = 4.0 Hz, 2H), 8.56-8.53 (m, 1H), 8.07 | M + 1]: 410.2 |
| | | (d, J = 7.6 Hz, 1H), 7.92 (br d, J = 7.2 Hz, 1H), 7.77-7.66 | |
| | | (m, 1H), 7.02 (d, J = 4.0 Hz, 1H), 3.82 (br d, J = 1.0 Hz, | |
| | | 2H), 3.67 (br s, 2H), 2.17-2.04 (m, 4H) | |
| 83 | | ¹H NMR (400 MHz, DMSO-d₆) δ = 14.91-13.75 (m, 1H), | LCMS (ESI, |
| | | 8.71-8.60 (m, 3H), 8.53 (d, J = 1.6 Hz, 1H), 7.93 (br d, | M + 1): m/z = |
| | | J = 10.0 Hz, 1H), 7.86-7.74 (m, 1H), 7.05 (d, J = 3.9 Hz, | 428.1 |
| | | 1H), 3.82 (br s, 2H), 3.66 (br s, 2H), 2.18-1.95 (m, 4H). | |
| 84 | | ¹H NMR (400 MHz, DMSO-d₆) δ = 13.16 (br s, 1H), 8.65 | LCMS (ESI, |
| | | (s, 1H), 8.62 (d, J = 4.0, 1H), 8.14 (d, J = 2.0 Hz, 4H), 7.05 | M + 1): m/z = |
| | | (d, J = 3.9 Hz, 1H), 3.82 (br s, 2H), 3.66 (br s, 2H), 2.22- | 387.1. |
| | | 1.94 (m, 4H). | |
| 85 | | ¹H NMR (400 MHz, DMSO-d6) δ = 8.60 (s, 1H), 8.49 (d, | LCMS [ESI, |
| | | J = 4.0 Hz, 1H), 8.17-8.10 (m, 2H), 8.08-8.02 (m, 2H), | M + 1]: 367.1. |
| | | 7.00 (d, J = 4.0 Hz, 1H), 4.46-4.21 (m, 1H), 3.97-3.79 | |
| | | (m, 2H), 3.62-3.47 (m, 4H), 2.86-2.77 (m, 1H), 1.24- | |
| | | 0.99 (m, 3H) | |
| 86 | | ¹H NMR (400 MHz, DMSO-d6) δ = 8.59-8.46 (m, 2H), | LCMS [ESI, |
| | | 8.18-8.06 (m, 4H), 6.99 (d, J = 4.0 Hz, 1H), 4.48-4.13 | M + 1]: 365.1. |
| | | (m, 1H), 3.87-3.64 (m, 1H), 2.83-2.63 (m, 2H), 1.88- | |
| | | 1.62 (m, 3H), 1.58-1.47 (m, 1H), 1.28-1.16 (m, 1H), | |
| | | 1.04-0.68 (m, 3H) | |
| 87 | | ¹H NMR (400 MHz, DMSO-d₆) δ = 14.47-14.28 (m, 1H), | LCMS (ESI, |
| | | 8.69-8.60 (m, 3H), 8.54 (s, 1H), 7.95-7.90 (m, 1H), | M + 1): m/z = |
| | | 7.83-7.76 (m, 1H), 7.05 (br d, J = 3.2 Hz, 1H), | 408.2 |
| | | 4.45-4.33 (m, 1H), 3.99-3.75 (m, 2H), 3.58-3.44 (m, 2H), | |
| | | 3.04-2.92 (m, 1H), 2.68-2.53 (m, 1H), 1.20-0.98 (m, 3H). | |

### Example 8. Representative Procedure H for Synthesis of Azaindole Sulfoxides: Methyl 4-[5-[(R)-cyclobutylsulfinyl]pyrrolo[2,3-b]pyridin-1-yl]benzoate and Methyl 4-[5-[(S)-cyclobutylsulfinyl]pyrrolo[2,3-b]pyridin-1-yl]benzoate (Compounds 49A and 49B)

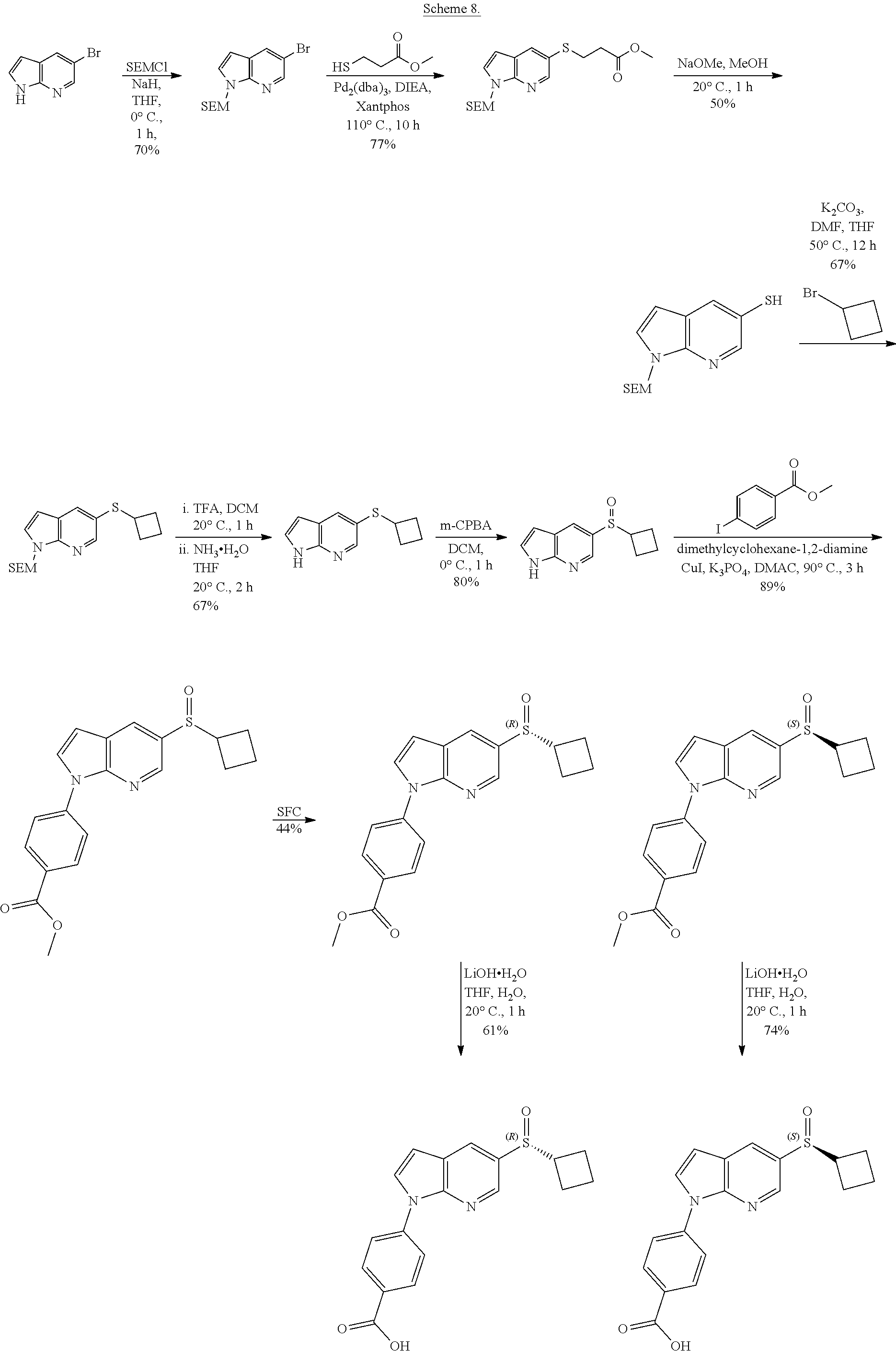

To a solution of 5-bromo-1H-pyrrolo[2,3-b]pyridine (10.0 g, 50.7 mmol, 1.00 eq.) in THF (100 mL) was added NaH (4.06 g, 101 mmol, 60% purity, 2.00 eq.) and SEM-Cl (16.9 g, 101 mmol, 17.9 mL, 2.00 eq.). The mixture was stirred at 0° C. for 1 hour. The mixture was poured into NH₄Cl (100 mL) and extracted with EA (200 ml×3). The combined organic layer was washed with brine (100 mL), dried over Na₂SO₄, then the mixture was filtered, and the filtrate was concentrated. The residue was purified by column chromatography (SiO₂, PE/EA=50/1 to 10/1) to give compound 2-[(5-bromopyrrolo[2,3-b]pyridin-1-yl)methoxy]ethyl-trimethyl-silane (12.0 g, 35.5 mmol, 70% yield, 97% purity) as a white solid. LCMS (ESI, M+1): m/z=329.0.

A mixture of 2-[(5-bromopyrrolo[2,3-b]pyridin-1-yl)methoxy]ethyl-trimethyl-silane (12.0 g, 36.6 mmol, 1.00 eq.), methyl 3-sulfanylpropanoate (6.61 g, 55.0 mmol, 5.95 mL, 1.50 eq.), Pd₂(dba)₃ (3.36 g, 3.67 mmol, 0.10 eq.), Xantphos (4.24 g, 7.33 mmol, 0.20 eq.) and DIEA (9.48 g, 73.3 mmol, 12.77 mL, 2.00 eq.) in dioxane (100 mL) was degassed and purged with N₂ for 3 times, and then the mixture was stirred at 110° C. for 10 hours under N₂ atmosphere (15 psi). The reaction was diluted with EA (200 mL) and quenched with H₂O (100 mL). The mixture was extracted with EtOAc (3×200 mL). The combined organic extracts were washed with H₂O (3×100 mL) and brine (200 mL), then dried over Na₂SO₄. The residue was purified by column chromatography (SiO₂, Petroleum ether/Ethyl acetate=10/1 to 1/1) to give compound methyl 3-[1-(2-trimethylsilylethoxymethyl)pyrrolo[2,3-b]pyridin-5-yl]sulfanylpropanoate (11.5 g, 28.5 mmol, 77% yield, 91% purity) as a yellow oil. LCMS (ESI, M+1): m/z=367.1.

To a solution of methyl 3-[1-(2-trimethylsilylethoxymethyl)pyrrolo[2,3-b]pyridin-5-yl]sulfanylpropanoate (5.00 g, 13.6 mmol, 1.00 eq.) in MeOH (50.0 mL) was added NaOMe (2.21 g, 40.9 mmol, 3.00 eq.). The mixture was stirred at 20° C. for 1 hour. The mixture was poured into NaHCO₃ (50 mL) and extracted with EA (50 mL×3). The combined organic layers were washed with brine (50 mL), dried over Na₂SO₄, then the mixture was filtered, and the filtrate was concentrated. The residue was purified by column chromatography (SiO₂, PE/EA=50/1 to 5/1) to give 1-(2-trimethylsilylethoxymethyl)pyrrolo [2,3-b]pyridine-5-thiol (2.40 g, 6.85 mmol, 50% yield, 80% purity) as a yellow oil. LCMS (ESI, M+1): m/z=281.1.

To a solution of 1-(2-trimethylsilylethoxymethyl)pyrrolo[2,3-b]pyridine-5-thiol (1.20 g, 4.28 mmol, 1.00 eq.) and bromocyclobutane (1.73 g, 12.8 mmol, 1.21 mL, 3.00 eq.) in THF (10.0 mL) and DMF (5.00 mL) was added K₂CO₃ (1.18 g, 8.56 mmol, 2.00 eq.) at 0° C. The mixture was stirred at 50° C. for 12 hours. The mixture was poured into H₂O (10 mL) and extracted with EA (10 mL×3). The combined organic layer was washed with brine (10 mL), dried over Na₂SO₄, then the mixture was filtered, and the filtrate was concentrated. The residue was purified by column chromatography (SiO₂, PE/EA=50/1 to 5/1) to give compound 2-[(5-cyclobutylsulfanylpyrrolo[2,3-b]pyridin-1-yl)methoxy]ethyl-trimethyl-silane (1.20 g, 2.87 mmol, 67% yield, 80% purity) as a yellow oil. LCMS (ESI, M+1): m/z=335.1.

To a solution of 2-[(5-cyclobutylsulfanylpyrrolo[2,3-b]pyridin-1-yl)methoxy]ethyl-trimethyl-silane (1.00 g, 2.99 mmol, 1.00 eq.) in DCM (10.0 mL) was added TFA (18.4 g, 161 mmol, 12.0 mL, 54.0 eq.). The mixture was stirred at 20° C. for 1 hour. The reaction mixture was concentrated under reduced pressure to give (5-cyclobutylsulfanylpyrrolo[2,3-b]pyridine-1-yl)methanol (900 mg, crude) as a yellow oil. LCMS (ESI, M+1): m/z=235.1.

To a solution of (5-cyclobutylsulfanylpyrrolo[2,3-b]pyridin-1-yl)methanol (900 mg, 3.84 mmol, 1.00 eq.) in THF (10.0 mL) was added NH₃·H₂O (20.4 g, 163 mmol, 22.5 mL, 28% purity, 42.5 eq.). The mixture was stirred at 20° C. for 2 hours. After being cooled to room temperature, the mixture was concentrated. The residue was diluted with EtOAc (10 mL) and water (10 mL). The layers were separated, and the aqueous phase was extracted with EtOAc (3×10 mL). The organic layer was dried over Na₂SO₄, filtered and concentrated. The residue was purified by column chromatography (SiO₂, PE/EA=20/1 to 1/1) to give 5-cyclobutylsulfanyl-1H-pyrrolo[2,3-b]pyridine (750 mg, 2.61 mmol, 67% yield, 71% purity) as a yellow oil. LCMS (ESI, M+1): m/z=205.2.

To a solution of 5-cyclobutylsulfanyl-1H-pyrrolo[2,3-b]pyridine (697 mg, 3.41 mmol, 1.00 eq.) in DCM (10.0 mL) was added m-CPBA (692 mg, 3.41 mmol, 85% purity, 1.00 eq.). The mixture was stirred at 0° C. for 1 hour. The reaction mixture was diluted with DCM (10 mL) and saturated Na₂SO₃ (10 mL) and extracted with DCM (20 mL×3). The combined organic layers were washed with brine (10 mL×1), dried over Na₂SO₄, filtered and concentrated under reduced pressure to give a residue. The residue was purified by column chromatography (SiO₂, PE/EA=10/1 to 1/1) to give 5-cyclobutylsulfinyl-1H-pyrrolo[2,3-b]pyridine (631 mg, 2.75 mmol, 80% yield, 96% purity) as a white solid. LCMS (ESI, M+1): m/z=221.1. ¹H NMR (400 MHz, DMSO-d₆) δ=12.01 (br s, 1H), 8.37 (d, J=2.0 Hz, 1H), 8.19 (d, J=2.0 Hz, 1H), 7.67-7.48 (m, 1H), 6.57 (dd, J=1.6, 3.2 Hz, 1H), 3.74 (t, J=8.0 Hz, 1H), 2.41-2.21 (m, 2H), 2.16-2.02 (m, 1H), 1.90 (td, J=8.8, 10.8 Hz, 1H), 1.85-1.76 (m, 1H), 1.65 (br dd, J=4.0, 8.4 Hz, 1H).

A mixture of 5-cyclobutylsulfinyl-1H-pyrrolo[2,3-b]pyridine (581 mg, 2.64 mmol, 1.00 eq.), methyl 4-iodobenzoate (760 mg, 2.90 mmol, 1.10 eq.), CuI (251 mg, 1.32 mmol, 0.50 eq.), K₃PO₄ (1.12 g, 5.27 mmol, 2.00 eq.) and (1R,2R)—N1,N2-dimethylcyclohexane-1,2-diamine (187 mg, 1.32 mmol, 0.50 eq.) in DMAC (10.0 mL) was degassed and purged with nitrogen for 3 times and then the mixture was stirred at 90° C. for 3 hours (15 psi). The mixture was poured into H₂O (10 mL) and extracted with EA (10 mL×3). The combined organic layer was washed with brine (10 mL), dried over Na₂SO₄, then the mixture was filtered, and the filtrate was concentrated. The residue was purified by column chromatography (SiO₂, PE/EA=10/1 to 0/1) to give methyl 4-(5-cyclobutylsulfinylpyrrolo[2,3-b]pyridin-1-yl)benzoate (900 mg, 2.36 mmol, 89% yield, 93% purity) as a yellow oil. LCMS (ESI, M+1): m/z=355.1.

5-cyclopentylsulfinyl-1-[3-(4H-1,2,4-triazol-3-yl) phenyl]pyrrolo[2,3-b]pyridine (900 mg, 89% yield, 93% purity) was purified by SFC (column: DAICEL CHIRALPAK AD (250 mm×30 mm, 10 um); mobile phase: [CO₂-EtOH (0.1% NH₃H₂OETOH)]; B %: 55%-55%, 6.6 min) to give Peak 1 (Compound 49A) (300 mg, 837 mol, 33.0% yield, 99% purity) as a yellow oil and Peak 2 (Compound 49B) (400 mg, 1.12 mmol, 44% yield, 99% purity) as an off-white gum.

Compound 49: To a solution of methyl 4-[5-[(R)-cyclobutylsulfinyl]pyrrolo [2,3-b]pyridine-1-yl]benzoate (100 mg, 282 mol, 1.00 eq.) in THF (1.00 mL) was added LiOH H₂O (59.2 mg, 1.41 mmol, 5.00 eq.) in H₂O (1.00 mL) slowly at 0° C. The mixture was stirred at 0° C. for 1 hour. The mixture was concentrated under reduced pressure. The pH was acidified to 6 with 1N HCl. Then the mixture was filtered to get the impure product. The impure product was triturated with MeCN (1.00 mL) to give the title compound (60.0 mg, 172 mol, 61% yield, 98% purity) as a white solid. LCMS (ESI, M+1): m/z=341.1.

¹H NMR (400 MHz, DMSO-d₆) δ=13.06 (br s, 1H), 8.52 (d, J=2.0 Hz, 1H), 8.35 (d, J=2.0 Hz, 1H), 8.21 (d, J=3.6 Hz, 1H), 8.12 (s, 4H), 6.93 (d, J=4.0 Hz, 1H), 3.81 (t, J=8.0 Hz, 1H), 2.44-2.26 (m, 21H), 2.14 (br dd, J=3.6, 8.0 Hz, 1H), 1.98-1.78 (i, 2H), 1.72-1.57 (i, 1H).

Compound 49B3: To a solution of methyl 4-[5-[(S)-cyclobutylsulfinyl]pyrrolo [2,3-b]pyridin-1-yl]benzoate (150 mg, 423 μmol, 1.00 eq.) in THF (1.00 mL) was added LiOH·H₂ (88.8 mg, 2.12 mmol, 5.00 eq.) in H₂H (1.00 mL) slowly at 0 MC. The mixture was stirred at 0° C. for 1 hour. The mixture was concentrated under reduced pressure. The pH was acidified to 6 with 1N HCL. Then the mixture was filtered to get the impure product. The impure product was triturated with MeCN (1.00 mL) to give the title compound (109 mg, 317 μmol, 74% yield, 99% purity) as a white solid. LCMS (ESI, M+1): m/z=341.1.

¹H NMR (400 MHz, DMSO-d₆) δ=13.70-12.59 (i, 1H), 8.52 (d, J=2. Hz, 1H), 8.35 (d, J=2.0 Hz, 1H), 8.20 (d, J=3.6 Hz, 1H), 8.11 (s, 4H), 6.92 (d, J=4.0 Hz, 1H), 3.81 (brt, J=8.0 Hz, 1H), 2.42-2.26 (m, 2H), 2.21-2.07 (m, 1H), 1.97-1.73 (m, 2H), 1.64 (br dd, J=2.8, 8.0 Hz, 1H).

The following compounds were by Example 8.

TABLE 4
Analytical data for representative compounds of the disclosure.

| Comp. No. | Chiral SFC | ¹H NMR | LCMS |
|---|---|---|---|
| 48B | 2nd peak | ¹H NMR (400 MHz, DMSO-d₆) δ = 8.58 (d, J = 1.6 Hz, | LCMS (ESI, |
| | | 1H), 8.43 (d, J = 1.6 Hz, 1H), 8.21 (d, J = 3.6 Hz, 1H), | M + 1): |
| | | 8.12 (s, 4H), 6.93 (d, J = 3.6 Hz, 1H), 3.08-3.00 (m, | m/z = 357.1 |
| | | 1H), 2.99-2.88 (m, 1H), 1.72-1.57 (m, 1H), 1.50 (br dd, | |
| | | J = 5.6, 10.8 Hz, 1H), 1.38-1.26 (m, 1H), 0.84 (t, J = 6.4 | |
| | | Hz, 6H) | |
| 48A | 1st peak | ¹H NMR (400 MHz, DMSO-d₆) δ = 8.59 (d, J = 2.0 Hz, | LCMS (ESI, |
| | | 1H), 8.43 (d, J = 2.0 Hz, 1H), 8.22 (d, J = 3.6 Hz, 1H), | M + 1): |
| | | 8.12 (s, 4H), 6.94 (d, J = 3.6 Hz, 1H), 3.10-3.02 (m, | m/z = 357.1 |
| | | 1H), 2.98-2.88 (m, 1H), 1.70-1.50 (m, 2H), 1.33 (br dd, | |
| | | J = 5.6, 11.7 Hz, 1H), 0.85 (t, J = 6.4 Hz, 6H). | |
| 49B | 2nd peak | ¹H NMR (400 MHz, DMSO-d₆) δ = 13.70-12.59 (m, | LCMS (ESI, |
| | | 1H), 8.52 (d, J = 2.0 Hz, 1H), 8.35 (d, J = 2.0 Hz, 1H), | M + 1): |
| | | 8.20 (d, J = 3.6 Hz, 1H), 8.11 (s, 4H), 6.92 (d, J = 4.0 Hz, | m/z = 341.1 |
| | | 1H), 3.81 (br t, J = 8.0 Hz, 1H), 2.42-2.26 (m, 2H), 2.21- | |
| | | 2.07 (m, 1H), 1.97-1.73 (m, 2H), 1.64 (br dd, J = 2.8, | |
| | | 8.0 Hz, 1H) | |
| 49A | 1st peak | ¹H NMR (400 MHz, DMSO-d₆) δ = 13.06 (br s, 1H), 8.52 | LCMS (ESI, |
| | | (d, J = 2.0 Hz, 1H), 8.35 (d, J = 2.0 Hz, 1H), 8.21 (d, J = | M + 1): |
| | | 3.6 Hz, 1H), 8.12 (s, 4H), 6.93 (d, J = 4.0 Hz, 1H), 3.81 | m/z = 341.1 |
| | | (t, J = 8.0 Hz, 1H), 2.44-2.26 (m, 2H), 2.14 (br dd, J = | |
| | | 3.6, 8.0 Hz, 1H), 1.98-1.78 (m, 2H), 1.72-1.57 (m, 1H) | |
| 50B | 2nd peak | ¹H NMR (400 MHz, DMSO-d₆) δ = 14.88-13.67 (m, | LCMS (ESI, |
| | | 1H), 8.52 (dd, J = 1.6, 12.0 Hz, 3H), 8.35 (d, J = 2.0 Hz, | M + 1): |
| | | 1H), 8.15 (d, J = 3.6 Hz, 1H), 8.03 (d, J = 8.0 Hz, 1H), | m/z = 364.1 |
| | | 7.90 (br d, J = 8.4 Hz, 1H), 7.72-7.64 (m, 1H), 6.90 (d, | |
| | | J = 3.6 Hz, 1H), 3.81 (quin, J = 8.4 Hz, 1H), 2.42-2.34 (m, | |
| | | 1H), 2.33-2.25 (m, 1H), 2.18-2.08 (m, 1H), 1.97-1.86 | |
| | | (m, 1H), 1.86-1.77 (m, 1H), 1.71-1.60 (m, 1H). | |
| 50A | 1st peak | ¹H NMR (400 MHz, DMSO-d₆) δ = 14.68-13.93 (m, 1H), | LCMS (ESI, |
| | | 8.64-8.43 (m, 3H), 8.35 (d, J = 2.0 Hz, 1H), 8.15 | M + 1): |
| | | (d, J = 3.6 Hz, 1H), 8.03 (d, J = 7.6 Hz, 1H), 7.90 (br d, | m/z = 364.1 |
| | | J = 7.6 Hz, 1H), 7.75-7.61 (m, 1H), 6.90 (d, J = 3.6 Hz, | |
| | | 1H), 3.82 (quin, J = 8.4 Hz, 1H), 2.40-2.24 (m, 2H), | |
| | | 2.14 (tt, J = 4.0, 12.0 Hz, 1H), 1.97-1.88 (m, 1H), 1.83 | |
| | | (ddd, J = 4.0, 10.0, 14.0 Hz, 1H), 1.67 (dtd, J = 4.0, 8.0, | |
| | | 11.6 Hz, 1H). | |
| 51B | 2nd peak | ¹H NMR (400 MHz, DMSO-d6) δ = 14.87-13.82 (m, 1H), | LCMS [ESI, |
| | | 8.68 (br s, 1H), 8.57 (d, J = 1.6 Hz, 1H), 8.55 (br s, | M + 1]: 378.3 |
| | | 1H), 8.43 (d, J = 1.6 Hz, 1H), 8.17 (d, J = 3.6 Hz, 1H), | |
| | | 8.05 (br d, J = 7.2 Hz, 1H), 7.87 (br d, J = 6.4 Hz, 1H), | |
| | | 7.79-7.58 (m, 1H), 6.91 (br s, 1H), 3.14 (qd, J = 6.4, 9.2 | |
| | | Hz, 1H), 3.07-2.93 (m, 1H), 1.61-1.49 (m, 1H), 1.47- | |
| | | 1.34 (m, 1H), 0.84-0.69 (m, 1H), 0.48-0.33 (m, 2H), | |
| | | 0.15-0.05 (m, 2H). | |
| 51A | 1st peak | ¹H NMR (400 MHz, DMSO-d6) δ = 14.70-13.93 (m, | LCMS [ESI, |
| | | 1H), 8.72-8.47 (m, 3H), 8.43 (d, J = 2.0 Hz, 1H), 8.17 | M + 1]: 378.3 |
| | | (d, J = 3.6 Hz, 1H), 8.04 (d, J = 7.6 Hz, 1H), 7.91 (br d, | |
| | | J = 7.2 Hz, 1H), 7.75-7.65 (m, 1H), 6.91 (d, J = 3.6 Hz, | |
| | | 1H), 3.14 (ddd, J = 6.4, 9.2, 13.2 Hz, 1H), 3.07-2.96 (m, | |
| | | 1H), 1.62-1.49 (m, 1H), 1.47-1.35 (m, 1H), 0.87-0.69 | |
| | | (m, 1H), 0.50-0.33 (m, 2H), 0.16--0.04 (m, 2H). | |
| 52B | 2nd peak | ¹H NMR (400 MHz, DMSO-d6) δ = 14.34 (br s, 1H), | LCMS [ESI, |
| | | 8.73-8.58 (m, 2H), 8.53 (s, 1H), 8.44 (s, 1H), 8.25 (br d, | M + 1]: 398.0 |
| | | J = 2.4 Hz, 1H), 7.93 (br d, J = 9.6 Hz, 1H), 7.76 (br d, | |
| | | J = 9.6 Hz, 1H), 6.96-6.92 (m, 1H), 3.16-3.00 (m, 1H), | |
| | | 2.99-2.86 (m, 1H), 1.68-1.58 (m, 1H), 1.57-1.47 (m, | |
| | | 1H), 1.39-1.27 (m, 1H), 0.85 (br t, J = 6.4 Hz, 6H). | |
| 52A | 1st peak | ¹H NMR (400 MHz, DMSO-d6) δ = 14.33 (br s, 1H), 8.61 (s, | LCMS [ESI, |
| | | 2H), 8.53 (s, 1H), 8.44 (s, 1H), 8.25 (br d, J = 3.2 | M + 1]: 398.0 |
| | | Hz, 1H), 7.93 (br d, J = 9.6 Hz, 1H), 7.76 (br d, J = 9.6 | |
| | | Hz, 1H), 7.02-6.90 (m, 1H), 3.16-3.02 (m, 1H), 3.01- | |
| | | 2.86 (m, 1H), 1.69-1.58 (m, 1H), 1.56-1.42 (m, 1H), | |
| | | 1.41-1.27 (m, 1H), 0.85 (br t, J = 6.4 Hz, 6H). | |
| 53B | 2nd peak | ¹H NMR (400 MHz, DMSO-d₆) δ = 9.18 (dd, J = 2.0, 8.8 Hz, | LCMS (ESI, |
| | | 2H), 8.97 (t, J = 2.0 Hz, 1H), 8.68 (s, 1H), 8.61 (d, | M + 1): |
| | | J = 1.6 Hz, 1H), 8.46 (d, J = 1.6 Hz, 1H), 8.31 (d, J = | m/z = 381.1 |
| | | 3.6 Hz, 1H), 6.97 (d, J = 3.6 Hz, 1H), 3.10-3.04 (m, 1H), | |
| | | 2.97-2.90 (m, 1H), 1.63 (td, J = 6.8, 13.2 Hz, 1H), 1.56- | |
| | | 1.45 (m, 1H), 1.39-1.29 (m, 1H), 0.85 (t, J = 6.4 Hz, | |
| | | 6H). | |
| 53A | 1st peak | ¹H NMR (400 MHz, DMSO-d₆) δ = 9.18 (dd, J = 2.0, 9.2 | LCMS (ESI, |
| | | Hz, 2H), 8.97 (t, J = 2.4 Hz, 1H), 8.68 (s, 1H), 8.61 (d, | M + 1): |
| | | J = 2.0 Hz, 1H), 8.46 (d, J = 2.0 Hz, 1H), 8.31 (d, J = 3.6 | m/z = 381.1 |
| | | Hz, 1H), 6.97 (d, J = 3.6 Hz, 1H), 3.10-3.05 (m, 1H), | |
| | | 2.97-2.91 (m, 1H), 1.63 (td, J = 6.8, 13.2 Hz, 1H), 1.57- | |
| | | 1.47 (m, 1H), 1.38-1.29 (m, 1H), 0.85 (t, J = 6.4 Hz, | |
| | | 6H). | |
| 54B | 2nd peak | ¹H NMR (400 MHz, DMSO-d6) δ = 14.26 (br dd, J = 4.2, | LCMS (ESI, |
| | | 8.4 Hz, 1H), 8.65-8.46 (m, 3H), 8.43 (d, J = 2.0 Hz, 1H), | M + 1): |
| | | 8.17 (d, J = 3.6 Hz, 1H), 8.04 (d, J = 8.0 Hz, 1H), 7.91 | m/z = 380.1 |
| | | (br d, J = 7.2 Hz, 1H), 7.74-7.63 (m, 1H), 6.92 (d, J = 3.6 | |
| | | Hz, 1H), 3.07 (qd, J = 5.2, 13.2 Hz, 1H), 2.93 (qd, J = | |
| | | 5.2, 13.2 Hz, 1H), 1.69-1.58 (m, 1H), 1.52 (br dd, J = | |
| | | 5.6, 11.2 Hz, 1H), 1.39-1.28 (m, 1H), 0.86 (t, J = 6.4 Hz, | |
| | | 6H | |
| 54A | 1 st peak | ¹H NMR (400 MHz, DMSO-d6) δ = 14.26 (br d, J = 4.0 | LCMS (ESI, |
| | | Hz, 1H), 8.64-8.47 (m, 3H), 8.44 (d, J = 2.0 Hz, 1H), | M + 1): |
| | | 8.17 (d, J = 3.6 Hz, 1H), 8.04 (d, J = 8.0 Hz, 1H), 7.91 | m/z = 380.1 |
| | | (br d, J = 7.2 Hz, 1H), 7.74-7.64 (m, 1H), 6.92 (d, J = | |
| | | 3.6 Hz, 1H), 3.07 (ddd, J = 5.2, 10.8, 13.2 Hz, 1H), 2.93 | |
| | | (ddd, J = 5.2, 10.8, 13.2 Hz, 1H), 1.63 (td, J = 6.4, 13.2 | |
| | | Hz, 1H), 1.58-1.46 (m, 1H), 1.40-1.28 (m, 1H), 0.86 (t, | |
| | | J = 6.4 Hz, 6H) | |
| 55 | racemate | ¹H NMR (400 MHz, DMSO-d₆) δ = 14.60-13.86 (m, 1H), | LCMS (ESI, |
| | | 8.57-8.53 (m, 3H), 8.43 (d, J = 2.0 Hz, 1H), 8.17 | M + 1): |
| | | (d, J = 3.6 Hz, 1H), 8.04 (d, J = 8.0 Hz, 1H), 7.91 (br d, | m/z = 382.2. |
| | | J = 8.0 Hz, 1H), 7.77-7.59 (m, 1H), 6.91 (d, J = 3.6 Hz, | |
| | | 1H), 3.38 (t, J = 6.4 Hz, 3H), 3.16-3.04 (m, 1H), 3.03- | |
| | | 2.91 (m, 1H), 1.93-1.79 (m, 1H), 1.74-1.58 (m, 1H) | |
| 56B | 2nd peak | ¹H NMR (400 MHz, DMSO-d₆) δ = 10.11 (s, 1H), 8.57 (d, | LCMS [ESI, |
| | | J = 2.0 Hz, 1H), 8.42 (d, J = 2.0 Hz, 1H), 8.05 (d, J = | M + 1]: |
| | | 3.6 Hz, 1H), 7.90 (s, 1H), 7.40 (br dd, J = 10.4, 19.6 Hz, | 404.1. |
| | | 2H), 6.90 (d, J = 3.6 Hz, 1H), 4.17 (q, J = 7.2 Hz, 2H), | |
| | | 3.11-2.98 (m, 1H), 2.97-2.85 (m, 1H), 1.75-1.52 (m, | |
| | | 1H), 1.51-1.33 (m, 3H), 1.27 (br t, J = 7.2 Hz, 3H), 0.87 | |
| | | (br t, J = 7.2 Hz, 3H) | |
| 56A | 1st peak | ¹H NMR (400 MHz, DMSO-d₆) δ = 10.11 (s, 1H), 8.57 (d, | LCMS [ESI, |
| | | J = 2.0 Hz, 1H), 8.42 (d, J = 2.0 Hz, 1H), 8.05 (d, J = | M + 1]: 404.1 |
| | | 3.6 Hz, 1H), 7.90 (s, 1H), 7.51-7.30 (m, 2H), 6.90 (d, | |
| | | J = 3.6 Hz, 1H), 4.17 (q, J = 7.2 Hz, 2H), 3.15-2.99 (m, | |
| | | 1H), 2.98-2.85 (m, 1H), 1.72-1.50 (m, 1H), 1.49-1.32 | |
| | | (m, 3H), 1.27 (br t, J = 7.2 Hz, 3H), 0.87 (br t, J = 7.2 Hz, | |
| | | 3H | |
| 57 | racemic | ¹H NMR (400 MHz, DMSO-d6) δ = 14.26 (br d, J = 4.0 | LCMS (ESI, |
| | | Hz, 1H), 8.64-8.47 (m, 3H), 8.44 (d, J = 2.0 Hz, 1H), | M + 1): |
| | | 8.17 (d, J = 3.6 Hz, 1H), 8.04 (d, J = 8.0 Hz, 1H), 7.91 | m/z = 380.1 |
| | | (br d, J = 7.2 Hz, 1H), 7.74-7.64 (m, 1H), 6.92 (d, J = 3.6 | |
| | | Hz, 1H), 3.07 (ddd, J = 5.2, 10.8, 13.2 Hz, 1H), 2.93 | |
| | | (ddd, J= 5.2, 10.8, 13.2 Hz, 1H), 1.63 (td, J = 6.4, 13.2 | |
| | | Hz, 1H), 1.58-1.46 (m, 1H), 1.40-1.28 (m, 1H), 0.86 (t, | |
| | | J = 6.4 Hz, 6H) | |
| 59B | 2nd peak | ¹H NMR (400 MHz, DMSO-d₆) δ = 14.20 (br d, J = 1.6 | LCMS [ESI, |
| | | Hz, 1H), 8.89-8.61 (m, 1H), 8.58 (d, J = 2.0 Hz, 1H), | M + 1]: |
| | | 8.54 (br s, 1H), 8.43 (d, J = 2.0 Hz, 1H), 8.17 (d, J = 3.6 | 378.2. |
| | | Hz, 1H), 8.04 (br d, J = 7.6 Hz, 1H), 7.96-7.77 (m, 1H), | |
| | | 7.68 (br s, 1H), 6.91 (d, J = 3.6 Hz, 1H), 3.52-3.37 (m, | |
| | | 1H), 2.01-1.86 (m, 1H), 1.85-1.67 (m, 2H), 1.66-1.44 | |
| | | (m, 5H) | |
| 59A | 1st peak | ¹H NMR (400 MHz, DMSO-d₆) δ = 14.67-13.84 (m, 1H), | LCMS [ESI, |
| | | 8.57 (d, J = 2.0 Hz, 1H), 8.55 (s, 2H), 8.46-8.34 | M + 1]: |
| | | (m, 1H), 8.16 (d, J = 3.6 Hz, 1H), 8.04 (d, J = 8.0 Hz, | 378.2. |
| | | 1H), 7.91 (br d, J = 8.4 Hz, 1H), 7.69 (t, J = 8.0 Hz, 1H), | |
| | | 6.91 (d, J = 3.6 Hz, 1H), 3.46-3.38 (m, 1H), 1.93 (br d, | |
| | | J = 6.8 Hz, 1H), 1.76 (td, J = 6.8, 18.4 Hz, 2H), | |
| | | 1.65-1.46 (m, 5H) | |
| 60 | | ¹H NMR (400 MHz, DMSO-d₆) δ = 14.67-13.82 (m, 1H), | LCMS (ESI, |
| | | 8.77 (d, J = 2.0 Hz, 1H), 8.65 (d, J = 2.4 Hz, 1H), | M + 1): |
| | | 8.54 (br s, 1H), 8.24 (d, J = 4.0 Hz, 1H), 8.07 (d, J = 8.0 | m/z = 412.2. |
| | | Hz, 1H), 7.90 (br d, J = 7.6 Hz, 1H), 7.76-7.62 (m, 1H), | |
| | | 7.00 (d, J = 3.6 Hz, 1H), 4.04-3.69 (m, 1H), 1.98-1.77 | |
| | | (m, 4H), 1.69-1.50 (m, 4H) | |
| 61 | | ¹H NMR (400 MHz, DMSO-d₆) δ = 14.67-13.82 (m, 1H), | LCMS (ESI, |
| | | 8.77 (d, J = 2.0 Hz, 1H), 8.65 (d, J = 2.4 Hz, 1H), | M + 1): |
| | | 8.54 (br s, 1H), 8.50 (s, 1H), 8.24 (d, J = 4.0 Hz, 1H), | m/z = 394.2. |
| | | 8.07 (d, J = 8.0 Hz, 1H), 7.90 (br d, J = 7.6 Hz, 1H), | |
| | | 7.76-7.62 (m, 1H), 7.00 (d, J = 3.6 Hz, 1H), 4.04-3.69 | |
| | | (m, 1H), 1.98-1.77 (m, 4H), 1.69-1.50 (m, 4H) | |
| 62 | | ¹H NMR (400 MHz, DMSO-d₆) δ = 14.09 (br s, 1H), 8.77 | LCMS [ESI, |
| | | (d, J = 2.0 Hz, 1H), 8.62 (d, J = 2.0 Hz, 1H), 8.57-8.29 | M + 1]: 394.1 |
| | | (m, 1H), 8.22 (d, J = 8.4 Hz, 2H), 8.18 (d, J = 3.6 Hz, | |
| | | 1H), 8.04 (br d, J = 7.6 Hz, 2H), 6.99 (d, J = 3.6 Hz, 1H), | |
| | | 3.91-3.76 (m, 1H), 2.06-1.81 (m, 4H), 1.72-1.58 (m, | |
| | | 4H) | |
| 63 | | ¹H NMR (400 MHz, DMSO-d₆) δ = 14.51-13.77 (m, 1H), | LCMS (ESI, |
| | | 8.73 (d, J = 2.0 Hz, 1H), 8.61 (d, J = 2.4 Hz, 1H), | M + 1): |
| | | 8.58-8.40 (m, 1H), 8.27 (d, J = 3.6 Hz, 1H), 8.22 (d, J = | m/z = 444.2. |
| | | 8.8 Hz, 2H), 8.04 (br d, J = 8.4 Hz, 2H), 7.02 (d, J = 3.6 | |
| | | Hz, 1H), 3.54 (br s, 1H), 2.17-1.99 (m, 4H), 1.99-1.78 | |
| | | (m, 2H), 1.73-1.59 (m, 2H). | |
| 64 | | ¹H NMR (400 MHz, DMSO-d₆) δ = 14.53-13.72 (m, | LCMS (ESI, |
| | | 1H), 8.54 (d, J = 2.0 Hz, 1H), 8.53-8.41 (m, 1H), 8.39 | M + 1): |
| | | (d, J = 2.0 Hz, 1H), 8.24-8.16 (m, 3H), 8.06 (br d, J = | m/z = 394.2. |
| | | 8.4 Hz, 2H), 6.92 (d, J = 3.6 Hz, 1H), 4.08-3.72 (m, 2H), | |
| | | 3.30-3.25 (m, 2H), 3.19-3.05 (m, 1H), 1.73-1.52 (m, | |
| | | 3H), 1.46 (br dd, J = 2.4, 13.6 Hz, 1H) | |
| 65 | | ¹H NMR (400 MHz, DMSO-d₆) δ = 10.17 (s, 1H), 8.80-8.60 | LCMS (ESI, |
| | | (m, 4H), 8.51 (t, J = 2.0 Hz, 1H), 8.22 (d, J = 4.0 Hz, | M + 1): |
| | | 1H), 7.03 (d, J = 3.6 Hz, 1H), 4.18 (d, J = 7.2 Hz, 2H), | m/z = 465.2. |
| | | 3.55 (br s, 1H), 2.18-2.00 (m, 4H), 1.97-1.78 (m, 2H), | |
| | | 1.60 (br d, J = 11.2 Hz, 2H), 1.27 (t, J = 7.2 Hz, 3H) | |
| 66 | | ¹H NMR (400 MHz, DMSO-d₆) δ = 10.13 (s, 1H), 8.70 | LCMS (ESI, |
| | | (d, J = 2.0 Hz, 1H), 8.63 (s, 1H), 8.56 (d, J = 2.0 Hz, 1H), | M + 1): |
| | | 8.54 (br s, 1H), 8.43 (d, J = 2.0 Hz, 1H), 8.13 (d, J = 3.6 | m/z = 399.1. |
| | | Hz, 1H), 6.92 (d, J = 4.0 Hz, 1H), 4.18 (q, J = 7.2 Hz, | |
| | | 2H), 3.47-3.37 (m, 1H), 1.92 (br d, J = 6.8 Hz, 1H), | |
| | | 1.86-1.69 (m, 2H), 1.64-1.47 (m, 5H), 1.27 (t, J = | |
| | | 7.2 Hz, 3H). | |
| 67 | | ¹H NMR (400 MHz, DMSO-d₆) δ = 10.13 (s, 1H), 8.70 (d, | LCMS (ESI, |
| | | J = 2.4 Hz, 1H), 8.65-8.59 (m, 2H), 8.55 (s, 1H), | M + 1): |
| | | 8.46 (d, J = 2.0 Hz, 1H), 8.14 (d, J = 3.6 Hz, 1H), 6.93 (d, | m/z = 371.1. |
| | | J = 3.6 Hz, 1H), 4.18 (d, J = 7.2 Hz, 2H), 2.63-2.57 (m, | |
| | | 1H), 1.27 (t, J = 7.2 Hz, 3H), 1.10 (br dd, J = 4.0, 6.8 Hz, | |
| | | 1H), 0.99-0.87 (m, 3H). | |
| 68 | | ¹H NMR (400 MHz, DMSO-d₆) δ = 14.56-13.96 (m, 1H), | LCMS (ESI, |
| | | 8.63 (d, J = 2.0 Hz, 1H), 8.45 (d, J = 2.0 Hz, 2H), | M + 1) = |
| | | 8.23-8.17 (m, 3H), 8.06 (br d, J = 8.8 Hz, 2H), 6.92 (d, | 350.0 |
| | | J = 3.6 Hz, 1H), 2.63-2.56 (m, 1H), 1.16-1.07 (m, 1H), | |
| | | 1.00-0.85 (m, 3H). | |
| 69 | | ¹H NMR (400 MHz, DMSO-d₆) δ = 9.33 (d, J = 1.6 Hz, | LCMS [ESI, |
| | | 1H), 8.61 (d, J = 2.0 Hz, 1H), 8.45 (d, J = 2.0 Hz, 1H), | M + 1]: 409.0 |
| | | 8.13 (d, J = 3.6 Hz, 1H), 8.04 (dd, J = 2.0, 9.2 Hz, 1H), | |
| | | 7.66 (d, J = 9.2 Hz, 1H), 6.91 (d, J = 3.6 Hz, 1H), 3.78- | |
| | | 3.68 (m, 4H), 3.53-3.46 (m, 4H), 2.62-2.54 (m, 1H), | |
| | | 1.14-1.07 (m, 1H), 0.98-0.92 (m, 2H), 0.92-0.84 (m, | |
| | | 1H). | |
| 70 | | ¹H NMR (400 MHz, DMSO-d₆) δ = 10.13 (br s, 1H), 8.70 | LCMS (ESI, |
| | | (s, 1H), 8.63 (s, 1H), 8.57-8.53 (m, 2H), 8.44 (t, J = 1.6 | M + 1): |
| | | Hz, 1H), 8.20-8.08 (m, 1H), 6.92 (dd, J = 1.2, 3.0 Hz, | m/z = 399.1. |
| | | 1H), 4.18 (d, J = 6.8 Hz, 2H), 3.20-3.06 (m, 2H), 2.67- | |
| | | 2.63 (m, 1H), 2.08-1.99 (m, 1H), 1.95-1.76 (m, 4H), | |
| | | 1.74-1.65 (m, 1H), 1.31-1.23 (m, 3H) | |
| 71 | | ¹H NMR (400 MHz, DMSO-d₆) δ = 9.33 (d, J = 1.6 Hz, | LCMS [ESI, |
| | | 1H), 8.56 (d, J = 2.0 Hz, 1H), 8.43 (d, J = 2.0 Hz, 1H), | M + 1]: |
| | | 8.13 (d, J = 3.6 Hz, 1H), 8.04 (dd, J = 2.0, 9.2 Hz, 1H), | 425.1. |
| | | 7.67 (d, J = 9.6 Hz, 1H), 6.90 (d, J = 3.6 Hz, 1H), 3.77- | |
| | | 3.68 (m, 4H), 3.56-3.45 (m, 4H), 3.09-2.99 (m, 1H), | |
| | | 2.98-2.86 (m, 1H), 1.76-1.42 (m, 2H), 1.42-1.29 (m, | |
| | | 2H), 0.90-0.83 (m, 3H). | |
| 72 | | ¹H NMR (400 MHz, DMSO-d₆) δ = 14.30-14.21 (m, 1H), 8.58 | LCMS (ESI, |
| | | (d, J = 2.0 Hz, 1H), 8.48 (br s, 1H), 8.43 (d, J = | M + 1): |
| | | 2.0 Hz, 1H), 8.26-8.14 (m, 3H), 8.06 (br d, J = 8.4 Hz, | m/z = 378.1. |
| | | 2H), 6.91 (d, J = 3.6 Hz, 1H), 3.49-3.37 (m, 1H), 1.93 | |
| | | (br d, J = 6.4 Hz, 1H), 1.86-1.68 (m, 2H), 1.64-1.48 | |
| | | (m, 5H). | |
| 73 | | ¹H NMR (400 MHz, DMSO-d₆) δ = 10.15 (s, 1H), 8.70 | LCMS (ESI, |
| | | (d, J = 2.4 Hz, 1H), 8.63 (d, J = 2.0 Hz, 1H), 8.56 (s, 1H), | M + 1): |
| | | 8.54 (d, J = 2.0 Hz, 1H), 8.41 (d, J = 2.0 Hz, 1H), 8.15 (d, | m/z = 449.1. |
| | | J = 3.6 Hz, 1H), 6.94 (d, J = 3.6 Hz, 1H), 4.18 (q, J = 7.2 | |
| | | Hz, 2H), 3.04 (br t, J = 11.6 Hz, 1H), 2.19-2.05 (m, 2H), | |
| | | 2.04-1.89 (m, 2H), 1.87-1.67 (m, 2H), 1.67-1.53 (m, | |
| | | 2H), 1.27 (t, J = 7.2 Hz, 3H). | |
| 74 | | ¹H NMR (400 MHz, DMSO-d₆) δ = 14.71-13.65 (m, 1H), | LCMS (ESI, |
| | | 8.59 (d, J = 2.0 Hz, 2H), 8.45 (d, J = 2.0 Hz, 1H), | M + 1): |
| | | 8.23-8.16 (m, 3H), 8.07 (br s, 2H), 6.91 (d, J = 3.6 Hz, | m/z = 364.0. |
| | | 1H), 3.04-2.89 (m, 2H), 0.97-0.87 (m, 1H), 0.54 (br dd, | |
| | | J = 1.6, 8.0 Hz, 2H), 0.29 (br t, J = 6.0 Hz, 2H). | |
| 75 | | ¹H NMR (400 MHz, DMSO-d₆) δ = 14.24 (br s, 1H), 8.55 | LCMS (ESI, |
| | | (d, J = 2.0 Hz, 2H), 8.40 (d, J = 2.0 Hz, 1H), 8.25-8.17 | M + 1): |
| | | (m, 3H), 8.06 (br d, J = 8.4 Hz, 2H), 6.93 (d, J = 3.6 Hz, | m/z = 428.0. |
| | | 1H), 3.09-2.99 (m, 1H), 2.09 (br d, J = 12.8 Hz, 2H), | |
| | | 2.02-1.70 (m, 4H), 1.68-1.52 (m, 2H). | |

### BIOLOGICAL EXAMPLES

### Example B-1. hPGDH Inhibitor Screening Biochemical Assay

A hydroxyprostaglandin dehydrogenase inhibition screening biochemical assay can be performed to assess the synthesized inhibitors provided herein. Provided herein is an exemplary biochemical assay for hPGDH inhibitor screening.

The in vitro biochemical assay can be performed in white, 384 plates in total 20 μl reaction volume consisting of 10 nM of 15-PGDH/HPGD (R&D System #5660-DH), 15 M Prostaglandin E2 (Sigma, Cat #P5640-10MG) and 0.25 mM β-Nicotinamide adenine dinucleotide sodium salt (Sigma, Cat #N0632-5G) made in reaction buffer (50 mM Tris-HCl, pH 7.5, 0.01% Tween 20) at 10-point dose response curve for test/tool compounds. Briefly, 5 μl (4×) of compounds solution and 5 μl (final concentration, 10 nM) of enzyme solution is added to white 384 well plates and incubated for 10 mins at 37° C. 5 μl (4×) of Prostaglandin E2 and 5 μl (4×) of β-Nicotinamide adenine dinucleotide sodium salt is added to the wells and incubated for 10 mins at room temperature. Fluorescence is recorded at ex/em=340 nm/485 nm. The percentage (%) inhibition of enzyme activity was determined relative to positive control (1% DMSO) and IC50 was calculated using GraphPad prism software (four parameter-variable slope equation). Exemplary data are shown in Table 5.

TABLE 5
hPGDH inhibition potency.

| Comp. No. | PGDH IC₅₀ (nM) | A549 EC₅₀ (nM) |
|---|---|---|
| 1B | A | B |
| 1A | A | A |
| 2B | A | B |
| 2A | B | — |
| 3B | A | C |
| 4B | A | C |
| 3A | C | C |
| 4A | C | — |
| 5C | A | B |
| 5A | B | C |
| 5D | A | — |
| 5B | C | — |
| 6 | B | C |
| 7B | A | A |
| 7A | B | — |
| 8B | B | — |
| 8A | C | — |
| 9B | A | A |
| 9A | B | — |
| 10B | A | B |
| 10A | B | C |
| 8D | A | A |
| 8C | A | A |
| 11D | A | C |
| 11C | B | C |
| 11B | B | C |
| 11A | A | C |
| 12B | A | C |
| 12A | A | A |
| 13 | A | C |
| 14B | B | — |
| 14A | A | A |
| 16B | A | C |
| 16A | A | A |
| 17B | A | A |
| 17A | B | C |
| 18B | A | A |
| 18A | A | C |
| 19B | A | C |
| 19A | A | A |
| 20B | A | A |
| 20A | B | C |
| 21B | A | A |
| 21A | B | C |
| 15 | A | B |
| 22B | A | B |
| 22A | B | C |
| 23B | A | B |
| 23A | C | — |
| 24B | A | B |
| 24A | B | C |
| 25 | A | C |
| 26B | B | B |
| 26A | A | A |
| 27B | C | — |
| 27A | A | C |
| 28 | A | B |
| 29 | A | B |
| 30 | B | C |
| 31 | A | A |
| 32 | A | C |
| 33 | A | B |
| 34 | A | B |
| 35 | B | C |
| 36B | A | B |
| 36A | B | C |
| 37 | C | — |
| 38 | B | C |
| 39 | A | B |
| 40 | A | B |
| 41 | A | B |
| 42 | A | — |
| 43 | A | B |
| 44 | A | B |
| 45 | A | B |
| 46 | A | B |
| 47 | A | B |
| 48B | A | C |
| 48A | A | A |
| 49B | A | A |
| 49A | A | C |
| 50B | A | B |
| 50A | A | A |
| 51B | A | A |
| 51A | A | C |
| 52B | A | A |
| 52A | A | B |
| 53B | A | A |
| 53A | A | B |
| 54B | A | A |
| 54A | A | B |
| 55 | A | B |
| 56B | B | C |
| 56A | A | B |
| 57 | A | A |
| 58 | A | B |
| 59B | A | A |
| 59A | A | B |
| 60 | A | A |
| 61 | A | B |
| 62 | A | B |
| 63 | A | B |
| 64 | A | B |
| 65 | B | B |
| 66 | A | A |
| 67 | A | — |
| 68 | A | C |
| 69 | A | C |
| 70 | A | C |
| 71 | A | B |
| 72 | A | A |
| 73 | A | A |
| 74 | A | C |
| 75 | A | A |
| 88A | — | A |
| 88B | — | B |
| 90A | A | B |
| 90B | B | — |
| 91A | A | — |
| 91B | B | — |
| 92A | B | — |
| 92B | A | B |
| 93A | A | B |
| 93B | C | — |
| 94A | A | B |
| 94B | B | — |
| 95A | A | — |
| 95B | B | — |
| 96A | A | C |
| 96B | B | C |
| 97A | A | B |
| 97B | B | C |
| 98A | A | C |
| 98B | A | C |
| 99A | A | B |
| 99B | B | C |
| 100A | B | C |
| 100B | C | C |
| 101A | C | C |
| 101B | A | C |
| 102A | A | B |
| 102B | B | C |
| 103A | B | — |
| 103B | A | A |
| 104A | A | B |
| 104B | C | C |
| 105A | B | C |
| 105B | C | C |
| 105C | A | C |
| 105D | A | C |
| 107A | B | C |
| 107B | C | C |
| 108A | C | C |
| 108B | A | C |
| 109C | C | C |
| 109D | C | C |
| 109A | B | C |
| 109B | B | C |
| 110A | A | A |
| 110B | A | C |
| 111A | A | C |
| 111B | A | C |
| 112A | A | B |
| 112B | B | C |
| 113A | A | B |
| 113B | B | C |
| 114A | A | C |
| 114B | C | C |
| 115A | A | C |
| 115B | C | C |
| 116A | A | C |
| 116B | C | C |
| 117A | A | — |
| 117B | B | — |
| 118A | A | A |
| 118B | B | C |
| 119A | A | B |
| 119B | C | C |
| 120A | A | A |
| 120B | A | C |
| 120C | A | B |
| 120D | A | A |
| 121A | A | C |
| 121B | B | C |
| 122A | A | B |
| 122B | B | C |
| 123A | A | C |
| 123B | A | A |
| 123C | A | C |
| 123D | A | A |
| 124A | B | C |
| 124B | A | A |
| 125A | A | C |
| 125B | A | C |
| 126A | C | — |
| 126B | A | — |
| 127B | A | — |
| 127A | B | — |

A < 0.1 μM; 0.1 μM ≤ B < 1 μM; 1 μM ≤ C

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of Formula (I), or a pharmaceutically acceptable salt thereof: 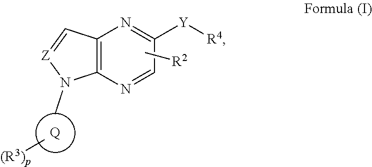
wherein,
ring Q is phenyl or 5- to 10-membered heteroaryl;
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
each R³ is independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
R⁴ is substituted or unsubstituted C₁-C₈ alkyl, substituted or unsubstituted C₂-C₈ alkenyl, substituted or unsubstituted C₁-C₈ aminoalkyl, substituted or unsubstituted C₁-C₈ heteroalkyl, substituted or unsubstituted C₁-C₈ haloalkyl, substituted or unsubstituted C₁-C₈ hydroxyalkyl, substituted or unsubstituted C₃-C₈ cycloalkyl, or substituted or unsubstituted C₃-C₈ heterocycloalkyl, each of which is substituted with one or more R⁶;
or R⁴ is 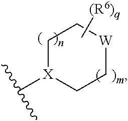
wherein
X is CH or N;
W is CR⁶R⁶, —NR^{6a}—, or —O—;
each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹⁰, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
R^{6a} is H or C₁-C₃ alkyl;
m and n are each independently 0, 1, or 2;
q is 0, 1, 2, 3, 4, 5, 6, 7, 8, 9, or 10;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R;
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R;
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R;
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃; and
p is 1, 2, 3, or 4.

2. The compound of claim 1, or a pharmaceutically acceptable salt thereof, wherein ring Q is a 6-membered monocyclic heteroaryl comprising 1, 2, or 3 N atoms.

3. The compound of claim 1, or a pharmaceutically acceptable salt thereof, wherein ring Q is a phenyl, pyrimidine, or pyridine.

4. The compound of claim 1 or 3, or a pharmaceutically acceptable salt thereof, wherein when ring Q is phenyl at least one R³ is not H.

5. The compound of claim 1, or a pharmaceutically acceptable salt thereof, wherein 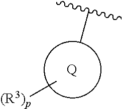
is 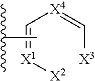
wherein,
X¹, X², X³ and X⁴ are each independently N or CR³;
each R³ is independently selected from H, halogen, —CN, —NO₂, —NR⁸R⁹, —OR¹⁰, —C(O)R¹, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a}; and
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

6. The compound of claim 5, or a pharmaceutically acceptable salt thereof, wherein X¹, X², X³ and X⁴ are each CR³.

7. The compound of claim 5, or a pharmaceutically acceptable salt thereof, wherein X¹ is N; and X², X³, and X⁴ are each CR³.

8. The compound of any one of claims 5-7 or a pharmaceutically acceptable salt thereof, wherein each R³ is independently selected from H, halogen, —NR⁸R⁹, —OR¹⁰, —C(O)R¹⁰, —C(O)OR¹⁰, —C(O)NR⁸R⁹, —NR¹²C(O)R¹⁰, —NR¹²C(O)OR¹⁰, —NR¹²C(O)NR⁸R⁹, substituted or unsubstituted C₁-C₆ alkyl, substituted or unsubstituted C₁-C₆ haloalkyl, substituted or unsubstituted C₃-C₆ heterocycloalkyl, substituted or unsubstituted phenyl, and substituted or unsubstituted 5- to 10-membered heteroaryl.

9. The compound of claim 1, wherein the compound has the structure of Formula (II), or a pharmaceutically acceptable salt thereof: 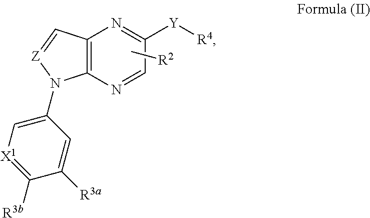
wherein,
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
R⁴ is substituted or unsubstituted C₁-C₈ alkyl, substituted or unsubstituted C₂-C₈ alkenyl, substituted or unsubstituted C₁-C₈ aminoalkyl, substituted or unsubstituted C₁-C₈ heteroalkyl, substituted or unsubstituted C₁-C₈ haloalkyl, substituted or unsubstituted C₁-C₈ hydroxyalkyl, substituted or unsubstituted C₃-C₈ cycloalkyl, or substituted or unsubstituted C₃-C₈ heterocycloalkyl, each of which is substituted with one or more R⁶;
or R⁴ is 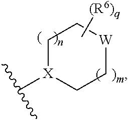
wherein
X is CH or N;
W is CR⁶R⁶, —NR^{6a}—, or —O—;
each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
R^{6a} is H or C₁-C₃ alkyl;
m and n are each independently 0, 1, or 2;
q is 0, 1, 2, 3, 4, 5, 6, 7, 8, 9, or 10;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a}; and
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

10. The compound of any one of claims 1-9, or a pharmaceutically acceptable salt thereof, wherein Y is —S(O)— or —S(O)₂—.

11. The compound of claim 9, wherein the compound of Formula (II) has the structure of Formula (III), or a pharmaceutically acceptable salt thereof:

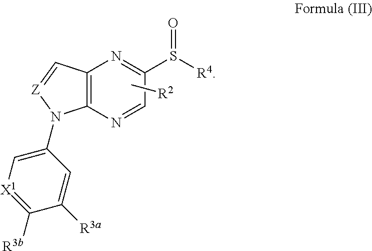

12. The compound of any one of claims 9-11, or a pharmaceutically acceptable salt thereof, wherein R⁴ is substituted or unsubstituted C₁-C₈ alkyl, substituted or unsubstituted C₂-C₈ alkenyl, substituted or unsubstituted C₁-C₈ haloalkyl, substituted or unsubstituted C₁-C₈ heteroalkyl, or substituted or unsubstituted C₁-C₈ hydroxyalkyl.

13. The compound of claim 12, or a pharmaceutically acceptable salt thereof, wherein R⁴ is C₁-C₈ alkyl which is substituted or unsubstituted with one or two halogen or —OR¹⁰.

14. The compound of any one of claims 9-11, or a pharmaceutically acceptable salt thereof, wherein R⁴ is 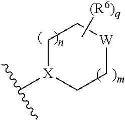
wherein,
X is CH or N;
W is CR⁶R⁶, —NR^{6a}—, or —O—;
each R⁶ is independently H, halogen, CN, —NO₂, —NR⁸R⁹, —OH, —OR¹⁰, —SR⁸, —C(O)R¹⁰, —C(O)OR¹⁰, —C(O)NR⁸R⁹, —SOR¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
R^{6a} is H or C₁-C₃ alkyl;
m and n are each independently 0, 1, or 2; and
q is 0, 1, 2, 3, 4, 5, 6, 7, 8, 9, or 10.

15. The compound of claim 9, wherein the compound of Formula (II) has the structure of Formula (IV), or a pharmaceutically acceptable salt thereof: 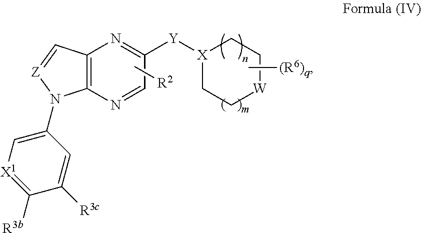
wherein,
Y is —C(O)—, —S(O)—, or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR⁸, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
X is CH or N;
W is CR⁶R⁶, —NR^{6a}—, or —O—;
each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
R^{6a} is H or C₁-C₃ alkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2; and
q is 0, 1, 2, 3, 4, 5, 6, 7, 8, 9, or 10.

16. The compound of claim 15, or a pharmaceutically acceptable salt thereof, wherein Y is —C(O)—.

17. The compound of claim 15, or a pharmaceutically acceptable salt thereof, wherein Y is —S(O)— or —S(O)₂—.

18. The compound of any one of claims 15-17, or a pharmaceutically acceptable salt thereof, wherein X is CH.

19. The compound of claim 15, wherein the compound of Formula (IV) has the structure of Formula (Va), or a pharmaceutically acceptable salt thereof:

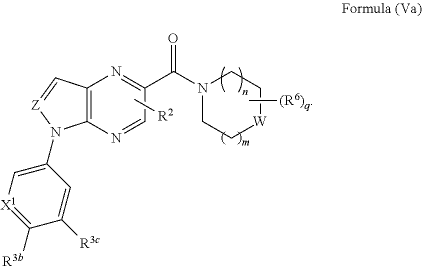

20. The compound of claim 15, wherein the compound of Formula (IV) has the structure of Formula (Vb), or a pharmaceutically acceptable salt thereof:

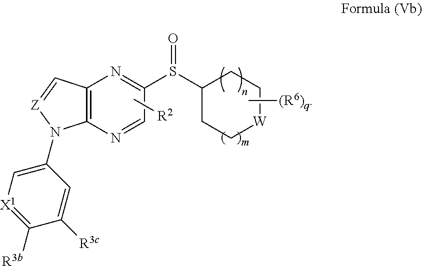

21. The compound of claim 15, wherein the compound of Formula (IV) has the structure of Formula (Vb), or a pharmaceutically acceptable salt thereof:

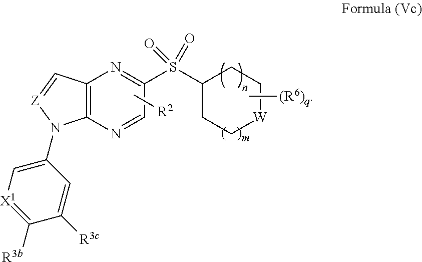

22. The compound of any one of claims 9-21, or a pharmaceutically acceptable salt thereof, wherein X¹ is CR^{3a}.

23. The compound of any one of claims 9-21, or a pharmaceutically acceptable salt thereof, wherein R^{3a} and R^{3a} and R^{3b} are each independently H or halogen; and R^{3c} is selected from —CN, —NO₂, —NR⁸R⁹, —OR¹⁰, —C(O)R¹⁰, —C(O)OR¹⁰, —C(O)NR⁸R⁹, —SOR¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl.

24. The compound of claim 23, or a pharmaceutically acceptable salt thereof, wherein R^{3a} and R^{3b} are both H.

25. The compound of any one of claims 9-21 or a pharmaceutically acceptable salt thereof, wherein X¹ is N.

26. The compound of any one of claims 9-21 or 25, or a pharmaceutically acceptable salt thereof, wherein R^{3b} is H; and R^{3c} is selected from —CN, —NO₂, —NR⁸R⁹, —OR¹⁰, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl.

27. The compound of claim 23 or 26, or a pharmaceutically acceptable salt thereof, wherein R^{3c} is —C(O)OR¹⁰, —C(O)NR⁸R⁹, —NR¹²C(O)R¹⁰, —NR¹²C(O)OR¹⁰, or a substituted or unsubstituted 5 to 6-membered heteroaryl.

28. The compound of any one of claims 14-27, or a pharmaceutically acceptable salt thereof, wherein W is —O—.

29. The compound of any one of claims 14-27, or a pharmaceutically acceptable salt thereof, wherein W is —CR⁶R⁶—.

30. The compound of any one of claims 14-29, or a pharmaceutically acceptable salt thereof, wherein m is 0 and n is 1.

31. The compound of any one of claims 14-29, or a pharmaceutically acceptable salt thereof, wherein m is 1 and n is 1.

32. The compound of any one of claims 14-31, or a pharmaceutically acceptable salt thereof, wherein each R⁶ is independently H, halogen, or C₁-C₃ alkyl; and q is 1 or 2.

33. The compound of any one of claims 1-32, or a pharmaceutically acceptable salt thereof, wherein Z is N.

34. The compound of any one of claims 1-32, or a pharmaceutically acceptable salt thereof, wherein Z is CR¹.

35. The compound of any one of claims 1-32, or a pharmaceutically acceptable salt thereof, wherein Z is CH.

36. The compound of any one of claims 1-35, or a pharmaceutically acceptable salt thereof, wherein R² is H.

37. A compound of Formula (VI), or a pharmaceutically acceptable salt thereof: 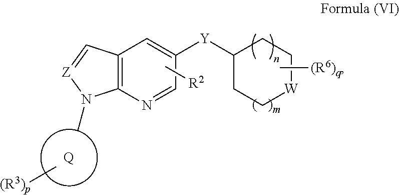
wherein,
Y is —S(O)— or —S(O)₂—;
Z is CR¹ or N;
ring Q is phenyl or 5- to 10-membered heteroaryl;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
each R³ is independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
W is CR⁶R⁶ or —O—;
each R⁶ is independently H, halogen, CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2;
p is 1, 2, 3, or 4; and
q is 0, 1, 2, 3, 4, 5, 6, 7, 8, 9, or 10.

38. The compound of claim 37, or a pharmaceutically acceptable salt thereof, wherein ring Q is a 6-membered monocyclic heteroaryl comprising 1, 2, or 3 N atoms.

39. The compound of claim 37, or a pharmaceutically acceptable salt or solvate thereof, wherein ring Q is a phenyl, pyrimidine, or pyridine.

40. The compound of claim 37 or 39, or a pharmaceutically acceptable salt thereof, wherein when ring Q is phenyl then at least one R³ is not H.

41. The compound of claim 37, or a pharmaceutically acceptable salt thereof, wherein ring Q is 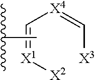
wherein,
X¹, X², X³ and X⁴ are each independently N or CR³;
each R³ is independently selected from H, halogen, —CN, —NO₂, —NR⁸R⁹, —OR¹⁰, —C(O)R¹, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a}; and
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

42. The compound of any one of claims 37-41, or a pharmaceutically acceptable salt thereof, wherein Y is —S(O)—.

43. The compound of any one of claims 37-41, or a pharmaceutically acceptable salt thereof, wherein Y is —S(O)₂—.

44. The compound of claim 37, wherein the compound has the structure of Formula (VIIa) or Formula (VIIb), or a pharmaceutically acceptable salt thereof: 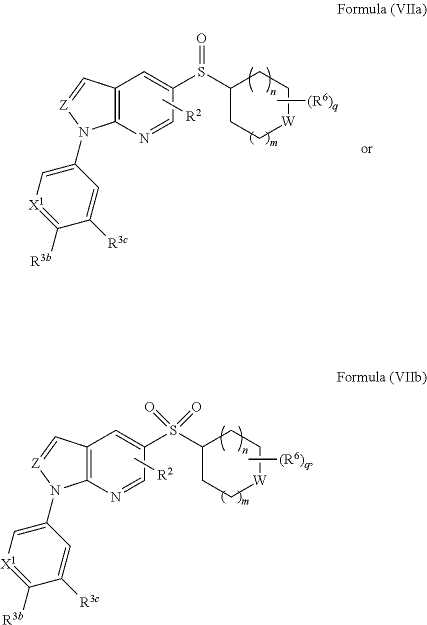
wherein,
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR⁸, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
W is CR⁶R⁶ or —O—;
each R⁶ is independently H, halogen, CN, —NO₂, —NR⁸R⁹, —OR¹⁰, —SR^{B}, —C(O)R¹⁰, —C(O)OR¹⁰, —C(O)NR⁸R⁹, —SOR¹¹, —SO₂R¹¹, —NR⁸C(O)R⁹, C₁-C₆ alkyl, C₁-C₆ haloalkyl, C₁-C₆ hydroxyalkyl, C₃-C₈ cycloalkyl, C₃-C₈ heterocycloalkyl, phenyl, or 5- to 8-membered heteroaryl;
or two R⁶ combine together with the atom(s) to which they are attached to form a C₃-C₆ cycloalkyl or C₃-C₆ heterocycloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m and n are each independently 0, 1, or 2; and
q is 0, 1, 2, 3, 4, 5, 6, 7, 8, 9, or 10.

45. The compound of any one of claims 37-44, or a pharmaceutically acceptable salt thereof, wherein Z is N.

46. The compound of any one of claims 37-44, or a pharmaceutically acceptable salt thereof, wherein Z is CR¹.

47. The compound of any one of claims 37-44, or a pharmaceutically acceptable salt thereof, wherein Z is CH.

48. The compound of claim 44, wherein the compound of Formula (VIIa) or (VIIb) has the structure of Formula (VIIIa) or (VIIIb), or a pharmaceutically acceptable salt thereof:

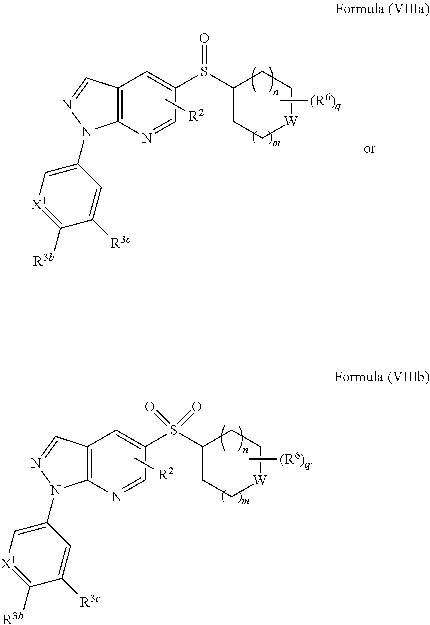

49. The compound of claim 44, wherein the compound of Formula (VIIa) or (VIIb) has the structure of Formula (VIIIc) or (VIIId), or a pharmaceutically acceptable salt thereof:

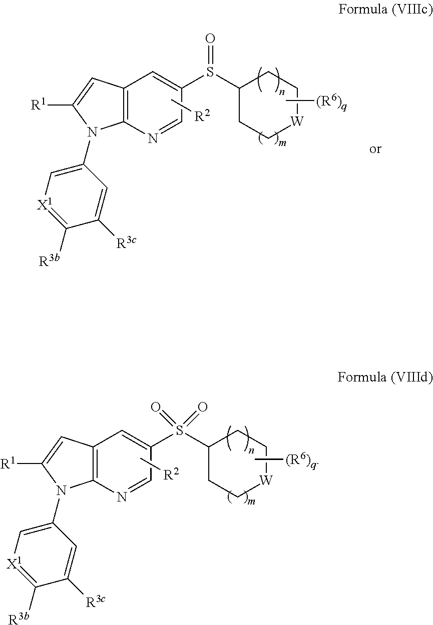

50. The compound of any one of claims 37-49, or a pharmaceutically acceptable salt thereof, wherein W is —O—.

51. The compound of any one of claims 37-49, or a pharmaceutically acceptable salt thereof, wherein W is —CR⁶R⁶—.

52. The compound of any one of claims 37-51, or a pharmaceutically acceptable salt thereof, wherein m is 0 and n is 1.

53. The compound of any one of claims 37-51, or a pharmaceutically acceptable salt thereof, wherein m is 1 and n is 1.

54. The compound of any one of claims 37-53, or a pharmaceutically acceptable salt thereof, wherein each R⁶ is independently H, halogen, —OR¹⁰, or C₁-C₃ alkyl; and q is 1 or 2.

55. A compound of Formula (IX), or a pharmaceutically acceptable salt thereof: 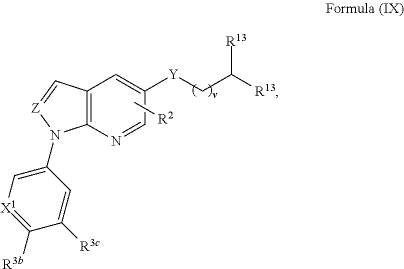
wherein,
Y is —S(O)— or —S(O)₂—;
Z is CR¹ or N;
R¹ is H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
R² is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
X¹ is N or CR^{3a};
R^{3a}, R^{3b}, and R^{3c} are each independently selected from H, halogen, —CN, —NO₂, —NR⁸R⁹, —OR¹⁰, —SR, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl, each of which is substituted with one or more R^{a};
each R¹³ is independently H, halogen, —CN, —NO₂, —NR⁸R⁹, —OR¹⁰, substituted or unsubstituted C₁-C₆ alkyl, substituted or unsubstituted C₁-C₆ haloalkyl, substituted or unsubstituted C₃-C₆ cycloalkyl, or substituted or unsubstituted C₃-C₆ heterocycloalkyl, each of which is substituted with one or more R^{13a};
or two R¹³ combine together with the atoms to which they are attached to form a substituted or unsubstituted C₃-C₆ cycloalkyl or substituted or unsubstituted C₃-C₆ heterocycloalkyl, each of which is substituted with one or more R^{13a}, wherein each R^{13a} is independently halogen, —CN, —NO₂, —NR % R₉, —OR¹⁰, alkyl, or haloalkyl;
each R⁸ and R⁹ is independently selected from H, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ heteroalkyl, substituted or unsubstituted C₁-C₆ haloalkyl, substituted or unsubstituted C₃-C₁₀ cycloalkyl, and substituted or unsubstituted C₃-C₁₀ heterocycloalkyl, each of which is substituted with one or more R^{a};
each R¹⁰ is independently selected from H, substituted or unsubstituted C₁-C₆ alkyl, substituted or unsubstituted C₂-C₆ alkenyl, substituted or unsubstituted C₁-C₆ heteroalkyl, substituted or unsubstituted C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹¹ is independently selected from substituted or unsubstituted C₁-C₆ alkyl, substituted or unsubstituted C₂-C₆ alkenyl, substituted or unsubstituted C₁-C₆ heteroalkyl substituted or unsubstituted, C₁-C₆ haloalkyl, substituted or unsubstituted C₃-C₁₀ cycloalkyl, substituted or unsubstituted C₃-C₁₀ heterocycloalkyl, substituted or unsubstituted C₆-C₁₀ aryl, and substituted or unsubstituted 5- to 10-membered heteroaryl, each of which is substituted with one or more R^{a};
each R¹² is independently selected from H, substituted or unsubstituted C₁-C₆ alkyl, substituted or unsubstituted C₂-C₆ alkenyl, substituted or unsubstituted C₁-C₆ haloalkyl, substituted or unsubstituted C₃-C₈ cycloalkyl, and substituted or unsubstituted C₃-C₁₀ heterocycloalkyl, each of which is substituted with one or more R^{a};
each R^{a} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃; and
v is 0-20.

56. The compound of claim 55, or a pharmaceutically acceptable salt thereof, wherein Z is N.

57. The compound of claim 55, or a pharmaceutically acceptable salt thereof, wherein Z is CR¹.

58. The compound of claim 55, or a pharmaceutically acceptable salt thereof, wherein Z is CH.

59. The compound of claim 55, wherein the compound of Formula (IX) has the structure of Formula (Xa) or (Xb), or a pharmaceutically acceptable salt thereof:

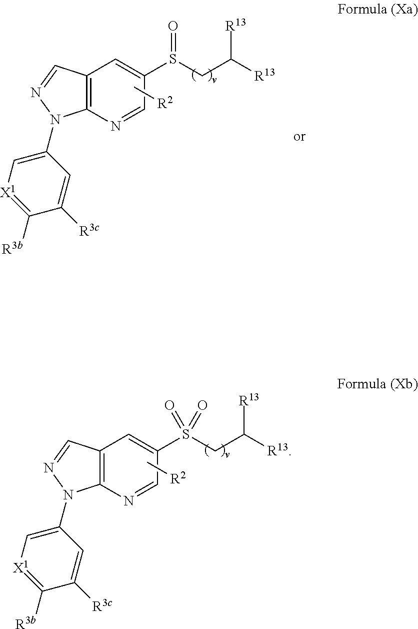

60. The compound of claim 55, wherein the compound of Formula (IX) has the structure of Formula (Xc) or (Xd), or a pharmaceutically acceptable salt thereof:

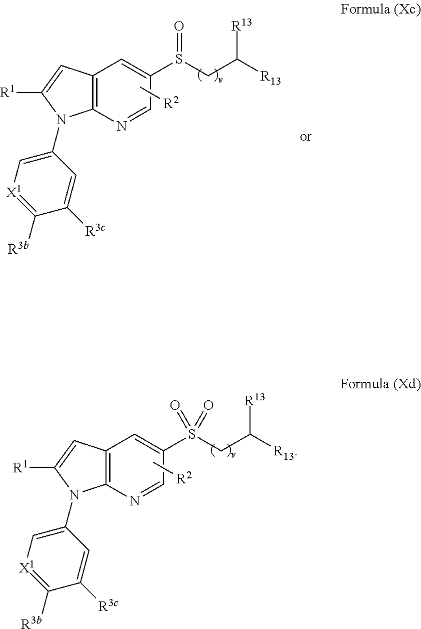

61. The compound of any one of claims 44-60, or a pharmaceutically acceptable salt thereof, wherein X¹ is CR^{3a}.

62. The compound of any one of claims 44-61, or a pharmaceutically acceptable salt thereof, wherein R^{3a} and R^{3b} are independently H or halogen; and R^{3c} is selected from halogen, —CN, —NO₂, —NR⁸R⁹, —OR¹⁰, —C(O)R¹⁰, —C(O)OR¹⁰, —C(O)NR⁸R⁹, —SOR¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl.

63. The compound of claim 62, or a pharmaceutically acceptable salt thereof, wherein R^{3a} and R^{3b} are each H.

64. The compound of any one of claims 44-60, or a pharmaceutically acceptable salt thereof, wherein X¹ is N.

65. The compound of any one of claims 44-60 or 64, or a pharmaceutically acceptable salt thereof, wherein R^{3b} is H; and R^{3c} is selected from halogen, —CN, —NO₂, —NR⁸R⁹, —OR¹⁰, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted phenyl, and substituted or unsubstituted 5- to 10-membered heteroaryl.

66. The compound of claim 62 or 65, or a pharmaceutically acceptable salt thereof, wherein R^{3c} is —C(O)OR¹⁰, —C(O)NR⁸R⁹, —NR¹²C(O)R¹⁰, —NR¹²C(O)OR¹⁰, or a substituted or unsubstituted 5 to 6-membered heteroaryl.

67. The compound of claim 62 or 65, or a pharmaceutically acceptable salt thereof, wherein R^{3c} is —NR¹²C(O)OR¹⁰, or a 5-membered heteroaryl.

68. The compound of any one of claims 55-67, or a pharmaceutically acceptable salt thereof, wherein v is 1-20.

69. The compound of any one of claims 55-67, or a pharmaceutically acceptable salt thereof, wherein v is 1-10.

70. The compound of any one of claims 55-67, or a pharmaceutically acceptable salt thereof, wherein v is 1-5.

71. The compound of any one of claims 37-70, or a pharmaceutically acceptable salt thereof, wherein R² is H.

72. The compound of any one of claims 1-71, wherein the compound is selected from Table 1, or a pharmaceutically acceptable salt thereof.

73. A pharmaceutical composition comprising a compound of any one of claims 1-72, or a pharmaceutically acceptable salt thereof; and a pharmaceutically acceptable excipient.

74. A method of inhibiting 15-hydroxyprostaglandin dehydrogenase (15-PGDH) in a subject in need thereof, comprising administering to the subject a therapeutically effective amount of a compound of any one of claims 1-72, or a pharmaceutically acceptable salt thereof, or a pharmaceutical composition of claim 73.

75. A method of promoting and/or stimulating skin pigmentation, comprising administering one or more of said compositions of any of the preceding claims to a subject in need thereof.

76. A method of inhibiting hair loss, comprising administering one or more of said compositions of any of the preceding claims to a subject in need thereof.

77. A method of preventing and/or treating skin inflammation and/or damage, comprising administering one or more of said compositions of any of the preceding claims to a subject in need thereof.

78. A method of preventing and/or treating vascular insufficiency, comprising administering one or more of said compositions of any of the preceding claims to a subject in need thereof.

79. A method of preventing, treating, minimizing and/or reversing congestive heart failure or cardiomyopathy, comprising administering one or more of said compositions of any of the preceding claims to a subject in need thereof.

80. A method of reducing cardiac ejection fraction, comprising administering one or more of said compositions of any of the preceding claims to a subject in need thereof.

81. A method of preventing and/or treating a gastrointestinal disease, comprising administering one or more of said compositions of any of the preceding claims to a subject in need thereof.

82. A method of preventing and/or treating renal dysfunction, comprising administering one or more of said compositions of any of the preceding claims to a subject in need thereof.

83. A method of stimulation bone resorption and bone formation, comprising administering one or more of said compositions of any of the preceding claims to a subject in need thereof.

84. A method of stimulating tissue regeneration by stimulating, comprising administering one or more of said compositions of any of the preceding claims to a subject in need thereof.

85. A method of modulating cervical ripening, comprising administering one or more of said compositions of any of the preceding claims to a subject in need thereof.

86. A method of promoting neuroprotection and/or stimulating neuronal regeneration, comprising administering one or more of said compositions of any of the preceding claims to a subject in need thereof.

87. A method of treating and/or preventing a neurological disorder, a neuropsychiatric disorder, a neural injury, a neural toxicity disorder, a neuropathic pain, or a neural degenerative disorder, comprising administering one or more of said compositions of any of the preceding claims to a subject in need thereof.

88. A method of treating and/or preventing fibrotic or adhesion disease, disorder or condition, comprising administering one or more of said compositions of any of the preceding claims to a subject in need thereof.

89. A method of reducing and/or preventing scar formation, comprising administering one or more of said compositions of any of the preceding claims to a subject in need thereof.

90. A method of treating and/or preventing muscle disorder, muscle injury and/or muscle atrophy, comprising administering one or more of said compositions of any of the preceding claims to a subject in need thereof.

91. A method of treating and/or preventing fibrosis, comprising administering one or more of said compositions of any of the preceding claims to a subject in need thereof.

92. A method of treating and/or preventing idiopathic pulmonary fibrosis, comprising administering one or more of said compositions of any of the preceding claims to a subject in need thereof.

93. A method of treating and/or preventing kidney fibrosis, comprising administering one or more of said compositions of any of the preceding claims to a subject in need thereof.

94. A method of stimulating muscle regeneration, comprising administering one or more of said compositions of any of the preceding claims to a subject in need thereof.

95. A method of promoting organ fitness, comprising administering one or more of said compositions of any of the preceding claims to a subject in need thereof.

96. A method of promoting wound healing, comprising administering one or more of said compositions of any of the preceding claims to a subject in need thereof.

97. A method of treating acute kidney injury, comprising administering one or more of said compositions of any of the preceding claims to a subject in need thereof.

98. A method of treating sarcopenia, comprising administering one or more of said compositions of any of the preceding claims to a subject in need thereof.

99. A method of treating a neuromuscular disease, comprising administering one or more of said compositions of any of the preceding claims to a subject in need thereof.
